(19) United States
(12) Patent Application Publication
(10) Pub. No.: US 2009/0099140 A1
(43) Pub. Date: Apr. 16, 2009

(54) 20-Alkyl, Gemini Vitamin D3 Compounds and Methods of Use Thereof

(75) Inventors: Pawel Jankowski, Wayne, NJ (US); Milan R. Uskokovic, Upper Montclair, NJ (US); Luciano Adorini, Milan (IT)

Correspondence Address: EDWARDS ANGELL PALMER & DODGE LLP, P.O. BOX 55874, BOSTON, MA, 02205, US

(73) Assignee: BioXell S.p.A., Milan (IT)

(21) Appl. No.: 11/886,826

(22) PCT Filed: Mar. 23, 2006
(86) PCT No.: PCT/US2006/011000

§ 371 Date: Jun. 13, 2008

Related U.S. Application Data
(60) Provisional application No. 60/664,397, filed on Mar. 23, 2005.
(60) Provisional application No. 60/664,367, filed on Mar. 23, 2005.

(51) Int. Cl. A61K 31/593; C07C 401/00; A61P 13/00

(52) U.S. Cl. 514/167; 552/653

(57) ABSTRACT

The invention provides 20-alkyl Gemini vitamin D₃ compounds, methods for using the compounds to treat vitamin D₃ associated states and pharmaceutical compositions containing the compounds.

### RELATED APPLICATIONS

This application claims the benefit of U.S. provisional patent application Ser. No. 60/664,397 filed 23 Mar. 2005 (Attorney Docket No. 49949-63097P1) and U.S. provisional patent application Ser. No. 60/664,367 filed 23 Mar. 2005 (Attorney Docket No. 49949-63097P2). This application is related to international patent application No. PCT/US2006/______, filed on Mar. 23, 2006 (Attorney Docket No. 49949-63097PCT(B), Express Mail Label No. EV 756031935 US). The disclosures of the all three of the aforementioned patent applications are incorporated herein in their entireties by this reference.

### BACKGROUND OF THE INVENTION

The importance of vitamin D (cholecalciferol) in the biological systems of higher animals has been recognized since its discovery by Mellanby in 1920 (Mellanby, E. (1921) Spec. Rep. Ser. Med. Res. Council (GB) SRS 61:4). It was in the interval of 1920-1930 that vitamin D officially became classified as a “vitamin” essential for the normal development of the skeleton and maintenance of calcium and phosphorous homeostasis.

Studies involving the metabolism of vitamin D₃ were initiated with the discovery and chemical characterization of the plasma metabolite, 25-hydroxyvitamin D₃ [25(OH)D₃] (Blunt, J. W. et al. (1968) Biochemistry 6:3317-3322) and the hormonally active form, 1α,25(OH)₂D₃ (Myrtle, J. F. et al. (1970) J. Biol. Chem. 245:1190-1196; Norman, A. W. et al. (1971) Science 173:51-54; Lawson, D. E. M. et al. (1971) Nature 230:228-230; Holick, M. F. (1971) Proc. Natl. Acad. Sci. USA 68:803-804). The formulation of the concept of a vitamin D endocrine system was dependent upon the appreciation of the key role of the kidney in producing 1α,25(OH)₂D₃ in a carefully regulated fashion Fraser, D. R. and Kodicek, E (1970) Nature 288:764-766; Wong, R. G. et al. (1972) J. Clin. Invest. 51:1287-1291), and the discovery of a nuclear receptor for 1α,25(OH)₂D₃ (VD₃R) in the intestine (Haussler, M. R. et al. (1969) Exp. Cell Res. 58:234-242; Tsai, H. C. and Norman, A. W. (1972) J. Biol. Chem. 248:5967-5975).

The operation of the vitamin D endocrine system depends on the following: first, on the presence of cytochrome P450 enzymes in the liver (Bergman, T. and Postlind, H. (1991) Biochem. J. 276:427-432; Ohyama, Y. and Okuda, K. (1991) J. Biol. Chem. 266:8690-8695) and kidney (Henry, H. L. and Norman, A. W. (1974) J. Biol. Chem. 249:7529-7535; Gray, R. W. and Ghazarian, J. G. (1989) Biochem. J 259:561-568), and in a variety of other tissues to effect the conversion of vitamin D₃ into biologically active metabolites such as 1α,25(OH)₂D₃ and 24R,25(OH)₂D₃; second, on the existence of the plasma vitamin D binding protein (DBP) to effect the selective transport and delivery of these hydrophobic molecules to the various tissue components of the vitamin D endocrine system (Van Baelen, H. et al. (1988) Ann NY Acad. Sci. 538:60-68; Cooke, N. E. and Haddad, J. G. (1989) Endocr. Rev. 10:294-307; Bikle, D. D. et al. (1986) J. Clin. Endocrinol. Metab. 63:954-959); and third, upon the existence of stereoselective receptors in a wide variety of target tissues that interact with the agonist 1α,25(OH)₂D₃ to generate the requisite specific biological responses for this secosteroid hormone (Pike, J. W. (1991) Annu. Rev. Nutr. 11:189-216). To date, there is evidence that nuclear receptors for 1α,25(OH)₂D₃ (VD₃R) exist in more than 30 tissues and cancer cell lines (Reichel, H. and Norman, A. W. (1989) Annu. Rev. Med. 40:71-78).

Vitamin D₃ and its hormonally active forms are well-known regulators of calcium and phosphorous homeostasis. These compounds are known to stimulate, at least one of, intestinal absorption of calcium and phosphate, mobilization of bone mineral, and retention of calcium in the kidneys. Furthermore, the discovery of the presence of specific vitamin D receptors in more than 30 tissues has led to the identification of vitamin D₃ as a pluripotent regulator outside its classical role in calcium/bone homeostasis. A paracrine role for 1α,25(OH)₂ D₃ has been suggested by the combined presence of enzymes capable of oxidizing vitamin D₃ into its active forms, e.g., 25-OHD-1α-hydroxylase, and specific receptors in several tissues such as bone, keratinocytes, placenta, and immune cells. Moreover, vitamin D₃ hormone and active metabolites have been found to be capable of regulating cell proliferation and differentiation of both normal and malignant cells (Reichel, H. et al. (1989) Ann. Rev. Med. 40: 71-78).

Given the activities of vitamin D₃ and its metabolites, much attention has focused on the development of synthetic analogs of these compounds. A large number of these analogs involve structural modifications in the A ring, B ring, C/D rings, and, primarily, the side chain (Bouillon, R. et al., Endocrine Reviews 16(2):201-204). Although a vast majority of the vitamin D₃ analogs developed to date involve structural modifications in the side chain, a few studies have reported the biological profile of A-ring diastereomers (Norman, A. W. et al. J. Biol. Chem. 268 (27): 20022-20030). Furthermore, biological esterification of steroids has been studied (Hochberg, R. B., (1998) Endocr Rev. 19(3): 331-348), and esters of vitamin D₃ are known (WO 97/11053).

Moreover, despite much effort in developing synthetic analogs, clinical applications of vitamin D and its structural analogs have been limited by the undesired side effects elicited by these compounds after administration to a subject for known indications/applications of vitamin D compounds. Therefore, structural analogs of vitamin D having improved therapeutic activity and/or reduced undesirable side effects are needed.

### SUMMARY OF THE INVENTION

In one aspect, the invention provides a vitamin D₃ compound having formula I:

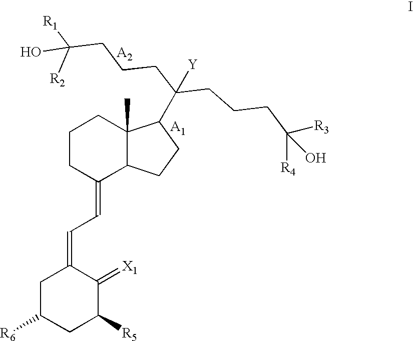

wherein:
A₁ is a single or double bond;
A₂ is a single, a double or a triple bond;
R₁, R₂, R₃ and R₄ are each independently alkyl, deuteroalkyl, hydroxyalkyl, or haloalkyl;
R₅ is halogen, hydroxyl, OC(O)alkyl, OC(O)hydroxyalkyl, or OC(O)haloalkyl;
R₆ is halogen, hydroxyl, OC(O)alkyl, OC(O)hydroxyalkyl, or OC(O)haloalkyl;

### X₁ is H₂ or CH₂;

Y is alkyl;
and pharmaceutically acceptable esters, salts, and prodrugs thereof.

In one aspect, the invention provides a vitamin D₃ compound having formula I-a:

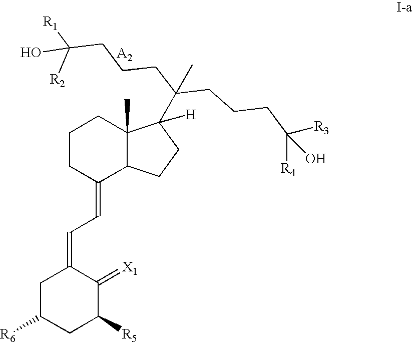

wherein:
A₂ is a single, a double or a triple bond;
R₁, R₂, R₃ and R₄ are each independently alkyl, hydroxyalkyl, or haloalkyl;
R₅ is halogen, hydroxyl, OC(O)alkyl, OC(O)hydroxyalkyl, or OC(O)haloalkyl;
R₆ is hydroxyl, OC(O)alkyl, OC(O)hydroxyalkyl, or OC(O)haloalkyl;

### X₁ is H₂ or CH₂;

and pharmaceutically acceptable esters, salts, and prodrugs thereof.

In certain aspects, the invention provides a compound having formula I-b:

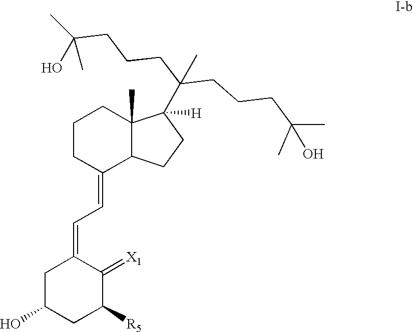

wherein:
R₅ is fluoro or hydroxyl;

### X₁ is H₂ or CH₂;

and pharmaceutically acceptable esters, salts, and prodrugs thereof.

In other aspects, the invention provides a compound having formula I-c:

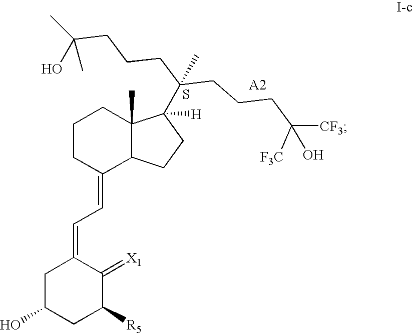

wherein:
A₂ is a single, a double or a triple bond;
R₅ is fluoro or hydroxyl;

### X₁ is H₂ or CH₂;

and pharmaceutically acceptable esters, salts, and prodrugs thereof.

In another aspect, the invention provides a compound having formula I-d:

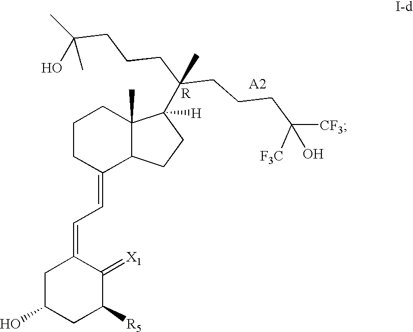

wherein:
A₂ is a single, a double or a triple bond;
R₅ is fluoro or hydroxyl;

### X₁ is H₂ or CH₂;

and pharmaceutically acceptable esters, salts, and prodrugs thereof.

In yet another aspect, the invention provides a compound having formula I-e:

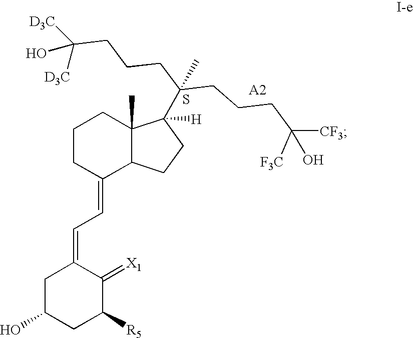

wherein:
A₂ is a single, a double or a triple bond;
R₅ is fluoro or hydroxyl;

### X₁ is H₂ or CH₂;

and pharmaceutically acceptable esters, salts, and prodrugs thereof.

In still another aspect, the invention provides a compound having formula I-f:

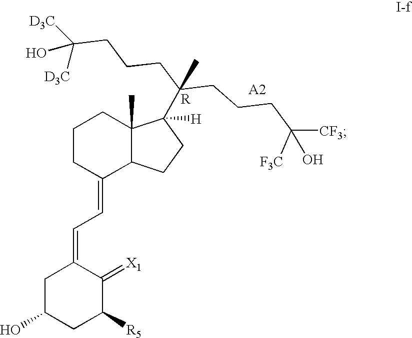

wherein:
A₂ is a single, a double or a triple bond;
R₅ is fluoro or hydroxyl;

### X₁ is H₂ or CH₂;

and pharmaceutically acceptable esters, salts, and prodrugs thereof.

The invention also provides methods for treating a subject for a vitamin D₃ associated state, by administering to the subject an effective amount of a vitamin D₃ compound of the invention or otherwise described herein.

Another aspect of the invention provides a method for treating a subject for a urogenital disorder, comprising administering to the subject an effective amount of a vitamin D₃ compound of the invention or otherwise described herein, such that said subject is treated for the urogential disorder.

Another aspect of the invention provides a method for treating bladder dysfunction in a subject in need thereof by administering an effective amount of a vitamin D₃ compound to treat bladder dysfunction.

In another aspect, the invention also provides a method of ameliorating a deregulation of calcium and phosphate metabolism. The method includes administering to a subject a therapeutically effective amount of a vitamin D₃ compound of the invention or otherwise described herein, so as to ameliorate the deregulation of the calcium and phosphate metabolism.

In a further aspect, the invention provides a method of modulating the expression of an immunoglobulin-like transcript 3 (ILT3) surface molecule in a cell. The method includes contacting the cell with a vitamin D₃ compound of the invention or otherwise described herein, in an amount effective to modulate the expression of an immunoglobulin-like transcript 3 (ILT3) surface molecule in the cell.

In another aspect, the invention provides a method of inducing immunological tolerance in a subject, by administering to the subject a vitamin D₃ compound of the invention or otherwise described herein, in an amount effective to modulate the expression of an ILT3 surface molecule, to thereby induce immunological tolerance in the subject.

In yet another aspect, the invention provides a method of inhibiting transplant rejection in a subject. The method includes administering to the subject a vitamin D₃ compound of the invention or otherwise described herein in an amount effective to modulate the expression of an ILT3 surface molecule.

In yet another aspect, the invention provides a method for modulating immunosuppressive activity by an antigen-presenting cell, by contacting an antigen-presenting cell with a vitamin D₃ compound of the invention or otherwise described herein, in an amount effective to modulate ILT3 surface molecule expression, to thereby modulating immunosuppressive activity by an antigen-presenting cell.

The invention also provides a pharmaceutical composition, comprising an effective amount a vitamin D₃ compound of the invention or otherwise described herein and a pharmaceutically acceptable carrier.

In another embodiment, the invention provides a packaged formulation which includes a pharmaceutical composition comprising a vitamin D₃ compound of the invention or otherwise described herein, and a pharmaceutically-acceptable carrier packaged with instructions for use in the treatment of a vitamin D₃ associated state.

### DETAILED DESCRIPTION OF THE INVENTION

### 1. Definitions

Before a further description of the present invention, and in order that the invention may be more readily understood, certain terms are first defined and collected here for convenience.

The term “administration” or “administering” includes routes of introducing the vitamin D₃ compound(s) to a subject to perform their intended function. Examples of routes of administration which can be used include injection (subcutaneous, intravenous, parenterally, intraperitoneally, intrathecal), oral, inhalation, rectal and transdermal. The pharmaceutical preparations are, of course, given by forms suitable for each administration route. For example, these preparations are administered in tablets or capsule form, by injection, inhalation, eye lotion, ointment, suppository, etc. administration by injection, infusion or inhalation; topical by lotion or ointment; and rectal by suppositories. Oral administration is preferred. The injection can be bolus or can be continuous infusion. Depending on the route of administration, the vitamin D₃ compound can be coated with or disposed in a selected material to protect it from natural conditions which may detrimentally effect its ability to perform its intended function. The vitamin D₃ compound can be administered alone, or in conjunction with either another agent as described above or with a pharmaceutically-acceptable carrier, or both. The vitamin D₃ compound can be administered prior to the administration of the other agent, simultaneously with the agent, or after the administration of the agent. Furthermore, the vitamin D₃ compound can also be administered in a proform which is converted into its active metabolite, or more active metabolite in vivo.

The term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. The term alkyl further includes alkyl groups, which can further include oxygen, nitrogen, sulfur or phosphorous atoms replacing one or more carbons of the hydrocarbon backbone, e.g., oxygen, nitrogen, sulfur or phosphorous atoms. In preferred embodiments, a straight chain or branched chain alkyl has 30 or fewer carbon atoms in its backbone (e.g., C₁-C₃₀ for straight chain, C₃-C₃₀ for branched chain), preferably 26 or fewer, and more preferably 20 or fewer. Likewise, preferred cycloalkyls have from 3-10 carbon atoms in their ring structure, and more preferably have 3, 4, 5, 6 or 7 carbons in the ring structure.

Moreover, the term alkyl as used throughout the specification and claims is intended to include both “unsubstituted alkyls” and “substituted alkyls,” the latter of which refers to alkyl moieties having substituents replacing a hydrogen on one or more carbons of the hydrocarbon backbone. Such substituents can include, for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. It will be understood by those skilled in the art that the moieties substituted on the hydrocarbon chain can themselves be substituted, if appropriate. Cycloalkyls can be further substituted, e.g., with the substituents described above. An “alkylaryl” moiety is an alkyl substituted with an aryl (e.g., phenylmethyl (benzyl)). The term “alkyl” also includes unsaturated aliphatic groups analogous in length and possible substitution to the alkyls described above, but that contain at least one double or triple bond respectively.

Unless the number of carbons is otherwise specified, “lower alkyl” as used herein means an alkyl group, as defined above, but having from one to ten carbons, more preferably from one to six, and most preferably from one to four carbon atoms in its backbone structure, which may be straight or branched-chain. Examples of lower alkyl groups include methyl, ethyl, n-propyl, i-propyl, tert-butyl, hexyl, heptyl, octyl and so forth. In preferred embodiment, the term “lower alkyl” includes a straight chain alkyl having 4 or fewer carbon atoms in its backbone, e.g., C₁-C₄ alkyl.

The terms “alkoxyalkyl,” “polyaminoalkyl” and “thioalkoxyalkyl” refer to alkyl groups, as described above, which further include oxygen, nitrogen or sulfur atoms replacing one or more carbons of the hydrocarbon backbone, e.g., oxygen, nitrogen or sulfur atoms.

The terms “alkenyl” and “alkynyl” refer to unsaturated aliphatic groups analogous in length and possible substitution to the alkyls described above, but that contain at least one double or triple bond, respectively. For example, the invention contemplates cyano and propargyl groups.

The term “antigen” includes a substance which elicits an immune response. The antigens of the invention to which tolerance is induced may or may not be exogenously derived relative to the host. For example, the method of the invention may be used to induce tolerance to an “autoantigen.” An autoantigen is a normal constituent of the body that reacts with an autoantibody. The invention also includes inducing tolerance to an “alloantigen.” Alloantigen refers to an antigen found only in some members of a species, for example the blood group substances. An allograft is a graft to a genetically different member of the same species. Allografts are rejected by virtue of the immunological response of T lymphocytes to histocompatibility antigens. The method of the invention also provides for inducing tolerance to a “xenoantigen.” Xenoantigens are substances that cause an immune reaction due to differences between different species. Thus, a xenograft is a graft from a member of one species to a member of a different species. Xenografts are usually rejected within a few days by antibodies and cytotoxic T lymphocytes to histocompatibility antigens.

The language “antigen-presenting cell” or “APC” includes a cell that is able to present an antigen to, for example, a T helper cell. Antigen-presenting cells include B lymphocytes, accessory cells or non-lymphocytic cells, such as dendritic cells, Langerhans cells, and mononuclear phagocytes that help in the induction of an immune response by presenting antigen to helper T lymphocytes. The antigen-presenting cell of the present invention is preferably of myeloid origin, and includes, but is not limited to, dendritic cells, macrophages, monocytes. APCs of the present invention may be isolated from the bone marrow, blood, thymus, epidermis, liver, fetal liver, or the spleen.

The terms “antineoplastic agent” and “antiproliferative agent” are used interchangeably herein and includes agents that have the functional property of inhibiting the proliferation of a vitamin D₃-responsive cell, e.g., inhibit the development or progression of a neoplasm having such a characteristic, particularly a hematopoietic neoplasm.

The term “aryl” as used herein, refers to the radical of aryl groups, including 5- and 6-membered single-ring aromatic groups that may include from zero to four heteroatoms, for example, benzene, pyrrole, furan, thiophene, imidazole, benzoxazole, benzothiazole, triazole, tetrazole, pyrazole, pyridine, pyrazine, pyridazine and pyrimidine, and the like. Aryl groups also include polycyclic fused aromatic groups such as naphthyl, quinolyl, indolyl, and the like. Those aryl groups having heteroatoms in the ring structure may also be referred to as “aryl heterocycles,” “heteroaryls” or “heteroaromatics.” The aromatic ring can be substituted at one or more ring positions with such substituents as described above, as for example, halogen, hydroxyl, alkoxy, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings which are not aromatic so as to form a polycycle (e.g., tetralin). The language “autoimmune disease” or “autoimmune disorder” refers to the condition where the immune system attacks the host's own tissue(s). In an autoimmune disease, the immune tolerance system of the patient fails to recognize self antigens and, as a consequence of this loss of tolerance, brings the force of the immune system to bear on tissues which express the antigen. Autoimmune disorders include, but are not limited to, type 1 insulin-dependent diabetes mellitus, adult respiratory distress syndrome, inflammatory bowel disease, dermatitis, meningitis, thrombotic thrombocytopenic purpura, Sjogren's syndrome, encephalitis, uveitic, leukocyte adhesion deficiency, rheumatoid arthritis, rheumatic fever, Reiter's syndrome, psoriatic arthritis, progressive systemic sclerosis, primary biliary cirrhosis, pemphigus, pemphigoid, necrotizing vasculitis, myasthenia gravis, multiple sclerosis, lupus erythematosus, polymyositis, sarcoidosis, granulomatosis, vasculitis, pernicious anemia, CNS inflammatory disorder, antigen-antibody complex mediated diseases, autoimmune haemolytic anemia, Hashimoto's thyroiditis, Graves disease, habitual spontaneous abortions, Reynard's syndrome, glomerulonephritis, dermatomyositis, chronic active hepatitis, celiac disease, autoimmune complications of AIDS, atrophic gastritis, ankylosing spondylitis and Addison's disease.

The language “biological activities” of vitamin D₃ includes all activities elicited by vitamin D₃ compounds in a responsive cell. It includes genomic and non-genomic activities elicited by these compounds (Gniadecki R. and Calverley M. J. (1998) Pharmacology & Toxicology 82:173-176; Bouillon, R. et al. (1995) Endocrinology Reviews 16(2):206-207; Norman A. W. et al. (1992) J. Steroid Biochem Mol. Biol. 41:231-240; Baran D. T. et al. (1991) J. Bone Miner Res. 6:1269-1275; Caffrey J. M. and Farach-Carson M. C. (1989) J. Biol. Chem. 264:20265-20274; Nemere I. et al. (1984) Endocrinology 115:1476-1483).

The term “bladder dysfunction” refers to bladder conditions associated with overactivity of the detrusor muscle, for example, clinical BPH or overactive bladder. In the context of the present invention “bladder dysfunction” excludes bladder cancer.

The language “bone metabolism” includes direct or indirect effects in the formation or degeneration of bone structures, e.g., bone formation, bone resorption, etc., which may ultimately affect the concentrations in serum of calcium and phosphate. This term is also intended to include effects of compounds of the invention in bone cells, e.g., osteoclasts and osteoblasts, that may in turn result in bone formation and degeneration.

The language “calcium and phosphate homeostasis” refers to the careful balance of calcium and phosphate concentrations, intracellularly and extracellularly, triggered by fluctuations in the calcium and phosphate concentration in a cell, a tissue, an organ or a system. Fluctuations in calcium levels that result from direct or indirect responses to compounds of the invention are intended to be included by these terms.

The term “cancer” refers to a malignant tumor of potentially unlimited growth that expands locally by invasion and systemically by metastasis.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “chiral” refers to molecules which have the property of non-superimposability of the mirror image partner, while the term “achiral” refers to molecules which are superimposable on their mirror image partner.

The term “diastereomers” refers to stereoisomers with two or more centers of dissymmetry and whose molecules are not mirror images of one another.

The term “deuteroalkyl” refers to alkyl groups in which one or more of the of the hydrogens has been replaced with deuterium.

The term “effective amount” includes an amount effective, at dosages and for periods of time necessary, to achieve the desired result, e.g., sufficient treat a vitamin D₃ associated state or to modulate ILT3 expression in a cell. An effective amount of vitamin D₃ compound may vary according to factors such as the disease state, age, and weight of the subject, and the ability of the vitamin D₃ compound to elicit a desired response in the subject. Dosage regimens may be adjusted to provide the optimum therapeutic response. An effective amount is also one in which any toxic or detrimental effects (e.g., side effects) of the angiogenesis inhibitor compound are outweighed by the therapeutically beneficial effects.

A therapeutically effective amount of vitamin D₃ compound (i.e., an effective dosage) may range from about 0.001 to 30 μg/kg body weight, preferably about 0.01 to 25 μg/kg body weight, more preferably about 0.1 to 20 μg/kg body weight, and even more preferably about 1 to 10 μg/kg, 2 to 9 μg/kg, 3 to 8 μg/kg, 4 to 7 μg/kg, or 5 to 6 μg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a vitamin D₃ compound can include a single treatment or, preferably, can include a series of treatments. In one example, a subject is treated with a vitamin D₃ compound in the range of between about 0.1 to 20 μg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of a vitamin D₃ compound used for treatment may increase or decrease over the course of a particular treatment.

The term “enantiomers” refers to two stereoisomers of a compound which are non-superimposable mirror images of one another. An equimolar mixture of two enantiomers is called a “racemic mixture” or a “racemate.”

The language “Gemini vitamin D₃ compounds” is intended to include vitamin D₃ compounds and analogs thereof having bis C20 side chains. Vitamin D₃ compounds are characterized by an “A” ring (monocycle) which is connected to a “B” ring (bicycle) which is connected to a side chain at carbon C20 of the side chain. The Gemini compounds of the invention have two side chains and are, therefore, conspicuously distinguishable from vitamin D₃ compounds having a single side chain. Candidate A and B rings for the Gemini compounds of the invention are disclosed in U.S. Pat. Nos. 6,559,138, 6,329,538, 6,331,642, 6,452,028, 6,492,353, 6,040,461, 6,030,963, 5,939,408, 5,872,113, 5,840,718, 5,612,328, 5,512,554, 5,451,574, 5,428,029, 5,145,846, and 4,225,525. Examples of Gemini compounds in accordance with the invention are disclosed in U.S. Pat. No. 6,030,962.

The language “genomic” activities or effects of vitamin D₃ is intended to include those activities mediated by the nuclear receptor for 1α,25(OH)₂D₃ (VD₃R), e.g., transcriptional activation of target genes.

The term “halogen” designates —F, —Cl, —Br or —I.

The term “haloalkyl” is intended to include alkyl groups as defined above that are mono-, di- or polysubstituted by halogen, e.g., fluoromethyl and trifluoromethyl. The term “hydroxyl” means —OH.

The term “heteroatom” as used herein means an atom of any element other than carbon or hydrogen. Preferred heteroatoms are nitrogen, oxygen, sulfur and phosphorus.

The term “homeostasis” is art-recognized to mean maintenance of static, or constant, conditions in an internal environment.

The language “hormone secretion” is art-recognized and includes activities of vitamin D₃ compounds that control the transcription and processing responsible for secretion of a given hormone e.g., a parathyroid hormone (PTH) of a vitamin D₃ responsive cell (Bouillon, R. et al. (1995) Endocrine Reviews 16(2):235-237).

The language “hypercalcemia” or “hypercalcemic activity” is intended to have its accepted clinical meaning, namely, increases in calcium serum levels that are manifested in a subject by the following side effects, depression of central and peripheral nervous system, muscular weakness, constipation, abdominal pain, lack of appetite and, depressed relaxation of the heart during diastole. Symptomatic manifestations of hypercalcemia are triggered by a stimulation of at least one of the following activities, intestinal calcium transport, bone calcium metabolism and osteocalcin synthesis (reviewed in Boullion, R. et al. (1995) Endocrinology Reviews 16(2): 200-257).

The terms “hyperproliferative” and “neoplastic” are used interchangeably, and include those cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

The term “interstitial cystitis” (IC) is a chronic inflammatory bladder disease characterized by pelvic pain, urinary urgency and frequency. Unlike other bladder dysfunction conditions, IC is characterized by chronic inflammation of the bladder wall which is responsible for the symptomatology.

An “ILT3-associated disorder” includes a disease, disorder or condition which is associated with an ILT3 molecule. ILT3 associated disorders include disorders in which ILT3 activity is aberrant or in which a non-ILT3 activity that would benefit from modulation of an ILT3 activity is aberrant. In one embodiment, the ILT3-associated disorder is an immune disorder, e.g., an autoimmune disorder, such as type 1 insulin-dependent diabetes mellitus, adult respiratory distress syndrome, inflammatory bowel disease, dermatitis, meningitis, thrombotic thrombocytopenic purpura, Sjogren's syndrome, encephalitis, uveitic, leukocyte adhesion deficiency, rheumatoid arthritis, rheumatic fever, Reiter's syndrome, psoriatic arthritis, progressive systemic sclerosis, primary biliary cirrhosis, pemphigus, pemphigoid, necrotizing vasculitis, myasthenia gravis, multiple sclerosis, lupus erythematosus, polymyositis, sarcoidosis, granulomatosis, vasculitis, pernicious anemia, CNS inflammatory disorder, antigen-antibody complex mediated diseases, autoimmune haemolytic anemia, Hashimoto's thyroiditis, Graves disease, habitual spontaneous abortions, Reynard's syndrome, glomerulonephritis, dermatomyositis, chronic active hepatitis, celiac disease, autoimmune complications of AIDS, atrophic gastritis, ankylosing spondylitis and Addison's disease; or transplant rejection, such as GVHD. In certain embodiments of the invention, the ILT3 associated disorder is an immune disorders, such as transplant rejections, graft versus host disease and autoimmune disorders.

The language “immunoglobulin-like transcript 3” or “ILT3” refers to a cell surface molecule of the immunoglobulin superfamily, which is expressed by antigen-presenting cells (APCs) such as monocytes, macrophages and dendritic cells. ILT3 is a member of the immunoglobulin-like transcript (ILT) family and displays a long cytoplasmic tail containing putative immunoreceptor tyrosine-based inhibitory motifs (ITIMs). ILT3 has been shown to behave as an inhibitory receptor when cross-linked to a stimulatory receptor. A cytoplasmic component of the ILT3-mediated signaling pathway is the SH2-containing phosphatase SHP-1, which becomes associated with ILT3 upon cross-linking. ILT3 is also internalized and ILT3 ligands are efficiently presented to specific T cells (see, e.g., Cella, M. et al. (1997) J. Exp. Med. 185:1743). The determination of whether the candidate vitamin D₃ compound modulates the expression of the ILT3 surface molecule can be accomplished, for example, by comparison of ILT3 surface molecule expression to a control, by measuring mRNA expression, or by measuring protein expression.

The term “immune response” includes T and/or B cell responses, e.g., cellular and/or humoral immune responses. The claimed methods can be used to reduce both primary and secondary immune responses. The immune response of a subject can be determined by, for example, assaying antibody production, immune cell proliferation, the release of cytokines, the expression of cell surface markers, cytotoxicity, and the like.

The terms “immunological tolerance” or “tolerance to an antigen” or “immune tolerance” include unresponsiveness to an antigen without the induction of a prolonged generalized immune deficiency. Consequently, according to the invention, a tolerant host is capable of reacting to antigens other than the tolerizing antigen. Tolerance represents an induced depression in the response of a subject that, had it not been subjected to the tolerance-inducing procedure, would be competent to mount an immune response to that antigen. In one embodiment of the invention, immunological tolerance is induced in an antigen-presenting cell, e.g., an antigen-presenting cell derived from the myeloid or lymphoid lineage, dendritic cells, monocytes and macrophages.

The language “immunosuppressive activity” refers to the process of inhibiting a normal immune response. Included in this response is when T and/or B clones of lymphocytes are depleted in size or suppressed in their reactivity, expansion or differentiation. Immunosuppressive activity may be in the form of inhibiting or blocking an immune response already in progress or may involve preventing the induction of an immune response. The functions of activated T cells may be inhibited by suppressing immune cell responses or by inducing specific tolerance, or both. Immunosuppression of T cell responses is generally an active, non-antigen-specific, process that requires continuous exposure of the T cells to the suppressive agent. Tolerance, which involves inducing non-responsiveness or anergy in T cells, is distinguishable from immunosuppression in that it is generally antigen-specific and persists after exposure to the tolerizing agent has ceased. Operationally, tolerance can be demonstrated by the lack of a T cell response upon re-exposure to specific antigen in the absence of the tolerizing agent.

The language “improved biological properties” refers to any activity inherent in a compound of the invention that enhances its effectiveness in vivo. In a preferred embodiment, this term refers to any qualitative or quantitative improved therapeutic property of a vitamin D₃ compound, such as reduced toxicity, e.g., reduced hypercalcemic activity.

The language “inhibiting the growth” of the neoplasm includes the slowing, interrupting, arresting or stopping its growth and metastases and does not necessarily indicate a total elimination of the neoplastic growth.

The phrase “inhibition of an immune response” is intended to include decreases in T cell proliferation and activity, e.g., a decrease in IL₂, interferon-γ, GM-CSF synthesis and secretion (Lemire, J. M. (1992) J. Cell Biochemistry 49:26-31, Lemire, J. M. et al. (1994) Endocrinology 135 (6): 2813-2821; Bouillon, R. et al. (1995) Endocrine Review 16 (2):231-32).

The term “isomers” or “stereoisomers” refers to compounds which have identical chemical constitution, but differ with regard to the arrangement of the atoms or groups in space.

The term “leukemia” is intended to have its clinical meaning, namely, a neoplastic disease in which white corpuscle maturation is arrested at a primitive stage of cell development. The disease is characterized by an increased number of leukemic blast cells in the bone marrow, and by varying degrees of failure to produce normal hematopoietic cells. The condition may be either acute or chronic. Leukemias are further typically categorized as being either lymphocytic i.e., being characterized by cells which have properties in common with normal lymphocytes, or myelocytic (or myelogenous), i.e., characterized by cells having some characteristics of normal granulocytic cells. Acute lymphocytic leukemia (“ALL”) arises in lymphoid tissue, and ordinarily first manifests its presence in bone marrow. Acute myelocytic leukemia (“AML”) arises from bone marrow hematopoietic stem cells or their progeny. The term acute myelocytic leukemia subsumes several subtypes of leukemia: myeloblastic leukemia, promyelocytic leukemia, and myelomonocytic leukemia. In addition, leukemias with erythroid or megakaryocytic properties are considered myelogenous leukemias as well.

The term “leukemic cancer” refers to all cancers or neoplasias of the hemopoietic and immune systems (blood and lymphatic system). The acute and chronic leukemias, together with the other types of tumors of the blood, bone marrow cells (myelomas), and lymph tissue (lymphomas), cause about 10% of all cancer deaths and about 50% of all cancer deaths in children and adults less than 30 years old. Chronic myelogenous leukemia (CML), also known as chronic granulocytic leukemia (CGL), is a neoplastic disorder of the hematopoietic stem cell. The term “leukemia” is art recognized and refers to a progressive, malignant disease of the blood-forming organs, marked by distorted proliferation and development of leukocytes and their precursors in the blood and bone marrow.

The term “modulate” refers to increases or decreases in the activity of a cell in response to exposure to a compound of the invention, e.g., the inhibition of proliferation and/or induction of differentiation of at least a sub-population of cells in an animal such that a desired end result is achieved, e.g., a therapeutic result. In preferred embodiments, this phrase is intended to include hyperactive conditions that result in pathological disorders.

The common medical meaning of the term “neoplasia” refers to “new cell growth” that results as a loss of responsiveness to normal growth controls, e.g. to neoplastic cell growth. A “hyperplasia” refers to cells undergoing an abnormally high rate of growth. However, as used herein, the terms neoplasia and hyperplasia can be used interchangably, as their context will reveal, referring to generally to cells experiencing abnormal cell growth rates. Neoplasias and hyperplasias include “tumors,” which may be either benign, premalignant or malignant.

The language “non-genomic” vitamin D₃ activities include cellular (e.g., calcium transport across a tissue) and subcellular activities (e.g., membrane calcium transport opening of voltage-gated calcium channels, changes in intracellular second messengers) elicited by vitamin D₃ compounds in a responsive cell. Electrophysiological and biochemical techniques for detecting these activities are known in the art. An example of a particular well-studied non-genomic activity is the rapid hormonal stimulation of intestinal calcium mobilization, termed “transcaltachia” (Nemere I. et al. (1984) Endocrinology 115:1476-1483; Lieberherr M. et al. (1989) J. Biol. Chem. 264:20403-20406; Wali R. K. et al. (1992) Endocrinology 131:1125-1133; Wali R. K. et al. (1992) Am. J. Physiol. 262:G945-G953; Wali R. K. et al. (1990) J. Clin. Invest. 85:1296-1303; Bolt M. J. G. et al. (1993) Biochem. J. 292:271-276). Detailed descriptions of experimental transcaltachia are provided in Norman, A. W. (1993) Endocrinology 268(27):20022-20030; Yoshimoto, Y. and Norman, A. W. (1986) Endocrinology 118:2300-2304. Changes in calcium activity and second messenger systems are well known in the art and are extensively reviewed in Bouillion, R. et al. (1995) Endocrinology Review 16(2): 200-257; the description of which is incorporated herein by reference.

The term “obtaining” as in “obtaining a vitamin D₃ compound” is intended to include purchasing, synthesizing or otherwise acquiring the compound.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The terms “polycyclyl” or “polycyclic radical” refer to the radical of two or more cyclic rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkyl, alkylaryl, or an aromatic or heteroaromatic moiety.

The term “prodrug” includes compounds with moieties which can be metabolized in vivo. Generally, the prodrugs are metabolized in vivo by esterases or by other mechanisms to active drugs. Examples of prodrugs and their uses are well known in the art (See, e.g., Berge et al. (1977) “Pharmaceutical Salts”, J. Pharm. Sci. 66:1-19). The prodrugs can be prepared in situ during the final isolation and purification of the compounds, or by separately reacting the purified compound in its free acid form or hydroxyl with a suitable esterifying agent. Hydroxyl groups can be converted into esters via treatment with a carboxylic acid. Examples of prodrug moieties include substituted and unsubstituted, branch or unbranched lower alkyl ester moieties, (e.g., propionoic acid esters), lower alkenyl esters, di-lower alkyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, di-lower alkyl amides, and hydroxy amides. Preferred prodrug moieties are propionoic acid esters and acyl esters. Prodrugs which are converted to active forms through other mechanisms in vivo are also included.

The language “a prophylactically effective anti-neoplastic amount” of a compound refers to an amount of a vitamin D₃ compound of the formula (I) or otherwise described herein which is effective, upon single or multiple dose administration to the patient, in preventing or delaying the occurrence of the onset of a neoplastic disease state.

The term “psoriasis” is intended to have its medical meaning, namely, a disease which afflicts primarily the skin and produces raised, thickened, scaling, nonscarring lesions. The lesions are usually sharply demarcated erythematous papules covered with overlapping shiny scales. The scales are typically silvery or slightly opalescent. Involvement of the nails frequently occurs resulting in pitting, separation of the nail, thickening and discoloration. Psoriasis is sometimes associated with arthritis, and it may be crippling.

The language “reduced toxicity” is intended to include a reduction in any undesired side effect elicited by a vitamin D₃ compound when administered in vivo, e.g., a reduction in the hypercalcemic activity.

The term “sarcoma” is art recognized and refers to malignant tumors of mesenchymal derivation.

The term “secosteroid” is art-recognized and includes compounds in which one of the cyclopentanoperhydro-phenanthrene rings of the steroid ring structure is broken. 1α,25(OH)₂D₃ and analogs thereof are hormonally active secosteroids. In the case of vitamin D₃, the 9-10 carbon-carbon bond of the B-ring is broken, generating a seco-B-steroid. The official IUPAC name for vitamin D₃ is 9,10-secocholesta-5,7,10(19)-trien-3B-ol. For convenience, a 6-s-trans conformer of 1α,25(OH)₂D₃ is illustrated herein having all carbon atoms numbered using standard steroid notation.

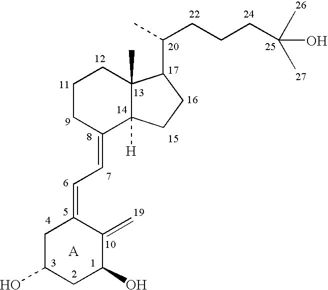

In the formulas presented herein, the various substituents on ring A are illustrated as joined to the steroid nucleus by one of these notations: a dotted line [symbol] indicating a substituent which is in the β-orientation (i.e., above the plane of the ring), a wedged solid line [symbol] indicating a substituent which is in the α-orientation (i.e., below the plane of the molecule), or a wavy line [symbol] indicating that a substituent may be either above or below the plane of the ring. In regard to ring A, it should be understood that the stereochemical convention in the vitamin D field is opposite from the general chemical field, wherein a dotted line indicates a substituent on Ring A which is in an α-orientation (i.e., below the plane of the molecule), and a wedged solid line indicates a substituent on ring A which is in the β-orientation (i.e., above the plane of the ring). As shown, the A ring of the hormone 1α,25(OH)₂D₃ contains two asymmetric centers at carbons 1 and 3, each one containing a hydroxyl group in well-characterized configurations, namely the 1α- and 3β-hydroxyl groups. In other words, carbons 1 and 3 of the A ring are said to be “chiral carbons” or “carbon centers”.

Also, throughout the patent literature, the A ring of a vitamin D compound is often depicted in generic formulae as any one of the following structures:

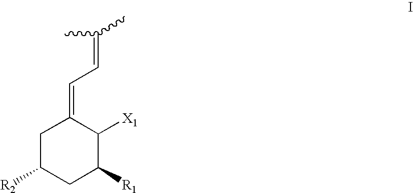

wherein X₁ is defined as H (or H₂) or ═CH₂; or

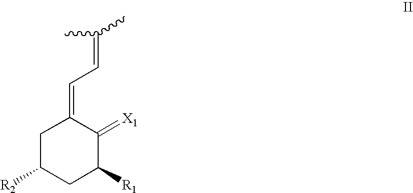

wherein X₁ is defined as H₂ or CH₂. Although there does not appear to be any set convention, it is clear that one of ordinary skill in the art understands either formula I or II to represent an A ring in which, for example, X₁ is ═CH₂, as follows:

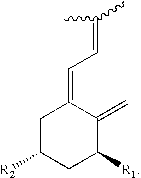

For purposes of the instant invention, the representation of the A ring as shown immediately above in formula II will be used in all generic structures.

Furthermore the indication of stereochemistry across a carbon-carbon double bond is also opposite from the general chemical field in that “Z” refers to what is often referred to as a “cis” (same side) conformation whereas “E” refers to what is often referred to as a “trans” (opposite side) conformation. As shown, the A ring of the hormone 1-alpha,25(OH)₂D₃ contains two asymmetric centers at carbons 1 and 3, each one containing a hydroxyl group in well-characterized configurations, namely the 1-alpha- and 3-beta-hydroxyl groups. In other words, carbons 1 and 3 of the A ring are said to be “chiral carbons” or “chiral carbon centers.” Regardless, both configurations, cis/trans and/or Z/E are encompassed by the compounds of the present invention. With respect to the nomenclature of a chiral center, the terms “d” and “l” configuration are as, defined by the IUPAC Recommendations. As to the use of the terms, diastereomer, racemate, epimer and enantiomer, these will be used in their normal context to describe the stereochemistry of preparations.

The term “subject” includes organisms which are capable of suffering from a vitamin D₃ associated state or who could otherwise benefit from the administration of a vitamin D₃ compound of the invention, such as human and non-human animals. Preferred human animals include human patients suffering from or prone to suffering from a vitamin D₃ associated state, as described herein. The term “non-human animals” of the invention includes all vertebrates, e.g., mammals, e.g., rodents, e.g., mice, and non-mammals, such as non-human primates, sheep, dog, cow, chickens, amphibians, reptiles, etc.

The term “sulfhydryl” or “thiol” means —SH.

The phrases “systemic administration,” “administered systemically”, “peripheral administration” and “administered peripherally” as used herein mean the administration of a vitamin D₃ compound(s), drug or other material, such that it enters the patient's system and, thus, is subject to metabolism and other like processes, for example, subcutaneous administration.

The language “therapeutically effective anti-neoplastic amount” of a vitamin D₃ compound of the invention refers to an amount of an agent which is effective, upon single or multiple dose administration to the patient, in inhibiting the growth of a neoplastic vitamin D₃-responsive cells, or in prolonging the survivability of the patient with such neoplastic cells beyond that expected in the absence of such treatment.

The language “transplant rejection” refers to an immune reaction directed against a transplanted organ(s) from other human donors (allografts) or from other species such as sheep, pigs, or non-human primates (xenografts). Therefore, the method of the invention is useful for preventing an immune reaction to transplanted organs from other human donors (allografts) or from other species (xenografts). Such tissues for transplantation include, but are not limited to, heart, liver, kidney, lung, pancreas, pancreatic islets, bone marrow, brain tissue, cornea, bone, intestine, skin, and hematopoietic cells. Also included within this definition is “graft versus host disease” of “GVHD,” which is a condition where the graft cells mount an immune response against the host. Therefore, the method of the invention is useful in preventing graft versus host disease in cases of mismatched bone marrow or lymphoid tissue transplanted for the treatment of acute leukemia, aplastic anemia, and enzyme or immune deficiencies, for example. The term “transplant rejection” also includes disease symptoms characterized by loss of organ function. For example, kidney rejection would be characterized by a rising creatine level in blood. Heart rejection is characterized by an endomyocardial biopsy, while pancreas rejection is characterized by rising blood glucose levels. Liver rejection is characterized by the levels of transaminases of liver origin and bilirubin levels in blood. Intestine rejection is determined by biopsy, while lung rejection is determined by measurement of blood oxygenation.

The terms “urogenital”, “urogenital system” and “urogential tract” are used interchangeably and are intended to include all organs involved in reproduction and in the formation and voidance of urine. Included with in these terms are the kidneys, bladder and prostate.

The term “VDR” is intended to include members of the type II class of steroid/thyroid superfamily of receptors (Stunnenberg, H. G. (1993) Bio Essays 15(5):309-15), which are able to bind and transactivate through the vitamin D response element (VDRE) in the absence of a ligand (Damm et al. (1989) Nature 339:593-97; Sap et al. Nature 343:177-180).

The term “VDRE” refers to DNA sequences composed of half-sites arranged as direct repeats. It is known in the art that type II receptors do not bind to their respective binding site as homodimers but require an auxiliary factor, RXR (e.g. RXRα, RXRβ, RXRγ) for high affinity binding Yu et al. (1991) Cell 67:1251-1266; Bugge et al. (1992) EMBO J. 11:1409-1418; Kliewer et al. (1992) Nature 355:446-449; Leid et al. (1992) EMBO J. 11:1419-1435; Zhang et al. (1992) Nature 355:441-446).

The language “vitamin D₃ associated state” is a state which can be prevented, treated or otherwise ameliorated by administration of one or more compounds of the invention. Vitamin D₃ associated states include ILT3-associated disorders, disorders characterized by an aberrant activity of a vitamin D₃-responsive cell, disorders characterized by a deregulation of calcium and phosphate metabolism, and other disorders or states described herein.

The term “vitamin D₃-responsive cell” includes any cell which is capable of responding to a vitamin D₃ compound having the formula I or I-a or otherwise described herein, or is associated with disorders involving an aberrant activity of hyperproliferative skin cells, parathyroid cells, neoplastic cells, immune cells, and bone cells. These cells can respond to vitamin D₃ activation by triggering genomic and/or non-genomic responses that ultimately result in the modulation of cell proliferation, differentiation survival, and/or other cellular activities such as hormone secretion. In a preferred embodiment, the ultimate responses of a cell are inhibition of cell proliferation and/or induction of differentiation-specific genes. Exemplary vitamin D₃ responsive cells include immune cells, bone cells, neuronal cells, endocrine cells, neoplastic cells, epidermal cells, endodermal cells, smooth muscle cells, among others. With respect to the nomenclature of a chiral center, terms “d” and “l” configuration are as defined by the IUPAC Recommendations. As to the use of the terms, diastereomer, racemate, epimer and enantiomer will be used in their normal context to describe the stereochemistry of preparations.

### 2. Gemini Vitamin D₃ Compounds

In the structure of vitamin D₃ gemini analogs, two full side chains are attached at the C-20 position. Gemini compounds exert a full spectrum of 1,25(OH)₂D₃ biological activities such as binding to the specific nuclear receptor VDR, suppression of the increased parathyroid hormone levels in 5,6-nephrectomized rats, suppression of INF-γ release in MLR cells, stimulation of HL-60 leukemia cell differentiation and inhibition of solid tumor cell proliferation (Uskokovic, M. R et al., “Synthesis and preliminary evaluation of the biological properties of a 1α,25-dihydroxyvitamin D₃ analogue with two sidechains.” Vitamin D: Chemistry, Biology and Clinical Applications of the Steroid Hormone; Norman, A. W., et al., Eds.; University of California: Riverside, 1997; pp 19-21; Norman et al., J. Med. Chem. 2000, Vol. 43, 2719-2730).

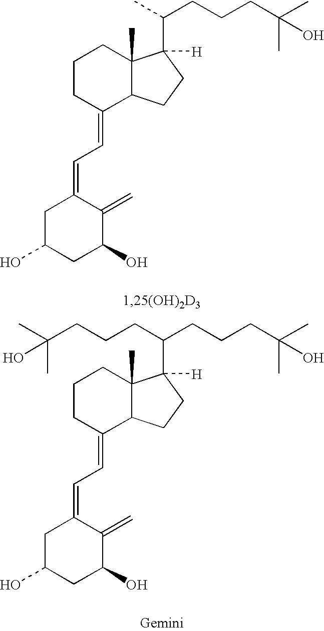

Both in vivo and in cellular cultures, 1,25-(OH)₂D₃ undergoes a cascade of metabolic modifications initiated by the influence of 24R-hydroxylase enzyme. First 24R-hydroxy metabolite is formed, which is oxydized to 24-keto intermediate, and then 23S-hydroxylation and fragmentation produce the fully inactive calcitroic acid.

In one aspect, the invention provides a vitamin D₃ compound having formula I:

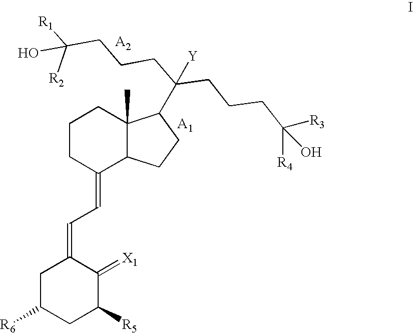

wherein:
A₁ is a single or double bond;
A₂ is a single, a double or a triple bond;
R₁, R₂, R₃ and R₄ are each independently alkyl, deuteroalkyl, hydroxyalkyl, or haloalkyl;
R₅ is halogen, hydroxyl, OC(O)alkyl, OC(O)hydroxyalkyl, or OC(O)haloalkyl;
R₆ is halogen, hydroxyl, OC(O)alkyl, OC(O)hydroxyalkyl, or OC(O)haloalkyl;

### X₁ is H₂ or CH₂;

Y is alkyl;
and pharmaceutically acceptable esters, salts, and prodrugs thereof.

Embodiments of the invention include compounds wherein, A₁ is a single bond, A₂ is a single bond, or A₂ is a triple bond. In other embodiments of the invention, R₁, R₂, R₃, and R₄ are each independently alkyl, or R₁ and R₂ are each independently haloalkyl, and R₃ and R₄ are each independently allyl. Preferably, R₁ and R₂ are trifluoromethyl, and R₃ and R₄ are methyl. In another embodiment, R₅ is hydroxyl. In another embodiment, R₅ is halogen, preferably F. In another embodiment, R₆ is hydroxyl.

In one embodiment, the invention provides a compound wherein X₁ is H₂. In another embodiment, the invention provides a compound wherein X₁ is CH₂.

In one embodiment, Y is lower alkyl. In another embodiment, Y is (C₁-C₄)alkyl, e.g., methyl.

In one aspect, the invention provides a compound having formula I-a:

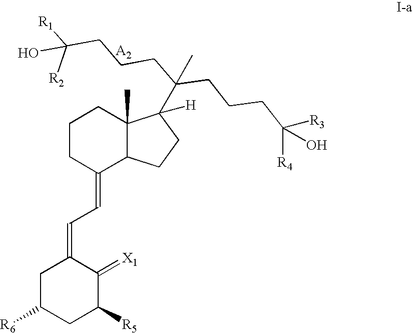

wherein:
A₂ is a single, a double or a triple bond;
R₁, R₂, R₃ and R₄ are each independently alkyl, hydroxyalkyl, or haloalkyl;
R₅ is halogen, hydroxyl, OC(O)alkyl, OC(O)hydroxyalkyl, or OC(O)haloalkyl;
R₆ is hydroxyl, OC(O)alkyl, OC(O)hydroxyalkyl, or OC(O)haloalkyl;

### X₁ is H₂ or CH₂;

and pharmaceutically acceptable esters, salts, and prodrugs thereof.

In a preferred embodiment, the invention provides a compound wherein R₆ is hydroxyl and A₂ is a single bond. In a further embodiment, X₁ is CH₂ and R₅ is halogen, preferably F. In another further embodiment, X₁ is CH₂ and R₅ is hydroxyl. In still another embodiment, X₁ is H₂ and R₅ is hydroxyl. In another embodiment, X₁ is H₂ and R₅ is halogen. In a further embodiment, R₁, R₂, R₃, and R₄ are alkyl, preferably methyl.

In one embodiment, the invention provides a compound wherein R₆ is hydroxyl and A₂ is a triple bond. In another embodiment, R₆ is hydroxyl and A₂ is a double bond. In one embodiment, X₁ is CH₂, and R₅ is hydroxyl. In a further embodiment, R₁, R₂, R₃ and R₄ are each independently alkyl or haloalkyl. In a further embodiment, R₁ and R₂ are haloalkyl, preferably trifluoromethyl. In a further embodiment, R₃ and R₄ are alkyl, preferably methyl. Preferably, R₁ and R₂ are haloalkyl, and R₃ and R₄ are alkyl. In a preferred embodiment, R₁ and R₂ are trifluoromethyl, and R₃ and R₄ are methyl. In another preferred embodiment, R₃ and R₄ are trifluoromethyl, and R₁ and R₂ are methyl.

In a further embodiment, the invention provides a compound wherein X₁ is H₂, and R₅ is hydroxyl. In another further embodiment, R₁, R₂, R₃ and R₄ are each independently alkyl or haloalkyl. In yet another further embodiment, R₁ and R₂ are haloalkyl, preferably trifluoromethyl. In still another embodiment, R₃ and R₄ are alkyl, preferably R₃ and R₄ are methyl.

In another embodiment, the invention provides a compound wherein X₁ is CH₂, and R₅ is halogen. Preferably, R₅ is F. In a further embodiment, R₁, R₂, R₃ and R₄ are each independently alkyl or haloalkyl. In one further embodiment, R₁ and R₂ are haloalkyl, preferably trifluoromethyl. In another further embodiment, R₃ and R₄ are alkyl, preferably methyl.

In certain aspects, the invention provides a compound having formula I-b:

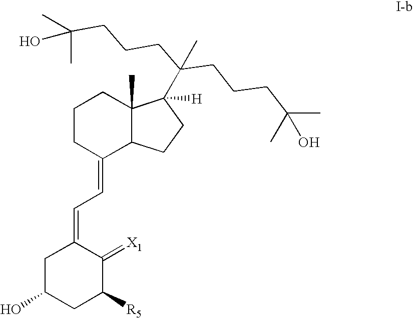

wherein:
R₅ is fluoro or hydroxyl;

### X₁ is H₂ or CH₂;

and pharmaceutically acceptable esters, salts, and prodrugs thereof.

In certain embodiments, X₁ is CH₂. In a further embodiment, R₅ is hydroxyl or fluoro. In other embodiments, X₁ is H₂ and R₅ is hydroxyl.

In other aspects, the invention provides a compound having formula I-c:

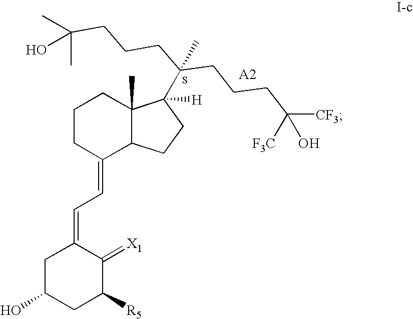

wherein:
A₂ is a single, a double or a triple bond;
R₅ is fluoro or hydroxyl;

### X₁ is H₂ or CH₂;

and pharmaceutically acceptable esters, salts, and prodrugs thereof.

In another aspect, the invention provides a compound having formula I-d:

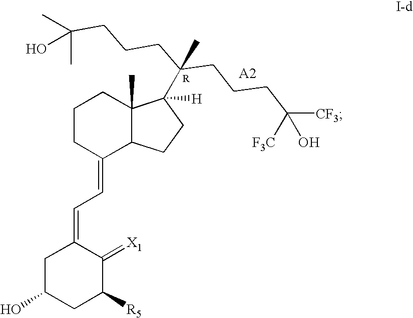

wherein:
A₂ is a single, a double or a triple bond;
R₅ is fluoro or hydroxyl;

### X₁ is H₂ or CH₂;

and pharmaceutically acceptable esters, salts, and prodrugs thereof.

In yet another aspect, the invention provides a compound having formula I-e:

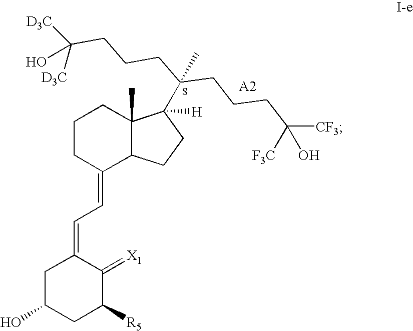

wherein:
A₂ is a single, a double or a triple bond;
R₅ is fluoro or hydroxyl;

### X₁ is H₂ or CH₂;

and pharmaceutically acceptable esters, salts, and prodrugs thereof.

In still another aspect, the invention provides a compound having formula I-f

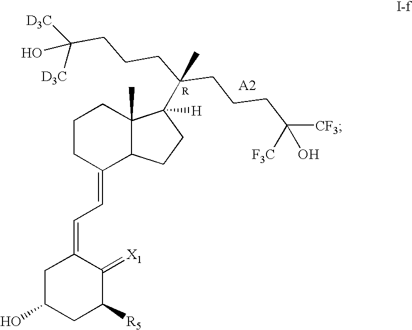

wherein:
A₂ is a single, a double or a triple bond;
R₅ is fluoro or hydroxyl;

### X₁ is H₂ or CH₂;

and pharmaceutically acceptable esters, salts, and prodrugs thereof.

In certain embodiments related to compounds of formulae I-c to I-f, A₂ is a triple bond. In a further embodiment, X₁ is CH₂. In still further embodiments, R₅ is hydroxyl or fluoro. In another embodiment, X₁ is H₂ and R₅ is hydroxyl.

In other embodiments, A₂ is a cis double bond. In a further embodiment, X₁ is CH₂. In still further embodiments, R₅ is hydroxyl or fluoro. In another embodiment, X₁ is H₂ and R₅ is hydroxyl.

In yet other embodiments, A₂ is a trans double bond. In a further embodiment, X₁ is CH₂. In still further embodiments, R₅ is hydroxyl or fluoro. In another embodiment, X₁ is H₂ and R₅ is hydroxyl.

Preferred compounds of the invention include the following compounds, which are further exemplified in Chart 1:
1,25-Dihydroxy-20-(4-hydroxy-4-methyl-pentyl)-cholecalciferol (1);
1,25-Dihydroxy-20-(4-hydroxy-4-methyl-pentyl)-19-nor-cholecalciferol (2);
1α-Fluoro-25-hydroxy-20-(4-hydroxy-4-methyl-pentyl)-cholecalciferol (3);
(20S)-1,25-Dihydroxy-20-(5,5,5-trifluoro-4-hydroxy-4-trifluoromethyl-pent-2-ynyl)-cholecalciferol (4);
(20S)-1,25-Dihydroxy-20-((2Z)-5,5,5-trifluoro-4-hydroxy-4-trifluoromethyl-pent-2-enyl)cholecalciferol (5);
(20S)-1,25-Dihydroxy-20-[(2E)-5,5,5-trifluoro-4-hydroxy-4-trifluoromethyl-pent-2-enyl]-cholecalciferol (6);
(20R)-1,25-Dihydroxy-20-(5,5,5-trifluoro-4-hydroxy-4-trifluoromethyl-pent-2-ynyl)-cholecalciferol (7);
(20R)-1,25-Dihydroxy-20-[(2Z)-5,5,5-trifluoro-4-hydroxy-4-trifluoromethyl-pent-2-enyl]-cholecalciferol (8);
(20R)-1,25-Dihydroxy-20-[(2E)-5,5,5-trifluoro-4-hydroxy-4-trifluoromethyl-pent-2-enyl]-cholecalciferol (9);
(20S)-1,25-Dihydroxy-20-(5,5,5-trifluoro-4-hydroxy-4-trifluoromethyl-pent-2-ynyl)-19-nor-cholecalciferol (10);
(20S)-1,25-Dihydroxy-20-[(2Z)-5,5,5-trifluoro-4-hydroxy-4-trifluoromethyl-pent-2-enyl]-19-nor-cholecalciferol (11);
(20S)-1,25-Dihydroxy-20-[(2E)-5,5,5-trifluoro-4-hydroxy-4-trifluoromethyl-pent-2-enyl]-19-nor-cholecalciferol-(12);
(20S)-1α-Fluoro-25-hydroxy-20-(5,5,5-trifluoro-4-hydroxy-4-trifluoromethyl-pent-2-ynyl)-cholecalciferol (13);
(20S)-1α-Fluoro-25-hydroxy-20-[(2Z)-5,5,5-trifluoro-4-hydroxy-4-trifluoromethyl-pent-2-enyl]-cholecalciferol (14);
(20S)-1α-Fluoro-25-hydroxy-20-[(2E)-5,5,5-trifluoro-4-hydroxy-4-trifluoromethyl-pent-2-enyl]-cholecalciferol (15);
(20S)-1,25-Dihydroxy-20-((2Z)-5,5,5-trifluoro-4-hydroxy-4-trifluoromethyl-pent-2-enyl)-26,27-hexadeutero-cholecalciferol (16);
(20S)-1,25-Dihydroxy-20-((2Z)-5,5,5-trifluoro-4-hydroxy-4-trifluoromethyl-pent-2-enyl)-26,27-hexadeutero-19-nor-cholecalciferol (17);
(20S)-1α-Fluoro-25-hydroxy-20-((2Z)-5,5,5-trifluoro-4-hydroxy-4-trifluoromethyl-pent-2-enyl)-26,27-hexadeutero-cholecalciferol (18);
(20S)-1,25-Dihydroxy-20-((2E)-5,5,5-trifluoro-4-hydroxy-4-trifluoromethyl-pent-2-enyl)-26,27-hexadeutero-cholecalciferol (19);
(20S)-1,25-Dihydroxy-20-((2E)-5,5,5-trifluoro-4-hydroxy-4-trifluoromethyl-pent-2-enyl)-26,27-hexadeutero-19-nor-cholecalciferol (20);
(20S)-1α-Fluoro-25-hydroxy-20-((2E)-5,5,5-trifluoro-4-hydroxy-4-trifluoromethyl-pent-2-enyl)-26,27-hexadeutero-cholecalciferol (21);
(20S)-1,25-Dihydroxy-20-(5,5,5-trifluoro-4-hydroxy-4-trifluoromethyl-pent-2-ynyl)-26,27-hexadeutero-cholecalciferol (22);
(20S)-1,25-Dihydroxy-20-(5,5,5-trifluoro-4-hydroxy-4-trifluoromethyl-pent-2-ynyl)-26,27-hexadeutero-19-nor-cholecalciferol (23);
(20S)-1α-Fluoro-25-hydroxy-20-(5,5,5-trifluoro-4-hydroxy-4-trifluoromethyl-pent-2-ynyl)-26,27-hexadeutero-cholecalciferol (24);
1,25-Dihydroxy-20R-20-(4-hydroxy-5,5,5-trideutero-4-trideuteromethyl-pentyl)-23Z-ene-26,27-hexafluorocholecalciferol (25);
1,25-Dihydroxy-20R-20-(4-hydroxy-5,5,5-trideutero-4-trideuteromethyl-pentyl)-23Z-ene-26,27-hexafluoro-19-nor-cholecalciferol (26);
1α-Fluoro-25-hydroxy-20R-20-(4-hydroxy-5,5,5-trideutero-4-trideuteromethyl-pentyl)-23Z-ene-26,27-hexafluorocholecalciferol (27);
1,25-Dihydroxy-20R-20-(4-hydroxy-5,5,5-trideutero-4-trideuteromethyl-pentyl)-23E-ene-26,27-hexafluorocholecalciferol (28);
1,25-Dihydroxy-20R-20-(4-hydroxy-5,5,5-trideutero-4-trideuteromethyl-pentyl)-23E-ene-26,27-hexafluoro-19-nor-cholecalciferol (29);
1α-Fluoro-25-hydroxy-20R-20-(4-hydroxy-5,5,5-trideutero-4-trideuteromethyl-pentyl)-23E-ene-26,27-hexafluorocholecalciferol (30);
1,25-Dihydroxy-20R-20-(4-hydroxy-5,5,5-trideutero-4-trideuteromethyl-pentyl)-23-yne-26,27-hexafluorocholecalciferol (31);
1,25-Dihydroxy-20R-20-(4-hydroxy-5,5,5-trideutero-4-trideuteromethyl-pentyl)-23-yne-26,27-hexafluoro-19-nor-cholecalciferol (32);
1α-Fluoro-25-hydroxy-20R-20-(4-hydroxy-5,5,5-trideutero-4-trideuteromethyl-pentyl)-23-yne-26,27-hexafluorocholecalciferol (33);
1,25-Dihydroxy-20R-20-(4-hydroxy-4-methyl-pentyl)-23-yne-26,27-hexafluoro-cholecalciferol (34);
1,25-Dihydroxy-20R-20-(4-hydroxy-4-methyl-pentyl)-23Z-ene-26,27-hexafluoro-cholecalciferol (35);
1,25-Dihydroxy-20R-20-(4-hydroxy-4-methyl-pentyl)-23E-ene-26,27-hexafluoro-cholecalciferol (36);
1α-Fluoro-25-hydroxy-20R-20-(4-hydroxy-4-methyl-pentyl)-23-yne-26,27-hexafluorocholecalciferol (37);
1-Fluoro-25-hydroxy-20R-20-(4-hydroxy-4-methyl-pentyl)-23Z-ene-26,27-hexafluorocholecalciferol (38); and
1α-Fluoro-25-hydroxy-20R-20-(4-hydroxy-4-methyl-pentyl)-23E-ene-26,27-hexafluorocholecalciferol (39).

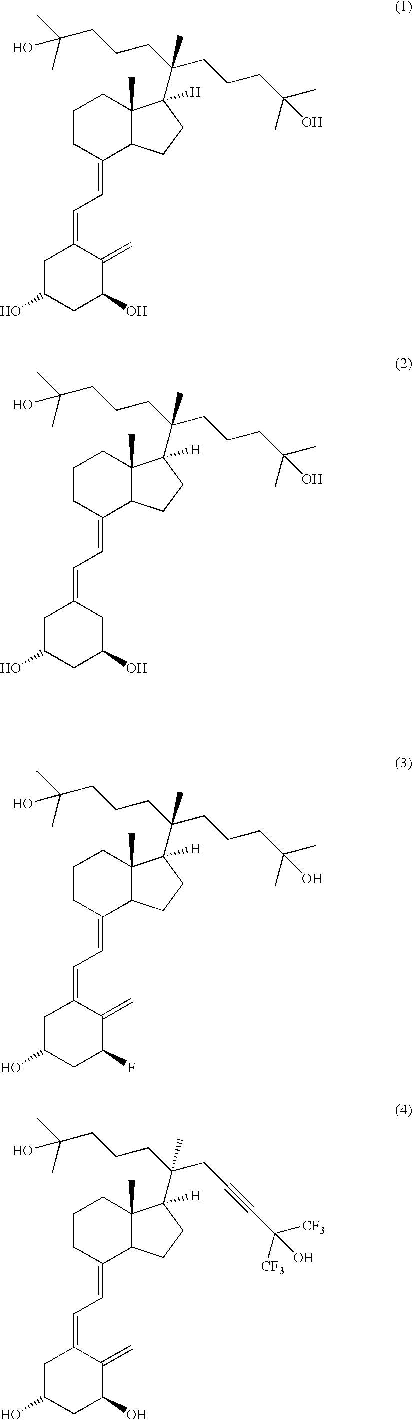

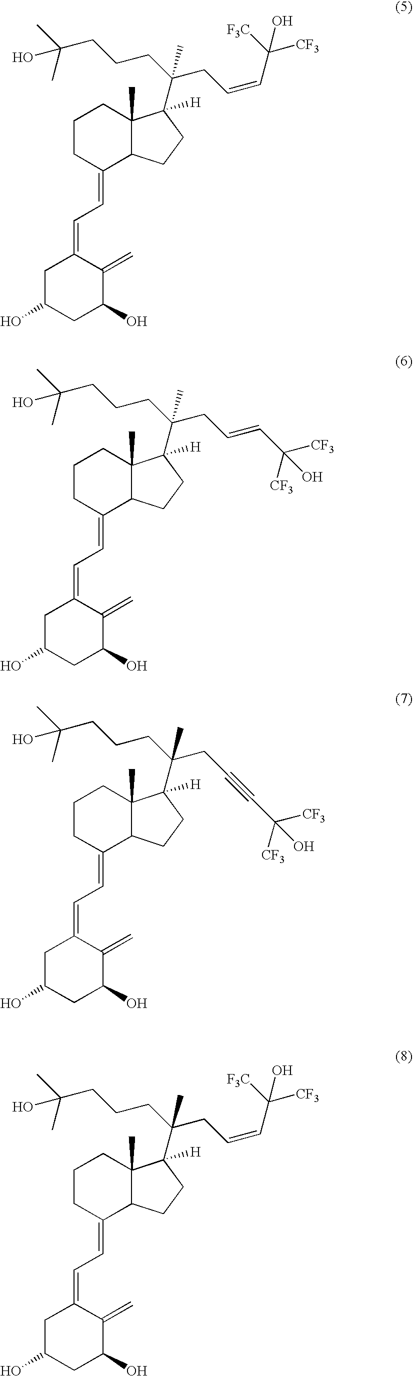

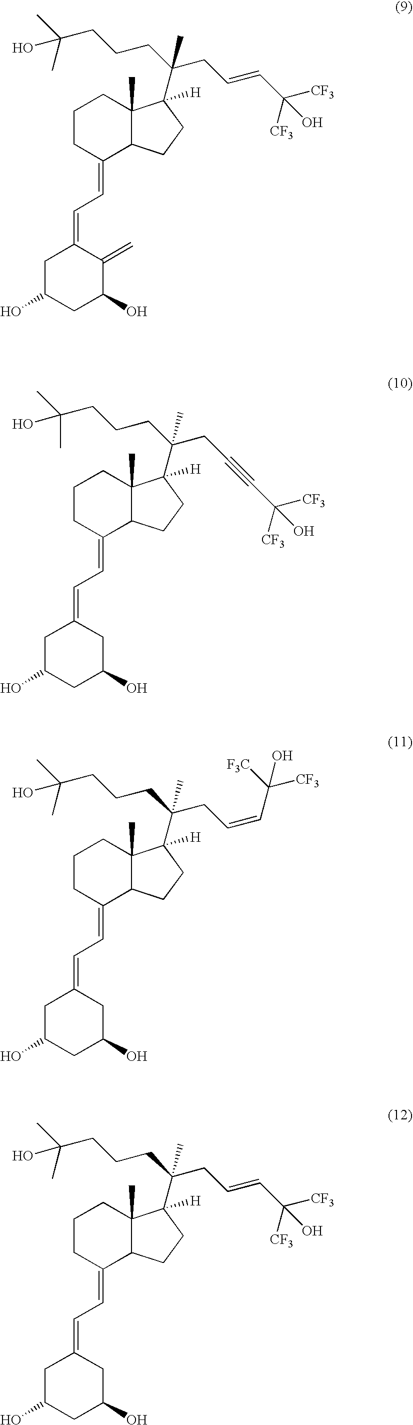

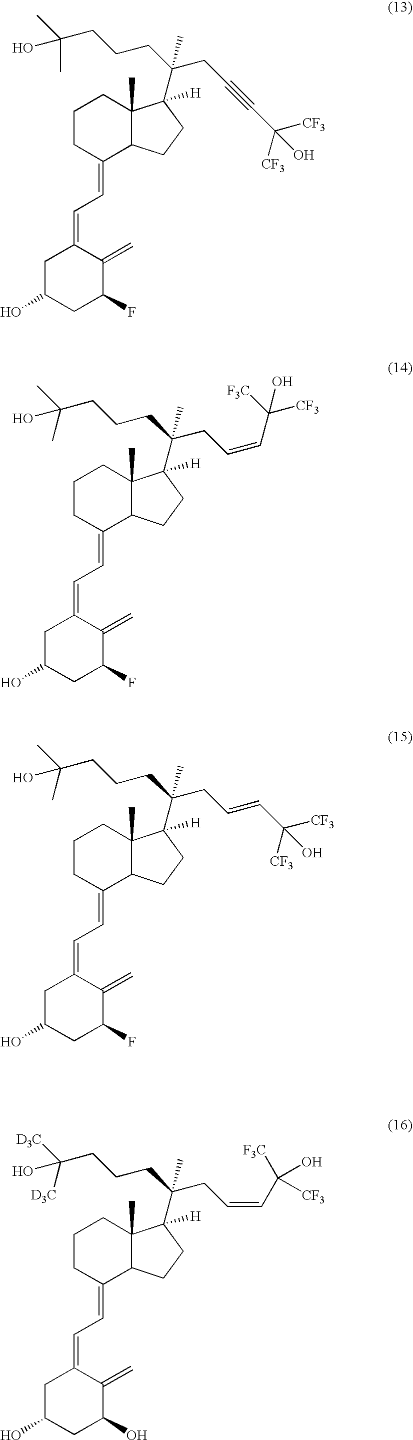

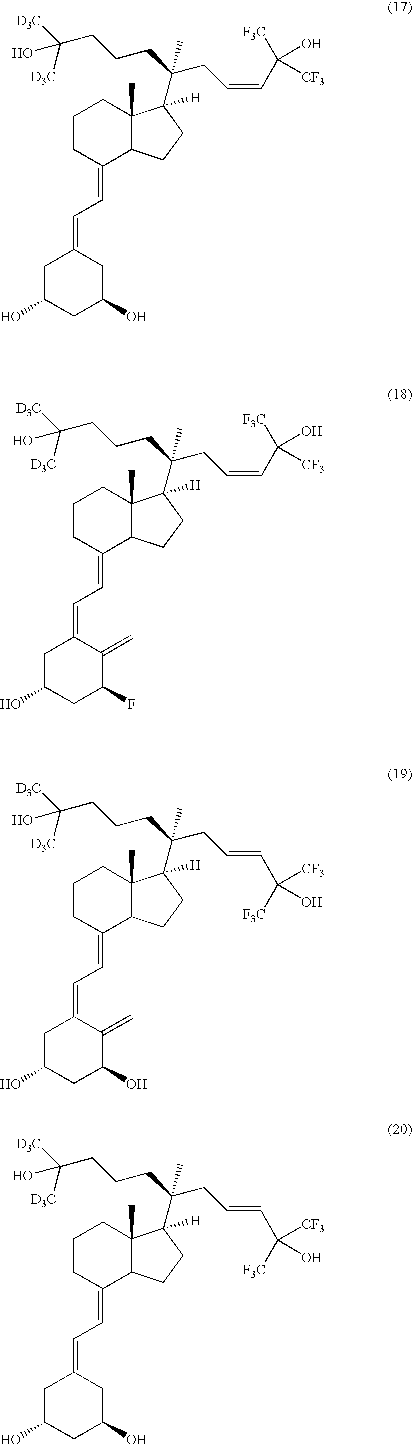

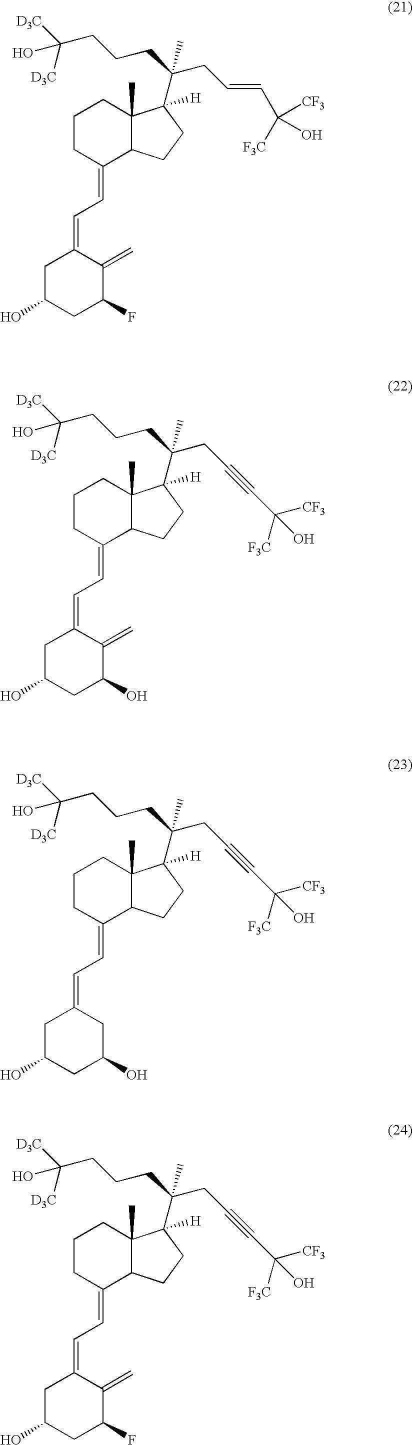

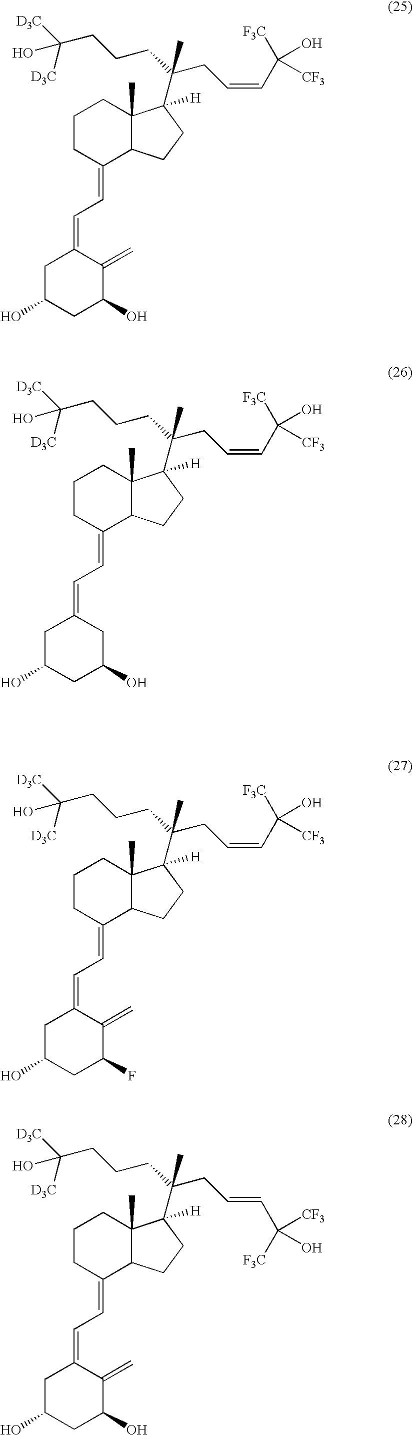

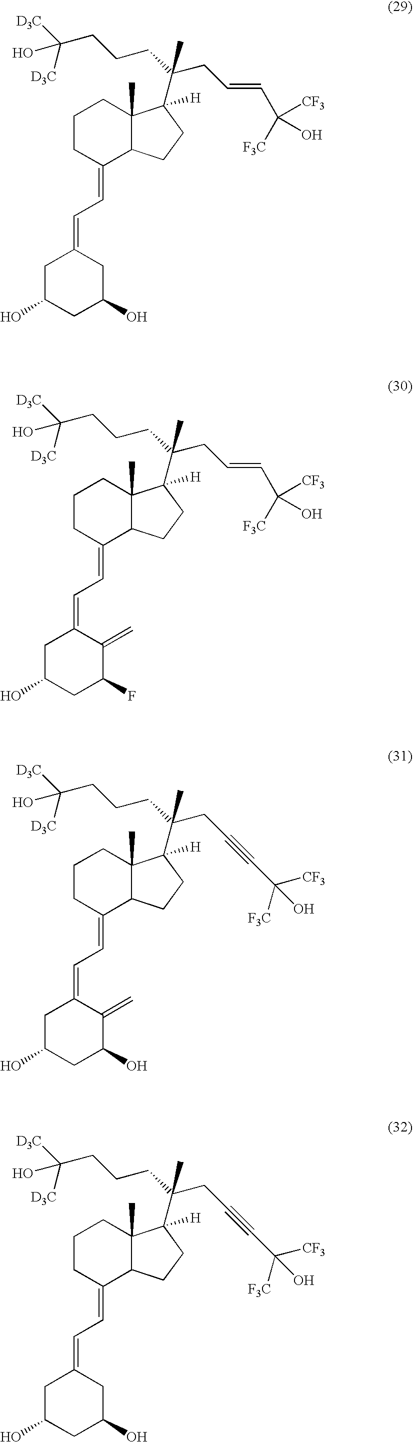

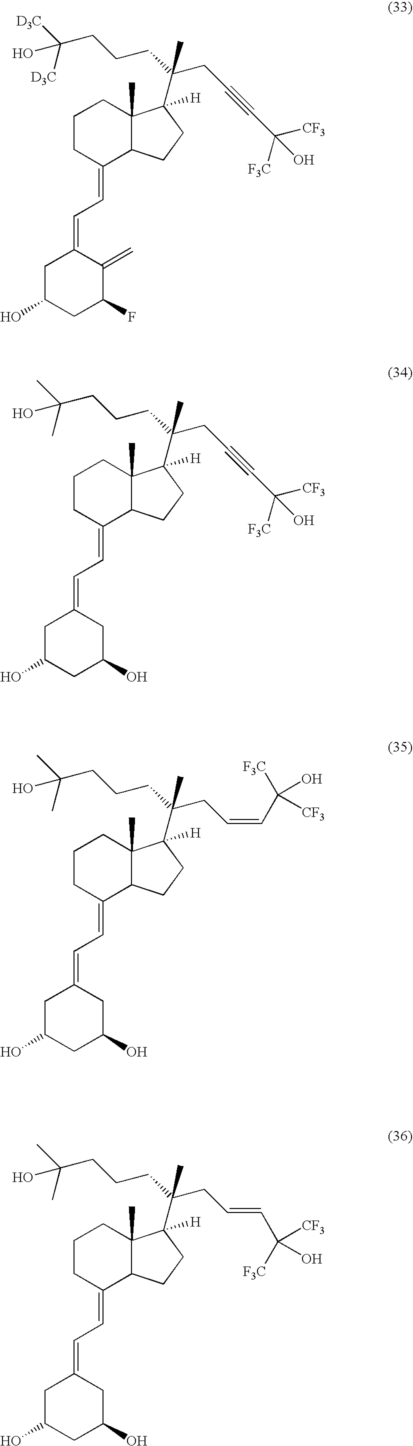

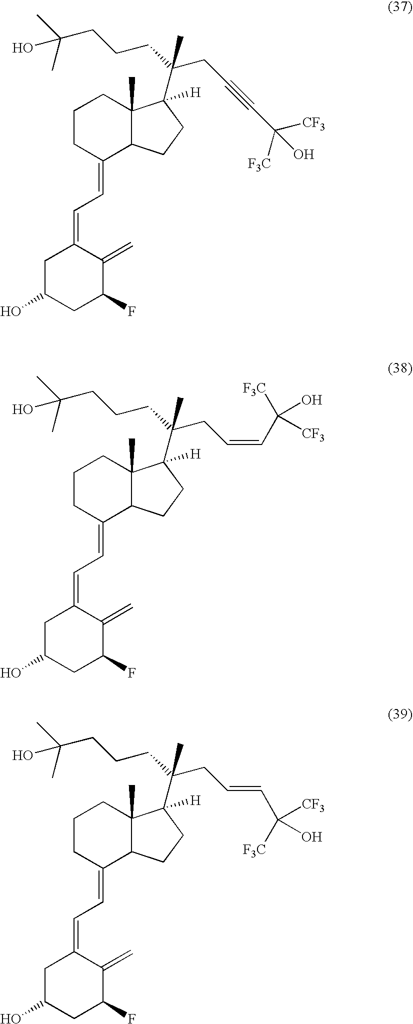

The structures of some of the compounds of the invention include asymmetric carbon atoms. Accordingly, the isomers arising from such asymmetry (e.g., all enantiomers and diastereomers) are included within the scope of this invention, unless indicated otherwise. Such isomers can be obtained in substantially pure form by classical separation techniques and/or by stereochemically controlled synthesis.

Naturally occurring or synthetic isomers can be separated in several ways known in the art. Methods for separating a racemic mixture of two enantiomers include chromatography using a chiral stationary phase (see, e.g., “Chiral Liquid Chromatography,” W. J. Lough, Ed. Chapman and Hall, New York (1989)). Enantiomers can also be separated by classical resolution techniques. For example, formation of diastereomeric salts and fractional crystallization can be used to separate enantiomers. For the separation of enantiomers of carboxylic acids, the diastereomeric salts can be formed by addition of enantiomerically pure chiral bases such as brucine, quinine, ephedrine, strychnine, and the like. Alternatively, diastereomeric esters can be formed with enantiomerically pure chiral alcohols such as menthol, followed by separation of the diastereomeric esters and hydrolysis to yield the free, enantiomerically enriched carboxylic acid. For separation of the optical isomers of amino compounds, addition of chiral carboxylic or sulfonic acids, such as camphorsulfonic acid, tartaric acid, mandelic acid, or lactic acid can result in formation of the diastereomeric salts.

### 3. Uses of the Vitamin D₃ Compounds of the Invention

In another aspect, the invention also provides methods for treating a subject for a vitamin D₃ associated state, by administering to the subject an effective amount of a vitamin D₃ compound of formula (I) or otherwise described herein. Vitamin D₃ associated states include disorders involving an aberrant activity of a vitamin D₃-responsive cell, e.g., neoplastic cells, hyperproliferative skin cells, parathyroid cells, immune cells and bone cells, among others. Vitamin D₃ associated states also include ILT3-associated disorders.

In current methods, the use of vitamin D₃ compounds has been limited because of their hypercalcemic effects. The Gemini vitamin D₃ compounds of the invention can provide a less toxic alternative to current methods of treatment.

In certain embodiments, the subject is a mammal, in particular a human.

In accordance with the methods of the invention, the Gemini vitamin D₃ compound can be administered in combination with a pharmaceutically diluent or acceptable carrier. In one embodiment, the vitamin D₃ compound can be administered using a pharmaceutically acceptable formulation. In advantageous embodiments, the pharmaceutically-acceptable carrier provides sustained delivery of the Gemini vitamin D₃ compound to a subject for at least four weeks after administration to the subject.

In certain embodiments, the Gemini vitamin D₃ compound is administered orally. In other embodiments, the vitamin D₃ compound is administered intravenously. In yet other embodiments, the vitamin D₃ compound is administered topically. In still other embodiments, the vitamin D₃ compound is administered topically is administered parenterally.

Although dosages may vary depending on the particular indication, route of administration and subject, the Gemini vitamin D₃ compounds are administered at a concentration of about 0.001 μg to about 100 μg/kg of body weight.

Another aspect of the invention comprises obtaining the vitamin D₃ compound of the invention.

### A. Hyperproliferative Conditions

In another aspect, the present invention provides a method of treating a subject for a disorder characterized by aberrant activity of a vitamin D₃-responsive cell. The method involves administering to the subject an effective amount of a pharmaceutical composition of a vitamin D₃ compound of the invention or otherwise described herein.

In certain embodiments, the cells to be treated are hyperproliferative cells. As described in greater detail below, the vitamin D₃ compounds of the invention can be used to inhibit the proliferation of a variety of hyperplastic and neoplastic tissues. In accordance with the present invention, vitamin D₃ compounds of the invention can be used in the treatment of both pathologic and non-pathologic proliferative conditions characterized by unwanted growth of vitamin D₃-responsive cells, e.g., hyperproliferative skin cells, immune cells, and tissue having transformed cells, e.g., such as carcinomas, sarcomas and leukemias. In other embodiments, the cells to be treated are aberrant secretory cells, e.g. parathyroid cells, immune cells.

In one embodiment, this invention features a method for inhibiting the proliferation and/or inducing the differentiation of a hyperproliferative skin cell, e.g., an epidermal or an epithelial cell, e.g. a keratinocytes, by contacting the cells with a vitamin D₃ compound of the invention. In general, the method includes a step of contacting a pathological or non-pathological hyperproliferative cell with an effective amount of such vitamin D₃ compound to promote the differentiation of the hyperproliferative cells The present method can be performed on cells in culture, e.g. in vitro or ex vivo, or can be performed on cells present in an animal subject, e.g., as part of an in vivo therapeutic protocol. The therapeutic regimen can be carried out on a human or any other animal subject.

The vitamin D₃ compounds of the present invention can be used to treat a hyperproliferative skin disorder. Exemplary disorders include, but are not limited to, psoriasis, basal cell carcinoma, keratinization disorders and keratosis. Additional examples of these disorders include eczema; lupus associated skin lesions; psoriatic arthritis; rheumatoid arthritis that involves hyperproliferation and inflammation of epithelial-related cells lining the joint capsule; dermatitides such as seborrheic dermatitis and solar dermatitis; keratoses such as seborrheic keratosis, senile keratosis, actinic keratosis, photo-induced keratosis, and keratosis follicularis; acne vulgaris; keloids and prophylaxis against keloid formation; nevi; warts including verruca, condyloma or condyloma acuminatum, and human papilloma viral (HPV) infections such as venereal warts; leukoplakia; lichen planus; and keratitis.

In an illustrative example, vitamin D₃ compounds of the invention can be used to inhibit the hyperproliferation of keratinocytes in treating diseases such as psoriasis by administering an effective amount of these compounds to a subject in need of treatment. The term “psoriasis” is intended to have its medical meaning, namely, a disease which afflicts primarily the skin and produces raised, thickened, scaling, nonscarring lesions. The lesions are usually sharply demarcated erythematous papules covered with overlapping shiny scales. The scales are typically silvery or slightly opalescent. Involvement of the nails frequently occurs resulting in pitting, separation of the nail, thickening and discoloration. Psoriasis is sometimes associated with arthritis, and it may be crippling. Hyperproliferation of keratinocytes is a key feature of psoriatic epidermal hyperplasia along with epidermal inflammation and reduced differentiation of keratinocytes. Multiple mechanisms have been invoked to explain the keratinocyte hyperproliferation that characterizes psoriasis. Disordered cellular immunity has also been implicated in the pathogenesis of psoriasis.

### B. Neoplasia

The invention also features methods for inhibiting the proliferation and/or reversing the transformed phenotype of vitamin D₃-responsive hyperproliferative cells by contacting the cells with a vitamin D₃ compound of formula (I) or otherwise described herein. In general, the method includes a step of contacting pathological or non-pathological hyperproliferative cells with an effective amount of a vitamin D₃ compound of the invention for promoting the differentiation of the hyperproliferative cells. The present method can be performed on cells in culture, e.g., in vitro or ex vivo, or can be performed on cells present in an animal subject, e.g., as part of an in vivo therapeutic protocol. The therapeutic regimen can be carried out on a human or other subject.

The vitamin D₃ compounds of the invention or otherwise described herein can be tested initially in vitro for their inhibitory effects in the proliferation of neoplastic cells. Examples of cell lines that can be used are transformed cells, e.g., the human promyeloid leukemia cell line HL-60, and the human myeloid leukemia U-937 cell line (Abe E. et al. (1981) Proc. Natl. Acad. Sci. USA 78:4990-4994; Song L. N. and Cheng T. (1992) Biochem Pharmacol 43:2292-2295; Zhou J. Y. et al. (1989) Blood 74:82-93; U.S. Pat. No. 5,401,733, U.S. Pat. No. 5,087,619). Alternatively, the antitumoral effects of vitamin D₃ compounds of the invention can be tested in vivo using various animal models known in the art and summarized in Bouillon, R. et al. (1995) Endocrine Reviews 16(2):233 (Table E), which is incorporated by reference herein. For example, SL mice are routinely used in the art to test vitamin D₃ compounds of the invention as models for MI myeloid leukemia (Honma et al. (1983) Cell Biol. 80:201-204; Kasukabe T. et al. (1987) Cancer Res. 47:567-572); breast cancer studies can be performed in, for example, nude mice models for human MX1 (ER) (Abe J. et al. (1991) Endocrinology 129:832-837; other cancers, e.g., colon cancer, melanoma osteosarcoma, can be characterized in, for example, nude mice models as describe in (Eisman J. A. et al. (1987) Cancer Res. 47:21-25; Kawaura A. et al. (1990) Cancer Lett 55:149-152; Belleli A. (1992) Carcinogenesis 13:2293-2298; Tsuchiya H. et al. (1993) J. Orthopaed Res. 11: 122-130).

The subject method may also be used to inhibit the proliferation of hyperplastic/neoplastic cells of hematopoietic origin, e.g., arising from myeloid, lymphoid or erythroid lineages, or precursor cells thereof. For instance, the present invention contemplates the treatment of various myeloid disorders including, but not limited to, acute promyeloid leukemia (APML), acute myelogenous leukemia (AML) and chronic myelogenous leukemia (CML) (reviewed in Vaickus, L. (1991) Crit. Rev. in Oncol./Hemotol. 11:267-97). Lymphoid malignancies which may be treated by the subject method include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas contemplated by the treatment method of the present invention include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF) and Hodgkin's disease.

In certain embodiments, the vitamin D₃ compounds of the invention can be used in combinatorial therapy with conventional cancer chemotherapeutics. Conventional treatment regimens for leukemia and for other tumors include radiation, drugs, or a combination of both. In addition to radiation, the following drugs, usually in combinations with each other, are often used to treat acute leukemias: vincristine, prednisone, methotrexate, mercaptopurine, cyclophosphamide, and cytarabine. In chronic leukemia, for example, busulfan, melphalan, and chlorambucil can be used in combination. All of the conventional anti-cancer drugs are highly toxic and tend to make patients quite ill while undergoing treatment. Vigorous therapy is based on the premise that unless every leukemic cell is destroyed, the residual cells will multiply and cause a relapse.

The subject method can also be useful in treating malignancies of the various organ systems, such as affecting lung, breast, lymphoid, gastrointestinal, and urogenital tract as well as adenocarcinomas which include malignancies such as most colon cancers, renal-cell carcinoma, prostate cancer and/or testicular tumors, bladder cancer, non-small cell carcinoma of the lung, cancer of the small intestine and cancer of the esophagus.

According to the general paradigm of vitamin D₃ involvement in differentiation of transformed cells, exemplary solid tumors that can be treated according to the method of the present invention include vitamin D₃-responsive phenotypes of sarcomas and carcinomas such as, but not limited to: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, bladder cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

Determination of a therapeutically effective anti-neoplastic amount or a prophylactically effective anti-neoplastic amount of the vitamin D₃ compound of the invention, can be readily made by the physician or veterinarian (the “attending clinician”), as one skilled in the art, by the use of known techniques and by observing results obtained under analogous circumstances. The dosages may be varied depending upon the requirements of the patient in the judgment of the attending clinician, the severity of the condition being treated and the particular compound being employed. In determining the therapeutically effective antineoplastic amount or dose, and the prophylactically effective antineoplastic amount or dose, a number of factors are considered by the attending clinician, including, but not limited to: the specific hyperplastic/neoplastic cell involved; pharmacodynamic characteristics of the particular agent and its mode and route of administration; the desirder time course of treatment; the species of mammal; its size, age, and general health; the specific disease involved; the degree of or involvement or the severity of the disease; the response of the individual patient; the particular compound administered; the mode of administration; the bioavailability characteristics of the preparation administered; the dose regimen selected; the kind of concurrent treatment (i.e., the interaction of the vitamin D₃ compounds of the invention with other co-administered therapeutics); and other relevant circumstances. U.S. Pat. No. 5,427,916, for example, describes method for predicting the effectiveness of antineoplastic therapy in individual patients, and illustrates certain methods which can be used in conjunction with the treatment protocols of the instant invention.

Treatment can be initiated with smaller dosages which are less than the optimum dose of the compound. Thereafter, the dosage should be increased by small increments until the optimum effect under the circumstances is reached. For convenience, the total daily dosage may be divided and administered in portions during the day if desired. A therapeutically effective antineoplastic amount and a prophylactically effective anti-neoplastic amount of a vitamin D₃ compound of the invention is expected to vary from about 0.1 milligram per kilogram of body weight per day (mg/kg/day) to about 100 mg/kg/day.

Compounds which are determined to be effective for the prevention or treatment of tumors in animals, e.g., dogs, rodents, may also be useful in treatment of tumors in humans. Those skilled in the art of treating tumor in humans will know, based upon the data obtained in animal studies, the dosage and route of administration of the compound to humans. In general, the dosage and route of administration in humans is expected to be similar to that in animals.

The identification of those patients who are in need of prophylactic treatment for hyperplastic/neoplastic disease states is well within the ability and knowledge of one skilled in the art. Certain of the methods for identification of patients which are at risk of developing neoplastic disease states which can be treated by the subject method are appreciated in the medical arts, such as family history of the development of a particular disease state and the presence of risk factors associated with the development of that disease state in the subject patient. A clinician skilled in the art can readily identify such candidate patients, by the use of, for example, clinical tests, physical examination and medical/family history.

### C. Immunological Activity

Healthy individuals protect themselves against foreign invaders using many different mechanisms, including physical barriers, phagocytic cells in the blood and tissues, a class of immune cells known as lymphocytes, and various blood-born molecules. All of these mechanisms participate in defending individuals from a potentially hostile environment. Some of these defense mechanisms, known as natural or innate immunity, are present in an individual prior to exposure to infectious microbes or other foreign macromolecules, are not enhanced by such exposures, and do not discriminate among most foreign substances. Other defense mechanisms, known as acquired or specific immunity, are induced or stimulated by exposure of foreign substances, are exquisitely specific for distinct macromolecules, and increase in magnitude and defensive capabilities with each successive exposure to a particular macromolecule. Substances that induce a specific immune response are known as antigens (see, e.g., Abbas, A. et al., Cellular and Molecular Immunology, W.B. Saunders Company, Philadelphia, 1991; Silverstein, A. M. A history of Immunology, San Diego, Academic Press, 1989; Unanue A. et al., Textbook of Immunology, 2^{nd} ed. Williams and Wilkens, Baltimore, 1984).

One of the most remarkable properties of the immune system is its ability to distinguish between foreign antigens and self-antigens. Therefore, the lymphocytes in each individual are able to recognize and respond to many foreign antigens but are normally unresponsive to the potentially antigenic substances present in the individual. This immunological unresponsiveness is referred to as immune tolerance (see, e.g., Burt R K et al. (2002) Blood 99:768; Coutinho, A. et al. (2001) Immunol. Rev. 182:89; Schwartz, R H (1990) Science 248:1349; Miller, J. F. et al. (1989) Immunology Today 10:53).

Self-tolerance is an acquired process that has to be learned by the lymphocytes of each individual. It occurs in part because lymphocytes pass through a stage in their development when an encounter with antigen presented by antigen-presenting cells (APCs) leads to their death or inactivation in a process known as positive and negative selection (see, e.g., Debatin K M (2001) Ann. Hematol. 80 Suppl 3:B29; Abbas, A. (1991), supra). Thus, potentially self-recognizing lymphocytes come into contact with self-antigens at this stage of functional immaturity and are prevented from developing to a stage at which they would be able to respond to self-antigens. Autoimmunity arises when abnormalities in the induction or maintenance of self-tolerance occur that result in a loss of tolerance to a particular antigen(s) and a subsequent attack by the host's immune system on the host's tissues that express the antigen(s) (see, e.g., Boyton R J et al. (2002) Clin. Exp. Immunol. 127:4; Hagiwara E. (2001) Ryumachi 41:888; Burt R K et al. (2992) Blood 99:768).

The ability of the immune system to distinguish between self and foreign antigens also plays a critical role in tissue transplantation. The success of a transplant depends on preventing the immune system of the host recipient from recognizing the transplant as foreign and, in some cases, preventing the graft from recognizing the host tissues as foreign. For example, when a host receives a bone marrow transplant, the transplanted bone marrow may recognize the new host as foreign, resulting in graft versus host disease (GVHD). Consequently, the survival of the host depends on preventing both the rejection of the donor marrow as well as rejection of the host by the graft immune reaction (see, e.g., Waldmann H et al. (2001) Int. Arch. Allergy Immunol. 126:11).

Currently, deleterious immune reactions that result in autoimmune diseases and transplant rejections are prevented or treated using agents such as steroids, azathioprine, anti-T cell antibodies, and more recently, monoclonal antibodies to T cell subpopulations. Immunosuppressive drugs such as cyclosporin A (CsA), rapamycin, desoxyspergualine and FK-506 are also widely used.

Nonspecific immune suppression agents, such as steroids and antibodies to lymphocytes, put the host at increased risk for opportunistic infection and development of tumors. Moreover, many immunosuppressive drugs result in bone demineralization within the host (see, e.g., Chhajed P N et al. (2002) Indian J. Chest Dis. Allied 44:31; Wijdicks E F (2001) Liver Transpl. 7:937; Karamehic J et al. (2001) Med. Arh. 55:243; U.S. Pat. No. 5,597,563 issued to Beschorner, W E and U.S. Pat. No. 6,071,897 issued to DeLuca H F et al.). Because of the major drawbacks associated with existing immunosuppressive modalities, there is a need for a new approach for treating immune disorders, e.g., for inducing immune tolerance in a host.

Thus, in another aspect, the invention provides a method for modulating the activity of an immune cell by contacting the cell with a vitamin D₃ compound of the invention or otherwise described herein.

In one embodiment the invention provides a method of modulating the expression of an immunoglobulin-like transcript 3 (ILT3) surface molecule in a cell, comprising contacting said cell with a vitamin D3 compound of described herein above in an amount effective to modulate the expression of an immunoglobulin-like transcript 3 (ILT3) surface molecule in said cell. In certain embodiments, the cell is within a subject.

A related embodiment of the invention provides a method of inducing immunological tolerance in a subject, comprising administering to said subject a vitamin D₃ compound described herein above in an amount effective to modulate the expression of an ILT3 surface molecule, thereby inducing immunological tolerance in said subject. Another embodiment of the invention provides a method for modulating immunosuppressive activity by an antigen-presenting cell, comprising contacting an antigen-presenting cell with a vitamin D3 compound described herein above in an amount effective to modulate ILT3 surface molecule expression, thereby modulating said immunosuppressive activity by said antigen-presenting cell.

In certain embodiments, the target of the methods is an antigen-presenting cell. Antigen-presenting cells include dendritic cells, monocytes, and macrophages.

In yet other embodiments, the expression of said immunoglobulin-like transcript 3 (ILT3) surface molecules is upregulated. In a further embodiment, the cell is an antigen-presenting cell. In a further embodiment, the cell is selected from the group consisting of dendritic cells, monocytes, and macrophages.

In one embodiment, the invention provides a method for treating a vitamin D3 associated state, wherein the associated state is an ILT3-associated disorder. The present invention provides a method for suppressing immune activity in an immune cell by contacting a pathological or non-pathological immune cell with an effective amount of a vitamin D₃ compound of the invention to thereby inhibit an immune response relative to the cell in the absence of the treatment. The present method can be performed on cells in culture, e.g., in vitro or ex vivo, or can be performed on cells present in an animal subject, e.g., as part of an in vivo therapeutic protocol. In vivo treatment can be carried out on a human or other animal subject.

In another embodiment, the invention provides a method of treating an ILT3-associated disorder, comprising administering to a subject a compound of formula I or I-a in an amount effective to modulate the expression of an ILT3 surface molecule. In a further embodiment, the ILT3-associated disorder is an immune disorder. In a further embodiment, the immune disorder is an autoimmune disorder. In a further embodiment, the disorder is type 1 insulin dependent diabetes mellitus.

In another embodiment, the invention provides a method of modulating immunosuppressive activity by an antigen-presented cell, comprising contacting an antigen-presenting cell with a compound of the invention.

In another embodiment, the invention provides a method of inhibiting transplant rejection in a subject comprising administering to the subject a compound of formula I or I-a in an amount effective to modulate the expression of an ILT3 surface molecule, thereby inhibiting transplant rejection. In a further embodiment, the transplant is a solid organ transplant, a pancreatic islet transplant, or a bone marrow transplant.

The vitamin D₃ compounds of the invention can be tested initially in vitro for their inhibitory effects on T cell proliferation and secretory activity, as described in Reichel, H. et al., (1987) Proc. Natl. Acad. Sci. USA 84:3385-3389; Lemire, J. M. et al. (1985) J. Immunol 34:2032-2035. Alternatively, the immunosuppressive effects can be tested in vivo using the various animal models known in the art and summarized by Bouillon, R. et al. (1995) Endocrine Reviews 16(2) 232 (Tables 6 and 7). For examples, animal models for autoimmune disorders, e.g., lupus, thyroiditis, encephalitis, diabetes and nephritis are described in (Lemire J. M. (1992) J. Cell Biochem. 49:26-31; Koizumi T. et al. (1985) Int. Arch. Allergy Appl. Immunol. 77:396-404; Abe J. et al. (1990) Calcium Regulation and Bone Metabolism 146-151; Fournier C. et al. (1990) Clin. Immunol Immunopathol. 54:53-63; Lemire J. M. and Archer D. C. (1991) J. Clin. Invest. 87:1103-1107); Lemire, J. M. et al., (1994) Endocrinology 135 (6):2818-2821; Inaba M. et al. (1992) Metabolism 41:631-635; Mathieu C. et al. (1992) Diabetes 41:1491-1495; Mathieu C. et al. (1994) Diabetologia 37:552-558; Lillevang S. T. et al. (1992) Clin. Exp. Immunol. 88:301-306, among others). Models for characterizing immunosuppressive activity during organ transplantation, e.g., skin graft, cardiac graft, islet graft, are described in Jordan S. C. et al. (1988) v Herrath D (eds) Molecular, Cellular and Clinical Endocrinology 346-347; Veyron P. et al. (1993) Transplant Immunol. 1:72-76; Jordan S. C. (1988) v Herrath D (eds) Molecular, Cellular and Clinical Endocrinology 334-335; Lemire J. M. et al. (1992) Transplantation 54:762-763; Mathieu C. et al. (1994) Transplant Proc. 26:3128-3129).

After identifying certain test compounds as effective suppressors of an immune response in vitro, these compounds can be used in vivo as part of a therapeutic protocol. Accordingly, another embodiment provides a method of suppressing an immune response, comprising administering to a subject a pharmaceutical preparation of a vitamin D₃ compounds of the invention, so as to inhibit immune reactions such as graft rejection, autoimmune disorders and inflammation.

For example, the subject vitamin D₃ compound of the invention can be used to inhibit responses in clinical situations where it is desirable to downmodulate T cell responses. For example, in graft-versus-host disease, cases of transplantation, autoimmune diseases (including, for example, diabetes mellitus, type-1 insulin dependent diabetes mellitus, adult respiratory distress syndrome, inflammatory bowel disease, meningitis, thrombotic thrombocytopenic purpura, encephalitis, uveitis, uveoretinitis, leukocyte adhesion deficiency, rheumatoid arthritis, rheumatic fever, Reiter's syndrome, psoriatic arthritis, progressive systemic sclerosis, primary biliary cirrhosis, pemphigus, pemphigoid, necrotizing vasculitis, myasthenia gravis, multiple sclerosis, lupus erythematosus, polymyositis, sarcoidosis, granulomatosis, vasculitis, pernicious anemia, CNS inflammatory disorder, antigen-antibody complex mediated diseases, autoimmune haemolytic anemia, Hashimoto's thyroiditis, Graves disease, habitual spontaneous abortions, Reynard's syndrome, glomerulonephritis, dermatomyositis, chronic active hepatitis, celiac disease, autoimmune complications of AIDS, atrophic gastritis, ankylosing spondylitis, Addison's disease, arthritis (including rheumatoid arthritis, juvenile rheumatoid arthritis, osteoarthritis, psoriatic arthritis), multiple sclerosis, encephalomyelitis, diabetes, myasthenia gravis, systemic lupus erythematosis, autoimmune thyroiditis, dermatitis (including atopic dermatitis and eczematous dermatitis), psoriasis, Sjögren's Syndrome, including keratoconjunctivitis sicca secondary to Sjögren's Syndrome, alopecia areata, allergic responses due to arthropod bite reactions, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Crohn's disease, Graves opthalmopathy, sarcoidosis, primary biliary cirrhosis, uveitis posterior, and interstitial lung fibrosis). Downmodulation of immune activity will also be desirable in cases of allergy such as, atopic allergy.

In such embodiments, the present invention provides methods and compositions for treating immune disorders, such as, for example, autoimmune disorders and transplant rejections, such as graft versus host disease (GVHD). These embodiments of the invention are based on the discovery that vitamin D compounds of the invention are able to modulate the expression of immunoglobulin-like transcript 3 (ILT3) on cells, e.g., antigen-presenting cells.

As described before, determination of a therapeutically effective immunosuppressive amount can be readily made by the attending clinician, as one skilled in the art, by the use of known techniques and by observing results obtained under analogous circumstances. Compounds which are determined to be effective in animals, e.g., dogs, rodents, may be extrapolated accordingly to humans by those skilled in the art. Starting dose/regimen used in animals can be estimated based on prior studies. For example, doses of vitamin D₃ compounds of the invention to treat autoimmune disorders in rodents can be initially estimated in the range of 0.1 g/kg/day to 1 g/kg/day, administered orally or by injection.

Those skilled in the art will know based upon the data obtained in animal studies, the dosage and route of administration in humans is expected to be similar to that in animals. Exemplary dose ranges to be used in humans are from 0.25 to 10 μg/day, preferably 0.5 to 5 μg/day per adult (U.S. Pat. No. 4,341,774).

### D. Calcium and Phosphate Homeostasis

The present invention also relates to a method of treating in a subject a disorder characterized by deregulation of calcium and phosphate metabolism. This method comprises contacting a pathological or non-pathological vitamin D₃ responsive cell with an effective amount of a vitamin D₃ compound of the invention to thereby directly or indirectly modulate calcium and phosphate homeostasis. Techniques for detecting calcium fluctuation in vivo or in vitro are known in the art. In one embodiment, the invention provides a method to ameliorate a deregulation of calcium and phosphate metabolism that leads to osteoporosis.

Exemplary Ca⁺⁺ homeostasis related assays include assays that focus on the intestine where intestinal ⁴⁵Ca²⁺ absorption is determined either 1) in vivo (Hibberd K. A. and Norman A. W. (1969) Biochem. Pharmacol. 18:2347-2355; Hurwitz S. et al. (1967) J. Nutr. 91:319-323; Bickle D. D. et al. (1984) Endocrinology 114:260-267), or 2) in vitro with everted duodenal sacs (Schachter D. et al. (1961) Am. J. Physiol 200:1263-1271), or 3) on the genomic induction of calbindin-D₂₈ₖ in the chick or of calbindin-D₉ₖ in the rat (Thomasset M. et al. (1981) FEBS Lett. 127:13-16; Brehier A. and Thomasset M. (1990) Endocrinology 127:580-587). The bone-oriented assays include: 1) assessment of bone resorption as determined via the release of Ca²⁺ from bone in vivo (in animals fed a zero Ca²⁺ diet) (Hibberd K. A. and Norman A. W. (1969) Biochem. Pharmacol. 18:2347-2355; Hurwitz S. et al. (1967) J. Nutr. 91:319-323), or from bone explants in vitro (Bouillon R. et al. (1992) J. Biol. Chem. 267:3044-3051), 2) measurement of serum osteocalcin levels [osteocalcin is an osteoblast-specific protein that after its synthesis is largely incorporated into the bone matrix, but partially released into the circulation (or tissue culture medium) and thus represents a good market of bone formation or turnover] (Bouillon R. et al. (1992) Clin. Chem. 38:2055-2060), or 3) bone ash content (Norman A. W. and Wong R. G. (1972) J. Nutr. 102:1709-1718). Only one kidney-oriented assay has been employed. In this assay, urinary Ca²⁺ excretion is determined (Hartenbower D. L. et al. (1977) Walter de Gruyter, Berlin pp 587-589); this assay is dependent upon elevations in the serum Ca²⁺ level and may reflect bone Ca²⁺ mobilizing activity more than renal effects. Finally, there is a “soft tissue calcification” assay that can be used to detect the consequences of administration of a compound of the invention. In this assay a rat is administered an intraperitoneal dose of ⁴⁵Ca²⁺, followed by seven daily relative high doses of a compound of the invention; in the event of onset of a severe hypercalcemia, soft tissue calcification can be assessed by determination of the ⁴⁵Ca²⁺ level. In all these assays, vitamin D₃ compounds of the invention are administered to vitamin D-sufficient or -deficient animals, as a single dose or chronically (depending upon the assay protocol), at an appropriate time interval before the end point of the assay is quantified.

In certain embodiments, vitamin D₃ compounds of the invention can be used to modulate bone metabolism. The language “bone metabolism” is intended to include direct or indirect effects in the formation or degeneration of bone structures, e.g., bone formation, bone resorption, etc., which may ultimately affect the concentrations in serum of calcium and phosphate. This term is also intended to include effects of vitamin D₃ compounds in bone cells, e.g. osteoclasts and osteoblasts, that may in turn result in bone formation and degeneration. For example, it is known in the art, that vitamin D₃ compounds exert effects on the bone forming cells, the osteoblasts through genomic and non-genomic pathways (Walters M. R. et al. (1982) J. Biol. Chem. 257:7481-7484; Jurutka P. W. et al. (1993) Biochemistry 32:8184-8192; Mellon W. S. and DeLuca H. F. (1980) J. Biol. Chem. 255:4081-4086). Similarly, vitamin D₃ compounds are known in the art to support different activities of bone resorbing osteoclasts such as the stimulation of differentiation of monocytes and mononuclear phagocytes into osteoclasts (Abe E. et al. (1988) J. Bone Miner Res. 3:635-645; Takahashi N. et al. (1988) Endocrinology 123:1504-1510; Udagawa N. et al. (1990) Proc. Natl. Acad. Sci. USA 87:7260-7264). Accordingly, vitamin D₃ compounds of the invention that modulate the production of bone cells can influence bone formation and degeneration.

The present invention provides a method for modulating bone cell metabolism by contacting a pathological or a non-pathological bone cell with an effective amount of a vitamin D₃ compound of the invention to thereby modulate bone formation and degeneration. The present invention provides a method for treating aberrant activity of a bone cell. The present method can be performed on cells in culture, e.g., in vitro or ex vivo, or can be performed in cells present in an animal subject, e.g., cells in vivo. Exemplary culture systems that can be used include osteoblast cell lines, e.g., ROS 17/2.8 cell line, monocytes, bone marrow culture system (Suda T. et al. (1990) Med. Res. Rev. 7:333-366; Suda T. et al. (1992) J. Cell Biochem. 49:53-58) among others. Selected compounds can be further tested in vivo, for example, animal models of osteopetrosis and in human disease (Shapira F. (1993) Clin. Orthop. 294:34-44).

In a preferred embodiment, a method for treating osteoporosis is provided, comprising administering to a subject a pharmaceutical preparation of a vitamin D₃ compound of the invention to thereby ameliorate the condition relative to an untreated subject.

Vitamin D₃ compounds of the invention can be tested in ovarectomized animals, e.g., dogs, rodents, to assess the changes in bone mass and bone formation rates in both normal and estrogen-deficient animals. Clinical trials can be conducted in humans by attending clinicians to determine therapeutically effective amounts of the vitamin D₃ compounds of the invention in preventing and treating osteoporosis.

In other embodiments, therapeutic applications of the vitamin D₃ compounds of the invention include treatment of other diseases characterized by metabolic calcium and phosphate deficiencies. Exemplary of such diseases are the following: osteoporosis, osteodystrophy, senile osteoporosis, osteomalacia, rickets, osteitis fibrosa cystica, renal osteodystrophy, osteosclerosis, anti-convulsant treatment, osteopenia, fibrogenesis-imperfecta ossium, secondary hyperparathyrodism, hypoparathyroidism, hyperparathyroidism, cirrhosis, obstructive jaundice, drug induced metabolism, medullary carcinoma, chronic renal disease, hypophosphatemic VDRR, vitamin D-dependent rickets, sarcoidosis, glucocorticoid antagonism, malabsorption syndrome, steatorrhea, tropical sprue, idiopathic hypercalcemia and milk fever.

### E. Hormone Secretion

In yet another aspect, the present invention provides a method for treating aberrant activity of an endocrine cell. In a further embodiment, the endocrine cell is aparathyroid cell and the aberrant activity is processing or section of parathyroid hormone. Hormone secretion includes both genomic and non-genomic activities of vitamin D₃ compounds of the invention that control the transcription and processing responsible for secretion of a given hormone e.g., parathyroid hormone (PTH), calcitonin, insulin, prolactin (PRL) and TRH in a vitamin D₃ responsive cell (Bouillon, R. et al. (1995) Endocrine Reviews 16(2):235-237).

The present method can be performed on cells in culture, e.g. in vitro or ex vivo, or on cells present in an animal subject, e.g., in vivo. Vitamin D₃ compounds of the invention can be initially tested in vitro using primary cultures of parathyroid cells. Other systems that can be used include the testing by prolactin secretion in rat pituitary tumor cells, e.g., GH4C1 cell line (Wark J. D. and Tashjian Jr. A. H. (1982) Endocrinology 111:1755-1757; Wark J. D. and Tashjian Jr. A. H. (1983) J. Biol. Chem. 258:2118-2121; Wark J. D. and Gurtler V. (1986) Biochem. J. 233:513-518) and TRH secretion in GH4C1 cells. Alternatively, the effects of vitamin D₃ compounds of the invention can be characterized in vivo using animals models as described in Nko M. et al. (1982) Miner Electrolyte Metab. 5:67-75; Oberg F. et al. (1993) J. Immunol. 150:3487-3495; Bar-Shavit Z. et al. (1986) Endocrinology 118:679-686; Testa U. et al. (1993) J. Immunol. 150:2418-2430; Nakamaki T. et al. (1992) Anticancer Res. 12:1331-1337; Weinberg J. B. and Larrick J. W. (1987) Blood 70:994-1002; Chambaut-Guérin A. M. and Thomopoulos P. (1991) Eur. Cytokine New. 2:355; Yoshida M. et al. (1992) Anticancer Res. 12:1947-1952; Momparler R. L. et al. (1993) Leukemia 7:17-20; Eisman J. A. (1994) Kanis J A (eds) Bone and Mineral Research 2:45-76; Veyron P. et al. (1993) Transplant Immunol. 1:72-76; Gross M. et al. (1986) J Bone Miner Res. 1:457-467; Costa E. M. et al. (1985) Endocrinology 117:2203-2210; Koga M. et al. (1988) Cancer Res. 48:2734-2739; Franceschi R. T. et al. (1994) J. Cell Physiol. 123:401-409; Cross H. S. et al. (1993) Naunyn Schmiedebergs Arch. Pharmacol. 347:105-110; Zhao X. and Feldman D. (1993) Endocrinology 132:1808-1814; Skowronski R. J. et al. (1993) Endocrinology 132:1952-1960; Henry H. L. and Norman A. W. (1975) Biochem. Biophys. Res. Commun. 62:781-788; Wecksler W. R. et al. (1980) Arch. Biochem. Biophys. 201:95-103; Brumbaugh P. F. et. al. (1975) Am. J. Physiol. 238:384-388; Oldham S. B. et al. (1979) Endocrinology 104:248-254; Chertow B. S. et al. (1975) J. Clin Invest. 56:668-678; Canterbury J. M. et al. (1978) J. Clin. Invest. 61:1375-1383; Quesad J. M. et al. (1992) J. Clin. Endocrinol. Metab. 75:494-501.

In certain embodiments, the vitamin D₃ compounds of the present invention can be used to inhibit parathyroid hormone (PTH) processing, e.g., transcriptional, translational processing, and/or secretion of a parathyroid cell as part of a therapeutic protocol. Therapeutic methods using these compounds can be readily applied to all diseases, involving direct or indirect effects of PTH activity, e.g., primary or secondary responses or secondary hyperparathyroidism.

Accordingly, therapeutic applications for the vitamin D₃ compounds of the invention include treating diseases such as secondary hyperparathyroidism of chronic renal failure (Slatopolsky E. et al. (1990) Kidney Int. 38:S41-S47; Brown A. J. et al. (1989) J. Clin. Invest. 84:728-732). Determination of therapeutically affective amounts and dose regimen can be performed by the skilled artisan using the data described in the art.

### F. Protection Against Neuronal Loss

In yet another aspect, the present invention provides a method of protecting against neuronal loss. The language “protecting against” is intended to include prevention, retardation, and/or termination of deterioration, impairment, or death of a neurons.

Neuron loss can be the result of any condition of a neuron in which its normal function is compromised. Neuron deterioration can be the result of any condition which compromises neuron function which is likely to lead to neuron loss. Neuron function can be compromised by, for example, altered biochemistry, physiology, or anatomy of a neuron. Deterioration of a neuron may include membrane, dendritic, or synaptic changes which are detrimental to normal neuronal functioning. The cause of the neuron deterioration, impairment, and/or death may be unknown. Alternatively, it may be the result of age- and/or disease-related changes which occur in the nervous system of a subject.

When neuron loss is described herein as “age-related”, it is intended to include neuron loss resulting from known and unknown bodily changes of a subject which are associated with aging. When neuron loss is described herein as “disease-related”, it is intended to include neuron loss resulting from known and unknown bodily changes of a subject which are associated with disease. It should be understood, however, that these terms are not mutually exclusive and that, in fact, many conditions that result in the loss of neurons are both age- and disease-related.

Exemplary age-related diseases associated with neuron loss and changes in neuronal morphology include, for example, Alzheimer's Disease, Pick's Disease, Parkinson's Disease, Vascular Disease, Huntington's Disease, and Age-Associated Memory Impairment. In Alzheimer's Disease patients, neuron loss is most notable in the hippocampus, frontal, parietal, and anterior temporal cortices, amygdala, and the olfactory system. The most prominently affected zones of the hippocampus include the CA1 region, the subiculum, and the entorhinal cortex. Memory loss is considered the earliest and most representative cognitive change because the hippocampus is well known to play a crucial role in memory. Pick's Disease is characterized by severe neuronal degeneration in the neocortex of the frontal and anterior temporal lobes which is sometimes accompanied by death of neurons in the striatum. Parkinson's Disease can be identified by the loss of neurons in the substantia nigra and the locus ceruleus. Huntington's Disease is characterized by degeneration of the intrastriatal and cortical cholinergic neurons and GABA-ergic neurons. Parkinson's and Huntington's Diseases are usually associated with movement disorders, but often show cognitive impairment (memory loss) as well.

Age-Associated Memory Impairment (AAMI) is another age-associated disorder that is characterized by memory loss in healthy, elderly individuals in the later decades of life. Crook, T. et al. (1986) Devel. Neuropsych. 2(4):261-276. Presently, the neural basis for AAMI has not been precisely defined. However, neuron death with aging has been reported to occur in many species in brain regions implicated in memory, including cortex, hippocampus, amygdala, basal ganglia, cholinergic basal forebrain, locus ceruleus, raphe nuclei, and cerebellum. Crook, T. et al. (1986) Devel. Neuropsych. 2(4):261-276.

Vitamin D₃ compounds of the invention can protect against neuron loss by genomic or non-genomic mechanisms. Nuclear vitamin D₃ receptors are well known to exist in the periphery but have also been found in the brain, particularly in the hippocampus and neocortex. Non-genomic mechanisms may also prevent or retard neuron loss by regulating intraneuronal and/or peripheral calcium and phosphate levels. Furthermore, vitamin D₃ compounds of the invention may protect against neuronal loss by acting indirectly, e.g., by modulating serum PTH levels. For example, a positive correlation has been demonstrated between serum-PTH levels and cognitive decline in Alzheimer's Disease.

The present method can be performed on cells in culture, e.g. in vitro or ex vivo, or on cells present in an animal subject, e.g., in vivo. Vitamin D₃ compounds of the invention can be initially tested in vitro using neurons from embryonic rodent pups (See e.g. U.S. Pat. No. 5,179,109-fetal rat tissue culture), or other mammalian (See e.g. U.S. Pat. No. 5,089,517-fetal mouse tissue culture) or non-mammalian animal models. These culture systems have been used to characterize the protection of peripheral, as well as, central nervous system neurons in animal or tissue culture models of ischemia, stroke, trauma, nerve crush, Alzheimer's Disease, Pick's Disease, and Parkinson's Disease, among others. Examples of in vitro systems to study the prevention of destruction of neocortical neurons include using in vitro cultures of fetal mouse neurons and glial cells previously exposed to various glutamate agonists, such as kainate, NMDA, and α-amino-3-hydroxy-5-methyl-4-isoxazolepronate (AMPA). U.S. Pat. No. 5,089,517. See also U.S. Pat. No. 5,170,109 (treatment of rat cortical/hippocampal neuron cultures with glutamate prior to treatment with neuroprotective compound); U.S. Pat. Nos. 5,163,196 and 5,196,421 (neuroprotective excitatory amino acid receptor antagonists inhibit glycine, kainate, AMPA receptor binding in rats).

Alternatively, the effects of vitamin D₃ compounds of the invention can be characterized ins vivo using animals models. Neuron deterioration in these model systems is often induced by experimental trauma or intervention (e.g. application of toxins, nerve crush, interruption of oxygen supply).

### G. Smooth Muscle Cells

In yet another aspect, the present invention provides a method of treating disorders characterized by the aberrant activity of a vascular smooth muscle cell by contacting a vitamin D₃-responsive smooth muscle cell with a vitamin D₃ compound of the invention to activate or, preferably, inhibit the activity of the cell. The language “activity of a smooth muscle cell” is intended to include any activity of a smooth muscle cell, such as proliferation, migration, adhesion and/or metabolism.

In certain embodiments, the vitamin D₃ compounds of the invention can be used to treat diseases and conditions associated with aberrant activity of a vitamin D₃-responsive smooth muscle cell. For example, the present invention can be used in the treatment of hyperproliferative vascular diseases, such as hypertension induced vascular remodeling, vascular restenosis and atherosclerosis. In other embodiments, the compounds of the present invention can be used in treating disorders characterized by aberrant metabolism of a vitamin D₃-responsive smooth muscle cell, e.g., arterial hypertension.

The present method can be performed on cells in culture, e.g. in vitro or ex vivo, or on cells present in an animal subject, e.g., in vivo. Vitamin D₃ compounds of the invention can be initially tested in vitro as described in Catellot et al. (1982), J. Biol. Chem. 257(19): 11256.

### H. Suppression of Renin Expression and Treatment of Hypertension

The compounds of the present invention control blood pressure by the suppression of rennin expression and are useful as antihypertensive agents. Renin-angiotensin regulatory cascade plays a significant role in the regulation of blood pressure, electrolyte and volume homeostasis (Y. C. Li, Abstract, DeLuca Symposium on Vitamin D₃, Tauc, N. Mex., Jun. 15-Jun. 19, 2002, p. 18). Thus, the invention provides a method of treating hypertension. The method comprises administering to said subject an effective amount of a Gemini vitamin D₃ compound, such that said subject is treated for hypertension. In accordance with an embodiment of the method, the Gemini vitamin D₃ compound suppresses expression of renin, thereby treating the subject for hypertension.

In a related embodiment, the invention provides a method of suppressing renin expression in a subject comprising administering to a subject an effective amount of a Gemini vitamin D₃ compound such that renin expression in said subject is suppressed.

### I. Treatment of Urogenital Disorders

The invention also provides a method for treating a subject for a urogenital disorder. The method comprises administering to the subject an effective amount of a vitamin D₃ compound of the invention, such that the subject is treated for the urogential disorder.

In one embodiment, the urogenital disorder comprises bladder dysfunction, especially bladder dysfunction related to morphological bladder changes. The term bladder dysfunction as used in this embodiment does not include cancer of the bladder and associated urogenital organs.

Morphological bladder changes, including a progressive de-nervation and hypertrophy of the bladder wall are frequent histological findings in patients with different bladder disorders such as overactive bladder and clinical BPH. The increase in tension and/or strain on the bladder observed in these conditions has been shown to be associated with cellular and molecular alterations, e.g., in cytoskeletal and contractile proteins, in mitochondrial function, and in various enzyme activities of the smooth muscle cells. The growth of the bladder wall also involves alterations in its extracellular matrix and non-smooth muscle components.

These changes in the bladder are associated with the storage (irritative) symptoms, in particular frequency, urgency and nocturia. These symptoms affect the social, psychological, domestic, occupational, physical and sexual lives of the patients leading to a profound, negative impact on their quality of life.

Included within urogenital disorders is bladder function characterized by the presence of bladder hypertrophy.

Also included within urogenital disorders is benign prostatic hyperplasia (BPH). Thus the invention also provides a method for treatment of BPH comprising administering to a subject an effective amount of a vitamin D₃ compound of formula I or I-a above, such that the subject is treated for BPH.

BPH is commonly associated with enlargement of the gland (prostate) leading to bladder outlet obstruction (BOO) and symptoms secondary to BOO. However, BPH is also associated with morphological bladder changes, including a progressive denervation and hypertrophy of the bladder wall, the latter possibly as a consequence of increased functional demands. Thus, the compounds of the invention are useful for the treatment of storage (initiative) symptoms of BPH, as well as for bladder outlet obstruction caused by BPH.

Urogenital disorders in accordance with the invention also include interstitial cystitis. Thus, in another embodiment, the invention also provides a method for treatment of interstitial cystitis comprising administering to a subject an effective amount of a vitamin D₃ compound of the invention, such that the subject is treated for interstitial cystitis.

Interstitial cystitis (IC) is a chronic inflammatory bladder disease characterized by pelvic pain, urinary urgency and frequency. Unlike other bladder dysfunction conditions, IC is characterized by chronic inflammation of the bladder wall which is responsible for the symptomatology. In other words, the cause of the abnormal bladder contractility is the chronic inflammation and as a consequence the treatment should target this etiological component. In fact, the traditional treatment of bladder dysfunctions, like overactive bladder, with smooth muscle relaxant agents, is not effective in patients with IC.

Another aspect of the invention is a method for treating bladder disfunction in a subject, by administering an effective amount of a compound of the invention. In one embodiment, the compound is a vitamin D receptor agonist. In another embodiment, the bladder disfunction is characterized by the presence of bladder hypertrophy. In another embodiment, the bladder disfunction is overactive bladder. In a further embodiment, the subject is male, and can currently suffer from BPH.

### 4. Pharmaceutical Compositions

The invention also provides a pharmaceutical composition, comprising an effective amount a vitamin D₃ compound of the invention or otherwise described herein and a pharmaceutically acceptable carrier. In a further embodiment, the effective amount is effective to treat a vitamin D₃ associated state, as described previously.

In an embodiment, the vitamin D₃ compound is administered to the subject using a pharmaceutically-acceptable formulation, e.g., a pharmaceutically-acceptable formulation that provides sustained delivery of the vitamin D₃ compound to a subject for at least 12 hours, 24 hours, 36 hours, 48 hours, one week, two weeks, three weeks, or four weeks after the pharmaceutically-acceptable formulation is administered to the subject.

In certain embodiments, these pharmaceutical compositions are suitable for topical or oral administration to a subject. In other embodiments, as described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound.

In certain embodiments, the subject is a mammal, e.g. a primate, e.g., a human.

The methods of the invention further include administering to a subject a therapeutically effective amount of a vitamin D₃ compound in combination with another pharmaceutically active compound. Examples of pharmaceutically active compounds include compounds known to treat autoimmune disorders, e.g., immunosuppressant agents such as cyclosporin A, rapamycin, desoxyspergualine, FK 506, steroids, azathioprine, anti-T cell antibodies and monoclonal antibodies to T cell subpopulations. Other pharmaceutically active compounds that may be used can be found in Harrison's Principles of Internal Medicine, Thirteenth Edition, Eds. T. R. Harrison et al. McGraw-Hill N.Y., NY; and the Physicians Desk Reference 50th Edition 1997, Oradell N.J., Medical Economics Co., the complete contents of which are expressly incorporated herein by reference. The angiogenesis inhibitor compound and the pharmaceutically active compound may be administered to the subject in the same pharmaceutical composition or in different pharmaceutical compositions (at the same time or at different times).

The phrase “pharmaceutically acceptable” is refers to those vitamin D₃ compounds of the present invention, compositions containing such compounds, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” includes pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water, (17) isotonic saline; (18) Ringer's solution; (19) ethyl alcohol; (20) phosphate buffer solutions; and (21) other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Compositions containing a vitamin D₃ compound(s) include those suitable for oral, nasal, topical (including buccal and sublingual), rectal, vaginal, aerosol and/or parenteral administration. The compositions may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these compositions include the step of bringing into association a vitamin D₃ compound(s) with the carrier and, optionally, one or more accessory ingredients. In general, the formulations are prepared by uniformly and intimately bringing into association a vitamin D₃ compound with liquid carriers, or finely divided solid carriers, or both, and then, if necessary, shaping the product.

Compositions of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a vitamin D₃ compound(s) as an active ingredient. A compound may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered active ingredient moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the vitamin D₃ compound(s) include pharmaceutically-acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

In addition to inert diluents, the oral compositions can include adjuvants such as wetting agents, emulsifying and suspending agents, sweetening, flavoring, coloring, perfuming and preservative agents.

Suspensions, in addition to the active vitamin D₃ compound(s) may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions of the invention for rectal or vaginal administration may be presented as a suppository, which may be prepared by mixing one or more vitamin D₃ compound(s) with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent.

Compositions of the present invention which are suitable for vaginal administration also include pessaries, tampons, creams, gels, pastes, foams or spray formulations containing such carriers as are known in the art to be appropriate.

Dosage forms for the topical or transdermal administration of a vitamin D₃ compound(s) include powders, sprays, ointments, pastes, creams, lotions, gels, solutions, patches and inhalants. The active vitamin D₃ compound(s) may be mixed under sterile conditions with a pharmaceutically-acceptable carrier, and with any preservatives, buffers, or propellants which may be required.

The ointments, pastes, creams and gels may contain, in addition to vitamin D₃ compound(s) of the present invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a vitamin D₃ compound(s), excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The vitamin D₃ compound(s) can be alternatively administered by aerosol. This is accomplished by preparing an aqueous aerosol, liposomal preparation or solid particles containing the compound. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of the agent together with conventional pharmaceutically-acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Transdermal patches have the added advantage of providing controlled delivery of a vitamin D₃ compound(s) to the body. Such dosage forms can be made by dissolving or dispersing the agent in the proper medium. Absorption enhancers can also be used to increase the flux of the active ingredient across the skin. The rate of such flux can be controlled by either providing a rate controlling membrane or dispersing the active ingredient in a polymer matrix or gel.

Ophthalmic formulations, eye ointments, powders, solutions and the like, are also contemplated as being within the scope of this invention.

Pharmaceutical compositions of this invention suitable for parenteral administration comprise one or more vitamin D₃ compound(s) in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of vitamin D₃ compound(s) in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the vitamin D₃ compound(s) are administered as pharmaceuticals, to humans and animals, they can be given per se or as a pharmaceutical composition containing, for example, 0.1 to 99.5% (more preferably, 0.5 to 90%) of active ingredient in combination with a pharmaceutically-acceptable carrier.

Regardless of the route of administration selected, the vitamin D₃ compound(s), which may be used in a suitable hydrated form, and/or the pharmaceutical compositions of the present invention, are formulated into pharmaceutically-acceptable dosage forms by conventional methods known to those of skill in the art.

Actual dosage levels and time course of administration of the active ingredients in the pharmaceutical compositions of this invention may be varied so as to obtain an amount of the active ingredient which is effective to achieve the desired therapeutic response for a particular patient, composition, and mode of administration, without being toxic to the patient. An exemplary dose range is from 0.1 to 10 mg per day.

A preferred dose of the vitamin D₃ compound for the present invention is the maximum that a patient can tolerate and not develop serious hypercalcemia. Preferably, the vitamin D₃ compound of the present invention is administered at a concentration of about 0.001 μg to about 100 μg per kilogram of body weight, about 0.001-about 10 μg/kg or about 0.001 μg-about 100 μg/kg of body weight. Ranges intermediate to the above-recited values are also intended to be part of the invention.

### 5. Synthesis of Compounds of the Invention

Compounds of the invention can be synthesized by methods described in this section, the examples, and the chemical literature.

### A. Synthesis of 20-Methyl Gemini Vitamin D₃ Compounds

Schemes 1-17 below depict the reaction steps for the synthesis of the 20-methyl gemini vitamin D₃ compounds of the invention. For the synthesis of compounds 1-17 the convergent and Wittig-Horner reaction coupling protocol was used.

Scheme 1 shows the synthetic route for the production of the disilyl protected Gemini diol 51. Alcohol 40 (H. Maehr; M. R. Uskokovic. Eur. J. Org. Chem., 2004, 1703-1713.) was protected to form compound 41, then cyclopropanated to provide cyclopropane 42. The cyclopropyl compound was deprotected with TBAF, and the ester 43 was reduced to alcohol 44. Oxidation to aldehyde 45 was followed by chain elongation using a modified Wittig-Horner reaction to provide 46. Reduction of the double bond and concomitant cyclopropane opening liberated ester 47, which was reduced and deprotected to form diol intermediate 49. Oxidation of the ring hydroxyl group to the corresponding ketone 50, was followed by protection to form intermediate 51.

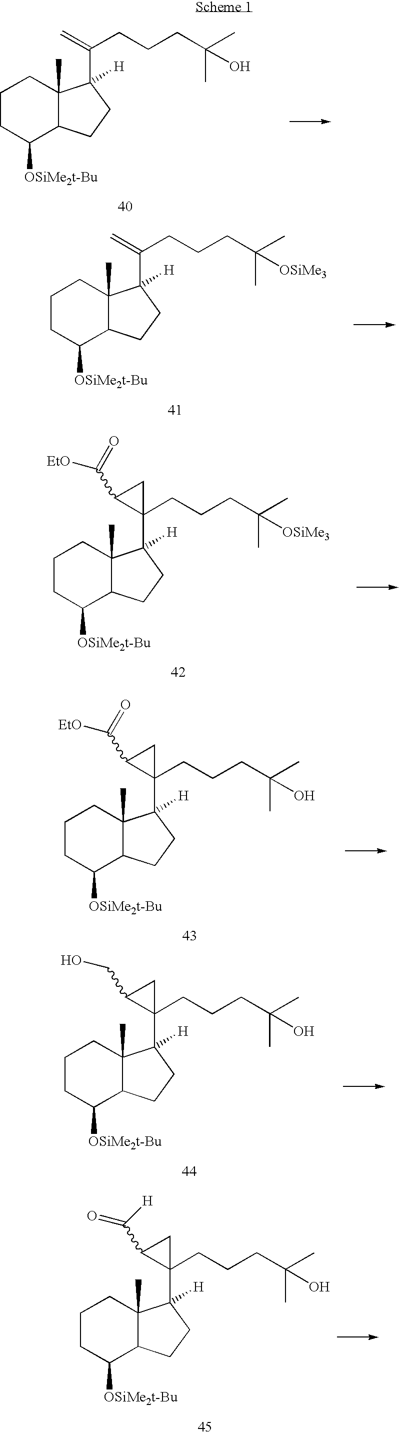

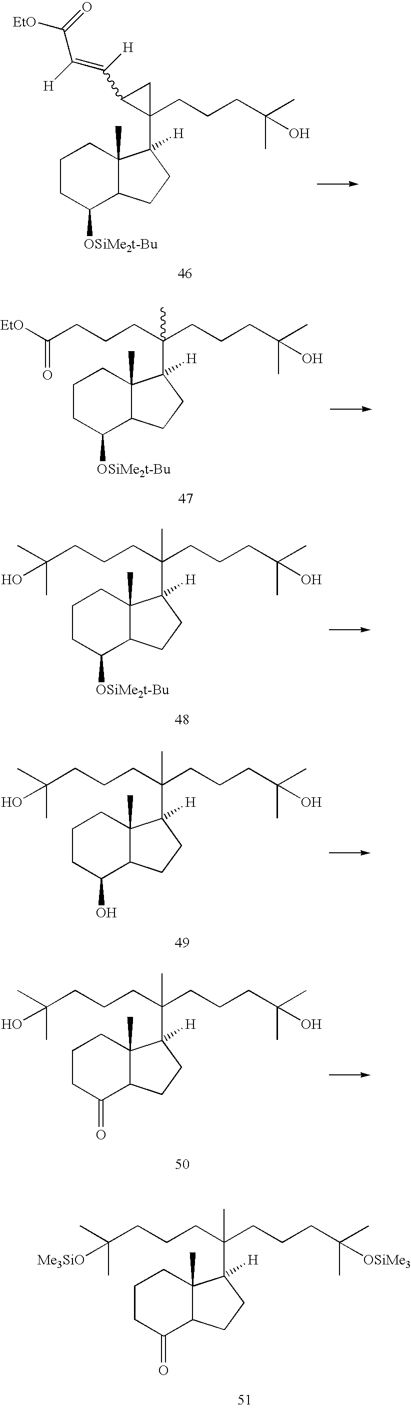

Scheme 2 shows the coupling of ketone 51 with phosphine oxides 52, 53, and 54, followed by deprotection with tetrabutyl ammonium fluoride (TBAF), to provide vitamin D compounds 1, 2, and 3, respectively.

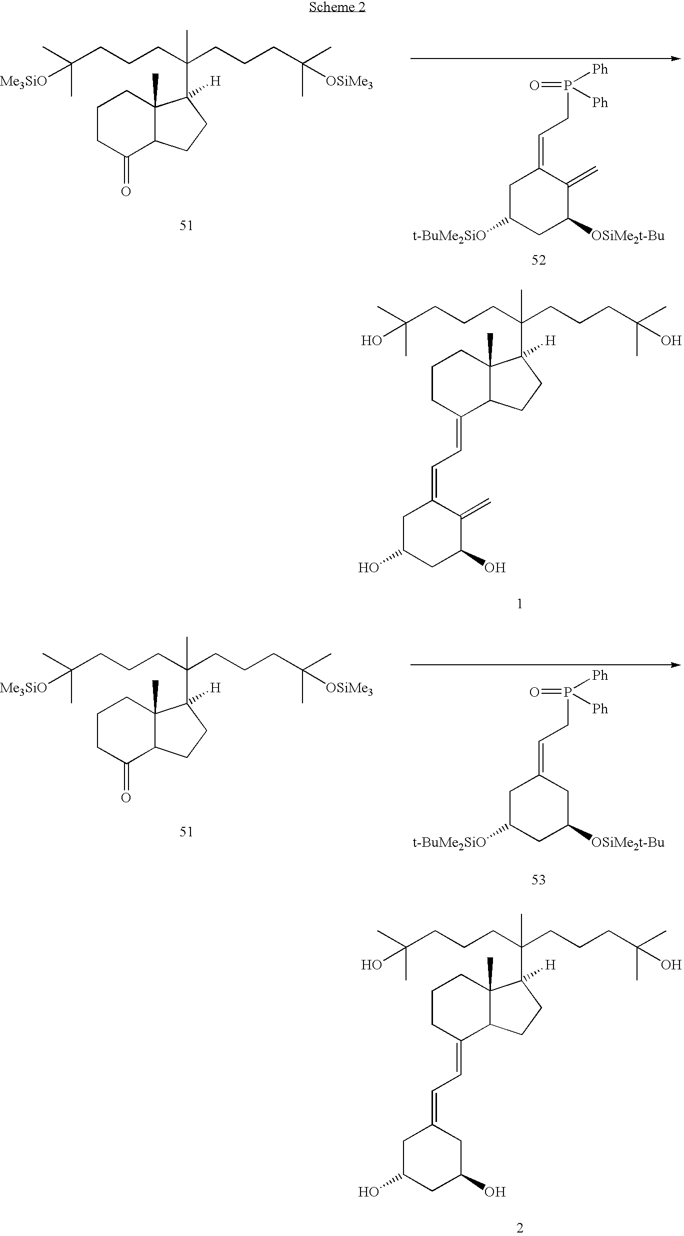

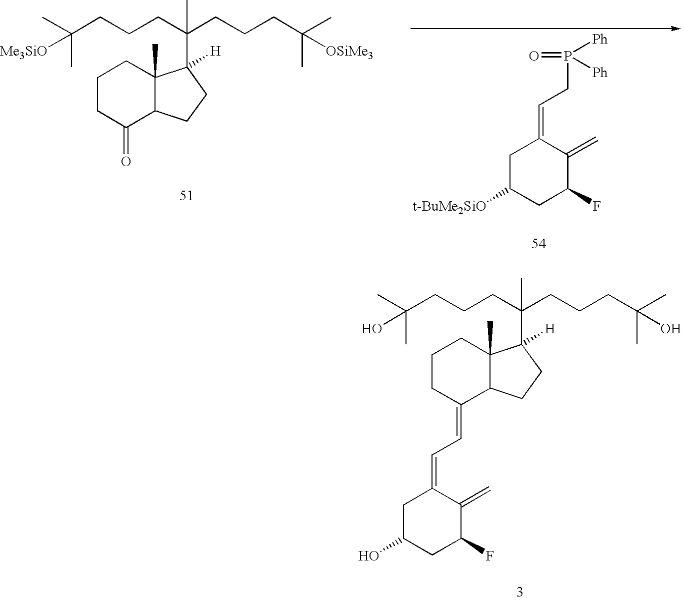

Schemes 3 and 4 demonstrate the synthetic route for the production of fluorinated intermediates 70, 72, and 74. Alcohol 55 was silyl protected then cyclopropanated to provide cyclopropane 57. The cyclopropyl compound was reduced to alcohol 58, then oxidized to aldehyde 59. Carbon chain elongation was accomplished by a modified Horner-Emmons reaction to provide 60, which was followed by reduction of the double bond and concomitant cyclopropane opening liberated ester 61, which was reduced and deprotected to form diol intermediates 63 and 64.

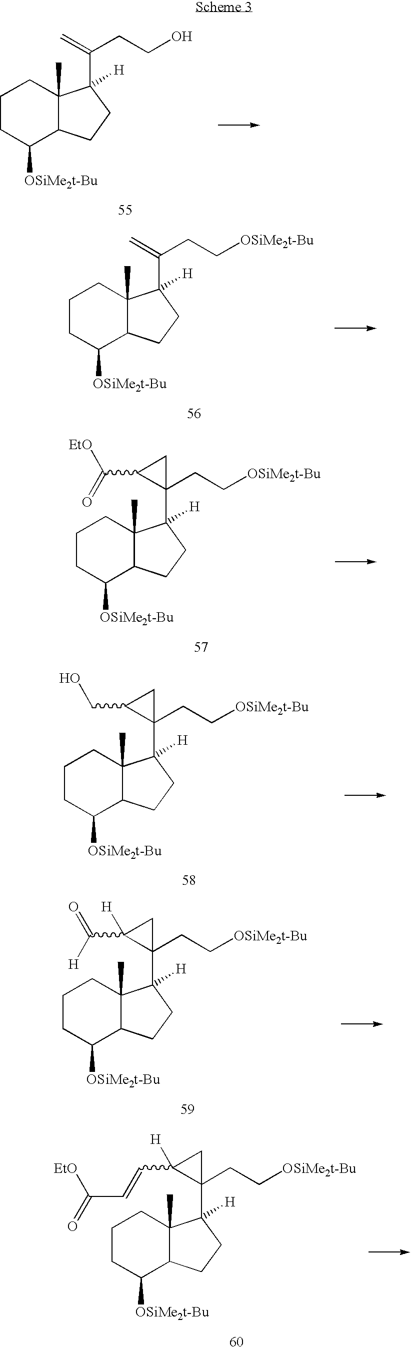

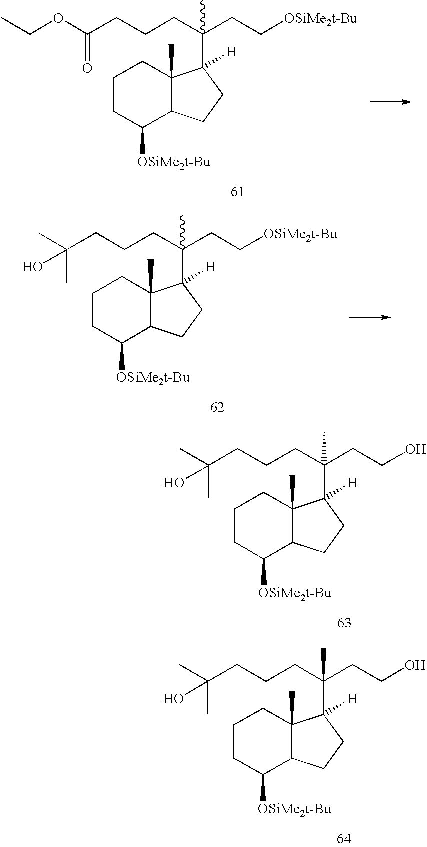

Scheme 4 shows the conversion of intermediate diol 63 to ketone intermediates 70, 72 and 74. Oxidation of 63 provided aldehyde 65, which was converted to alkyne 66. Protection of the hydroxyl group of 66 was followed by base-mediated addition of hexafluoroacetone to afford 68. Deprotection of 68 provided triol 69. Oxidation of 69 provided ketone 70. Alkyne reduction of 68 to the cis olefin was accomplished to provide compound 71. Oxidation of 71 provided ketone 72. Alkyne reduction of 68 to the trans olefin was accomplished to provide compound 73, which was followed by oxidation to form ketone 74.

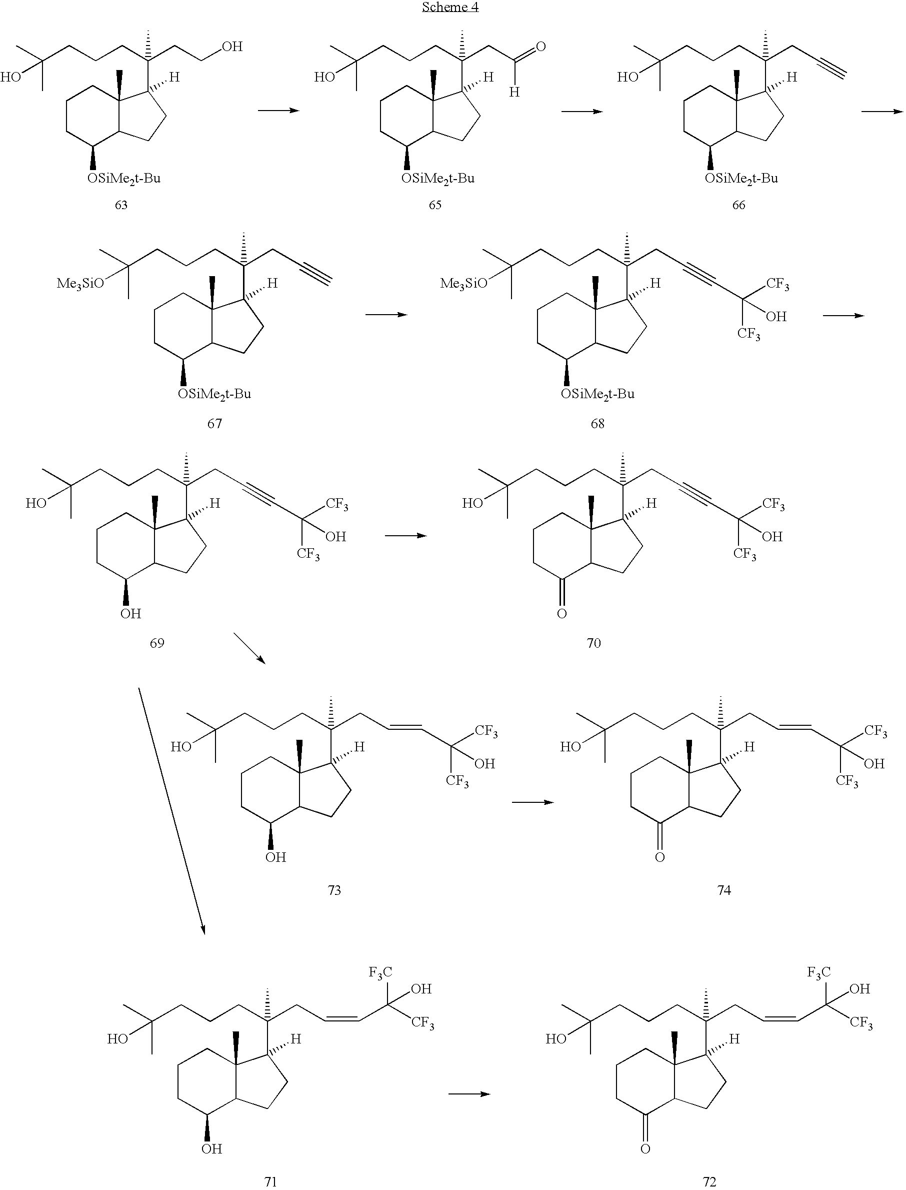

Scheme 5 shows the coupling of ketone 70 with phosphine oxide 52 to provide vitamin D compound 4.

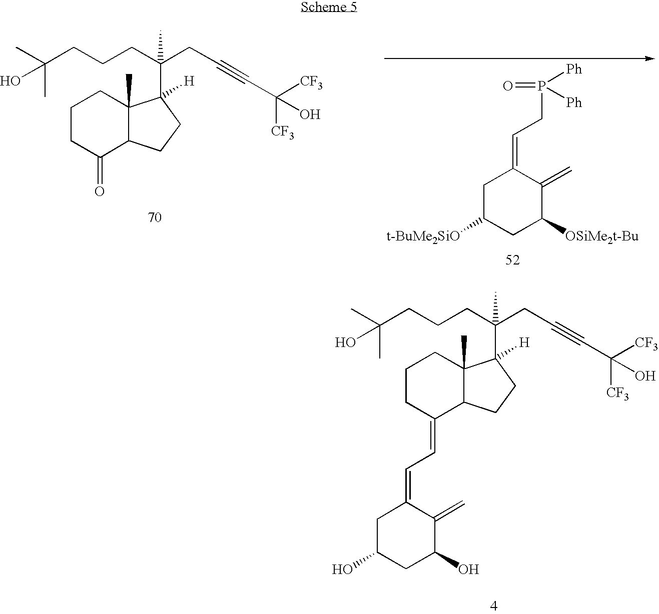

Scheme 6 provides for the silyl protection of 70 to form compound 75, which was then coupled with phosphine oxides 53, and 54, followed by deprotection with tetrabutyl ammonium fluoride (TBAF), to provide vitamin D compounds 10 and 13, respectively.

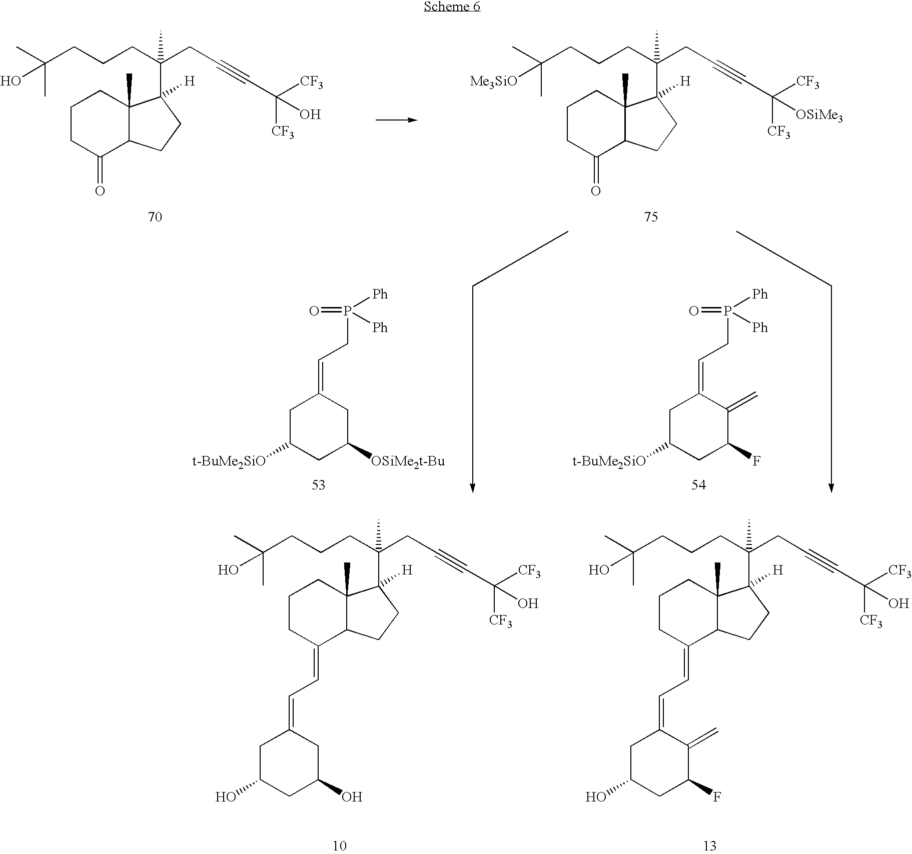

Scheme 7 shows the coupling of ketone 72 with phosphine oxide 52 to provide vitamin D compound 5.

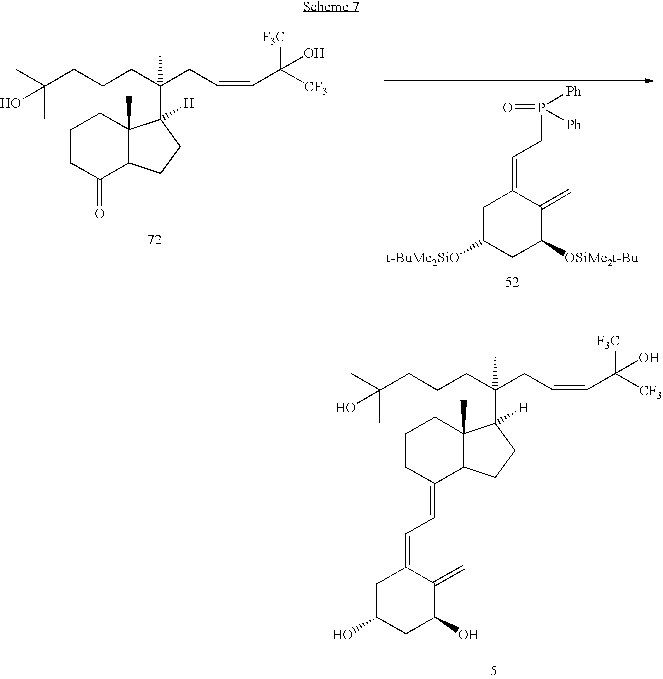

Scheme 8 provides for the silyl protection of 72 to form compound 76, which was then coupled with phosphine oxides 53, and 54, followed by deprotection with tetrabutyl ammonium fluoride (TBAF), to provide vitamin D compounds 11 and 14, respectively.

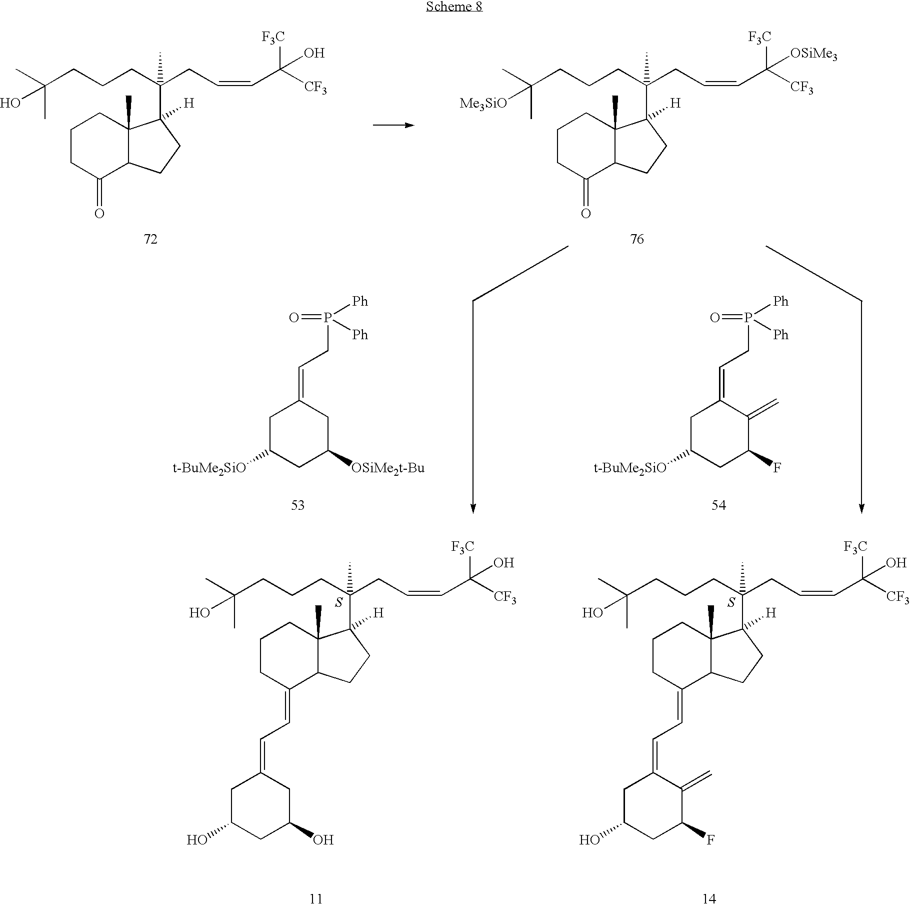

Scheme 9 shows the coupling of ketone 74 with phosphine oxide 52 to provide vitamin D compound 6.

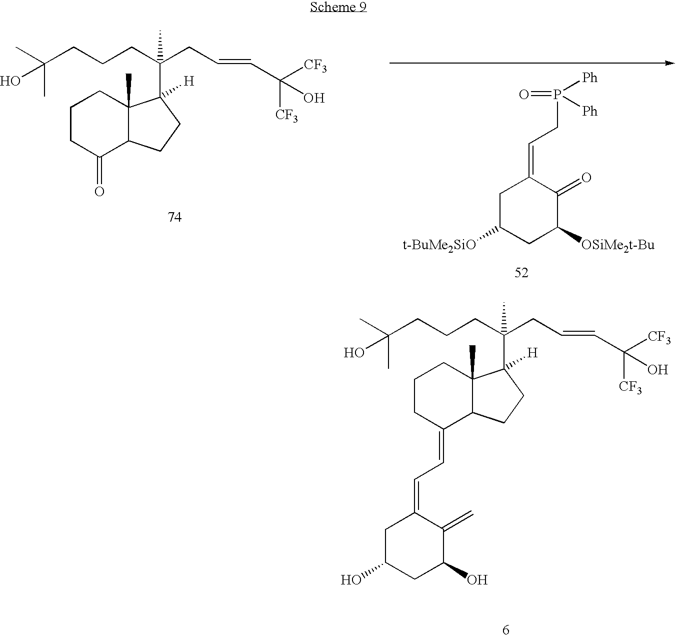

Scheme 10 provides for the silyl protection of 74 to form compound 77, which was then coupled with phosphine oxides 53, and 54, followed by deprotection with tetrabutyl ammonium fluoride (TBAF), to provide vitamin D compounds 12 and 15, respectively.

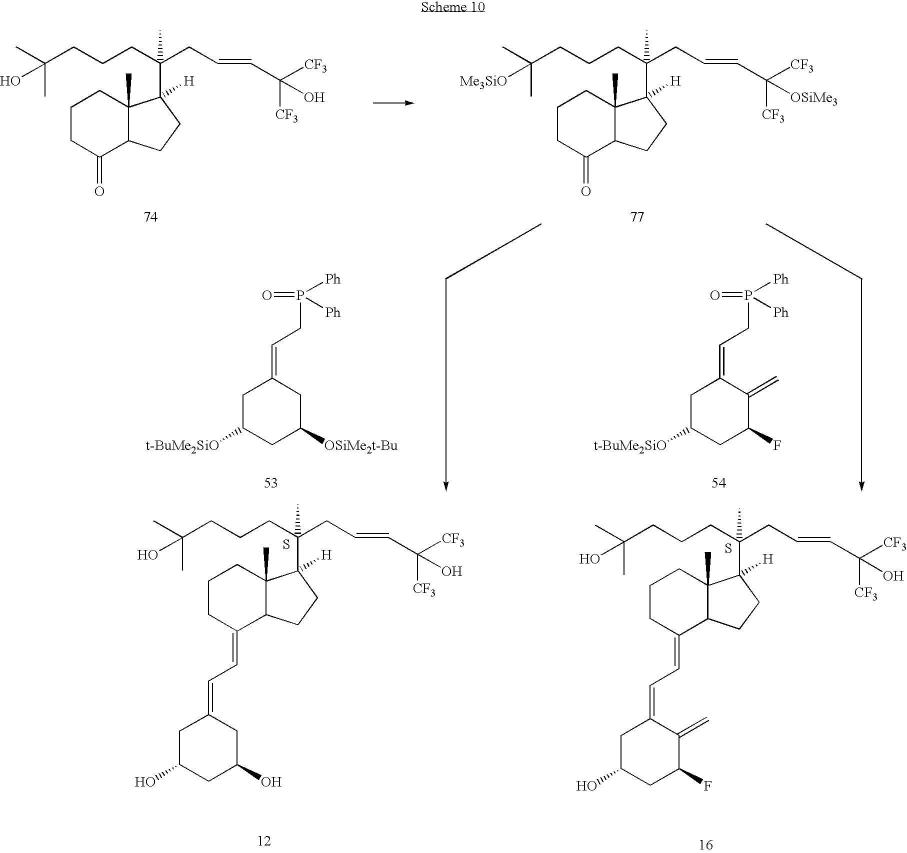

Scheme 11 shows the conversion of the epimer of 63, diol 64, to ketone intermediates 83, 85 and 87. Oxidation of 64 provided aldehyde 78, which was converted to alkyne 79. Protection of the hydroxyl group of 79 was followed by base-mediated addition of hexafluoroacetone to afford 81. Deprotection of 81 provided triol 82. Oxidation of 82 provided ketone 83. Alkyne reduction of 82 to the cis olefin was accomplished to provide compound 84. Oxidation of 84 provided ketone 85. Alkyne reduction of 82 to the trans olefin was accomplished to provide compound 86, which was followed by oxidation to form ketone 87.

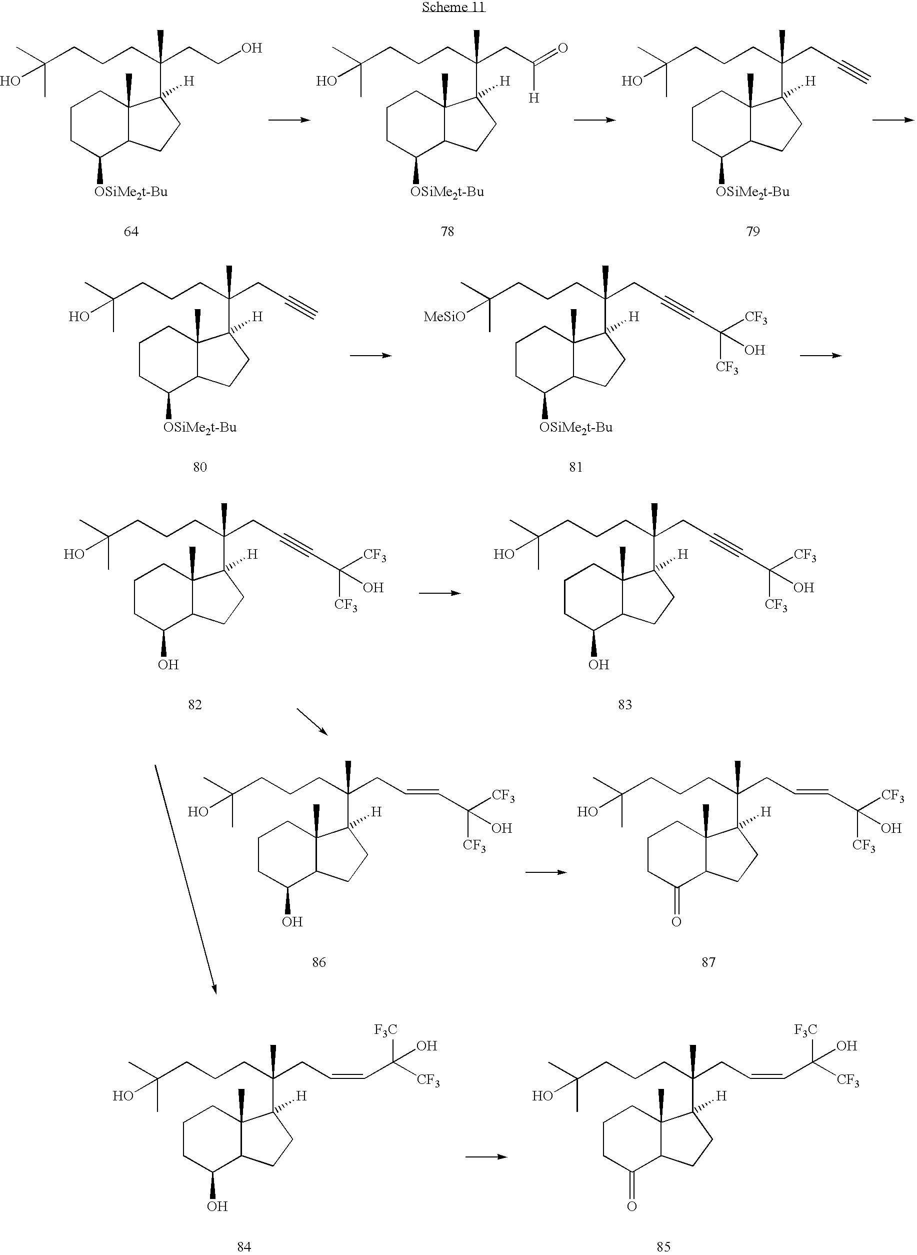

Scheme 12 shows the coupling of ketone 83 with phosphine oxide 52 to provide vitamin D compound 7.

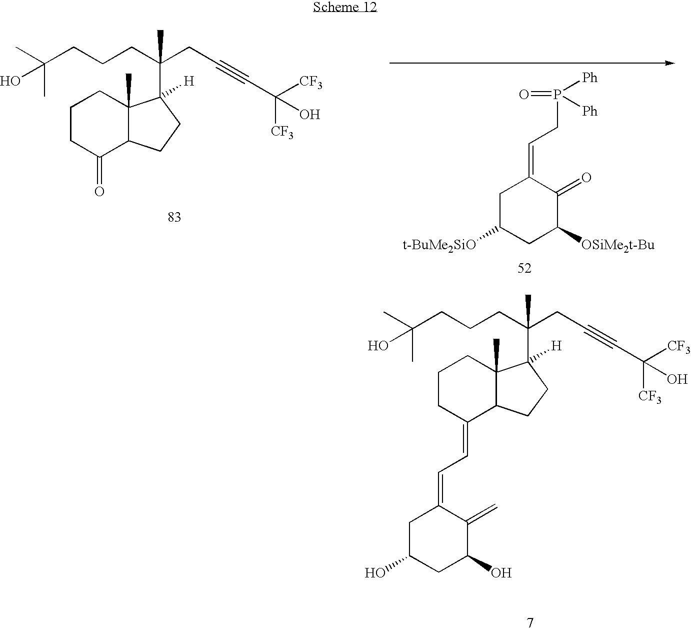

Scheme 13 provides for the silyl protection of 83 to form compound 88, which was then coupled with phosphine oxides 53, and 54, followed by deprotection with tetrabutyl ammonium fluoride (TBAF), to provide vitamin D compounds 34 and 37, respectively.

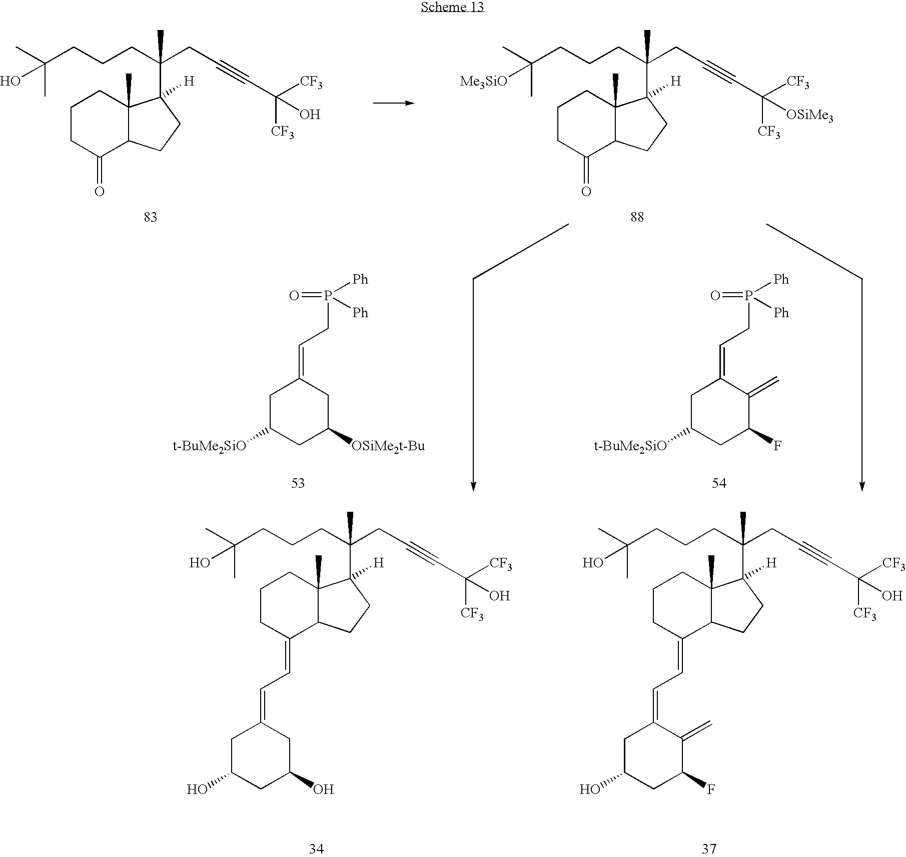

Scheme 14 shows the protection of ketone 85 to provide compound 89, which was subjected to coupling conditions.

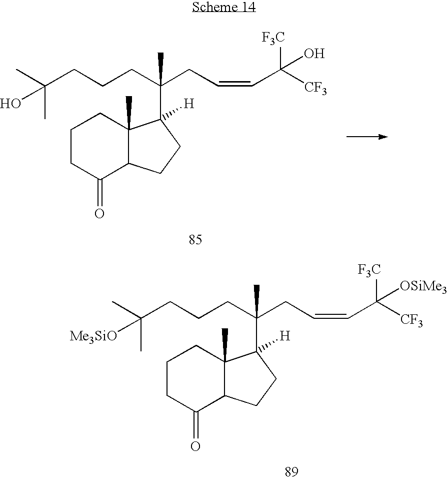

Scheme 15 provides for the coupling of ketone 89 with phosphine oxides 52, 53, and 54, followed by deprotection with tetrabutyl ammonium fluoride (TBAF), to provide vitamin D compounds 8, 35 and 38, respectively.

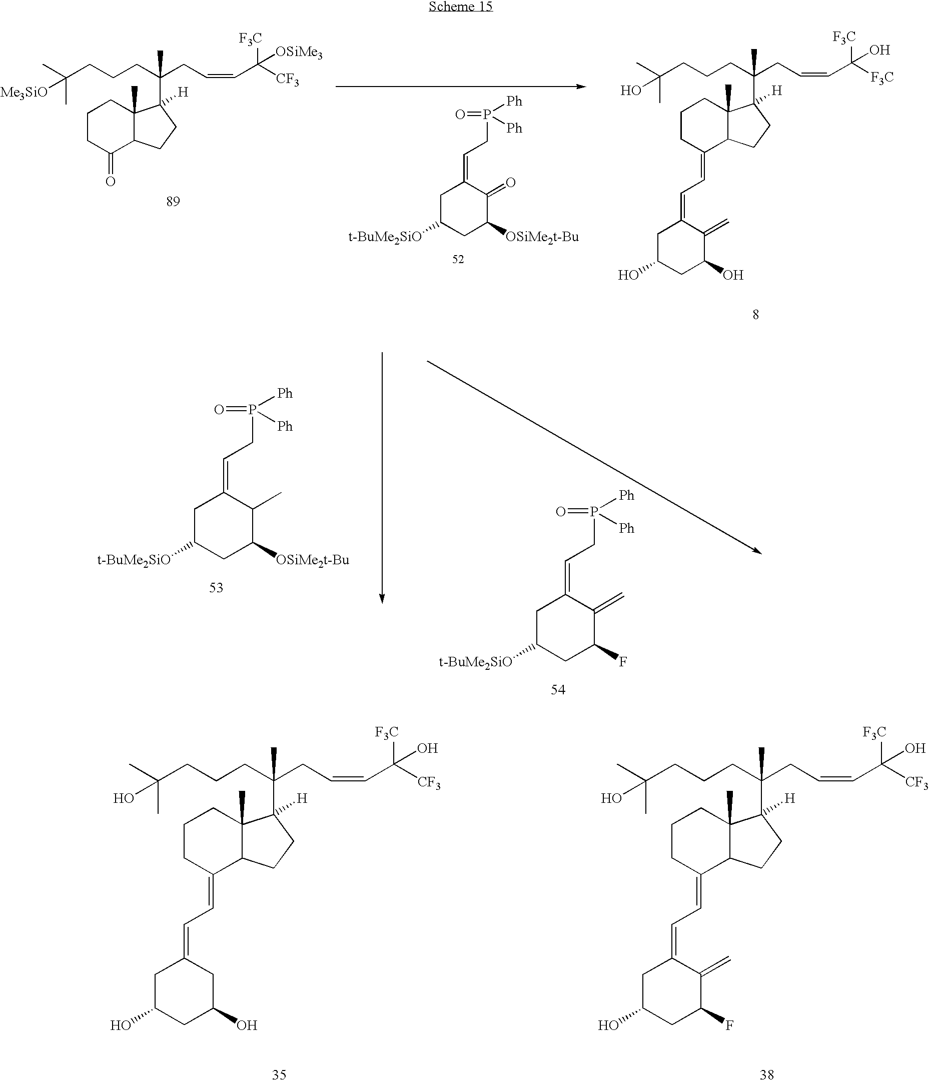

Scheme 16 shows the protection of ketone 87 to provide compound 90, which was subjected to coupling conditions.

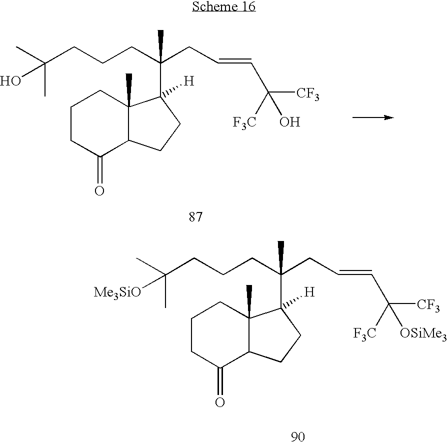

Scheme 17 provides for the coupling of ketone 90 with phosphine oxides 52, 53, and 54, followed by deprotection with tetrabutyl ammonium fluoride (TBAF), to provide vitamin D compounds 9, 36 and 39, respectively.

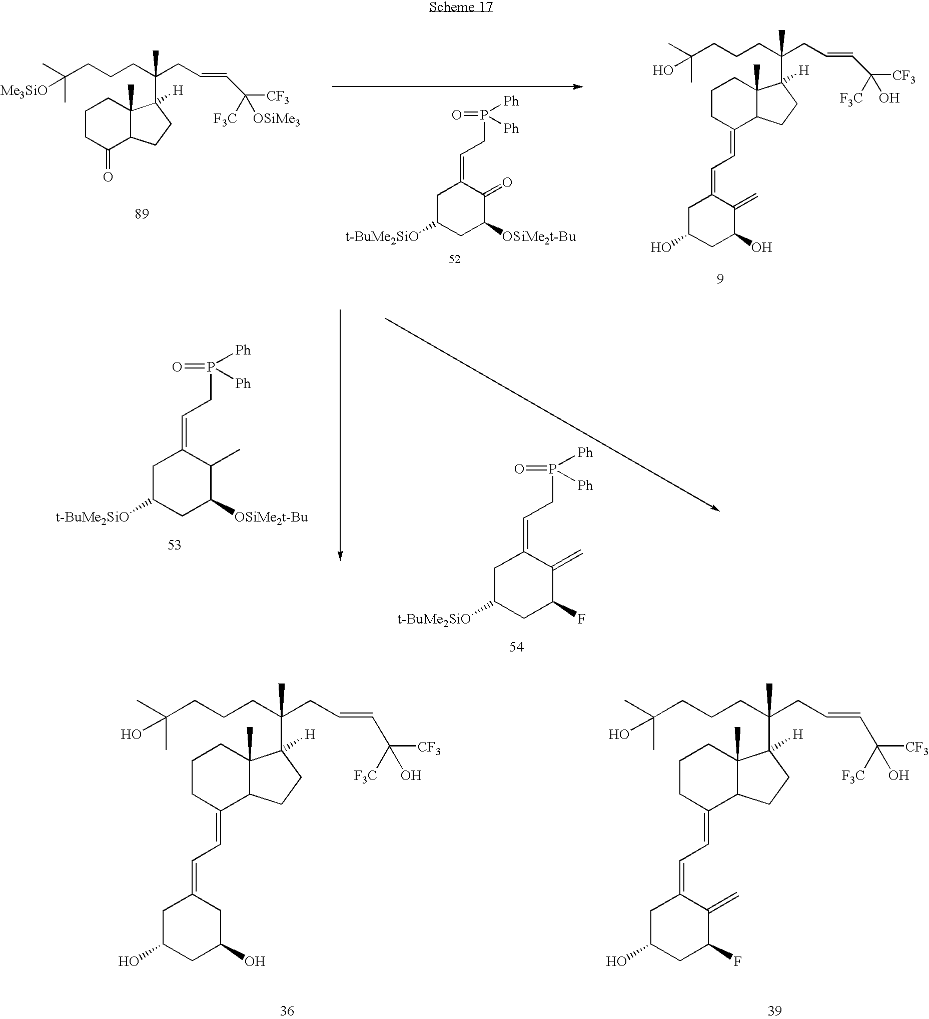

### B. Synthesis of Deuterated-20-Methyl Gemini Vitamin D₃ Compounds

Schemes 18-28 below depict the reaction steps for the synthesis of the hexadeuterated-20-methyl gemini vitamin D₃ compounds of the invention.

Scheme 18 shows the synthetic route for the production of the epimers 92 and 93. Compound 61 (from Scheme 3 above) was converted to the hexadeuterated compound 91. Deprotection of the silyl groups, followed by chromatographic separation, provided epimers 92 and 93.

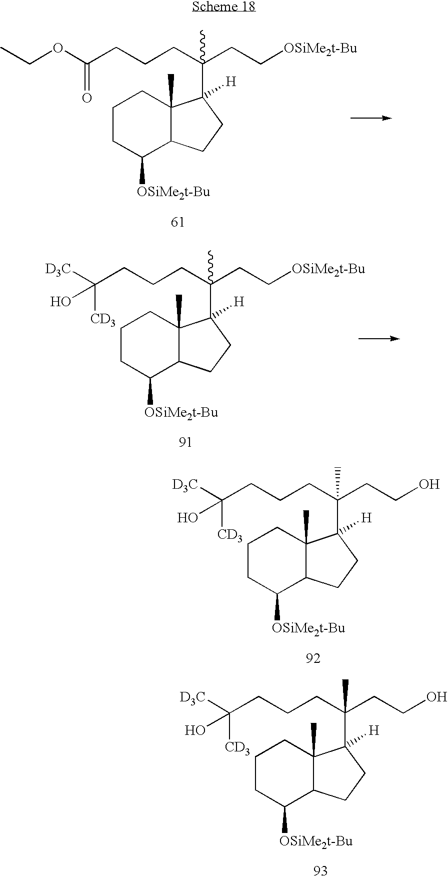

Scheme 19 shows the conversion of 92, to triol intermediates 98. Oxidation of 92 provided aldehyde 94, which was converted to alkyne 95. Protection of the hydroxyl group of 95 was followed by base-mediated addition of hexafluoroacetone to afford 97. Deprotection of 97 provided triol 98.

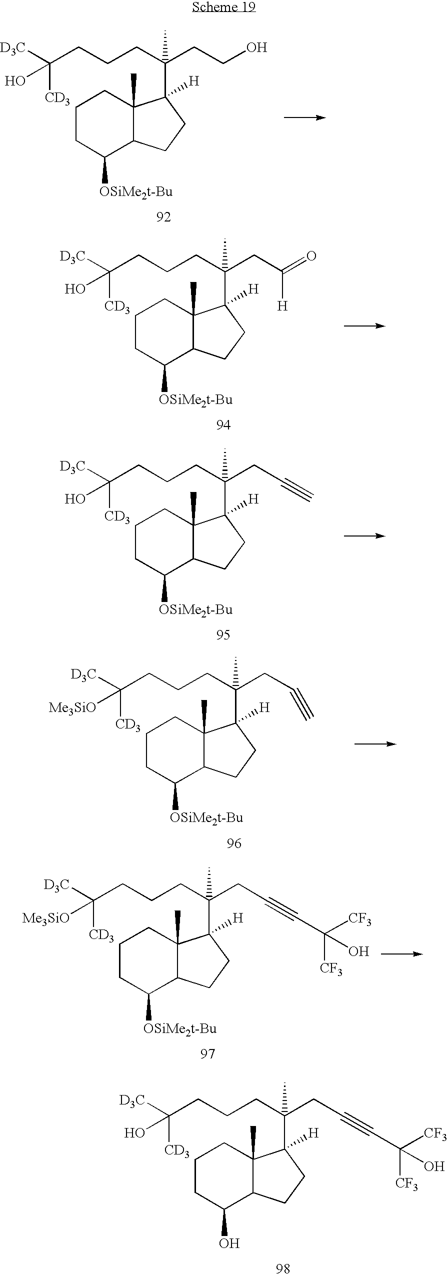

Scheme 20 shows the oxidation of 98 to provide ketone 99. Alkyne reduction of 98 to the cis olefin was accomplished to provide compound 100, which was followed by oxidation to provide ketone 101. Alkyne reduction of 98 to the trans olefin was accomplished to provide compound 102, which was followed by oxidation to form ketone 103.

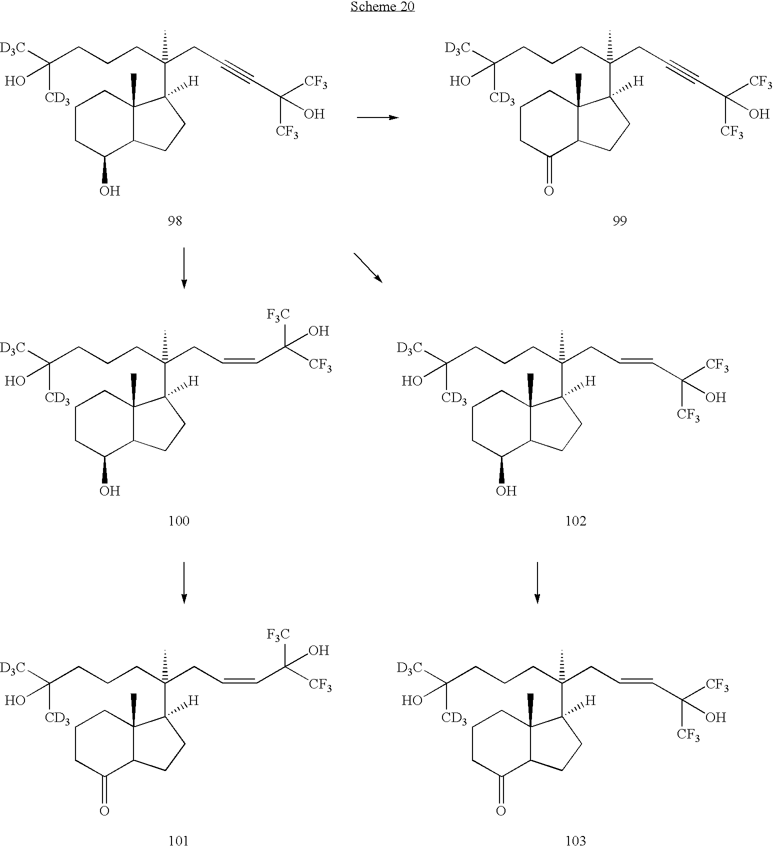

Scheme 21 provides for the silyl protection of 99 to form compound 104, which was then coupled with phosphine oxides 52, 53, and 54, followed by deprotection with tetrabutyl ammonium fluoride (TBAF), to provide vitamin D compounds 22, 23 and 24, respectively.

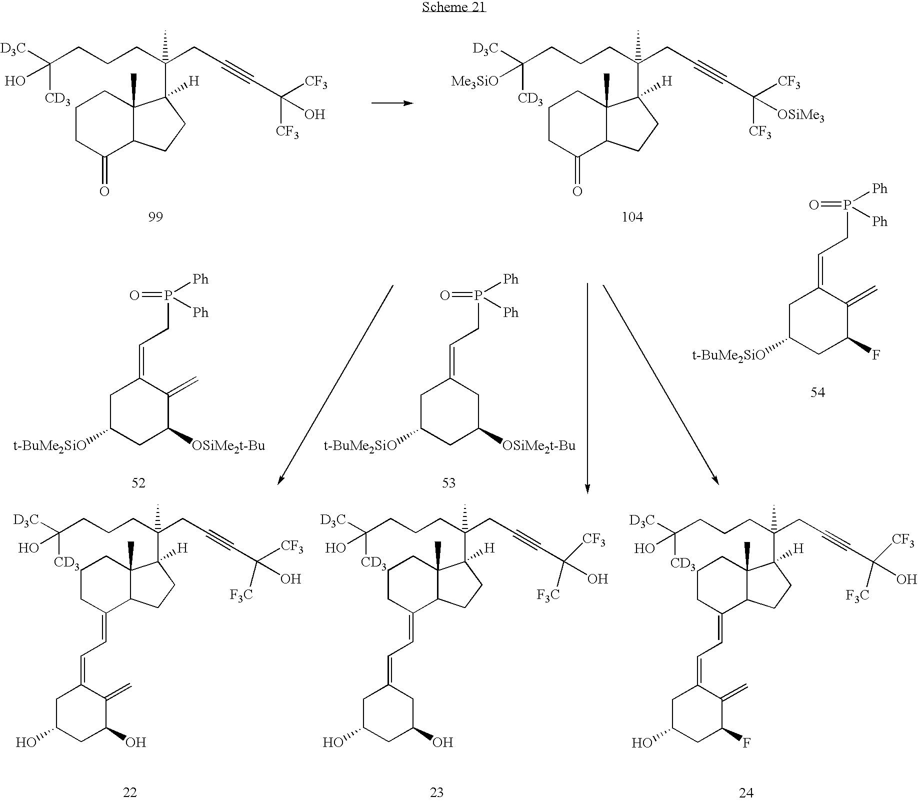

In Scheme 22, compound 101 is initially silyl protected to form compound 105, which then undergoes the coupling reaction with phosphine oxides 52, 53, and 54, to form vitamin D compounds 16, 17, and 18, respectively.

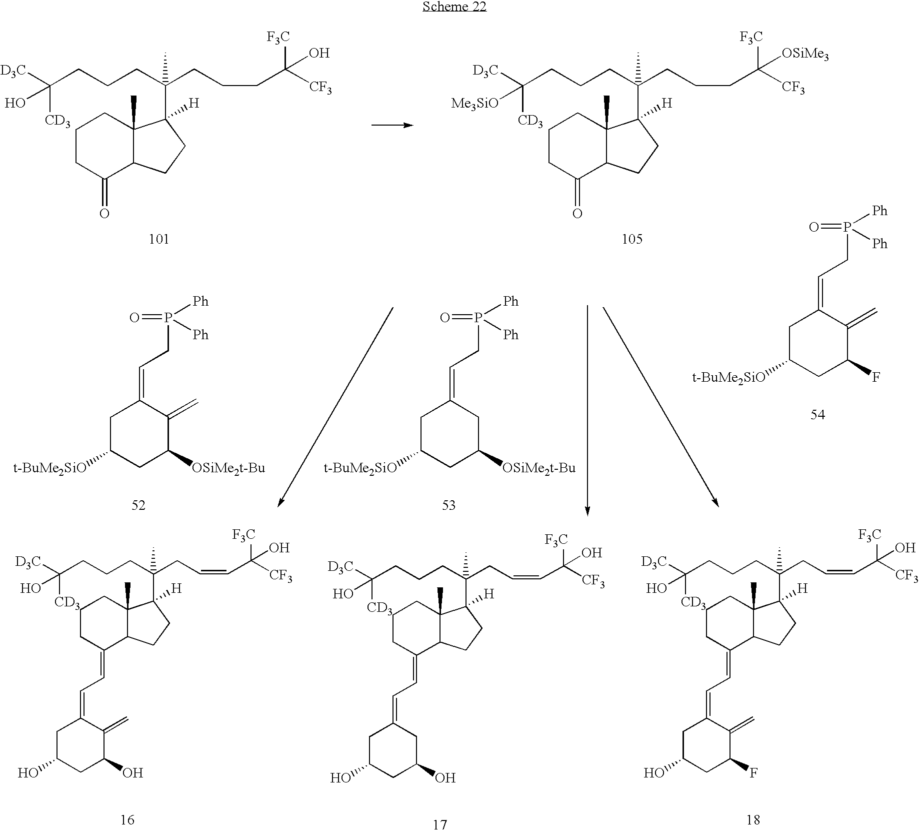

In Scheme 23, compound 103 is initially silyl protected to form compound 106, which then undergoes the coupling reaction with phosphine oxides 52, 53, and 54, to form vitamin D compounds 19, 20, and 21, respectively.

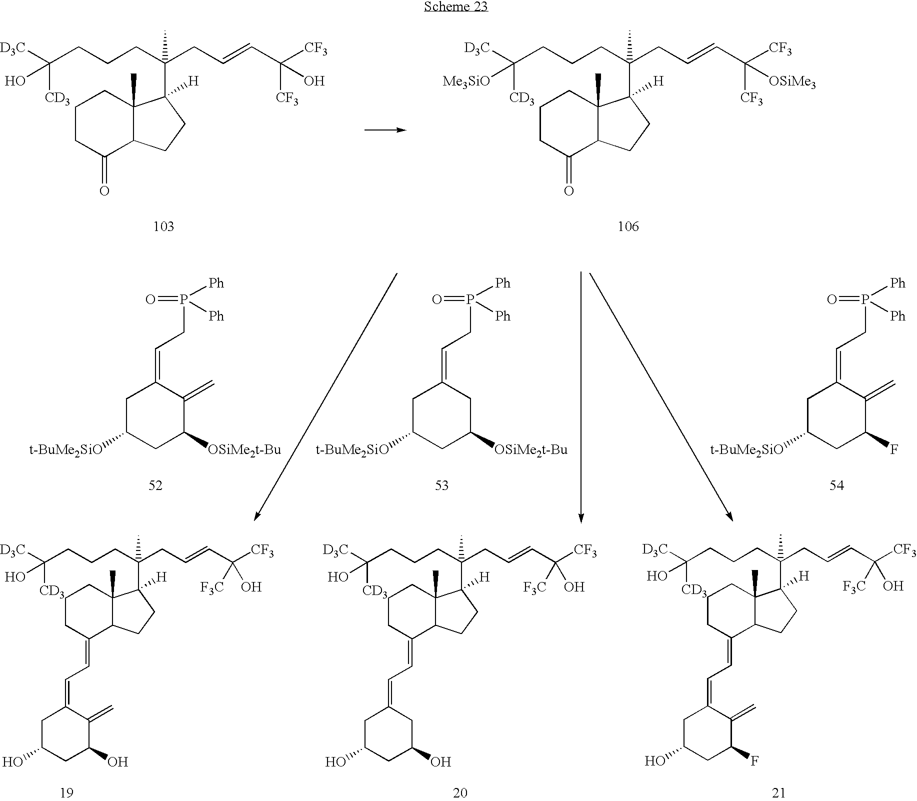

Scheme 24 shows the conversion of 93, to triol intermediates 111. Oxidation of 93 provided aldehyde 107, which was converted to alkyne 108. Protection of the hydroxyl group of 108 was followed by base-mediated addition of hexafluoroacetone to afford 110. Deprotection of 110 provided triol 111.

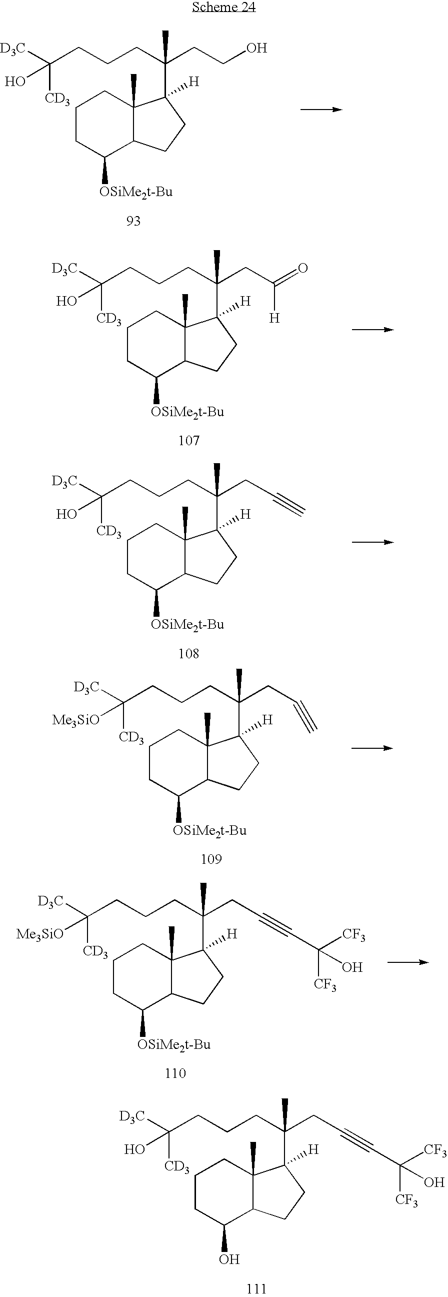

Scheme 25 shows the conversion of 98 to ketones 112, 114, and 116. Oxidation of triol 111 provided alkyne ketone 112. Alkyne reduction of 111 to the cis olefin was accomplished to provide compound 113, which was followed by oxidation to provide ketone 114. Alkyne reduction of 111 to the trans olefin was accomplished to provide compound 115, which was followed by oxidation to form ketone 116.

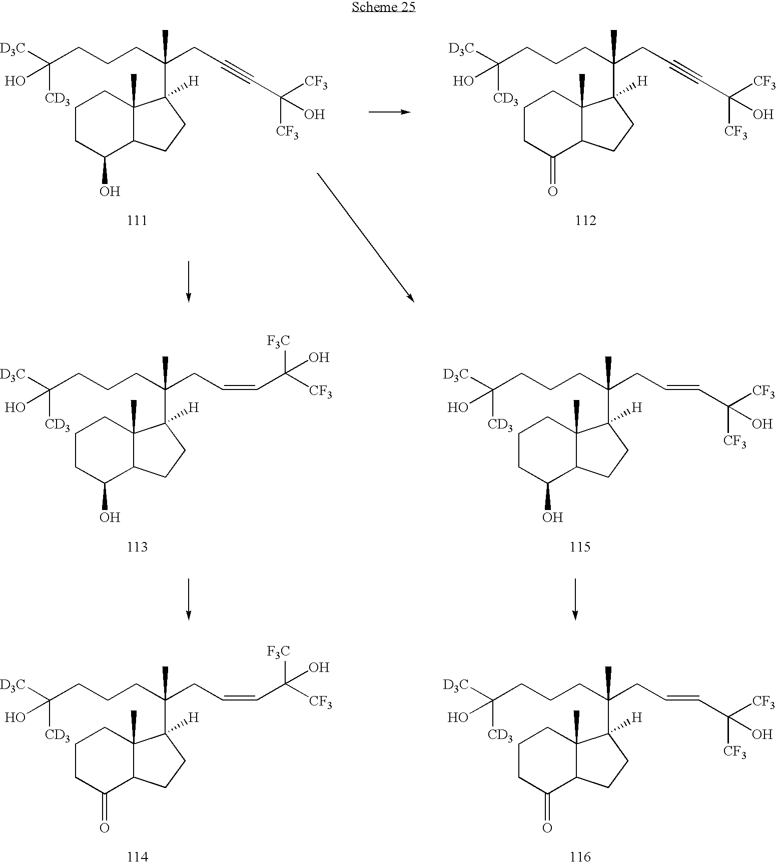

Scheme 26 provides for the silyl protection of 112 to form compound 117, which was then coupled with phosphine oxides 52, 53, and 54, followed by deprotection with tetrabutyl ammonium fluoride (TBAF), to provide vitamin D compounds 31, 32 and 33, respectively.

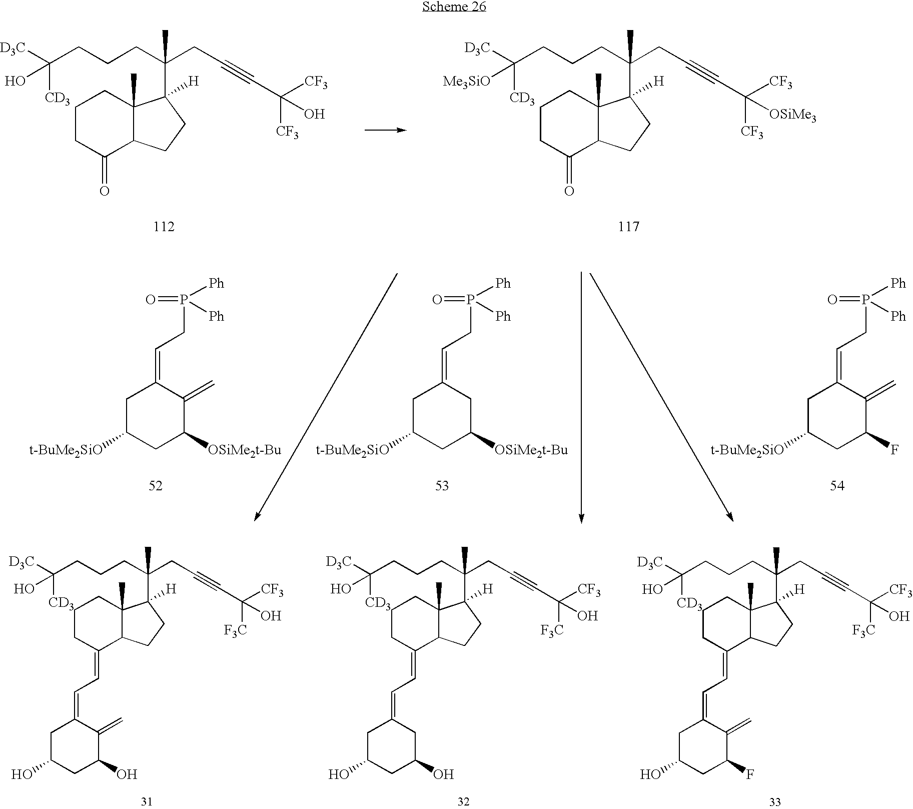

In Scheme 27, compound 114 is initially silyl protected to form compound 118, which then undergoes the coupling reaction with phosphine oxides 52, 53, and 54, to form vitamin D compounds 25, 26, and 27, respectively.

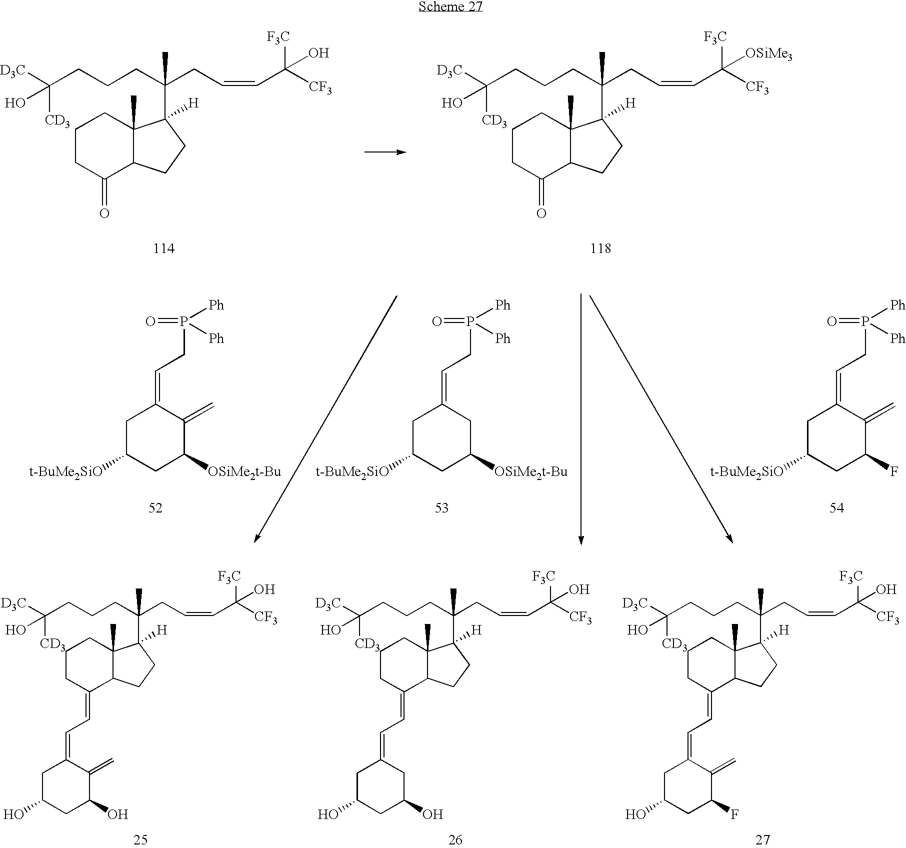

In Scheme 28, compound 116 is initially silyl protected to form compound 119, which then undergoes the coupling reaction with phosphine oxides 52, 53, and 54, to form vitamin D compounds 28, 29, and 30, respectively.

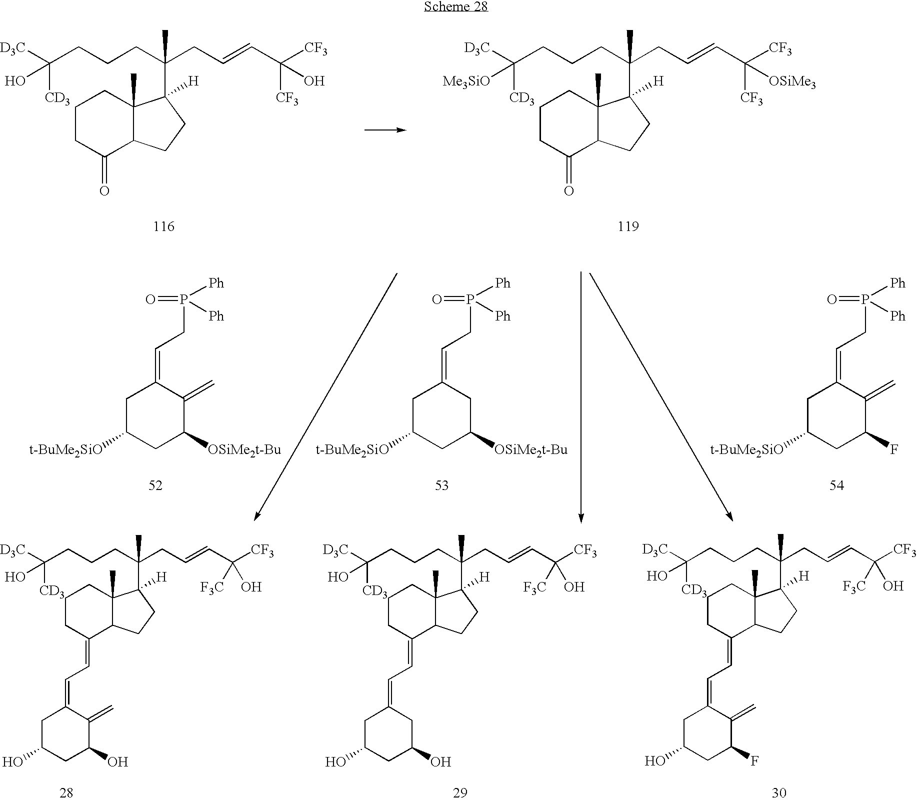

Chiral syntheses can result in products of high stereoisomer purity. However, in some cases, the stereoisomer purity of the product is not sufficiently high. The skilled artisan will appreciate that the separation methods described herein can be used to further enhance the stereoisomer purity of the vitamin D₃-epimer obtained by chiral synthesis.

Any novel syntheses, described herein, of the compounds of the invention, and of intermediates thereof, are also intended to be included within the scope of the present invention.

### EXEMPLIFICATION OF THE INVENTION

The invention is further illustrated by the following examples which should in no way should be construed as being further limiting.

### Synthesis of Compounds of the Invention

All operations involving vitamin D₃ analogs were conducted in amber-colored glassware in a nitrogen atmosphere. Tetrahydrofuran was distilled from sodium-benzophenone ketyl just prior to its use and solutions of solutes were dried with sodium sulfate. Melting points were determined on a Thomas-Hoover capillary apparatus and are uncorrected. Optical rotations were measured at 25° C. ¹H NMR spectra were recorded at 400 MHz in CDCl₃ unless indicated otherwise. TLC was carried out on silica gel plates (Merck PF-254) with visualization under short-wavelength UV light or by spraying the plates with 10% phosphomolybdic acid in methanol followed by heating. Flash chromatography was carried out on 40-65 μm mesh silica gel. Preparative HPLC was performed on a 5×50 cm column and 15-30 μm mesh silica gel at a flow rate of 100 mL/min.

### Example 1

### Synthesis of 1,25-Dihydroxy-20-(4-hydroxy-4-methyl-pentyl)cholecalciferol (1)

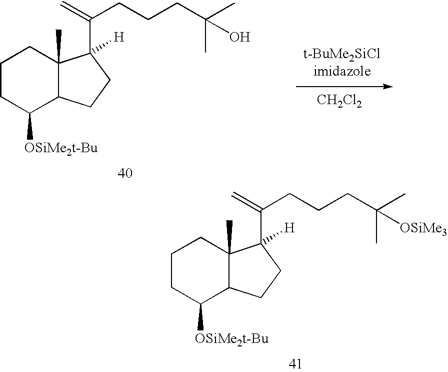

### (1R,3aR,4S,7aR)-4-(tert-Butyl-dimethyl-silanyloxy)-7a-methyl-1-(5-methyl-1-methylene-5-trimethylsilanyloxy-hexyl)-octahydro-indene (41)

A 50 ml round bottom flask equipped with stir bar and Claisen adapter with rubber septum was charged with 1.78 g (4.510 mmol) of 6-[(1R,3aR,4S,7aR)-4-(tert-butyl-dimethyl-silanyloxy)-7a-methyl-octahydro-inden-1-yl]-2-methyl-hept-6-en-2-ol (40) and 15 ml of dichloromethane. A 1.98 ml (13.53 mmol) of 1-(trimethylsilyl)imidazole was added dropwise. The mixture was stirred at room temperature for 2 h. A 15 ml of water was added and the mixture was stirred for 10 min. The resulting mixture was dissolved by the addition of 100 ml of water. The aqueous layer was extracted three times with 50 ml of dichloromethane. The combined organic layers were washed with 30 ml of brine dried over Na₂SO₄ and evaporated. The oil residue was chromatographed on column (75 cm³) using hexane:ethyl acetate (10:1) as mobile phase. Fractions containing product were pooled and evaporated to give 2.037 g (96%) of product 41 as colorless oil.

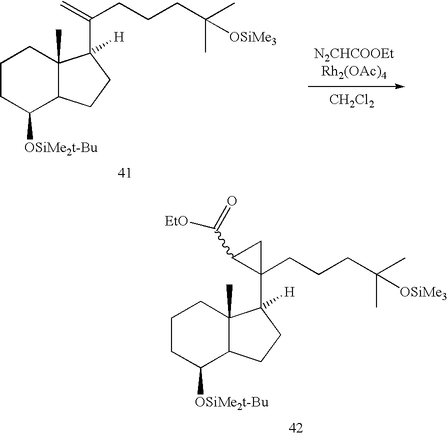

### 2-[(1S,3aR,4S,7aR)-4-(tert-Butyl-dimethyl-silanyloxy)-7a-methyl-octahydro-inden-1-yl]-2-(4-methyl-4-trimethylsilanyloxy-pentyl)-cyclopropanecarboxylic acid ethyl ester (42)

A 100 ml round bottom flask equipped with stir bar and Claisen adapter with rubber septum was charged with 1.275 g (2.731 mmol) of (1R,3aR,4S,7aR)-4-(tert-butyl-dimethyl-silanyloxy)-7a-methyl-1-(5-methyl-1-methylene-5-trimethylsilanyloxy-hexyl)-octahydro-indene (41), 25 mg of Rh₂(OAc)₄ and 10 ml of dichloromethane. A solution of 935 mg (8.202 mmol) of ethyl diazoacetate in 20 ml of dichloromethane was added dropwise (5 ml/h) at room temperature. The mixture was stirred for 30 min. The reaction mixture was concentrated in vacuo and the remaining residue was chromatographed on column (100 cm³) using dichloromethane as mobile phase to give 1.236 g (82%) of products 42 as mixture of isomers.

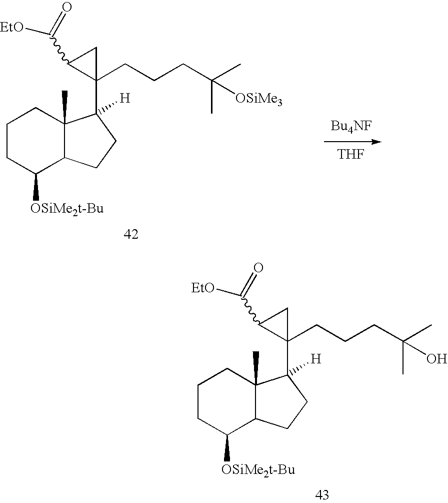

### 2-[(1S,3aR,4S,7aR)-4-(tert-Butyl-dimethyl-silanyloxy)-7a-methyl-octahydro-inden-1-yl]-2-(4-hydroxy-4-methyl-pentyl)-cyclopropanecarboxylic acid ethyl ester (43)

A 25 ml round bottom flask equipped with stir bar and Claisen adapter with rubber septum was charged with 1.236 g (2.235 mmol) of 2-[(1S,3aR,4S,7aR)-4-(tert-butyl-dimethyl-silanyloxy)-7a-methyl-octahydro-inden-1-yl]-2-(4-methyl-4-trimethylsilanyloxy-pentyl)-cyclopropanecarboxylic acid ethyl ester (42), 4 ml of 1M tetrabutylammonium fluoride in tetrahydrofurane and 4 ml of tetrahydrofurane. The reaction mixture was stirred at room temperature for 2 h. The mixture was dissolved by the addition of 100 ml of ethyl acetate and extracted five times with 50 ml of water:brine (2:1) and 50 ml of brine, dried over Na₂SO₄ and evaporated to give 1.081 g of product 43 as colorless oil (product was used to the next reaction without purification).

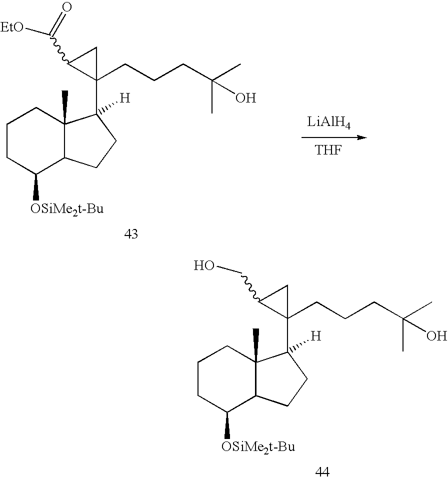

### 5-{1-[(1S,3aR,4S,7aR)-4-(tert-Butyl-dimethyl-silanyloxy)-7a-methyl-octahydro-inden-1-yl]-2-hydroxymethyl-cyclopropyl}-2-methyl-pentan-2-ol (44)

A 50 ml round bottom flask equipped with stir bar and Claisen adapter with rubber septum was charged with crude (ca. 2.2 mmol) of 2-[(1S,3aR,4S,7aR)-4-(tert-butyl-dimethyl-silanyloxy)-7a-methyl-octahydro-inden-1-yl]-2-(4-hydroxy-4-methyl-pentyl)-cyclopropanecarboxylic acid ethyl ester (43) and 6 ml of tetrahydrofurane. A 6 ml of 1M lithium aluminium hydride in tetrahydrofurane was added dropwise and the reaction mixture was stirred at room temperature for 1.5 h. Then the flask was placed into an ice bath and 5 ml of water was added dropwise. The mixture was dissolved by the addition of 50 ml of saturated solution of ammonium chloride, 50 ml of water and 25 ml of 1M H₂SO₄, extracted three times with 50 ml of ethyl acetate, dried over Na₂SO₄ and evaporated. The residue was purified over silica gel (350 cm³) using hexane:ethyl acetate (2:1, 1:1) to give 876 mg (90%) of products 44 as a mixture of isomers.

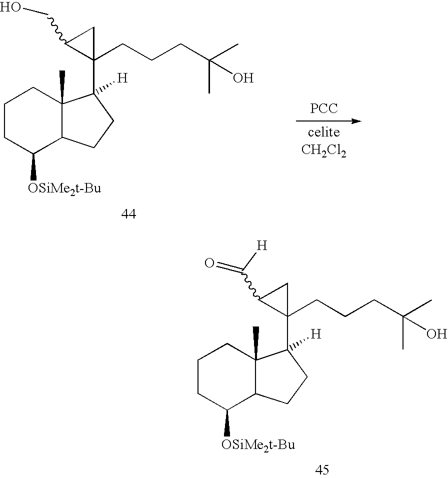

### 2-[(1S,3aR,4S,7aR)-4-(tert-Butyl-dimethyl-silanyloxy)-7a-methyl-octahydro inden-1-yl]-2-(4-hydroxy-4-methyl-pentyl)-cyclopropanecarbaldehyde (45)

A 25 ml round bottom flask equipped with stir bar and Claisen adapter with rubber septum was charged with 575 mg (2.667 mmol) of pyridinium chlorochromate, 650 mg of celite and 12 ml of dichloromethane. The 562 mg (1.128 mmol) of 5-{1-[(1S,3aR,4S,7aR)-4-(tert-butyl-dimethyl-silanyloxy)-7a-methyl-octahydro-inden-1-yl]-2-hydroxymethyl-cyclopropyl}-2-methyl-pentan-2-ol (44) in 4 ml of dichloromethane was added dropwise and mixture was stirred in room temperature for 2 h. The reaction mixture was filtrated through column with silica gel (50 cm³) and celite (3 cm) using dichloromethane, dichloromethane:ethyl acetate (4:1, 3:1). The fractions containing product were pooled and evaporated to give 550 mg of product 45 as yellow oil (product was used to the next reaction without purification).

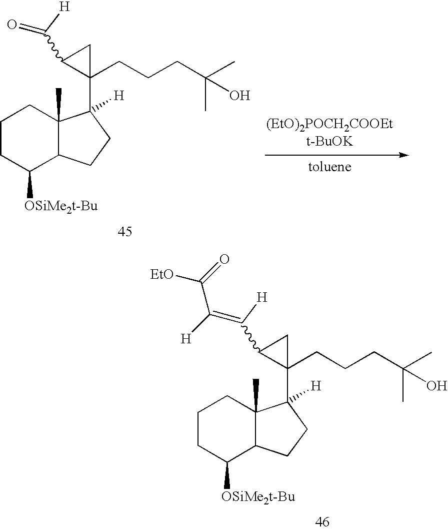

### 3-[2-[(1S,3aR,4S,7aR)-4-(tert-Butyl-dimethyl-silanyloxy)-7a-methyl-octahydro-inden-1-yl]-2-(4-hydroxy-4-methyl-pentyl)-cyclopropyl]-acrylic acid ethyl ester (46)

A 50 ml round bottom flask equipped with stir bar and Claisen adapter with rubber septum was charged with 15 ml of toluene and 4.5 ml of 1M potassium tert-butoxide in tetrahydrofurane was added. A 1.005 g (4.482 mmol) of triethyl phosphonoacetate in 0.5 ml of toluene was added dropwise at ca. 5° C. The mixture was stirred at room temperature for 1 h. Then the mixture was cooled to −15° C. and crude (ca. 1.281 mmol) of 2-[(1S,3aR,4S,7aR)-4-(tert-butyl-dimethyl-silanyloxy)-7a-methyl-octahydro-inden-1-yl]-2-(4-hydroxy-4-methyl-pentyl)-cyclopropanecarbaldehyde (45) in 4 ml of toluene was added and stirring was continued at −10° C. for 4 h. The reaction mixture was quenched with 50 ml of saturated solution of ammonium chloride and diluted with 50 ml of ethyl acetate and the inorganic layer was extracted twice with 50 ml of ethyl acetate, washed with 25 ml of brine, dried and evaporated. The residue was purified over silica gel (150 cm³) using hexane:ethyl acetate (5:1, 3:1) as a mobile phase to give 518 mg (80% for two steps) of products 46 as a mixture of isomers.

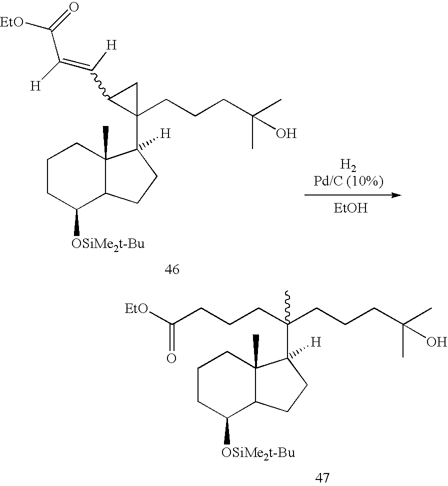

### 5-[(1R,3aR,4S,7aR)-4-(tert-Butyl-dimethyl-silanyloxy)-7a-methyl-octahydro-inden-1-yl]-9-hydroxy-5,9-dimethyl-decanoic acid ethyl ester (47)

A 550 mg (1.085 mmol) of 3-[2-[(1S,3aR,4S,7aR)-4-(tert-butyl-dimethyl-silanyloxy)-7a-methyl-octahydro-inden-1-yl]-2-(4-hydroxy-4-methyl-pentyl)-cyclopropyl]-acrylic acid ethyl ester (46) was hydrogenated over 200 mg of 10% Pd/C in 4 ml of ethanol at ambident temperature and atmospheric pressure of hydrogen. The reaction was monitoring by TLC (hexane:ethyl acetate-3:1). After 16 h the catalyst was filtered off and solvent evaporated. The residue was purified over silica gel (100 cm³) using hexane:ethyl acetate (10:1, 8:1, 3:1) as a mobile phase to give 549 mg (99%) of product 47 as a colorless oil (mixture of isomers).

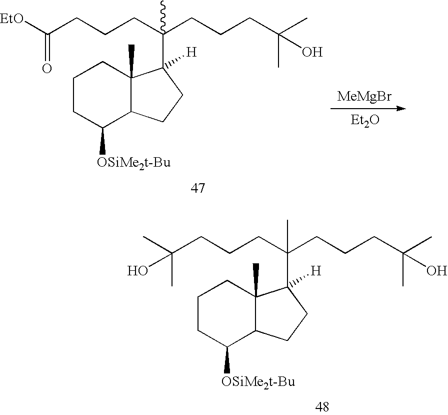

### 6-[(1R,3aR,4S,7aR)-4-(tert-Butyl-dimethyl-silanyloxy)-7a-methyl-octahydro-inden-1-yl]-2,6,10-trimethyl-undecane-2,10-diol (48)

A 50 ml round bottom flask equipped with stir bar, Claisen adapter with rubber septum was charged with 1.099 mg (2.151 mmol) of 5-[(1R,3aR,4S,7aR)-4-(tert-butyl-dimethyl-silanyloxy)-7a-methyl-octahydro-inden-1-yl]-9-hydroxy-5,9-dimethyl-decanoic acid ethyl ester (47) and 15 ml of diethyl ether. The solution was cooled in ace-water bath and 4.10 ml (12.792 mmol) of 3.12M solution of methylmagnesium bromide in diethyl ether was added dropwise. After completion of the addition the mixture was stirred at room temperature for 3.5 h then cooled again in an ice bath. A 10 ml of saturated solution of ammonium chloride was added dropwise. The resulting precipitate was dissolved by the addition of 50 ml of water. The aqueous layer was re-extracted three times with 50 ml of ethyl acetate. The combined ether layers were dried over Na₂SO₄ and evaporated. The oil residue was chromatographed on column (200 cm³) using hexane:ethyl acetate (3:1, 2:1, 1:1) as mobile phase. The chromatography (200 cm³) was repeated for mixture fractions to give 1.017 g (95%) of product 48 as colorless oil.

[α]_{D}³¹=36° c=0.36, CHCl₃

¹H NMR (CDCl₃): 3.98 (1H, br s), 2.00-1.95 (1H, m), 1.84-1.73 (1H, m), 1.66-1.63 (1H, m), 1.60-1.47 (4H, m), 1.43-1.30 (11H, m), 1.29-1.14 (8H, m), 1.20 (12H, s), 1.04 (3H, s), 0.90 (3H, s), 0.88 (9H, s), 0.00 (3H, s), −0.01 (3H, s)

¹³C NMR (CDCl₃): 71.07, 71.05, 69.67, 57.05, 53.05, 45.03, 44.98, 43.82, 41.63, 39.87, 39.37, 39.31, 34.44, 29.45, 29.39, 29.36, 29.33, 25.89, 23.09, 22.87, 21.99, 18.47, 18.11, 17.97, 17.86, 16.78, −4.69, −5.04

| | | | | |
|---|---|---|---|---|
| MS HRES | Calculated for: | C₃₀H₆₀O₃Si | [M + Na]⁺ | 519.4204 |
| | Observed: | | [M + Na]⁺ | 519.4203 |

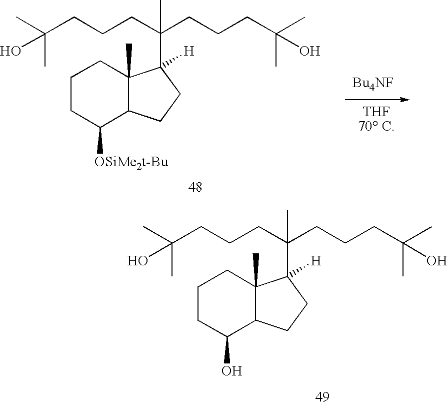

### 6-[(1R,3aR,4S,7aR)-4-Hydroxy-7a-methyl-octahydro-inden-1-yl]-2,6,10-trimethyl-undecane-2,10-diol (49)

A 25 ml round bottom flask equipped with stir bar and Claisen adapter with rubber septum was charged with 884 mg (1.779 mmol) of 6-[(1R,3aR,4S,7aR)-4-(tert-butyl-dimethyl-silanyloxy)-7a-methyl-octahydro-inden-1-yl]-2,6,10-trimethyl-undecane-2,10-diol (48) and 10 ml of 1M tetrabutylammonium fluoride in tetrahydrofurane. The reaction mixture was stirred at 70° C. for 48 h. (The new portion 5 ml of 1M tetrabutylammonium fluoride in tetrahydrofurane was added after 24 h). The mixture was dissolved by the addition of 150 ml of ethyl acetate and extracted six times with 50 ml of water:brine (1:1) and 50 ml of brine, dried over Na₂SO₄22 and evaporated. The oil residue was chromatographed on column (175 cm³) using hexane:ethyl acetate (2:1, 1:1) as mobile phase to give 590 mg (87%) of product 49 as colorless oil.

[α]_{D}³²=+11.4° c=0.35, CHCl₃

¹H NMR (CDCl₃): 4.07 (1H, br s), 2.02 (1H, br d, J=12.6 Hz), 1.84-1.76 (2H, m), 1.64-1.16 (24H, m), 1.21 (12H, s), 1.06 (3H, s), 0.91 (3H, s)

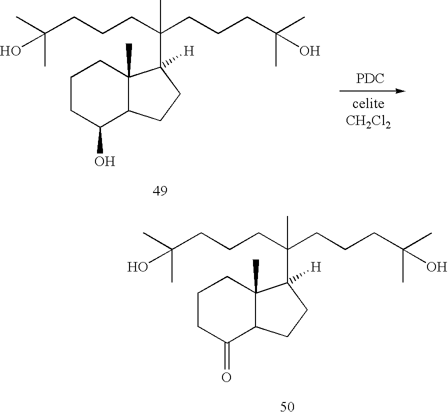

### (1R,3aR,4S,7aR)-1-[5-Hydroxy-1-(4-hydroxy-4-methyl-pentyl)-1,5-dimethyl-hexyl]-7a-methyl-octahydro-inden-4-one (50)

A 25 ml round bottom flask equipped with stir bar and Claisen adapter with rubber septum was charged with 1.745 g (4.638 mmol) of pyridinium dichromate, 2.00 g of celite and 15 ml of dichloromethane. A 590 mg (1.542 mmol) of 6-[(1R,3aR,4S,7aR)-4-hydroxy-7a-methyl-octahydro-inden-1-yl]-2,6,10-trimethyl-undecane-2,10-diol (49) in 4 ml of dichloromethane was added dropwise and mixture was stirred in room temperature for 5 h. The reaction mixture was filtrated through column with silica gel (50 cm³) and celite (3 cm) using dichloromethane, dichloromethane:ethyl acetate (2:1, 1:1) as a mobile phase. The fractions containing product were pooled and evaporated to give 577 mg (98%) of ketone 50.

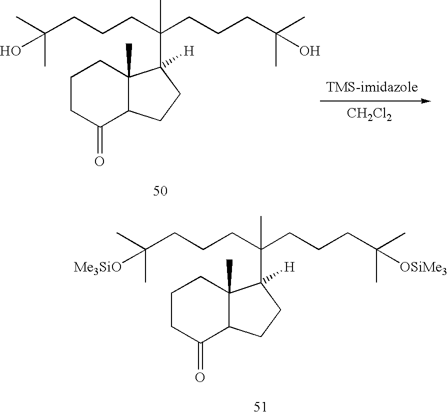

### (1R,3aR,4S,7aR)-1-[1,5-Dimethyl-1-(4-methyl-4-trimethylsilanyloxy-pentyl)-5-trimethylsilanyloxy-hexyl]-7a-methyl-octahydro-inden-4-one (51)

A 25 ml round bottom flask equipped with stir bar and Claisen adapter with rubber septum was charged with 577 mg (1.516 mmol) of (1R,3aR,4S,7aR)-1-[5-hydroxy-1-(4-hydroxy-4-methyl-pentyl)-1,5-dimethyl-hexyl]-7a-methyl-octahydro-inden-4-one (50) and 10 ml of dichloromethane. A 1.80 ml (12.269 mmol) of 1-(trimethylsilyl) imidazole was added dropwise. The mixture was stirred at room temperature for 2 h 30 min. The resulting mixture was dissolved by the addition of 100 ml of water. The aqueous layer was extracted four times with 50 ml of ethyl acetate. The combined organic layers were washed with 50 ml of brine, dried over Na₂SO₄ and evaporated. The residue was purified over silica gel (50 cm³) using hexane:ethyl acetate (10:1) as a mobile phase to give a 739 mg (93%) of product 51 as colorless oil.

¹H NMR (CDCl₃): 2.42 (1H, dd, J=9.9, 7.3 Hz), 2.30-2.13 (3H, m), 2.04-1.50 (9H, m), 1.42-1.14 (1H, m), 1.21 (6H, s), 1.20 (6H, s), 0.90 (3H, s), 0.73 (3H, s), 0.11 (9H, s), 0.10 (9H, s)

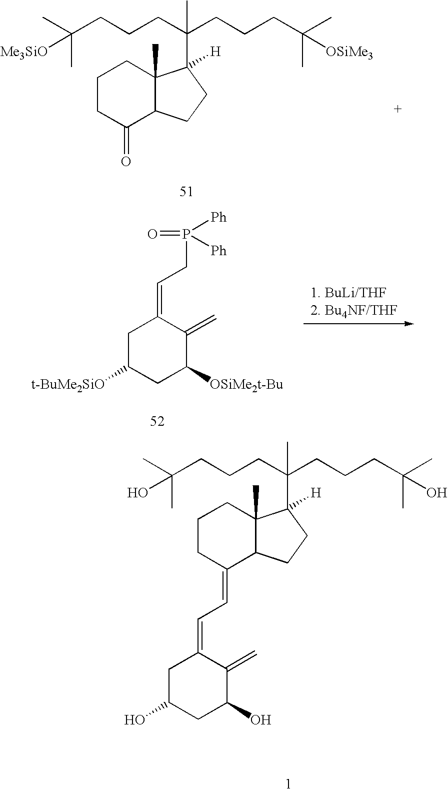

### 1,25-Dihydroxy-20-(4-hydroxy-4-methyl-pentyl)cholecalciferol (1)

A 25 ml round bottom flask equipped with stir bar and Claisen adapter with rubber septum was charged with 700 mg (1.201 mmol) of (1S,5R)-1,5-bis-((tert-butyldimethyl)silanyloxy)-3-[2-(diphenylfosphinoyl)-eth-(Z)-ylidene]-2-methylene-cyclohexane (52) and 5 ml of tetrahydrofurane. The reaction mixture was cooled to −70° C. and 0.75 ml (1.200 mmol) of 1.6M n-butyllithium was added dropwise. The resulting deep red solution was stirred at −78° C. for 25 min and 300 mg (0.571 mmol) of (1R,3aR,4S,7aR)-1-[1,5-dimethyl-1-(4-methyl-4-trimethylsilanyloxy-pentyl)-5-trimethylsilanyloxy-hexyl]-7a-methyl-octahydro-inden-4-one (51) was added dropwise in 1 ml of tetrahydrofurane. The reaction mixture was stirred for 5 h and then the bath was removed and the mixture was poured into 50 ml of ethyl acetate and 100 ml of brine. The water fraction was extracted four times with 50 ml of ethyl acetate, dried over Na₂SO₄ and evaporated. The oil residue was chromatographed on column (50 cm³, protected from light) using hexane:ethyl acetate (20:1) as mobile phase. Fractions containing product were pooled and evaporated to give colorless oil (ca. 430 mg) which was treated with 5 ml of 1M tetrabutylammonium fluoride in tetrahydrofurane. The reaction mixture was stirred at room temperature for 24 h. The mixture was dissolved by the addition of 150 ml ethyl acetate and extracted six times with 50 ml of water:brine (1:1) and 50 ml of brine, dried over Na₂SO₄ and evaporated. The oil residue was chromatographed on column (50 cm³, protected from light) using ethyl acetate as mobile phase. Fractions containing product were pooled and evaporated to give colorless oil. Oil was crystallized from methyl acetate to give 183 mg (62%) of product 1.

[α]_{D}²⁹=+12.3° c=0.40, EtOH

UV λmax (EtOH): 213 nm (ε 14606), 264 nm (ε 17481)

¹H NMR (CDCl₃): 6.18 (1H, d, J=11.1 Hz), 5.97 (1H, d, J=11.3 Hz), 5.23 (1H, d, J=1.3 Hz), 4.86 (1H, d, J=4.7 Hz), 4.75 (1H, d, J=1.7 Hz), 4.54 (1H, d, J=3.8 Hz), 4.20-4.16 (1H, m), 4.05 (1H, s), 4.04 (1H, s), 4.01-3.96 (1H, m), 2.77 (1H, br d, J=11.7 Hz), 2.35 (1H, br d, J=11.5 Hz), 2.17 (1H, dd, J=13.5, 5.2 Hz), 2.01-1.94 (2H, m), 1.83-1.78 (1H, m), 1.68-1.52 (6H, m), 1.48-1.05 (16H, m), 1.06 (12H, s), 0.86 (3H, s), 0.60 (3H, s)

¹³C NMR (CDCl₃): 149.41, 139.87, 135.74, 122.37, 117.81, 109.72, 68.72, 68.69, 68.34, 65.07, 56.64, 56.05, 46.17, 44.85, 44.79, 43.11, 40.53, 40.12, 39.56, 38.89, 29.48, 29.45, 29.18, 28.34, 23.15, 22.98, 21.89, 21.59, 18.07, 17.56, 14.70

| | | | | |
|---|---|---|---|---|
| MS HRES | Calculated for: | C₃₃H₅₆O₄ | [M + Na]⁺ | 539.4071 |
| | Observed: | | [M + Na]⁺ | 539.4066 |

### Example 2

### Synthesis of 1,25-Dihydroxy-20-(4-hydroxy-4-methyl-pentyl)-19-nor-cholecalciferol (2)

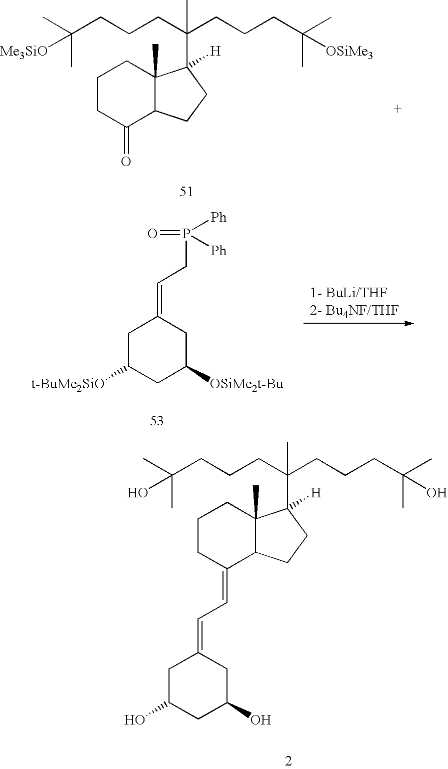

### 1,25-Dihydroxy-20-(4-hydroxy-4-methyl-pentyl)-19-nor-cholecalciferol (2)

A 25 ml round bottom flask equipped with stir bar and Claisen adapter with rubber septum was charged with 1.023 g (1.792 mmol) of (1R,3R)-1,3-bis-((tert-butyldimethyl)silanyloxy)-5-[2-(diphenylfosphinoyl)ethylidene]-cyclohexane (53) and 5 ml of tetrahydrofurane. The reaction mixture was cooled to −70° C. and 1.12 ml (1.792 mmol) of 1.6M n-butyllithium BuLi was added dropwise. The resulting deep red solution was stirred at −78° C. for 25 min and 350 mg (0.667 mmol) of (1R,3aR,4S,7aR)-1-[1,5-dimethyl-1-(4-methyl-4-trimethylsilanyloxy-pentyl)-5-trimethylsilanyloxy-hexyl]-7a-methyl-octahydro-inden-4-one (51) in 1 ml of tetrahydrofurane. The reaction mixture was stirred for 5 h and then the dry ice was removed from bath and the solution was allowed to warm up to −40° C. in 1 h. The mixture was poured into 50 ml of ethyl acetate and 100 ml of brine. The water fraction was extracted four times with 50 ml of ethyl acetate, dried over Na₂SO₄ and evaporated. The oil residue was chromatographed on column (50 cm³, protected from light) using hexane:ethyl acetate (30:1 and 10:1) as mobile phase. Fractions containing product were pooled and evaporated to give colorless oil (ca. 500 mg) which was treated with 6 ml of 1M tetrabutylammonium fluoride in tetrahydrofurane. The reaction mixture was stirred at room temperature for 20 h. The new portion 3 ml of 1M tetrabutylammonium fluoride in tetrahydrofurane was added and the mixture was stirred for 22 h. The mixture was dissolved by the addition of 150 ml of ethyl acetate and extracted six times with 50 ml of water:brine (1:1) and 50 ml of brine, dried over Na₂SO₄ and evaporated. The oil residue was chromatographed on column (50 cm³, protected from light) using ethyl acetate as mobile phase. Fractions containing product were pooled and evaporated to give product as colorless oil. Oil was dissolved in methyl acetate and evaporated (2 times) to give 285 mg (85%) of product 2 as a white solid.

[α]_{D}²³=+38.2° c=0.38, CHCl₃

UV λmax (EtOH): 243 nm (ε 33019), 251 nm (ε 38843), 261 nm (ε 26515)

¹H NMR (CDCl₃): 6.29 (1H, d, J=1.1 Hz), 5.83 (1H, d, J=1.1 Hz), 4.12-4.09 (1H, m), 4.06-4.00 (1H, m), 2.80-2.71 (2H, m), 2.47 (1H, dd, J=13.3, 3.1 Hz), 2.23-2.17 (2H, m), 2.05-1.91 (3H, m), 1.78 (1H, ddd, J=13.1, 8.3, 3.1 Hz), 1.67-1.16 (24H, m), 1.21 (12H, s), 0.89 (3H, s), 0.63 (3H, s)

¹³C NMR (CDCl₃): 142.76, 131.16, 123.67, 115.63, 71.04, 67.38, 67.15, 57.18, 56.69, 46.73, 44.97, 44.92, 44.66, 42.20, 41.15, 39.70, 39.54, 39.37, 37.22, 29.44, 29.39, 29.36, 28.90, 23.48, 23.14, 22.41, 21.97, 18.44, 17.95, 15.12

| | | | | |
|---|---|---|---|---|
| MS HRES | Calculated for: | C₃₂H₅₆O₄ | [M + Na]⁺ | 527.4071 |
| | Observed: | | [M + Na]⁺ | 527.4073 |

### Example 3

### Synthesis of 1α-Fluoro-25-hydroxy-20-(4-hydroxy-4-methyl-pentyl-cholecalciferol (3)

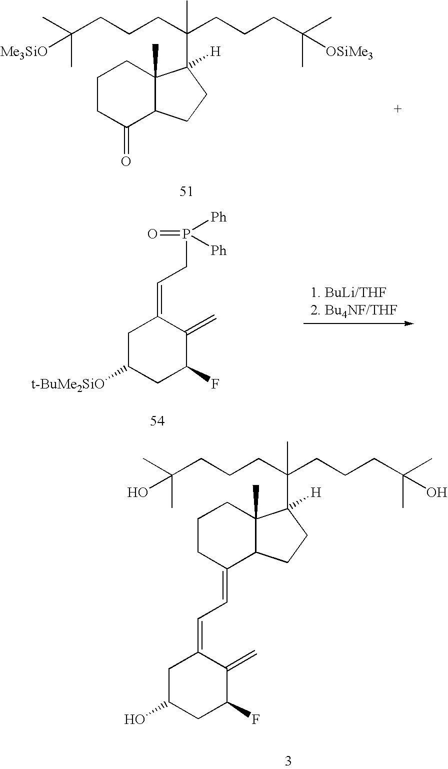

### 1α-Fluoro-25-hydroxy-20-(4-hydroxy-4-methyl-pentyl)-cholecalciferol (3)

A 25 ml round bottom flask equipped with stir bar and Claisen adapter with rubber septum was charged with 680 mg (1.445 mmol) of (1S,5R)-1-((tert-butyldimethyl)silanyloxy)-3-[2-(diphenylfosphinoyl)-eth-(Z)-ylidene]-5-fluoro-2-methylene-cyclohexane (54) and 5 ml of tetrahydrofurane. The reaction mixture was cooled to −70° C. and 0.9 ml (1.44 mmol) of 1.6M n-butyllithium was added dropwise. The resulting deep red solution was stirred at −78° C. for 25 min and 300 mg (0.571 mmol) of (1R,3aR,4S,7aR)-1-[1,5-dimethyl-1-(4-methyl-4-trimethylsilanyloxy-pentyl)-5-trimethylsilanyl oxy-hexyl]-7a-methyl-octahydro-inden-4-one (51) was added dropwise in 1 ml of tetrahydrofurane. The reaction mixture was stirred for 4 h and then the dry ice was removed from bath and the solution was allowed to warm up to −40° C. in 1 h. The mixture was poured into 50 ml of ethyl acetate and 100 ml of brine. The water fraction was extracted three times with 50 ml of ethyl acetate, dried over Na₂SO₄ and evaporated. The oil residue was chromatographed on column (50 cm³, protected from light) using hexane:ethyl acetate (30:1 and 10:1) as mobile phase. Fractions containing product were pooled and evaporated to give colorless oil (ca. 399 mg) which was treated with 5 ml of 1M tetrabutylammonium fluoride in tetrahydrofurane. The reaction mixture was stirred at room temperature for 20 h. The mixture was dissolved by the addition of 150 ml of ethyl acetate and extracted six times with 50 ml of water:brine (1:1) and 50 ml of brine, dried over Na₂SO₄ and evaporated. The oil residue was chromatographed on column (50 cm³, protected from light) using ethyl acetate:hexane (2:1 and 3:1) as mobile phase. Fractions containing product were pooled and evaporated to give product as colorless oil. The product was dissolved in methyl acetate and evaporated (2 times) to give 243 mg (82%) of product 3 as white foam.

[α]_{D}²⁸=+9.3° c=0.40, CHCl₃

UV λmax (EtOH): 208 nm (ε 16024), 242 nm (ε 14965), 270 nm (ε 15024)

¹H NMR (CDCl₃): 6.39 (1H, d, J=11.1 Hz), 6.01 (1H, d, J=11.3 Hz), 5.38 (1H, s), 5.13 (1H, ddd, J=49.9, 6.8, 3.7 Hz), 5.09 (1H, s), 4.25-4.18 (1H, m), 2.82-2.77 (1H, m), 2.61 (1H, dd, J=13.3, 3.7 Hz), 2.30 (1H, dd, J=13.3, 7.6 Hz), 2.22-2.13 (1H, m), 2.07-1.94 (3H, m), 1.76-1.15 (24H, m), 1.21 (12H, s), 0.89 (3H, s), 0.63 (3H, s)

¹³C NMR (CDCl₃): 143.30, 143.06 (d, J=16.7 Hz), 131.40, 125.47, 117.37, 114.71 (d, J=9.9 Hz), 91.53 (d, J=172.6 Hz), 71.05, 71.05, 66.53, 66.47, 57.17, 56.74, 46.89, 44.96, 44.90, 41.17, 40.87, 40.67, 39.67, 39.51, 39.36, 29.41, 29.35, 29.07, 23.56, 23.11, 22.37, 21.90, 18.43, 17.94, 15.05

### Example 4

### Synthesis of (20S)-1,25-Dihydroxy-20-(5,5,5-trifluoro-4-hydroxy-4-trifluoromethyl-pent-2-ynyl)cholecalciferol (4)

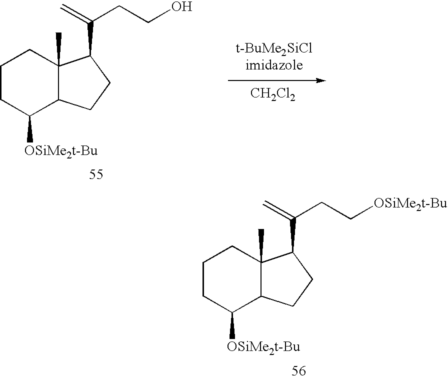

### (1R,3aR,4S,7aR)-4-(tert-Butyl-dimethyl-silanyloxy)-1-[3-(tert-butyl-dimethyl-silanyloxy)-1-methylene-propyl]-7a-methyl-octahydro-indene (56)

A 250 ml round bottom flask equipped with stir bar, Claisen adapter with rubber septum and nitrogen sweep was charged with 17.53 g (51.77 mmol) of 3-[(1R,3aR,4S,7aR)-4-(tert-butyl-dimethyl-silanyloxy)-7a-methyl-octahydro-inden-1-yl]-but-3-en-1-ol (55) and 75 ml of dichloromethane. A 7.05 g (103.54 mmol) imidazole was added followed by 9.36 g (62.124 mmol) of t-butyldimethylsilyl chloride. The mixture was stirred for 2.5 h. The mixture was then diluted with 100 ml of water and extracted four times with 50 ml of dichloromethane. The combined organic layers were dried over Na₂SO₄ and evaporated. The oil residue was chromatographed on column (400 cm³) using hexane, hexane:ethyl acetate (50:1, 25:1) as mobile phase and collecting ca. 40 ml fractions to give 22.32 g (95%) of product 56 as a colorless oil.

¹H NMR (CDCl₃): 4.87 (1H, s), 4.80 (1H, s), 4.02 (1H, br s), 3.67 (2H, t, J=7.3 Hz), 2.34-2.14 (2H, m), 2.06-2.00 (1H, m), 1.85-1.27 (9H, m), 1.20-1.08 (2H, m), 0.89 (18H, s), 0.79 (3H, s), 0.05 (6H, s), 0.02 (3H, s), 0.01 (3H, s).

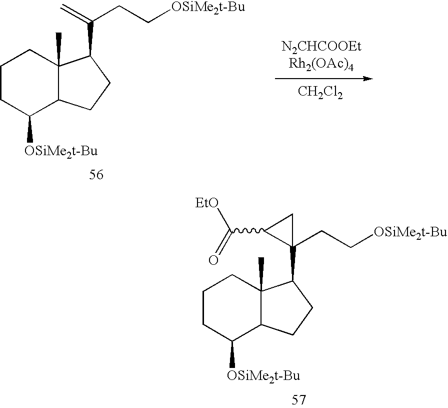

### 2-[2-(tert-Butyl-dimethyl-silanyloxy)-ethyl]-2-[(1S,3aR,4S,7aR)-4-(tert-butyl-dimethyl-silanyloxy)-7a-methyl-octahydro-inden-1-yl]-cyclopropanecarboxylic acid ethyl ester (57)

A 250 ml round bottom flask equipped with stir bar and Claisen adapter with rubber septum was charged with 10.00 g (22.08 mmol) of (1R,3aR,4S,7aR)-4-(tert-butyl-dimethyl-silanyloxy)-1-[3-(tert-butyl-dimethyl-silanyloxy)-1-methylene-propyl]-7a-methyl-octahydro-indene (56), 200 mg of Rh₂(OAc)₄ and 40 ml of dichloromethane. A solution of 5.304 g (46.486 mmol) of ethyl diazoacetate in 30 ml of dichloromethane was added dropwise (12 ml/h) at room temperature. The reaction mixture was concentrated in vacuo and the remaining residue was filtrated on column (200 cm³) using hexane:ethyl acetate (1:1) as mobile phase. The solvent was evaporated and the oil residue was chromatographed on column (250 cm³) using hexane:ethyl acetate (25:1, 10:1 and 5:1) as mobile phase to give 8.44 g (71%) of products 57 as a mixture of isomers.

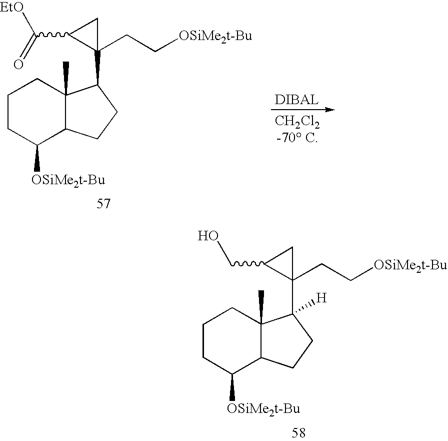

### {2-[2-(tert-Butyl-dimethyl-silanyloxy)-ethyl]-2-[(1S,3aR,4S,7aR)-4-(tert-butyl-dimethyl-silanyloxy)-7a-methyl-octahydro-inden-1-yl]-cyclopropyl}-methanol (58)

A 50 ml round bottom flask equipped with stir bar and Claisen adapter with rubber septum was charged with 4.140 g (7.682 mmol) of 2-[2-(tert-butyl-dimethyl-silanyloxy)-ethyl]-2-[(1S,3aR,4S,7aR)-4-(tert-butyl-dimethyl-silanyloxy)-7a-methyl-octahydro-inden-1-yl]-cyclopropanecarboxylic acid ethyl ester (57) and 20 ml of dichloromethane. The reaction mixture was cooled to −70° C. and 10.0 ml (15.0 mmol) of 1.5M DIBAL-H in toluene was added dropwise during 45 min. The reaction was stirred at −70° C. for 1 h and then 5 ml of saturated solution of ammonium chloride was added dropwise. The mixture was dissolved by the addition of 100 ml of water and 50 ml of 1N HCl, extracted three times with 50 ml of ethyl acetate, dried over Na₂SO₄ and evaporated. The oil residue was chromatographed on column (200 cm³) using hexane:ethyl acetate (10:1, 3:1) as mobile phase. The fractions containing product were pooled and evaporated to give 3.610 g, (94%) of products 58 (mixture of isomers) as colorless oil.

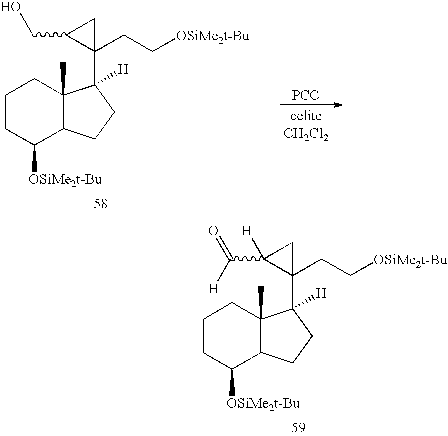

### 2-[2-(tert-Butyl-dimethyl-silanyloxy)-ethyl]-2-[(1S,3aR,4S,7aR)-4-(tert-butyl-dimethyl-silanyloxy)-7a-methyl-octahydro-inden-1-yl]-cyclopropanecarbaldehyde (59)

A 250 ml round bottom flask equipped with stir bar and Claisen adapter with rubber septum was charged with 6.074 g (28.178 mmol) of pyridinium chlorochromate, 7.00 g of celite and 100 ml of dichloromethane. A 6.970 g (14.027 mmol) of {2-[2-(tert-butyl-dimethyl-silanyloxy)-ethyl]-2-[(1S,3aR,4S,7aR)-4-(tert-butyl-dimethyl-silanyloxy)-7a-methyl-octahydro-inden-1-yl]-cyclopropyl}-methanol (58) in 10 ml of dichloromethane was added dropwise and mixture was stirred in room temperature for 1 h. The reaction mixture was filtrated through column with silica gel (200 cm³) and celite (2 cm) and using dichloromethane as a mobile phase. The fractions containing product were pooled and evaporated to give oil (ca. 5.71 g). Product 59 was used to the next reaction without purification.

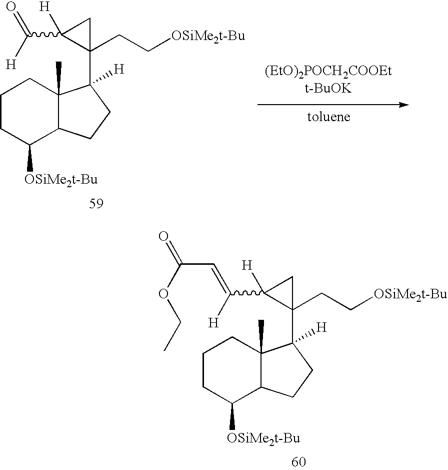

### 3-{2-[2-(tert-Butyl-dimethyl-silanyloxy)-ethyl]-2-[(1S,3aR,4S,7aR)-4-(tert-butyl-dimethyl-silanyloxy)-7a-methyl-octahydro-inden-1-yl]-cyclopropyl}-acrylic acid ethyl ester (60)

A 250 ml round bottom flask equipped with stir bar and Claisen adapter with rubber septum was charged with 80 ml of toluene and 35.0 ml (35.0 mmol) of 1M potassium tert-butoxide in tetrahydrofurane was added. A 7.850 g (35.015 mmol) of triethyl phosphonoacetate in 5 ml of toluene was added dropwise at ca. 5° C. The mixture was stirred at room temperature for 1 h. Then the mixture was cooled to −15° C. and crude (ca. 11.54 mmol) 2-[2-(tert-butyldimethyl-silanyloxy)-ethyl]-2-[(1S,3aR,4S,7aR)-4-(tert-butyl-dimethyl-silanyloxy)-7a-methyl-octahydro-inden-1-yl]-cyclopropanecarbaldehyde (59) in 5 ml of toluene was added and stirring was continued at −10° C. for 3 h. The reaction mixture was quenched with 10 ml of aqueous saturated solution of ammonium chloride, diluted with 100 ml of saturated solution of ammonium chloride and extracted four times with 50 ml of toluene and then 50 ml of ethyl acetate. The organic layer was washed with 50 ml of brine, dried and evaporated. The residue was purified over silica gel (200 cm³) using hexane:ethyl acetate (20:1) as a mobile phase to give 5.750 g (88%) of products 60 (mixture of isomers).

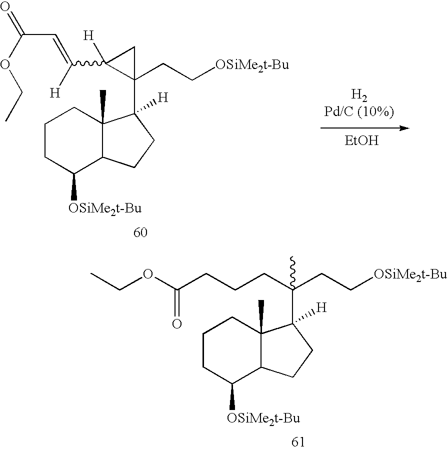

### 7-(tert-Butyl-dimethyl-silanyloxy)-5-[(1R,3aR,4S,7aR)-4-(tert-butyl-dimethyl-silanyloxy)-7a-methyl-octahydro-inden-1-yl]-5-methyl-heptanoic acid ethyl ester (61)

A 5.750 g (10.177 mmol) of 3-{2-[2-(tert-butyl-dimethyl-silanyloxy)-ethyl)-2-[(1S,3aR,4S,7aR)-4-(tert-butyl-dimethyl-silanyloxy)-7a-methyl-octahydro-inden-1-yl]cyclopropyl}-acrylic acid ethyl ester (60) was hydrogenated over 1.60 g of 10% Pd/C in 40 ml of ethanol at room temperature and atmospheric pressure of hydrogen. The reaction was monitoring by TLC (hexane:ethyl acetate-50:1). After 18 h the catalyst was filtered off and solvent evaporated. The residue was purified over silica gel (300 cm³) using hexane:ethyl acetate (100:1, 50:1, 20:1) as a mobile phase to give 5.150 g (89%) of products 61 (mixture of isomers).

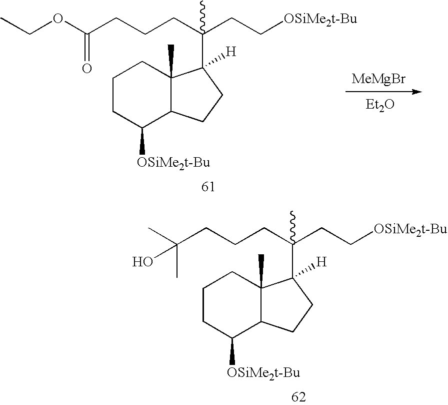

### 8-(tert-Butyl-dimethyl-silanyloxy)-6-[(1R,3aR,4S,7aR)-4-(tert-butyl-dimethyl-silanyloxy)-7a-methyl-octahydro-inden-1-yl]-2,6-dimethyl-octan-2-ol (62)

A 250 ml round bottom flask equipped with stir bar, Claisen adapter with rubber septum was charged with 5.110 g (8.980 mmol) of 7-(tert-butyl-dimethyl-silanyloxy)-5-[(1R,3aR,4S,7aR)-4-(tert-butyl-dimethyl-silanyloxy)-7a-methyl-octahydro-inden-1-yl]-5-methyl-heptanoic acid ethyl ester ester (61) and 80 ml of diethyl ether. The solution was cooled in ace-water bath and 17.4 ml (54.3 mmol) of 3.12M solution of methyl magnesium bromide in diethyl ether was added dropwise. After completion of the addition the mixture was stirred at room temperature for 2.5 h then cooled again in an ice bath. A 10 ml of saturated solution of ammonium chloride was added dropwise. The resulting precipitate was dissolved by the addition of 50 ml of saturated solution of ammonium chloride. The aqueous layer was extracted three times with 100 ml of ethyl acetate. The combined organic layers were dried (Na₂SO₄) and evaporated. The product 62 was used to the next reaction without farther purification.

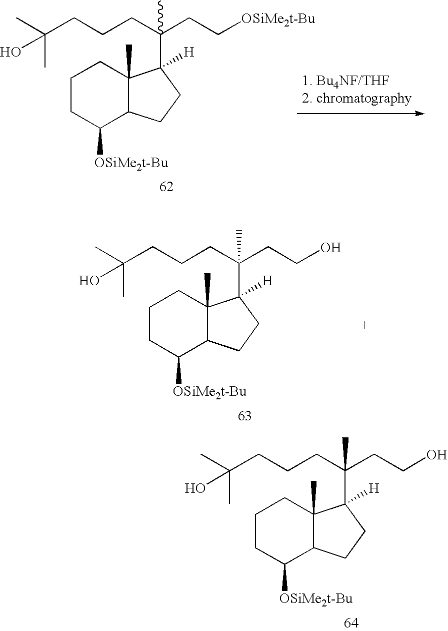

### 3-[(1R,3aR,4S,7aR)-4-(tert-Butyl-dimethyl-silanyloxy)-7a-methyl-octahydro-inden-1-yl]-3,7-dimethyl-octane-1,7-diol (63 and 64)

A 50 ml round bottom flask equipped with stir bar and Claisen adapter with rubber septum was charged with crude (ca. 8.98 mmol) 8-(tert-butyl-dimethyl-silanyloxy)-6-[4-(tert-butyl-dimethyl-silanyloxy)-7a-methyl-octahydro-inden-1-yl]-2,6-dimethyl-octan-2-ol (62), 10 ml of tetrahydrofurane and 15.0 ml (15.0 mmol) of 1M tetrabutylammonium fluoride in tetrahydrofurane. The reaction mixture was stirred at room temperature for 2.5 h. The mixture was dissolved by the addition of 150 ml of ethyl acetate and extracted six times with 50 ml of water:brine (1:1) and 50 ml of brine, dried over Na₂SO₄ and evaporated. The oil residue was chromatographed four times on columns (400 cm³) using hexane:ethyl acetate (1:1) as a mobile phase to give: 1^{st}—1.456 g (low polar epimer); 2^{nd} —0.852 g, (mixture of epimers)' 3^{rd}—1.132 g (more polar epimer)' All products 3.440 g (88% two steps).

### Low polar epimer: (3S)-3-[(1R,3aR,4S,7aR)-4-(tert-Butyl-dimethyl-silanyloxy)-7a-methyl-octahydro-inden-1-yl]-3,7-dimethyl-octane-1,7-diol (63)

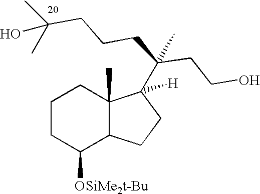

[α]_{D}³¹=+26.1° c=0.44, CHCl₃

¹H NMR (CDCl₃): 3.90 (1H, br s), 3.67 (2H, br t, J=8.1 Hz), 2.06-1.99 (1H, m), 1.87-1.50 (4H, m), 1.73 (2H, t, J=7.9 Hz), 1.40-1.06 (14H, m), 1.22 (6H, s), 1.06 (3H, s), 0.95 (3H, s), 1.95-0.82 (1H, m), 0.88 (9H, s), 0.00 (3H, s), −0.01 (3H, s)

¹³C NMR (CDCl₃): 71.03, 69.58, 59.79, 57.32, 52.99, 44.78, 43.81, 41.64, 41.58, 40.26, 38.68, 34.37, 29.48, 29.36, 25.86, 23.49, 22.78, 21.72, 18.18, 18.09, 17.78, 16.78, −4.70, −5.07

| | | | | |
|---|---|---|---|---|
| MS HRES | Calculated for: | C₂₆H₅₂O₃Si | [M + Na]⁺ | 463.3578 |
| | Observed: | | [M + Na]⁺ | 463.3580 |

### More polar epimer: (3R)-3-[(1R,3aR,4S,7aR-4-(tert-Butyl-dimethyl-silanyloxy)-7a-methyl-octahydro-inden-1-yl]-3,7-dimethyl-octane-1,7-diol (64)

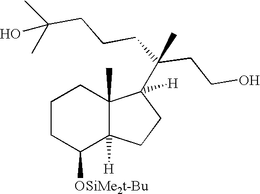

[α]_{D}³¹=+22.7° c=0.44, CHCl₃

¹H NMR (CDCl₃): 3.99-3.97 (1H, m), 3.65-3.61 (2H, m), 1.97 (1H, br d, J=12.3 Hz), 1.84-1.72 (1H, m), 1.66-1.50 (6H, m), 1.45-1.15 (14H, m), 1.21 (6H, s), 1.05 (3H, s), 0.95 (3H, s), 0.87 (9H, s), −0.01 (3H, s), −0.02 (3H, s)

¹³C NMR (CDCl₃): 71.05, 69.57, 59.47, 57.46, 53.02, 44.87, 43.90, 41.83, 41.61, 39.99, 38.93, 34.37, 29.43, 29.42, 25.87, 23.42, 22.84, 22.12, 18.57, 18.09, 17.81, 16.79, −4.69, −5.06

| | | | | |
|---|---|---|---|---|
| MS HRES | Calculated for: | C₂₆H₅₂O₃Si | [M + Na]⁺ | 463.3578 |
| | Observed: | | [M + Na]⁺ | 463.3575 |

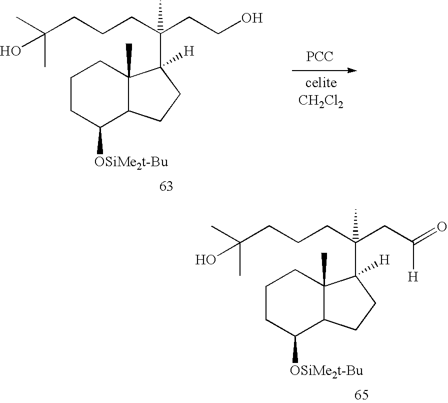

### (3S)-3-[(1R,3aR,4S,7aR)-4-(tert-Butyl-dimethyl-silanyloxy)-7a-methyl-octahydro-inden-1-yl]-7-hydroxy-3,7-dimethyl-octanal (65)

A 25 ml round bottom flask equipped with stir bar and Claisen adapter with rubber septum was charged with 1.572 g (7.292 mmol) of pyridinium chlorochromate, 1.60 g of celite and 25 ml of dichloromethane. A 1.607 g (3.646 mmol) of (3S)-3-[(1R,3aR,4S,7aR)-4-(tert-butyl-dimethyl-silanyloxy)-7a-methyl-octahydro-inden-1-yl]-3,7-dimethyl-octane-1,7-diol (63) in 6 ml of dichloromethane was added dropwise and mixture was stirred at room temperature for 1 h 45 min and additional portion 300 mg (1.392 mmol) of pyridinium chlorochromate was added. The reaction was stirred for next 1 h 15 min. The reaction mixture was filtrated through column with silica gel (50 cm³) and celite (1 cm) using dichloromethane, dichloromethane:ethyl acetate (4:1). The fractions containing product were pooled and evaporated to give 1.58 g of product as yellow oil. The product 65 was used to the next reaction without further purification.

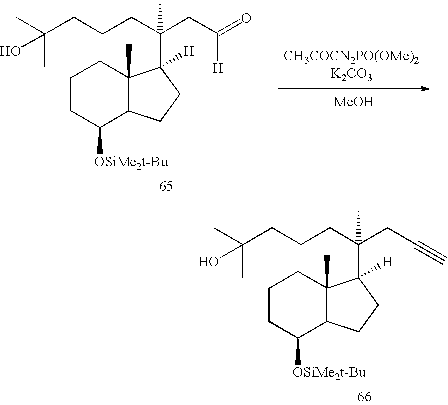

### (6S)-6-[(1R,3aR,4S,7aR)-4-(tert-Butyl-dimethyl-silanyloxy)-7a-methyl-octahydro-inden-1-yl]-2,6-dimethyl-non-8-yn-2-ol (66)

A 50 ml round bottom flask equipped with stir bar and Claisen adapter with rubber septum was charged with 1.58 g (3.601 mmol) of (3S)-3-[(1R,3aR,4S,7aR)-4-(tert-butyl-dimethyl-silanyloxy)-7a-methyl-octahydro-inden-1-yl]-7-hydroxy-3,7-dimethyl-octanal (65) and 30 ml of methanol. A 1.416 g (7.37 mmol) of 1-diazo-2-oxo-propyl)-phosphonic acid dimethyl ester in 3 ml of methanol was added and the resulting mixture was cooled in an ice bath. A 1.416 g (10.245 mmol) of potassium carbonate was added and the reaction mixture was stirred in the ice bath for 30 min and then at room temperature for 3 h. A 100 ml of water was added and the mixture was extracted three times with 80 ml of ethyl acetate, dried over Na₂SO₄ and evaporated. The oil residue was chromatographed on column (250 cm³) using hexane:ethyl acetate (7:1) as mobile phase. Fractions containing product were pooled and evaporated to give 1.310 g (83%, 2 steps) of product 66 as colorless oil.

[α]_{D}³⁰=+15.7° c=0.61, CHCl₃

¹H NMR (CDCl₃): 3.98 (1H, br s), 2.28 (2H, d, J=2.1 Hz), 1.95-1.91 (2H, m), 1.78 (1H, dt, J=13.4, 3.8 Hz), 1.68-1.62 (1H, m), 1.58-1.48 (6H, m), 1.44-1.17 (15H, m), 1.22 (6H, s), 1.04 (3H, s), 1.00 (3H, s), 0.93-0.83 (1H, m), 0.88 (9H, s), −0.00 (3H, s), −0.01 (3H, s)

¹³C NMR (CDCl₃): δ3.09, 71.03, 69.84, 69.64, 56.68, 52.95, 44.80, 43.71, 41.31, 40.21, 39.28, 34.33, 29.44, 29.29, 28.80, 25.85, 22.74, 22.69, 22.18, 18.14, 18.05, 17.73, 16.68, −4.77, −5.13

| | | | | |
|---|---|---|---|---|
| MS HRES | Calculated for: | C₂₇H₅₀O₂Si | [M + Na]⁺ | 457.3472 |
| | Observed: | | [M + Na]⁺ | 457.3473 |

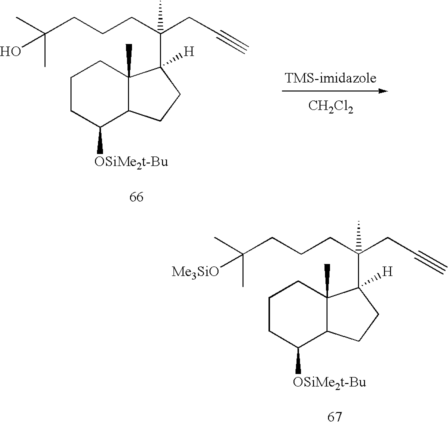

### (1R,3aR,4S,7aR)-4-(tert-Butyl-dimethyl-silanyloxy)-1-[(1S)-1,5-dimethyl-1-prop-2-ynyl-5-trimethylsilanyloxy-hexyl]-7a-methyl-octahydro-indene (67)

A 50 ml round bottom flask equipped with stir bar and Claisen adapter with rubber septum was charged with 1.300 g (2.990 mmol) of (6S)-6-[(1R,3aR,4S,7aR)-4-(tert-butyl-dimethyl-silanyloxy)-7a-methyl-octahydro-inden-1-yl]-2,6-dimethyl-non-8-yn-2-ol (66) and 25 ml of dichloromethane. A 2.00 ml (13.63 mmol) of 1-(trimethylsilyl) imidasole was added dropwise. The mixture was stirred at room temperature for 1 h. A 100 ml of water was added and the mixture was extracted three times with 80 ml of hexane, dried over Na₂SO₄ and evaporated. The oil residue was chromatographed on column (75 cm³) using hexane:ethyl acetate (25:1) as mobile phase. Fractions containing product were pooled and evaporated to give 1.409 g (93%) of product 67 as colorless oil.

¹H NMR (CDCl₃): 3.98 (1H, br s), 2.27 (2H, d, J=2.9 Hz), 1.97-1.91 (2H, m), 1.82-1.75 (1H, m), 1.69-1.62 (1H, m), 1.59-1.50 (2H, m), 1.42-1.20 (12H, m), 1.20 (6H, s), 1.05 (3H, s), 1.00 (3H, s), 0.93-0.85 (1H, m), 0.88 (9H, s), 0.10 (9H, s), 0.00 (3H, s), −0.01 (3H, s)

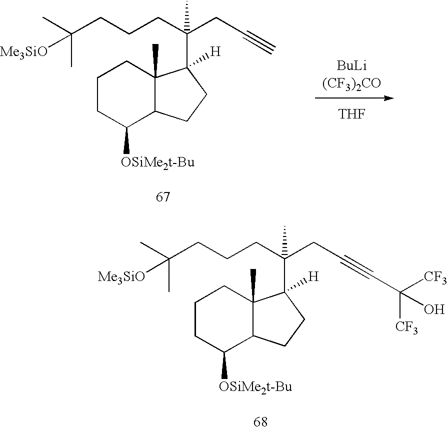

### (6S)-6-[(1R,3aR,4S,7aR)-4-(tert-Butyl-dimethyl-silanyloxy)-7a-methyl-octahydro-inden-1-yl]-1,1,1-trifluoro-6,10-dimethyl-2-trifluoromethyl-10-trimethylsilanyloxy-undec-3-yn-2-ol (68)

A two neck 50 ml round bottom flask equipped with stir bar, Claisen adapter with rubber septum and funnel (with cooling bath) was charged with 1.390 g (2.742 mmol) of (1R,3aR,4S,7aR)-4-(tert-butyl-dimethyl-silanyloxy)-1-[(1S)-1,5-dimethyl-1-prop-2-ynyl-5-trimethylsilanyloxy-hexyl]-7a-methyl-octahydro-indene (67) and 30 ml of tetrahydrofurane. The funnel was connected to container with hexafluoroacetone and cooled (acetone, dry ice). The reaction mixture was cooled to −70° C. and 5.00 ml (8.00 mmol) of 1.6M n-butyllithium in tetrahydrofurane was added dropwise. After 30 min hexafluoroacetone was added (the container's valve was opened three times). The reaction was stirred at −70° C. for 2 h then 5.0 ml of saturated solution of ammonium chloride was added. The mixture was dissolved by the addition of 100 ml of saturated solution of ammonium chloride and extracted three times with 80 ml of ethyl acetate, dried over Na₂SO₄ and evaporated. The oil residue was chromatographed twice to remove a large amount of polymer compounds. The first column (100 cm³) using hexane:ethyl acetate (10:1) as mobile phase. The second column (100 cm³) using hexane:ethyl acetate (25:1, 15:1) as mobile phase. Fractions containing product were pooled and evaporated to give 1.959 g of colorless oil. Product 68 was used to the next reaction without farther purification.

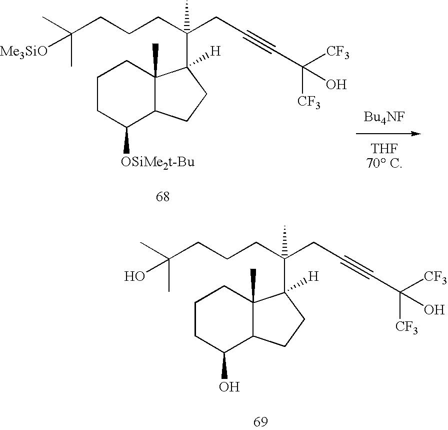

### (6S)-1,1,1-Trifluoro-6-[(1R,3aR,4S,7aR)-4-hydroxy-7a-methyl-octahydro-inden-1-yl]-6,10-dimethyl-2-trifluoromethyl-undec-3-yne-2,10-diol (69)

A 25 ml round bottom flask equipped with stir bar and Claisen adapter with rubber septum was charged with crude (ca. 2.74 mmol) (6S)-6-[(1R,3aR,4S,7aR)-4-(tert-butyl-dimethyl-silanyloxy)-7a-methyl-octahydro-inden-1-yl]-1,1,1-trifluoro-6,10-dimethyl-2-trifluoromethyl-10-trimethylsilanyloxy-undec-3-yn-2-ol (68) and 12.0 ml (12.0 mmol) of 1M tetrabutylammonium fluoride in tetrahydrofurane and reaction was stirred at 70° C. After 18 h new portion 5.0 ml of 1M tetrabutylammonium fluoride in tetrahydrofurane was added. The reaction mixture was stirred at 70° C. for next 80 h. The mixture was dissolved by the addition of 150 ml of ethyl acetate and extracted six times with 50 ml of water:brine (1:1) and 50 ml of brine and dried over Na₂SO₄ and evaporated. The oil residue was chromatographed on column (200 cm³) using hexane:ethyl acetate (3:1, 2:1) as mobile phase. The fractions containing product were pooled and evaporated. The residue was crystallized from hexane-ethyl acetate to give 917 mg (69%, two steps) of product 69 as a white crystal.

m.p. 146-147° C.

[α]_{D}³⁰=−3.5° c=0.43, CHCl₃

¹H NMR (CDCl₃): 4.08 (1H, br s), 2.45 (1H, AB, J=17 Hz), 2.36 (1H, AD, J=17 Hz), 1.98-1.92 (1H, m), 1.85-1.74 (2H, m), 1.67-1.18 (18H, m), 1.25 (6H, s), 1.07 (3H, s), 1.02 (3H, s)

| | | | | |
|---|---|---|---|---|
| MS HRES | Calculated for: | C₂₄H₃₆F₆O₃ | [M + Na]⁺ | 509.2461 |
| | Observed: | | [M + Na]⁺ | 509.2459 |

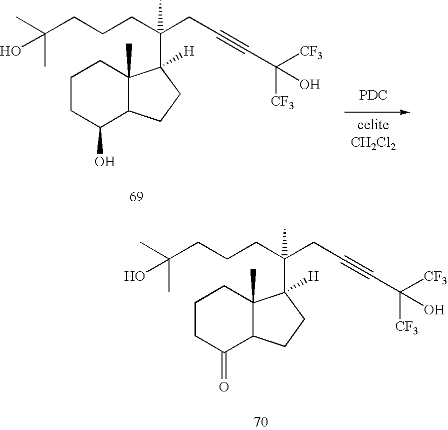

### (1R,3aR,4S,7aR)-7a-Methyl-1-[(1S)-6,6,6-trifluoro-5-hydroxy-1-(4-hydroxy-4-methyl-pentyl)-1-methyl-5-trifluoromethyl-hex-3-ynyl]-octahydro-inden-4-one (70)

A 25 ml round bottom flask equipped with stir bar and Claisen adapter with rubber septum was charged with 300 mg (0.617 mmol) of (6S)-1,1,1-trifluoro-6-[(1R,3aR,4S,7aR)-4-hydroxy-7a-methyl-octahydro-inden-1-yl]-6,10-dimethyl-2-trifluoromethyl-undec-3-yne-2,10-diol (69) and 10 ml of dichloromethane. A 696 mg (1.851 mmol) of pyridinium dichromate and 710 mg of celite were added and mixture was stirred in room temperature for 3 h. The reaction mixture was filtrated through column with silica gel (50 cm³) and celite (2 cm) and using dichloromethane:ethyl acetate (4:1) as a mobile phase. The fractions containing product were pooled and evaporated to give yellow oil. The product 70 was used to the next reaction without farther purification.

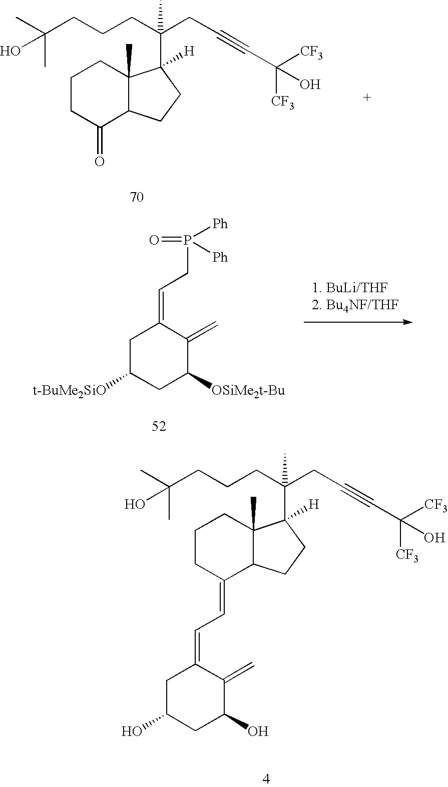

### (20S)-1,25-Dihydroxy-20-(5,5,5-trifluoro-4-hydroxy-4-trifluoromethyl-pent-2-ynyl)cholecalciferol (4)

A 25 ml round bottom flask equipped with stir bar and Claisen adapter with rubber septum was charged with 1.798 g (3.084 mmol) of (1S,5R)-1,5-bis-((tert-butyldimethyl)silanyloxy)-3-[2-(diphenylfosphinoyl)-eth-(Z)-ylidene]-2-methylene-cyclohexane (52) and 12 ml of tetrahydrofurane. The reaction mixture was cooled to −78° C. and 1.9 ml (3.04 mmol) of 1.6M n-butyllithium in tetrahydrofurane was added dropwise. The resulting deep red solution was stirred at −78° C. for 20 min and crude (ca 0.617 mmol) (1R,3aR,4S,7aR)-7a-methyl-1-[(1S)-6,6,6-trifluoro-5-hydroxy-1-(4-hydroxy-4-methyl-pentyl)-1-methyl-5-trifluoromethyl-hex-3-ynyl]-octahydro-inden-4-one (70) was added dropwise in 1.5 ml of tetrahydrofurane. The reaction mixture was stirred for 5 h and then the bath was removed and the mixture was poured into 50 ml of ethyl acetate and 100 ml of brine. The water fraction was extracted three times with 50 ml of ethyl acetate, dried over Na₂SO₄ and evaporated. The oil residue was chromatographed on column (75 cm³, protected from light) using hexane:ethyl acetate (5:1) as mobile phase. Fractions containing product were pooled and evaporated to give colorless oil (293 mg) which was treated with 5 ml of 1M tetrabutylammonium fluoride in tetrahydrofurane. The reaction mixture was stirred at room temperature for 40 h. The mixture was dissolved by the addition of 150 ml of ethyl acetate and extracted six times with 50 ml of water:brine (1:1) and 50 ml of brine, dried over Na₂SO₄ and evaporated. The oil residue was chromatographed on column (50 cm³, protected from light) using ethyl acetate as mobile phase. Fractions containing product were pooled and evaporated to give product as colorless oil. Oil was dissolved in methyl acetate and evaporated (4 times) to give 190 mg (50% three steps) of product 4 as white foam.

[α]_{D}³⁰=−4.6° c=0.35, CHCl₃

UV λmax (EtOH): 205.50 nm (ε 16586), 266.00 nm (ε 143.19)

¹H NMR (CDCl₃): 6.36 (1H, d, J=11.3 Hz), 6.23 (1H, br s), 6.00 (1H, d, J=1.1 Hz), 5.32 (1H, s), 4.98 (1H, s), 4.43 (1H, dd, J=7.7, 4.3 Hz), 4.25-4.20 (1H, m), 2.82-2.79 (1H, m), 2.59 (1H, dd, J=13.1, 3.1 Hz), 2.44 (1H, AB, J=17.2 Hz), 2.37 (1H, AB, J=17.2 Hz), 2.30 (1H, dd, J=13.2, 6.2 Hz,), 2.06-1.87 (4H, m), 1.72-1.36 (11H, m), 1.26-1.21 (1H, m), 1.24 (6H, s), 0.99 (3H, s), 0.64 (3H, s)

¹³C NMR (CDCl₃): 147.48, 142.29, 133.16, 124.72, 121.32 (q, J=287.1 Hz), 117.59, 11.68, 90.08, 72.62, 71.39, 70.73, 66.89, 57.28, 56.52, 46.65, 45.18, 43.20, 42.81, 41.04, 40.89, 40.03, 29.79, 29.35, 28.95, 23.45, 22.86, 22.60, 21.84, 17.77, 14.93

| | | | | |
|---|---|---|---|---|
| MS HRES | Calculated for: | C₃₃H₄₆F₆O₄ | [M + Na]⁺ | 643.3192 |
| | Observed: | | [M + Na]⁺ | 643.3192 |

### Example 5

### Synthesis of (20S)-1,25-Dihydroxy-20-(5,5,5-trifluoro-4-hydroxy-4-trifluoromethyl-pent-2-ynyl)-19-nor-cholecalciferol (10)

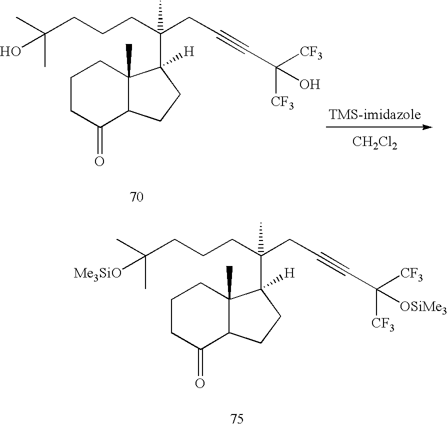

### (1R,3aR,4S,7aR)-7a-Methyl-1-[(1S)-6,6,6-trifluoro-1-methyl-1-(4-methyl-4-trimethylsilanyloxy-pentyl)-5-trifluoromethyl-5-trimethylsilanyloxy-hex-3-ynyl]-octahydro-inden-4-one (75)

A 25 ml round bottom flask equipped with stir bar, and Claisen adapter with rubber septum was charged with 585 mg (1.207 mmol) of (1R,3aR,4S,7aR)-7a-methyl-1-[(1S)-6,6,6-trifluoro-5-hydroxy-1-(4-hydroxy-4-methyl-pentyl)-1-methyl-5-trifluoromethyl-hex-3-ynyl]-octahydro-inden-4-one (70) and 10 ml of dichloromethane. A 1.5 ml (10.2 mmol) of 1-(trimethylsilyl)imidazole was added dropwise. The mixture was stirred at room temperature for 3 h. A 150 ml of ethyl acetate was added and the mixture was washed three times with 50 ml of water, dried over Na₂SO₄ and evaporated. The oil residue was chromatographed on column (50 cm³) using hexane:ethyl acetate (10:1) as mobile phase. Fractions containing product were pooled and evaporated to give 660 mg (87%) of product 75 as colorless oil.

¹H NMR (CDCl₃): 2.44-2.39 (3H, m), 2.32-2.16 (2H, m), 2.10-1.99 (2H, m), 1.95-1.84 (2H, m), 1.77-1.56 (4H, m), 1.38-1.19 (7H, m), 1.20 (6H, s), 1.03 (3H, s), 0.74 (3H, s), 0.28 (9H, s), 0.10 (9H, s)

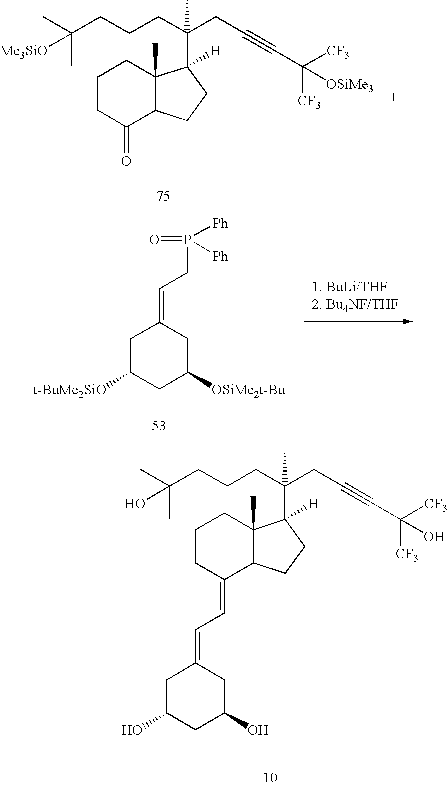

### (20S)-1,25-Dihydroxy-20-(5,5,5-trifluoro-4-hydroxy-4-trifluoromethyl-pent-2-ynyl)-19-nor-cholecalciferol (10)

A 25 ml round bottom flask equipped with stir bar and Claisen adapter with rubber septum was charged with 618 mg (1.083 mmol) of (1R,3R)-1,3-bis-((tert-butyldimethyl)silanyloxy)-5-[2-(diphenylfosphinoyl)ethylidene]-cyclohexane (53) and 10 ml of tetrahydrofurane. The reaction mixture was cooled to −70° C. and 0.67 ml (1.07 mmol) of 1.6M n-butyllithium BuLi was added dropwise. The resulting deep red solution was stirred at −70° C. for 20 min and 335 mg (0.532 mmol) of (1R,3aR,4S,7aR)-7a-methyl-1-[(1S)-6,6,6-trifluoro-1-methyl-1-(4-methyl-4-trimethylsilanyloxy-pentyl)-5-trifluoromethyl-5-trimethylsilanyloxy-hex-3-ynyl]-octahydro-inden-4-one (75) in 1.5 ml of tetrahydrofurane. The reaction mixture was stirred for 5 h and then the dry ice was removed from bath and the solution was allowed to warm up to −40° C. in 1 h. The mixture was poured into 50 ml of ethyl acetate and 100 ml of brine. The water fraction was extracted four times with 50 ml of ethyl acetate, dried over Na₂SO₄ and evaporated. The oil residue was chromatographed on column (50 cm³, protected from light) using hexane:ethyl acetate (10:1) as mobile phase. Fractions containing product were pooled and evaporated to give colorless oil (ca. 440 mg) which was treated with 10 ml of 1M tetrabutylammonium fluoride in tetrahydrofurane. The reaction mixture was stirred at room temperature for 29 h. The mixture was dissolved by the addition of 150 ml of ethyl acetate and extracted six times with 50 ml of water:brine (1:1) and 50 ml of brine, dried over Na₂SO₄ and evaporated. The oil residue was chromatographed on column (50 cm³, protected from light) using ethyl acetate as mobile phase. Fractions containing product were pooled and evaporated to give product as colorless oil. Oil was dissolved in methyl acetate and evaporated (2 times) to give 308 mg (95%) of product 10 as white foam.

[α]_{D}²⁶=+38.8° c=0.42, EtOH

UV λmax (EtOH): 243 nm (ε 29530), 252 nm (ε 33645), 261 nm (ε 23156)

¹H NMR (CDCl₃): 6.28 (1H, d, J=11.3 Hz), 5.83 (1H, d, J=11.1 Hz), 4.12-4.09 (1H, m), 4.05-4.01 (1H, m), 2.80-2.72 (2H, m), 2.46 (1H, dd, J=13.4, 3.0 Hz), 2.42 (1H, AB, J=16.8 Hz), 2.36 (1H, AB, J=16.8 Hz), 2.22-2.16 (2H, m), 2.04-1.86 (6H, m), 1.80-1.38 (17H, m), 1.23 (6H, s), 0.99 (3H, s), 0.63 (3H, s)

¹³C NMR (CDCl₃): 142.13, 131.41, 123.55, 121.36 (q, J=286.9 Hz, 115.88, 72.40, 71.40, 67.40, 67.15, 27.19, 56.47, 46.50, 44.44, 43.40, 41.94, 40.91, 40.83, 39.97, 37.09, 29.65, 29.29, 29.26, 28.79, 23.35, 22.79, 22.60, 21.81, 17.79, 15.00

| | | | | |
|---|---|---|---|---|
| MS HRES | Calculated for: | C₃₂H₄₆F₆O₄ | [M + Na]⁺ | 631.3192 |
| | Observed: | | [M + Na]⁺ | 631.3191 |

### Example 6

### Synthesis of (20S)-1α-Fluoro-25-hydroxy-20-(5,5,5-trifluoro-4-hydroxy-4-trifluoromethyl-pent-2-ynyl)-cholecalciferol (13)

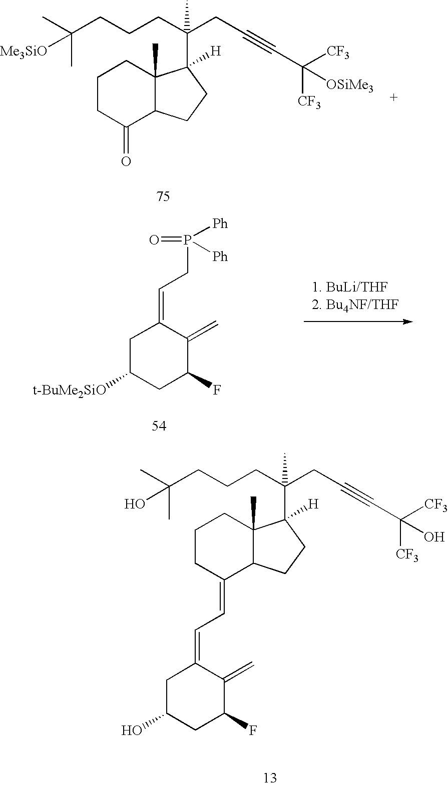

### (20S)-1α-Fluoro-25-hydroxy-20-(5,5,5-trifluoro-4-hydroxy-4-trifluoromethyl-pent-2-ynyl)-cholecalciferol (13)

A 25 ml round bottom flask equipped with stir bar and Claisen adapter with rubber septum was charged with 495 mg (1.052 mmol) of (1S,5R)-1-((tert-butyldimethyl)silanyloxy)-3-[2-(diphenylfosphinoyl)-eth-(Z)-ylidene]-5-fluoro-2-methylene-cyclohexane (54) and 10 ml of tetrahydrofurane. The reaction mixture was cooled to −70° C. and 0.65 ml (1.04 mmol) of 1.6M n-butyllithium was added dropwise. The resulting deep red solution was stirred at −70° C. for 20 min and 300 mg (0.477 mmol) of (1R,3aR,4S,7aR)-7a-methyl-1-[(1S)-6,6,6-trifluoro-1-methyl-1-(4-methyl-4-trimethylsilanyloxy-pentyl)-5-trifluoromethyl-5-trimethylsilanyloxy-hex-3-ynyl]-octahydro-inden-4-one (75) was added dropwise in 1.5 ml of tetrahydrofurane. The reaction mixture was stirred for 4 h and then the dry ice was removed from bath and the solution was allowed to warm up to −40° C. in 1 h. The mixture was poured into 50 ml of ethyl acetate and 100 ml of brine. The water fraction was extracted three times with 50 ml of ethyl acetate, dried over Na₂SO₄ and evaporated. The oil residue was chromatographed on column (50 cm³, protected from light) using hexane:ethyl acetate (10:1) as mobile phase. Fractions containing product were pooled and evaporated to give colorless oil (ca. 429 mg) which was treated with 10 ml of 1M tetrabutylammonium fluoride in tetrahydrofurane. The reaction mixture was stirred at room temperature for 18 h. The mixture was dissolved by the addition of 150 ml of ethyl acetate and extracted six times with 50 ml of water:brine (1:1) and 50 ml of brine, dried over Na₂SO₄ and evaporated. The oil residue was chromatographed on column (50 cm³, protected from light) using ethyl acetate/hexane (1:1) as mobile phase. Fractions containing product were pooled and evaporated to give product as colorless oil. The product was dissolved in methyl acetate and evaporated (2 times) to give 274 mg 92%) of product 13 as white foam.

[α]_{D}³⁰=+27.0 c=0.50, EtOH

UV λmax (EtOH): 212 nm (ε 34256), 243 nm (ε 15866), 271 nm (ε 16512)

¹H NMR (CDCl₃): 6.38 (1H, d, J=11.3 Hz), 6.01 (1H, d, J=11.3 Hz), 5.38 (1H, s), 5.13 (1H, ddd, J=49.9, 6.6, 3.6 Hz), 5.09 (1H, s), 4.23-4.19 (1H, m), 2.80 (1H, dd, J=12.0, 3.5 Hz), 2.61 (1H, dd, J=13.3, 3.7 Hz), 2.43 (1H, AB, J=16.9 Hz), 2.36 (1H, AB, J=16.9 Hz), 2.30 (1H, dd, J=13.4, 7.9 Hz), 2.24-2.15 (1H, m), 2.04-1.92 (3H, m), 1.73-1.35 (17H, m), 1.26-1.21 (1H, m), 1.24 (6H, s), 0.99 (3H, s), 0.64 (3H, s)

¹³C NMR (CDCl₃): 142.97 (d, J=16.8 Hz), 142.69, 131.68 (d, J=2.2 Hz), 125.37, 121.34 (q, J=286.9 Hz), 117.63, 114.99 (d, J=10.0 Hz), 91.61 (d, J=172.4 Hz), 90.07, 72.62, 71.38, 66.56 (d, J=6.0 Hz), 57.26, 56.53, 46.68, 44.91, 43.31, 40.97, 40.89, 40.68 (d, J=20.6 Hz), 40.01, 29.67, 29.28, 28.98, 23.43, 22.81, 22.60, 21.78, 17.79, 14.96

| | | | | |
|---|---|---|---|---|
| MS HRES | Calculated for: | C₃₃H₄₅F₇O₃ | [M + Na]⁺ | 645.3149 |
| | Observed: | | [M + Na]⁺ | 645.3148 |

### Example 7

### Synthesis of (20S)-1,25-Dihydroxy-20-(5,5,5-trifluoro-4-hydroxy-4-trifluoromethyl-pent-(2Z)-enyl)cholecalciferol (5)

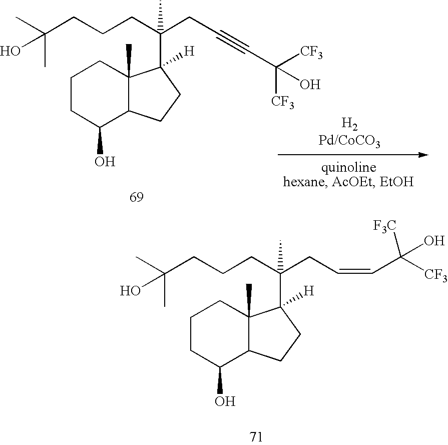

### (3Z,6S)-1,1,1-Trifluoro-6-[(1R,3aR,4S,7aR)-4-hydroxy-7a-methyl-octahydro-inden-1-yl]-6,10-dimethyl-2-trifluoromethyl-undec-3-ene-2,10-diol (71)

A 25 ml round bottom flask was charged with 250 mg (0.514 mmol) of (6S)-1,1,1-trifluoro-6-[(1R,3aR,4S,7aR)-4-hydroxy-7a-methyl-octahydro-inden-1-yl]-6,10-dimethyl-2-trifluoromethyl-undec-3-yne-2,10-diol (69), 70 mg of 5% Pd/CaCO₃, 6.0 ml of hexane, 2.4 ml of ethyl acetate and 0.23 ml of solution of quinoline in ethanol (prepared from 3.1 ml of ethanol and 168 μl of quinoline). The substrate was hydrogenated at ambient temperature and atmospheric pressure of hydrogen. The reaction was monitoring by TLC (hexane:ethyl acetate—2:1). After 7 h the catalyst was filtered off and solvent evaporated. The residue was purified over silica gel (125 cm³) using hexane:ethyl acetate (2:1) as a mobile phase. Fractions containing product were pooled and evaporated to give 243 mg (97%) of product 71 as colorless oil.

¹H NMR (CDCl₃): 6.14-6.05 (1H, m), 5.49 (1H, d, J=12.5 Hz), 4.08 (1H, br s), 2.83 (1H, dd, J=15.9, 9.7 Hz), 2.48-2.38 (1H, m), 1.85-1.75 (2H, m), 1.65-1.20 (17H, m), 1.22 (3H, s), 1.20 (3H, s), 1.08 (3H, s), 1.03-0.96 (1H, m), 1.00 (3H, s)

¹³C NMR (CDCl₃): 140.22, 117.44, 71.79, 69.66, 56.74, 52.58, 44.11, 43.45, 41.19, 40.24, 39.64, 36.88, 33.44, 30.09, 28.88, 22.55, 22.21, 21.70, 17.63, 17.58, 16.54

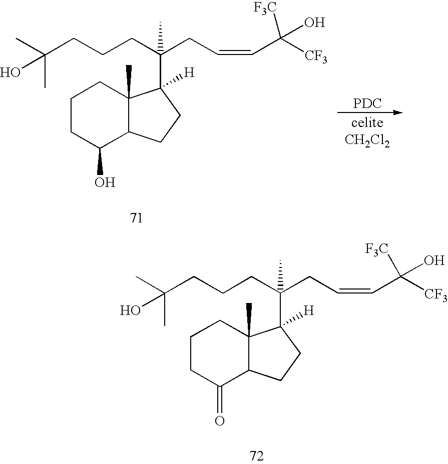

### (1R,3aR,4S,7aR)-7a-Methyl-1-[(1S,3Z)-6,6,6-trifluoro-5-hydroxy-1-(4-hydroxy-4-methyl-pentyl)-1-methyl-5-trifluoromethyl-hex-3-enyl]-octahydro-inden-4-one (72)

A 25 ml round bottom flask equipped with stir bar and Claisen adapter with rubber septum was charged with 290 mg (0.594 mmol) of (3Z,6S)-1,1,1-trifluoro-6-[(1R,3aR,4S,7aR)-4-hydroxy-7a-methyl-octahydro-inden-1-yl]-6,10-dimethyl-2-trifluoromethyl-undec-3-ene-2,10-diol (71) and 10 ml of dichloromethane. A 700 mg (1.861 mmol) pyridinium dichromate and 750 mg of celite was added and mixture was stirred in room temperature for 3 h. The reaction mixture was filtrated through column with silica gel (75 cm³) and celite (2 cm) and using dichloromethane:ethyl acetate (4:1) as a mobile phase. The fractions containing product were pooled and evaporated to give yellow oil. The product 72 was used to the next reaction without farther purification.

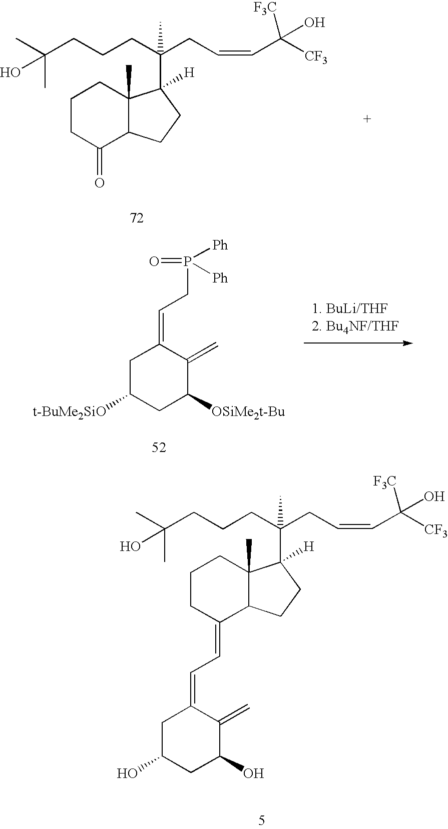

### (20S)-1,25-Dihydroxy-20-(5,5,5-trifluoro-4-hydroxy-4-trifluoromethyl-pent-(2Z)-enyl)cholecalciferol (5)

A 25 ml round bottom flask equipped with stir bar and Claisen adapter with rubber septum was charged with 1.800 g (3.088 mmol) of (1S,5R)-1,5-bis-((tert-butyldimethyl)silanyloxy)-3-[2-(diphenylfosphinoyl)-eth-(Z)-ylidene]-2-methylene-cyclohexane (52) and 10.0 ml of tetrahydrofurane. The reaction mixture was cooled to −78° C. and 1.9 ml (3.04 mmol) of 1.6M n-butyllithium in tetrahydrofurane was added dropwise. The resulting deep red solution was stirred at −78° C. for 20 min and 278 mg (0.571 mmol) of (1R,3aR,4S,7aR)-7a-methyl-1-[(1S,3Z)-6,6,6-trifluoro-5-hydroxy-1-(4-hydroxy-4-methyl-pentyl)-1-methyl-5-trifluoromethyl-hex-3-enyl]-octahydro-inden-4-one (72) was added dropwise in 1.5 ml of tetrahydrofurane. The reaction mixture was stirred for 5 h (last 0.5 h at −20° C.) and then the bath was removed and the mixture was poured into 50 ml of ethyl acetate and 100 ml of brine. The water fraction was extracted three times with 50 ml of ethyl acetate, dried over Na₂SO₄ and evaporated. The oil residue was chromatographed on column (75 cm³, protected from light) using hexane:ethyl acetate (4:1) as mobile phase. Fractions containing product were pooled and evaporated to give colorless oil (309 mg) which was treated with 5 ml of 1M tetrabutylammonium fluoride in tetrahydrofurane. The reaction mixture was stirred at room temperature for 22 h. The mixture was dissolved by the addition of 150 ml of ethyl acetate and extracted six times with 50 ml of water:brine (1:1) and 50 ml of brine, dried over Na₂SO₄ and evaporated. The oil residue was chromatographed on column (50 cm³, protected from light) using ethyl acetate as mobile phase. Fractions containing product were pooled and evaporated to give product as colorless oil. Oil was dissolved in methyl acetate and evaporated (4 times) to give 192 mg (54%, two steps) of product 5 as white foam.

UV λmax (EtOH): 204.08 nm (ε 27522), 266.03 nm (ε 20144)

¹H NMR (CDCl₃): 6.37 (1H, d, J=11.1 Hz), 6.10 (1H, ddd, J=12.5, 9.0, 6.0 Hz), 6.00 (1H, d, J=11.3 Hz), 5.47 (1H, d, J=12.2 Hz), 5.32 (1H, s), 5.07 (1H, br, s), 4.99 (1H, s), 4.43 (1H, dd, J=7.8, 4.2 Hz), 4.25-4.20 (1H, m), 2.85-2.79 (2H, m), 2.59 (1H, dd, J=13.4, 3.0 Hz), 2.46 (1H, dd, J=16.4, 4.9 Hz), 2.31 (1H, dd, J=13.4, 6.4 Hz), 2.04-1.97 (3H, m), 1.90 (1H, ddd, J=12.0, 8.2, 3.2 Hz), 1.76-1.20 (17H, m), 1.21 (3H, s), 1.20 (3H, s), 1.06-1.00 (1H, m), 0.96 (3H, s), 0.64 (3H, s)

¹³C NMR (CDCl₃): 147.51, 142.74, 140.17, 132.92, 124.88, 122.95 (q, J=286.9 Hz), 122.80 (q, J=285.5 Hz), 117.52, 117.39, 111.65, 71.94, 70.73, 66.88, 56.86, 56.65, 46.79, 45.20, 43.95, 42.83, 41.06, 40.09, 39.75, 37.22, 30.35, 29.05, 28.82, 23.58, 22.50, 22.19, 21.93, 17.53, 15.04

| | | | | |
|---|---|---|---|---|
| MS HRES | Calculated for: | C₃₃H₄₈F₆O₄ | [M + Na]⁺ | 645.3349 |
| | Observed: | | [M + Na]⁺ | 645.3350 |

### Example 8

### Synthesis of (20S)-1,25-Dihydroxy-20-[(2Z)-5,5,5-trifluoro-4-hydroxy-4-trifluoromethyl-pent-2-enyl]-19-nor-cholecalciferol (11)

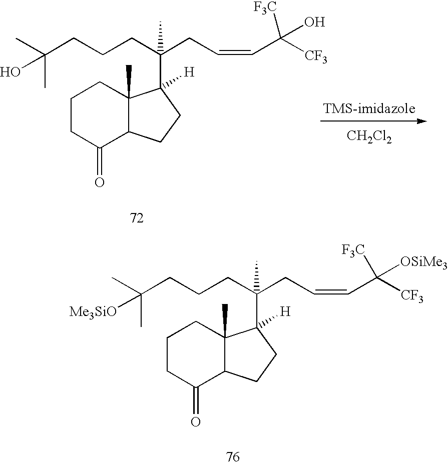

### (1R,3aR,4S,7aR)-7a-Methyl-1-[(1S,3Z)-6,6,6-trifluoro-1-methyl-1-(4-methyl-4-trimethylsilanyloxy-pentyl)-5-trifluoromethyl-5-trimethylsilanyloxy-hex-3-enyl]-octahydro-inden-4-one (76)

A 25 ml round bottom flask equipped with stir bar and Claisen adapter with rubber septum was charged with 590 mg (1.213 mmol) of (1R,3aR,4S,7aR)-7a-methyl-1-[(1S,3Z)-6,6,6-trifluoro-5-hydroxy-1-(4-hydroxy-4-methyl-pentyl)-1-methyl-5-trifluoromethyl-hex-3-enyl]-octahydro-inden-4-one (72) and 15 ml of dichloromethane. A 1.4 ml (9.5 mmol) of 1-(trimethylsilyl)imidazole was added dropwise. The mixture was stirred at room temperature for 4 h. A 150 ml of ethyl acetate was added and the mixture was washed three times with 50 ml of water, dried over Na₂SO₄ and evaporated. The oil residue was chromatographed on column (50 cm³) using hexane:ethyl acetate (10:1) as mobile phase. Fractions containing product were pooled and evaporated to give 726 mg (95%) of product 76 as colorless oil.

¹H NMR (CDCl₃): 6.07-5.99 (1H, m), 5.41 (1H, d, J=11.4 Hz), 2.52 (2H, dd, J=6.2, 2.6 Hz), 2.44-2.38 (1H, m), 2.31-1.54 (11H, m), 1.36-1.14 (6H, m), 1.19 (6H, s), 0.97 (3H, s), 0.74 (3H, s), 0.25 (9H, s), 0.09 (9H, s)

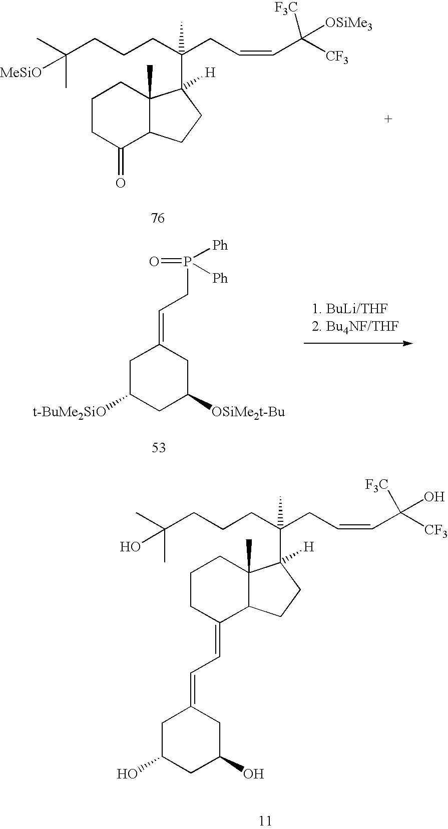

### (20S)-1,25-Dihydroxy-20-[(2Z)-5,5,5-trifluoro-4-hydroxy-4-trifluoromethyl-pent-2-enyl]-19-nor-cholecalciferol (11)

A 25 ml round bottom flask equipped with stir bar and Claisen adapter with rubber septum was charged with 841 mg (1.473 mmol) of (1R,3R)-1,3-bis-((tert-butyldimethyl)silanyloxy)-5-[2-(diphenylfosphinoyl)ethylidene]-cyclohexane (53) and 10 ml of tetrahydrofurane. The reaction mixture was cooled to −70° C. and 0.88 ml (1.41 mmol) of 1.6M n-butyllithium BuLi was added dropwise. The resulting deep red solution was stirred at −70° C. for 20 min and 369 mg (0.585 mmol) of (1R,3aR,4S,7aR)-7a-methyl-1-[(1S,3Z)-6,6,6-trifluoro-1-methyl-1-(4-methyl-4-trimethylsilanyloxy-pentyl)-5-trifluoromethyl-5-trimethylsilanyloxy-hex-3-enyl]-octahydro-inden-4-one (76) in 1.5 ml of tetrahydrofurane. The reaction mixture was stirred for 5 h and then the dry ice was removed from bath and the solution was allowed to warm up to −40° C. in 1 h. The mixture was poured into 50 ml of ethyl acetate and 100 ml of brine. The water fraction was extracted three times with 50 ml of ethyl acetate, dried over Na₂SO₄ and evaporated. The oil residue was chromatographed on column (50 cm³, protected from light) using hexane:ethyl acetate (10:1) as mobile phase. Fractions containing product were pooled and evaporated to give colorless oil (ca. 560 mg) which was treated with 8 ml of 1M tetrabutylammonium fluoride in tetrahydrofurane. The reaction mixture was stirred at room temperature for 8 h. The new portion 7 ml of 1M tetrabutylammonium fluoride in tetrahydrofurane was added and the mixture was stirred for 40 h. The mixture was dissolved by the addition of 150 ml of ethyl acetate and extracted six times with 50 ml of water:brine (1:1) and 50 ml of brine, dried over Na₂SO₄ and evaporated. The oil residue was chromatographed on column (50 cm³, protected from light) using ethyl acetate as mobile phase. Fractions containing product were pooled and evaporated to give product as colorless oil. Oil was dissolved in methyl acetate and evaporated (2 times) to give 327 mg (92%) of product 11 as white foam.

[α]_{D}²⁸=+32° c=0.43, EtOH

UV λmax (EtOH): 243.67 nm (ε 36197), 252.00 nm (ε 41649), 261.83 nm (ε 28455)

¹H NMR (CDCl₃): 6.31 (1H, d, J=11.2 Hz), 6.11 (H, ddd, J=12.4, 9.3, 5.7 Hz), 5.84 (1H, d, J=10.7 Hz), 5.48 (1H, d, J=11.7 Hz), 4.12 (1H, br s), 4.05 (1H, br s), 2.86-2.72 (3H, m), 2.50-2.46 (2H, m), 2.24-2.18 (2H, m), 2.08-1.94 (3H, m), 1.88-1.22 (18H, m), 1.22 (6H, s), 1.06-0.91 (2H, m), 0.97 (3H, s), 0.65 (3H, s)

| | | | | |
|---|---|---|---|---|
| MS HRES | Calculated for: | C₃₂H₄₈F₆O₄ | [M + Na]⁺ | 633.3349 |
| | Observed: | | [M + Na]⁺ | 633.3348 |

### Example 9

### Synthesis of 20S)-1α-Fluoro-25-hydroxy-20-[(2Z)-5,5,5-trifluoro-4-hydroxy-4-trifluoromethyl-pent-2-enyl]-cholecalciferol (14)

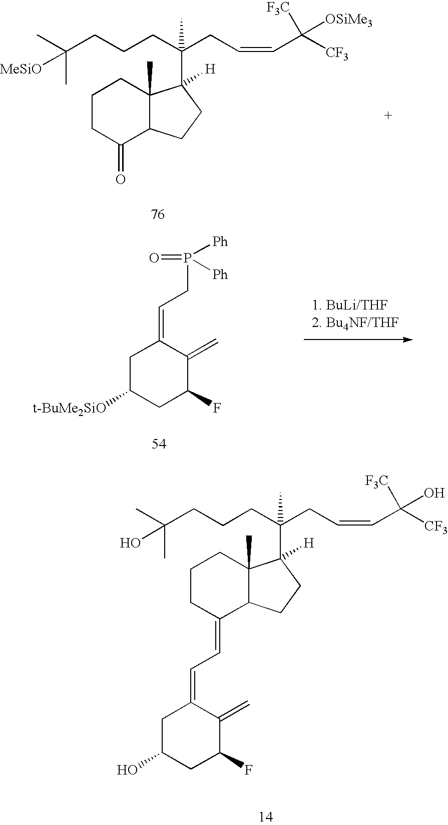

### (20S)-1α-Fluoro-25-hydroxy-20-[(2Z)-5,5,5-trifluoro-4-hydroxy-4-trifluoromethyl-pent-2-enyl]-cholecalciferol (14)

A 25 ml round bottom flask equipped with stir bar and Claisen adapter with rubber septum was charged with 712 mg (1.513 mmol) of (1S,5R)-1-((tert-butyldimethyl)silanyloxy)-3-[2-(diphenylfosphinoyl)-eth-(Z)-ylidene]-5-fluoro-2-methylene-cyclohexane (54) and 10 ml of tetrahydrofurane. The reaction mixture was cooled to −70° C. and 0.90 ml (1.44 mmol) of 1.6M n-butyllithium was added dropwise. The resulting deep red solution was stirred at −70° C. for 20 min and 320 mg (0.507 mmol) of (1R,3aR,4S,7aR)-7a-methyl-1-[(1S,3Z)-6,6,6-trifluoro-1-methyl-1-(4-methyl-4-trimethylsilanyloxy-pentyl)-5-trimethylsilanyloxy-hex-3-enyl]-octahydro-inden-4-one (76) was added dropwise in 1.5 ml of tetrahydrofurane. The reaction mixture was stirred for 4 h and then the dry ice was removed from bath and the solution was allowed to warm up to −40° C. in 1 h. The mixture was poured into 50 ml of ethyl acetate and 100 ml of brine. The water fraction was extracted three times with 50 ml of ethyl acetate, dried over Na₂SO₄ and evaporated. The oil residue was chromatographed on column (50 cm³, protected from light) using hexane:ethyl acetate (10:1) as mobile phase. Fractions containing product were pooled and evaporated to give colorless oil which was treated with 10 ml of 1M tetrabutylammonium fluoride in tetrahydrofurane. The reaction mixture was stirred at room temperature for 6 h 30 min. The mixture was dissolved by the addition of 150 ml of ethyl acetate and extracted six times with 50 ml of water:brine (1:1) and 50 ml of brine, dried over Na₂SO₄ and evaporated. The oil residue was chromatographed on column (50 cm³, protected from light) using ethyl acetate:hexane (1:1 and 2:1) as mobile phase. Fractions containing product were pooled and evaporated to give product as colorless oil. The product was dissolved in methyl acetate and evaporated (2 times) to give 300 mg 95%) of product 14 as white foam.

[α]_{D}²⁸=+20.2° c=0.55, EtOH

UV λmax (EtOH): 207.67 nm (ε 20792), 242.33 nm (ε 17972), 270.00 nm (ε 18053)

¹H NMR (CDCl₃): 6.40 (1H, d, J=11.1 Hz), 6.11 (1H, ddd, J=12.4, 9.5, 6.0 Hz), 6.02 (1H, d, J=11.1 Hz), 5.49 (1H, d, J=12.1 Hz), 5.39 (1H, s), 5.14 (1H, ddd, J=49.5, 7.2, 4.2 Hz), 5.10 (1H, s), 4.23 (1H, br s), 2.87-2.80 (2H, m), 2.62 (1H, br d, J=12.1 Hz), 2.48-2.43 (1H, m), 2.31 (1H, dd, J=12.9, 7.5 Hz), 2.22-2.14 (1H, m), 2.06-1.97 (3H, m), 1.70-1.12 (16H, m), 1.22 (3H, s), 1.21 (3H, m), 1.05-0.91 (2H, m), 0.97 (3H, s), 0.65 (3H, s)

| | | | | |
|---|---|---|---|---|
| MS HRES | Calculated for: | C₃₃H₄₇F₇O₃ | [M + Na]⁺ | 647.3305 |
| | Observed: | | [M + Na]⁺ | 647.3304 |

### Example 10

### Synthesis of (20S)-1,25-Dihydroxy-20-[(2E)-5,5,5-trifluoro-4-hydroxy-4-trifluoromethyl-pent-2-enyl]-cholecalciferol (6)

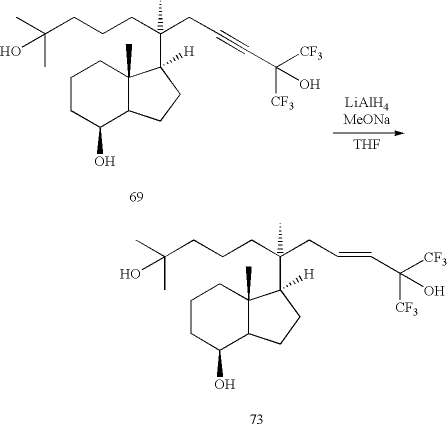

### (3E,6S)-1,1,1-Trifluoro-6-[(1R,3aR,4S,7aR)-4-hydroxy-7a-methyl-octahydro-inden-1-yl]-6,10-dimethyl-2-trifluoromethyl-undec-3-ene-2,10-diol (73)

A 25 ml round bottom flask equipped with stir bar and condenser with nitrogen sweep was charged with 4.0 ml (4.0 mmol) of 1M lithium aluminum hydride in tetrahydrofurane. The mixture was cooled to 0° C. and 216 mg (4.00 mmol) of sodium methoxide was added slowly followed by 300 mg (0.617 mmol) of (6S)-1,1,1-trifluoro-6-([(1R,3aR,4S,7aR)-4-hydroxy-7a-methyl-octahydro-inden-1-yl]-6,10-dimethyl-2-trifluoromethyl-undec-3-yne-2,10-diol (69) in 4.0 ml of tetrahydrofurane. The reaction mixture was stirred at 80° C. for 5 h and then was cooled to 0° C. A 1.0 ml of water, 1.0 ml of 2N NaOH and 20.0 ml of diethyl ether were added. The mixture was stirred at room temp for 30 min, 2.2 g of MgSO₄ was added and mixture was stirred for next 15 min. The suspension was filtrated and solvent evaporated. The oil residue was chromatographed on columns (100 cm³ and 30 cm³) using dichloromethane:ethyl acetate (4:1) as mobile phase. Fractions containing product were pooled and evaporated to give 279 mg (93%) of product 73 as colorless oil.

¹H NMR (CDCl₃): 6.32 (1H, dt, J=15.7, 7.8 Hz), 5.59 (1H, 15.7 Hz), 4.09 (1H, br s), 2.29 (2H, d, J=7.6 Hz), 2.01 (1H, br d, J=3.3 Hz), 1.86-1.75 (2H, m), 1.63-1.04 (18H, m), 1.21 (6H, s), 1.09 (3H, s), 0.98 (3H, s)

¹³C NMR (CDCl₃): 137.07, 119.81, 71.52, 69.54, 69.57, 57.20, 52.53, 44.16, 43.50, 42.29, 41.43, 40.10, 40.04, 33.39, 29.33, 29.29, 23.01, 22.17, 21.69, 17.86, 17.51, 16.58

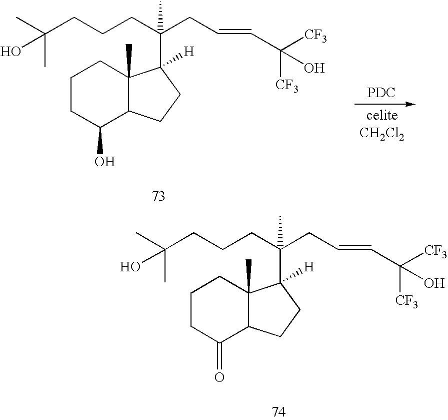

### (1R,3aR,4S,7aR-7a-Methyl-1-[(1S,3E)-6,6,6-trifluoro-5-hydroxy-1-(4-hydroxy-4-methyl-pentyl)-1-methyl-5-trifluoromethyl-hex-3-enyl]-octahydro-inden-4-one (74)

A 25 ml round bottom flask equipped with stir bar and Claisen adapter with rubber septum was charged with 274 mg (0.561 mmol) of (6S,3E)-1,1,1-trifluoro-6-[(1R,3aR,4S,7aR)-4-hydroxy-7a-methyl-octahydro-inden-1-yl]-6,10-dimethyl-2-trifluoromethyl-undec-3-ene-2,10-diol (73) and 10 ml of dichloromethane. A 704 mg (1.871 mmol) of pyridium dichromate and 740 mg of celite was added and mixture was stirred in room temperature for 2 h. The reaction mixture was filtrated through column with silica gel (100 cm³) using dichloromethane:ethyl acetate (4:1) as a mobile phase. The fractions containing product were pooled and evaporated to give 253 mg of yellow oil. The product 74 was used to the next reaction without farther purification.

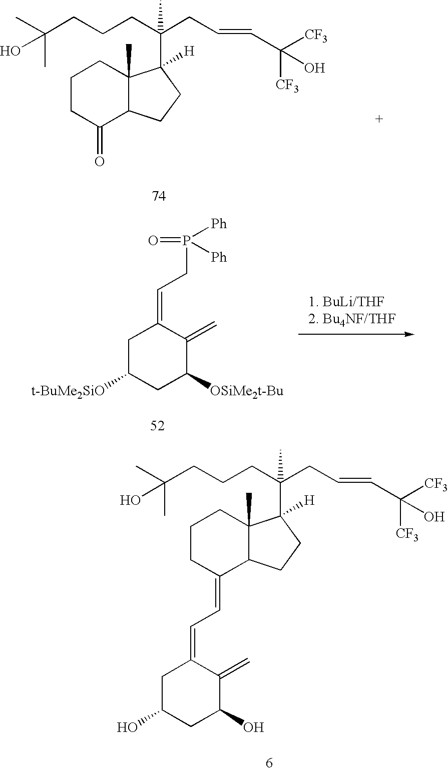

### (20S)-1,25-Dihydroxy-20-[(2E)-5,5,5-trifluoro-4-hydroxy-4-trifluoromethyl-pent-2-enyl]-cholecalciferol (6)

A 25 ml round bottom flask equipped with stir bar and Claisen adapter with rubber septum was charged with 1.765 g (3.028 mmol) of (1S,5R)-1,5-bis-((tert-butyldimethyl)silanyloxy)-3-[2-(diphenylfosphinoyl)-eth-(Z)-ylidene]-2-methylene-cyclohexane (52) and 10.0 ml of tetrahydrofurane. The reaction mixture was cooled to −78° C. and 1.8 ml (2.88 mmol) of 1.6M n-butyllithium in tetrahydrofurane was added dropwise. The resulting deep red solution was stirred at −78° C. for 20 min and 253 mg (0.520 mmol) of (1R,3aR,4S,7aR)-7a-methyl-1-[(1S,3E)-6,6,6-trifluoro-5-hydroxy-1-(4-hydroxy-4-methyl-pentyl)-1-methyl-5-trifluoromethyl-hex-3-enyl]-octahydro-inden-4-one (74) was added dropwise in 1.5 ml of tetrahydrofurane. The reaction mixture was stirred for 5 h (last 0.5 h at −20° C.) and then the bath was removed and the mixture was poured into 50 ml of ethyl acetate and 100 ml of brine. The water fraction was extracted three times with 50 ml of ethyl acetate, dried over Na₂SO₄ and evaporated. The oil residue was chromatographed on column (60 cm³, protected from light) using hexane:ethyl acetate (4:1) as mobile phase. Fractions containing product were pooled and evaporated to give colorless oil (304 mg) which was treated with 5 ml of 1M tetrabutylammonium fluoride in tetrahydrofurane. The reaction mixture was stirred at room temperature for 21 h. The mixture was dissolved by the addition of 150 ml of ethyl acetate and extracted six times with 50 ml of water:brine (1:1) and 50 ml of brine, dried over Na₂SO₄ and evaporated. The oil residue was chromatographed on column (50 cm³, protected from light) using ethyl acetate as mobile phase. Fractions containing product were pooled and evaporated to give product as colorless oil. Oil was dissolved in methyl acetate and evaporated (4 times) to give 176 mg (54%, two steps) of product 6 as white foam.

[α]_{D}²⁹=−4.5° c=0.33, CHCl₃

UV λmax (EtOH): 204.50 nm (ε 17846), 266.17 nm (ε 16508)

¹H NMR (CDCl₃): 6.36 (1H, d, J=11.3 Hz), 6.32 (1H, dt, J=15.1, 7.5 Hz), 6.00 (1H, d, J=11.1 Hz), 5.59 (1H, d, J=15.8 Hz, 5.33 (1H, s), 4.99 (1H, s), 4.53 (1H, br s), 4.43 (1H, dd, J=7.7, 4.3 Hz), 4.25-4.00 (1H, m), 2.81 (1H, dd, J=12.1, 3.8 Hz), 2.59 (1H, dd, J=13.3, 2.9 Hz), 2.34-2.29 (3H, m), 2.05-1.96 (3H, m), 1.93-1.87 (1H, m), 1.71-1.21 (17H, m), 1.21 (6H, s), 1.12-1.05 (1H, m), 0.95 (3H, s), 0.66 (3H, s)

¹³C NMR (CDCl₃): 147.48, 142.53, 136.92, 133.05, 124.83, 122.39 (q, J=284.7 Hz), 119.76, 117.58, 117.49, 111, 71, 71.61, 70.73, 66.90, 57.39, 56.62, 46.79, 45.18, 43.99, 42.83, 42.48, 41.29, 40.13, 40.04, 29.62, 29.28, 28.98, 23.50, 23.06, 22.24, 21.90, 17.74, 15.11

| | | | | |
|---|---|---|---|---|
| MS HRES | Calculated for: | C₃₃H₄₈F₆O₄ | [M + Na]⁺ | 645.3349 |
| | Observed: | | [M + Na]⁺ | 645.3346 |

### Example 11

### Synthesis of (20S)-1,25-Dihydroxy-20-[(2E)-5,5,5-trifluoro-4-hydroxy-4-trifluoromethyl-pent-2-enyl]-19-nor-cholecalciferol (12)

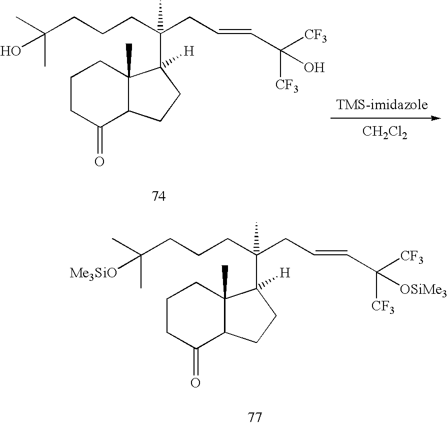

### (1R,3aR,4S,7aR)-7a-Methyl-1-[(1S,3E-6,6,6-trifluoro-1-methyl-1-(4-methyl-4-trimethylsilanyloxy-pentyl)-5-trifluoromethyl-5-trimethylsilanyloxy-hex-3-enyl]-octahydro-inden-4-one (77)

A 25 ml round bottom flask equipped with stir bar and Claisen adapter with rubber septum was charged with 577 mg (1.186 mmol) of (1R,3aR,4S,7aR)-7a-methyl-1-[(1S,3E)-6,6,6-trifluoro-5-hydroxy-1-(4-hydroxy-4-methyl-pentyl)-1-methyl-5-trifluoromethyl-hex-3-enyl]-octahydro-inden-4-one (74) and 20 ml of dichloromethane. A 1.5 ml (10.2 mmol) of 1-(trimethylsilyl)imidazole was added dropwise. The mixture was stirred at room temperature for 5 h 30 min. A 150 ml of ethyl acetate was added and the mixture was washed three times with 50 ml of water, dried over Na₂SO₄ and evaporated. The oil residue was chromatographed on column (75 cm³) using hexane:ethyl acetate (10:1) as mobile phase. Fractions containing product were pooled and evaporated to give 710 mg (95%) of product 77 as colorless oil.

¹H NMR (CDCl₃): 6.21 (1H, dt, J=15.1, 7.2 Hz), 5.56 (1H, d, J=15.4 Hz), 1.22-1.19 (1H, m), 2.32-1.06 (2H, m), 2.27 (2H, d, J=7.0 Hz), 2.06-1.52 (9H, m), 1.34-1.08 (6H, m), 1.20 (3H, s), 1.19 (3H, s), 0.96 (3H, s), 0.73 (3H, s), 0.22 (9H, s), 0.0 (9H, s)

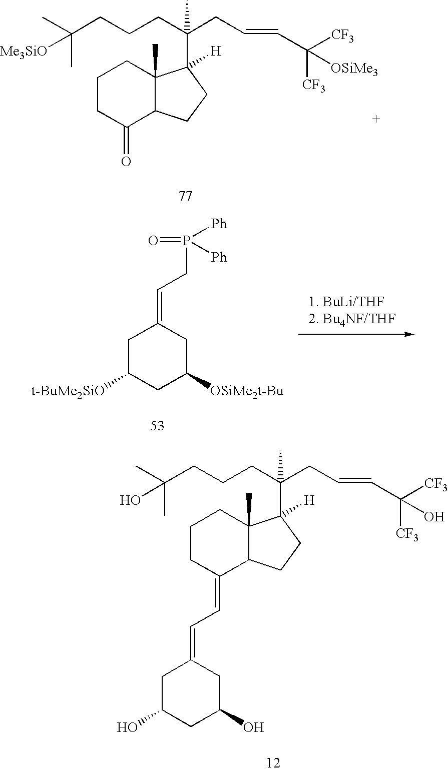

### (20S)-1,25-Dihydroxy-20-[(2E)-5,5,5-trifluoro-4-hydroxy-4-trifluoromethyl-pent-2-enyl]-19-nor-cholecalciferol (12)

A 25 ml round bottom flask equipped with stir bar and Claisen adapter with rubber septum was charged with 836 mg (1,464 mmol) of (1R,3R)-1,3-bis-((tert-butyldimethyl)silanyloxy)-5-[2-(diphenylfosphinoyl)ethylidene]-cyclohexane (53) and 10 ml of tetrahydrofurane. The reaction mixture was cooled to −70° C. and 0.89 ml (1.42 mmol) of 1.6M n-butyllithium BuLi was added dropwise. The resulting deep red solution was stirred at −70° C. for 20 min and 360 mg (0.571 mmol) of (1R,3aR,4S,7aR)-7a-methyl-1-[(1S,3E)-6,6,6-trifluoro-1-methyl-1-(4-methyl-4-trimethylsilanyloxy-pentyl)-5-trifluoromethyl-5-trimethylsilanyloxy-hex-3-enyl]-octahydro-inden-4-one (77) in 1.5 ml of tetrahydrofurane. The reaction mixture was stirred for 5 h and then the dry ice was removed from bath and the solution was allowed to warm up to −40° C. in 1 h. The mixture was poured into 50 ml of ethyl acetate and 100 ml of brine. The water fraction was extracted three times with 50 ml of ethyl acetate, dried over Na₂SO₄ and evaporated. The oil residue was chromatographed on column (50 cm³, protected from light) using hexane:ethyl acetate (10:1) as mobile phase. Fractions containing product were pooled and evaporated to give colorless oil (ca. 440 mg) which was treated with 10 ml of 1M tetrabutylammonium fluoride in tetrahydrofurane. The reaction mixture was stirred at room temperature for 26 h. The new portion 2.5 ml of 1M tetrabutylammonium fluoride in tetrahydrofurane was added and the mixture was stirred for next 6 h.

The mixture was dissolved by the addition of 150 ml of ethyl acetate and extracted six times with 50 ml of water:brine (1:1) and 50 ml of brine, dried over Na₂SO₄ and evaporated. The oil residue was chromatographed on column (50 cm³, protected from light) using ethyl acetate as mobile phase. Fractions containing product were pooled and evaporated to give product as colorless oil. Oil was dissolved in methyl acetate and evaporated (2 times) to give 303 mg (87%) of product 12 as white foam.

[α]_{D}²⁶=+41.8 c=0.44, EtOH

UV λmax (EtOH): 244 nm (ε 27480), 252 nm (εα32212), 262 nm (ε 21694)

¹H NMR (CDCl₃): 6.33 (1H, dt, J=15.6, 7.8 Hz), 6.29 (1H, d, J=9.0 Hz), 5.83 (1H, d, J=11.1 Hz), 5.58 (1H, d, J=15.6 Hz), 4.12-4.09 (1H, m), 4.05-4.02 (1H, m), 2.79-2.71 (2H, m), 2.46 (1H, dd, J=13.2, 3.0 Hz), 2.29 (2H, d, J=7.5 Hz), 2.20 (2H, dd, J=13.3, 7.1 Hz), 2.04-1.75 (7H, m), 1.68-1.46 (9H, m), 1.41-1.21 (6H, m), 1.21 (6H, s), 1.12-1.05 (11H, m), 0.95 (3H, s), 0.65 (3H, s)

¹³C NMR (CDCl₃): 142.40, 136.79, 131.25, 123.64, 122.4 (q, J=286.96 Hz), 119.83, 115.76, 71.59, 67.42, 67.18, 57.33, 56.56, 46.64, 44.52, 44.04, 42.40, 42.02, 41.24, 40.10, 40.01, 37.13, 29.54, 29.26, 28.83, 23.39, 23.07, 22.25, 21.87, 17.79, 15.17

| | | | | |
|---|---|---|---|---|
| MS HRES | Calculated for: | C₃₂H₄₈F₆O₄ | [M + Na]⁺ | 633.3349 |
| | Observed: | | [M + Na]⁺ | 633.3349 |

### Example 12

### Synthesis of (20S)-1α-Fluoro-25-hydroxy-20-[(2E)-5,5,5-trifluoro-4-hydroxy-4-trifluoromethyl-pent-2-enyl]-cholecalciferol (15)

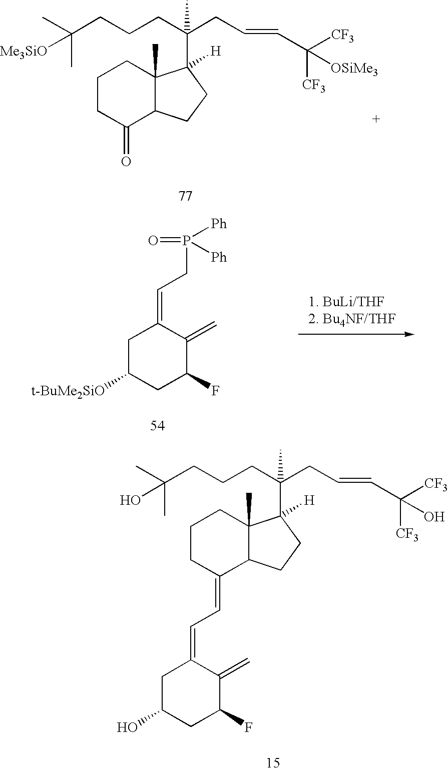

### (20S)-1α-Fluoro-25-hydroxy-20-[(2E)-5,5,5-trifluoro-4-hydroxy-4-trifluoromethyl-pent-2-enyl]-cholecalciferol (15)

A 25 ml round bottom flask equipped with stir bar and Claisen adapter with rubber septum was charged with 521 mg (1.107 mmol) of (1S,5R)-1-((tert-butyldimethyl)silanyloxy)-3-[2-(diphenylfosphinoyl)-eth-(Z-ylidene]-5-fluoro-2-methylene-cyclohexane (54) and 10 ml of tetrahydrofurane. The reaction mixture was cooled to −70° C. and 0.69 ml (1.10 mmol) of 1.6M n-butyllithium was added dropwise. The resulting deep red solution was stirred at −70° C. for 20 min and 324 mg (0.514 mmol) of (1R,3aR,4S,7aR)-7a-methyl-1-[(1S,3E)-6,6,6-trifluoro-1-methyl-1-(4-methyl-4-trimethylsilanyloxy-pentyl)-5-trifluoromethyl-5-trimethylsilanyloxy-hex-3-enyl]-octahydro-inden-4-one (77) was added dropwise in 1.5 ml of tetrahydrofurane. The reaction mixture was stirred for 4 h and then the dry ice. was removed from bath and the solution was allowed to warm up to −40° C. in 1 h. The mixture was poured into 50 ml of ethyl acetate and 100 ml of brine. The water fraction was extracted three times with 50 ml of ethyl acetate, dried over Na₂SO₄ and evaporated. The oil residue was chromatographed on column (50 cm³, protected from light) using hexane:ethyl acetate (10:1) as mobile phase. Fractions containing product were pooled and evaporated to give colorless oil which was treated with 8 ml of 1M tetrabutylammonium fluoride in tetrahydrofurane. The reaction mixture was stirred at room temperature for 9 h.

The mixture was dissolved by the addition of 150 ml of ethyl acetate and extracted six times with 50 ml of water:brine (1:1) and 50 ml of brine, dried over Na₂SO₄ and evaporated. The oil residue was chromatographed on column (50 cm³, protected from light) using ethyl acetate:hexane (1:1) as mobile phase. Fractions containing product were pooled and evaporated to give product as colorless oil. The product was dissolved in methyl acetate and evaporated (2 times) to give 305 mg 95%) of product 15 as white foam.

[α]_{D}²⁶=+29.3 c=0.43, EtOH

UV λmax (EtOH): 210 nm (ε 13484), 243 nm (ε 13340), 271 nm (ε 13609)

¹H NMR (CDCl₃): 6.39 (1H, d, J=11.3 Hz), 6.32 (1H, dt, J=15.6, 7.6 Hz), 6.01 (1H, d, J=11.3 Hz), 5.58 (1H, d, J=15.8 Hz), 2.39 (1H, s), 5.13 (1H, ddd, J=49.9, 6.3, 3.8 Hz), 5.09 (1H, s), 4.21 (1H, br s), 2.81 (1H, dd, J=11.8, 3.5 Hz), 2.61 (1H, dd, J=13.2, 3.2 Hz), 2.32-2.28 (3H, m), 2.23-2.15 (1H, m), 2.04-1.93 (3H, m), 1.70-1.48 (9H, m), 1.41-1.21 (8H, m), 1.21 (6H, s), 1.12-1.05 (1H, m), 0.95 (3H, s), 0.65 (3H, s)

¹³C NMR (CDCl₃): 142.95 (d, J=16.0 Hz), 136.84, 131.54, 125.42, 122.42 (q, J=286.9 Hz), 119.78, 117.53, 114.96 (d, J=10.0 Hz), 71.74, 66.56 (d, J=6.0 Hz), 57.35, 56.61, 46.82, 44.91, 44.04, 42.40, 41.29, 40.69 (d, J=20.6 Hz), 40.10, 39.98, 29.47, 29.20, 29.01, 23.47, 23.07, 22.22, 21.82, 17.79, 15.13

| | | | | |
|---|---|---|---|---|
| MS HRES | Calculated for: | C₃₃H₄₇F₇O₃ | [M + Na]⁺ | 647.3305 |
| | Observed: | | [M + Na]⁺ | 647.3302 |

### Example 13

### Synthesis of (20R)-1,25-Dihydroxy-20-(5,5,5-trifluoro-4-hydroxy-4-trifluoromethyl-pent-2-ynyl)-cholecalciferol (7)

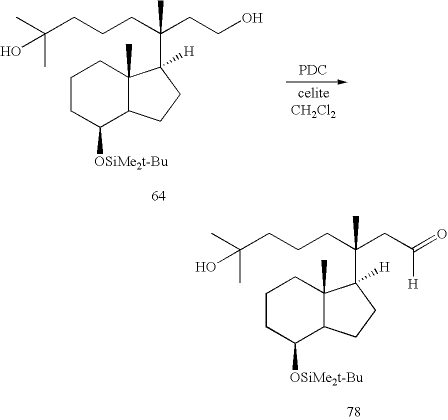

### (3R)-3-[(1R,3aR,4S,7aR)-4-(tert-Butyl-dimethyl-silanyloxy)-7a-methyl-octahydro-inden-1-yl]-7-hydroxy-3,7-dimethyl-octanal (78)

A 50 ml round bottom flask equipped with stir bar and Claisen adapter with rubber septum was charged with 1.558 g (7.228 mmol) of pyridinium chlorochromate, 1.60 g of celite and 20 ml of dichloromethane. A 1.440 g (3.267 mmol) of (3R)-3-[(1R,3aR,4S,7aR)-4-(tert-butyl-dimethyl-silanyloxy)-7a-methyl-octahydro-inden-1-yl]-3,7-dimethyl-octane-1,7-diol in 10 ml of dichloromethane was added dropwise and mixture was stirred in room temperature for 2 h 50 min. The reaction mixture was filtrated through column with silica gel (75 cm³) and celite (2 cm) and using dichloromethane, dichloromethane:ethyl acetate (4:1) as a mobile phase. The fractions containing product were pooled and evaporated to give 1.298 g of yellow oil. The product was used to the next reaction without farther purification.

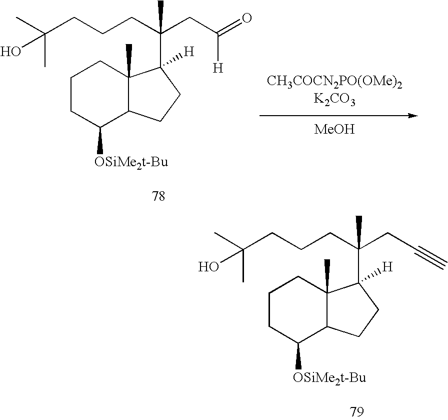

### (6R)-6-[(1R,3aR,4S,7aR)-4-(tert-Butyl-dimethyl-silanyloxy)-7a-methyl-octahydro-inden-1-yl]-2,6-dimethyl-non-8-yn-2-ol (79)

A 50 ml round bottom flask equipped with stir bar and Claisen adapter with rubber septum was charged with 1.298 g (2.958 mmol) of (3R)-3-[(1R,3aR,4S,7aR)-4-(tert-butyl-dimethyl-silanyloxy)-7a-methyl-octahydro-inden-1-yl]-7-hydroxy-3,7-dimethyl-octanal and 30 ml of methanol. A 1.137 g (5.916 mmol) of 1-diazo-2-oxo-propyl)-phosphonic acid dimethyl ester in 3 ml of methanol was added and the resulting mixture was cooled in an ice bath to 0° C. A 1.140 g (8.248 mmol) of potassium carbonate was added and the reaction mixture was stirred in the ice bath for 30 min and then at room temperature for 2 h 50 min. A 100 ml of water was added and the mixture was extracted three times with 80 ml of ethyl acetate, dried over Na₂SO₄ and evaporated. The oil residue was chromatographed on column (200 cm³) using hexane:ethyl acetate (7:1) as mobile phase. Fractions containing product were pooled and evaporated to give 1.151 g (81%) of product as colorless oil.

[α]_{D}²⁹=+18.3° c=0.54, CHCl₃

¹H NMR (CDCl₃): 3.99 (1H, br s), 2.16-2.07 (2H, m), 2.00-1.97 (1H, m), 1.92 (1H, t, J=2.6 Hz), 1.84-1.74 (1H, m), 1.67-1.64 (1H, m), 1.58-1.22 (16H, m), 1.22 (6H, s), 1.04 (3H, s), 0.99 (3H, s), 0.88 (9H, s), 0.00 (3H, s), −0.01 (3H, s)

| | | | | |
|---|---|---|---|---|
| MS HRES | Calculated for: | C₂₇H₅₀O₂Si | [M + Na]⁺ | 457.3472 |
| | Observed: | | [M + Na]⁺ | 457.3473 |

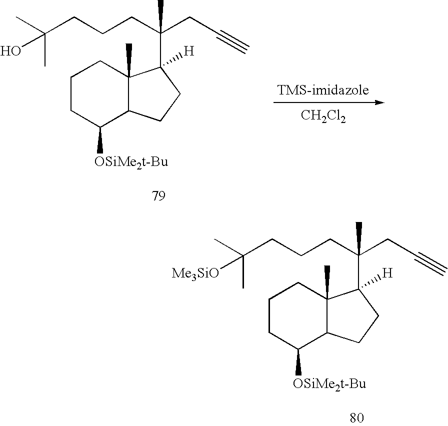

### (1R,3aR,4S,7aR)-4-(tert-Butyl-dimethyl-silanyloxy)-1-[(1R)-1,5-dimethyl-1-prop-2-ynyl-5-trimethylsilanyloxy-hexyl]-7a-methyl-octahydro-indene (80)

A 50 ml round bottom flask equipped with stir bar and Claisen adapter with rubber septum was charged with 1.151 g (2.647 mmol) of (6R)-6-[(1R,3aR,4S,7aR)-4-(tert-butyldimethyl-silanyloxy)-7a-methyl-octahydro-inden-1-yl]-2,6-dimethyl-non-8-yn-2-ol and 20 ml of dichloromethane. A 2.0 ml (13.63 mmol) of 1-(trimethylsilyl)imidazole was added dropwise. The mixture was stirred at room temperature for 1 h. A 100 ml of water was added and the mixture was extracted three times with 50 ml of ethyl acetate, dried over Na₂SO₄ and evaporated. The oil residue was chromatographed on column (75 cm³) using hexane:ethyl acetate (25:1) as mobile phase. Fractions containing product were pooled and evaporated to give 1.260 g (94%) of product as colorless oil.

[α]_{D}²⁹=+18.5° c=0.46, CHCl₃

¹H NMR (CDCl₃): 3.98 (1H, br s), 2.12-2.08 (2H, m), 20.5-1.95 (2H, m), 1.92-1.90 (1H, m), 1.83-1.21 (16H, m), 1.21 (6H, s), 1.04 (3H, s), 0.98 (3H, s), 0.88 (9H, s), 0.11 (9H, s), 0.00 (3H, s), −0.01 (3H, s)

¹³C NMR (CDCl₃): δ3.00, 74.07, 69.70, 69.50, 56.63, 53.03, 45.66, 43.74, 41.35, 39.59, 39.45, 34.38, 29.99, 29.60, 25.85, 22.81, 22.43, 22.06, 18.56, 18.05, 17.76, 16.49, 2.65, −4.77, −5.13

| | | | | |
|---|---|---|---|---|
| MS HRES | Calculated for: | C₃₀H₅₈O₂Si₂ | [M + Na]⁺ | 529.3867 |
| | Observed: | | [M + Na]⁺ | 529.3868 |

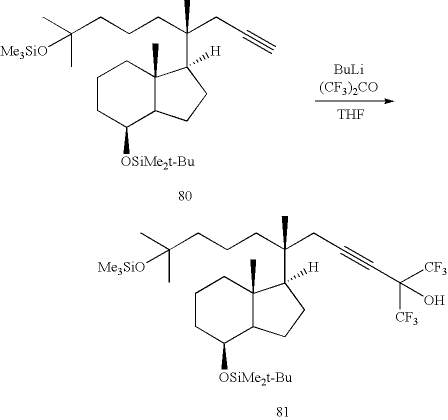

### (6R)-6-[(1R,3aR,4S,7aR)-4-(tert-Butyl-dimethyl-silanyloxy)-7a-methyl-octahydro-inden-1-yl]-1,1,1-trifluoro-6,10-dimethyl-2-trifluoromethyl-10-trimethylsilanyloxy-undec-3-yn-2-ol (81)

A two neck 50 ml round bottom flask equipped with stir bar, Claisen adapter with rubber septum and funnel (with cooling bath) was charged with 1.252 g (2.470 mmol) of (1R,3aR,4S,7aR)-4-(tert-butyl-dimethyl-silanyloxy)-1-[(1R)-1,5-dimethyl-1-prop-2-ynyl-5-trimethylsilanyloxy-hexyl]-7a-methyl-octahydro-indene and 25 ml of tetrahydrofurane. The funnel was connected to container with hexafluoroacetone and cooled (acetone, dry ice). The reaction mixture was cooled to −70° C. and 2.4 ml (3.84 mmol) of 1.6M n-butyllithium in tetrahydrofurane was added dropwise. After 30 min hexafluoroacetone was added (the container's valve was opened three times). The reaction was stirred at −70° C. for 2 h then 5.0 ml of saturated solution of ammonium chloride was added. The mixture was dissolved by the addition of 100 ml of saturated solution of ammonium chloride and extracted three times with 80 ml of ethyl acetate, dried over Na₂SO₄ and evaporated. The residue was chromatographed twice on columns (75 cm³) using hexane:ethyl acetate (10:1) as mobile phase to give 1.711 g of mixture of product and polymer (from hexafluoroacetone).

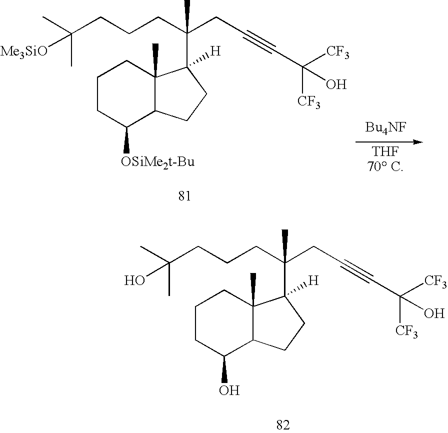

### (6R)-1,1,1-Trifluoro-6-[(1R,3aR,4S,7aR)-4-hydroxy-7a-methyl-octahydro-inden-1-yl]-6,10-dimethyl-2-trifluoromethyl-undec-3-yne-2,10-diol (82)

A 25 ml round bottom flask equipped with stir bar and Claisen adapter with rubber septum was charged with crude (ca 2.470 mmol) (6R)-6-[(1R,3aR,4S,7aR)-4-(tert-butyldimethyl-silanyloxy)-7a-methyl-octahydro-inden-1-yl]-1,1,1-trifluoro-6,10-dimethyl-2-trifluoromethyl-10-trimethylsilanyloxy-undec-3-yn-2-ol and 15.0 ml (15.0 mmol) of 1M tetrabutylammonium fluoride in tetrahydrofurane. The reaction mixture was stirred at 70° C. for 96 h. The mixture was dissolved by the addition of 150 ml of ethyl acetate and extracted six times with 50 ml of water:brine (1:1) and 50 ml of brine, dried over Na₂SO₄ and evaporated. The oil residue was chromatographed on columns, 200 cm³ and 75 cm³ using hexane:ethyl acetate (2:1). The fractions containing product were pooled and evaporated to give 979 mg (81%) of product as colorless oil.

[α]_{D}³⁰=+1.04° c=0.48, CHCl₃

¹H NMR (CDCl₃): 4.08 (1H, br s), 2.24 (1H, AB, J=17.2 Hz), 2.17 (1H, AB, J=17.2 Hz), 2.05-2.02 (1H, m), 1.85-1.76 (2H, m), 1.66-1.20 (18H, m), 1.26 (3H, s), 1.25 (3H, s), 1.07 (3H, s), 1.01 (3H, s)

| | | | | |
|---|---|---|---|---|
| MS HRES | Calculated for: | C₂₄H₃₆F₆O₃ | [M + Na]⁺ | 509.2461 |
| | Observed: | | [M + Na]⁺ | 509.2463 |

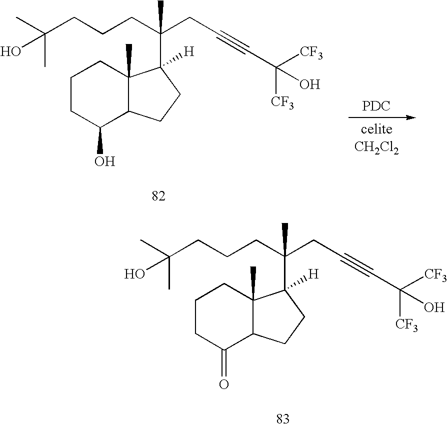

### (1R,3aR,4S,7aR)-7a-Methyl-1-[(1R)-6,6,6-trifluoro-5-hydroxy-1-(4-hydroxy-4-methyl-pentyl)-1-methyl-5-trifluoromethyl-hex-3-ynyl]-octahydro-inden-4-one (83)

A 25 ml round bottom flask equipped with stir bar and Claisen adapter with rubber septum was charged with 291 mg (0.598 mmol) of (6R)-1,1,1-trifluoro-6-[(1R,3aR,4S,7aR)-4-hydroxy-7a-methyl-octahydro-inden-1-yl]-6,10-dimethyl-2-trifluoromethyl-undec-3-yne-2,10-diol and 10 ml of dichloromethane. A 700 mg (1.861 mmol) of pyridinium dichromate and 720 mg of celite was added and mixture was stirred in room temperature for 3 h. The reaction mixture was filtrated through column with silica gel (75 cm³) using dichloromethane, dichloromethane:ethyl acetate (4:1, 3:1). The fractions containing product were pooled and evaporated to give 271 mg (94%) of product as yellow oil.

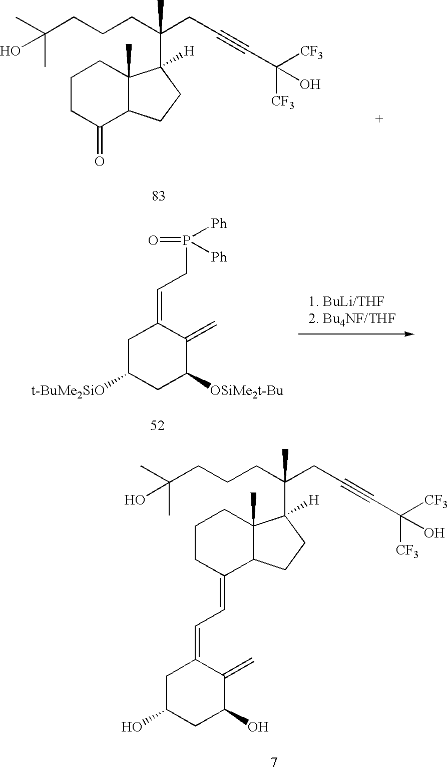

### (20R)-1,25-Dihydroxy-20-(5,5,5-trifluoro-4-hydroxy-4-trifluoromethyl-pent-2-ynyl)-cholecalciferol (7)

A 25 ml round bottom flask equipped with stir bar and Claisen adapter with rubber septum was charged with 2.118 g (3.634 mmol) of (1S,5R)-1,5-bis-((tert-butyldimethyl)silanyloxy)-3-[2-(diphenylfosphinoyl)-eth-(Z)-ylidene]-2-methylene-cyclohexane and 10 ml of tetrahydrofurane. The reaction mixture was cooled to −78° C. and 2.2 ml (3.52 mmol) of 1.6M n-butyllithium in tetrahydrofurane was added dropwise. The resulting deep red solution was stirred at −78° C. for 20 min and 271 mg, (0.559 mmol) of (1R,3aR,4S,7aR)-7a-methyl-1-[(1R,3E)-6,6,6-trifluoro-5-hydroxy-1-(4-hydroxy-4-methyl-pentyl)-1-methyl-5-trifluoromethyl-hex-3-ynyl]-octahydro-inden-4-one was added dropwise in 1.5 ml of tetrahydrofurane. The reaction mixture was stirred at −78° C. for 5 h and then the bath was removed and the mixture was poured into 100 ml of saturated solution of ammonium chloride and extracted three times with 50 ml of ethyl acetate, dried over Na₂SO₄ and evaporated. The oil residue was chromatographed on column (50 cm³, protected from light) using hexane:ethyl acetate (4:1) as mobile phase. The fractions contains impurities was chromatographed on column (50 cm³, protected from light) using hexane:ethyl acetate (5:1) as mobile phase. Fractions containing product were pooled and evaporated to give colorless oil (250 mg) which was treated with 5 ml of 1M tetrabutylammonium fluoride in tetrahydrofurane. The reaction mixture was stirred at room temperature for 18 h. The mixture was dissolved by the addition of 150 ml of ethyl acetate and extracted six times with 50 ml of water:brine (1:1) and 50 ml of brine, dried over Na₂SO₄ and evaporated. The oil residue was chromatographed on column (50 cm³, protected from light) using ethyl acetate as mobile phase. Fractions containing product were pooled and evaporated to give product as colorless oil. Oil was dissolved in methyl acetate and evaporated (4 times) to give 194 mg (56%) of product as white foam.

[α]_{D}³⁰=+7.9° c=0.38, EtOH

UV λmax (EtOH): 212.33 nm (ε 14113), 265.00 nm (ε 15960)

¹H NMR (D6-DMSO): 8.93 (1H, s), 6.18 (1H, d, J=11.3 Hz), 5.96 (1H, d, J=11.3 Hz), 5.22 (1H, s), 4.86 (1H, d, J=4.83 Hz), 4.75 (1H, s), 4.54 (1H, d, J=3.63 Hz), 4.20-4.15 (1H, m), 4.06 (1H, s), 3.98 (1H, br s), 2.77 (1H, d, J=13.7 Hz), 2.40-2.33 (1H, m), 2.27-2.14 (3H, m), 2.00-1.90 (2H, m), 1.82-1.78 (2H, m), 1.64-1.54 (5H, m), 1.47-1.18 (10H, m), 1.05 (3H, s), 1.05 (3H, s), 0.95 (3H, s), 0.59 (3H, s)

¹³C NMR (D6-DMSO): 149.38, 139.51, 135.94, 122.32, 121.47 (q, J=287.5 Hz), 117.99, 109.77, 89.53, 70.58, 68.72, 68.35, 65.06, 56.02, 55.91, 46.06, 44.85, 44.65, 43.11, 29.30, 29.03, 28.78, 28.32, 23.05, 22.40, 21.90, 21.52, 18.27, 14.29

| | | | | |
|---|---|---|---|---|
| MS HRES | Calculated for: | C₃₃H₄₆F₆O₄ | [M + Na]⁺ | 643.3192 |
| | Observed: | | [M + Na]⁺ | 643.3190 |

### Example 14

### Synthesis of (20R)-1,25-Dihydroxy-20-(5,5,5-trifluoro-4-hydroxy-4-trifluoromethyl-pent-2-ynyl)-19-nor-cholecalciferol (34)

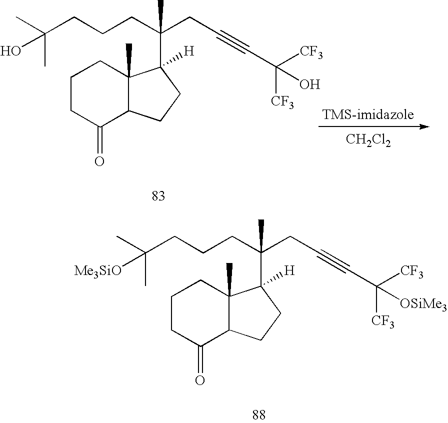

### (1R,3aR,4S,7aR-7a-Methyl-1-[(1R)-6,6,6-trifluoro-1-methyl-1-(4-methyl-4-trimethylsilanyloxy-pentyl)-5-trifluoromethyl-5-trimethylsilanyloxy-hex-3-ynyl]-octahydro-inden-4-one (88)

A 25 ml round bottom flask equipped with stir bar and Claisen adapter with rubber septum was charged with 399 mg (0.823 mmol) of (1R,3aR,4S,7aR)-7a-methyl-1-[(1R)-6,6,6-trifluoro-5-hydroxy-1-(4-hydroxy-4-methyl-pentyl)-1-methyl-5-trifluoromethyl-hex-3-ynyl]-octahydro-inden-4-one and 8.0 ml of dichloromethane. A 0.9 ml (6.2 mmol) of 1-trimethylsilyl)imidazole was added dropwise. The mixture was stirred at room temperature for 4 h. A 150 ml of hexane was added and the mixture was washed three times with 50 ml of water, dried over Na₂SO₄ and evaporated. The oil residue was chromatographed on column (50 cm³) using hexane:ethyl acetate (5:1) as mobile phase. Fractions containing product were pooled and evaporated to give 492 mg (95%) of product as oil.

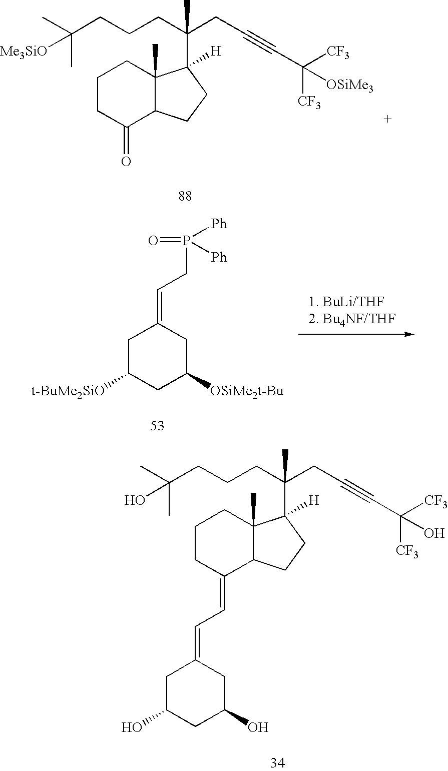

### (20R)-1,25-Dihydroxy-20-(5,5,5-trifluoro-4-hydroxy-4-trifluoromethyl-pent-2-ynyl)-19-nor-cholecalciferol (34)

A 25 ml round bottom flask equipped with stir bar and Claisen adapter with rubber septum was charged with 490 mg (0.858 mmol) of (1R,3R)-1,3-bis-((tert-butyl dimethyl)silanyloxy)-5-[2-(diphenylfosphinoyl)ethylidene]-cyclohexane and 8 ml of tetrahydrofurane. The reaction mixture was cooled to −70° C. and 0.53 ml (0.848 mmol) of 1.6M n-butyllithium BuLi was added dropwise. The resulting deep red solution was stirred at −70° C. for 30 min and 249 mg (0.396 mmol) of (1R,3aR,4S,7aR)-7a-methyl-1-[(1R)-6,6,6-trifluoro-1-methyl-1-(4-methyl-4-trimethylsilanyloxy-pentyl)-5-trifluoromethyl-5-trimethylsilanyloxy-hex-3-ynyl]-octahydro-inden-4-one in 1.5 ml of tetrahydrofurane. The reaction mixture was stirred for 4.5 h and then the dry ice was removed from bath and the solution was allowed to warm up to −55° C. in 1 h. The mixture was poured into ethyl acetate (50 ml) and saturated solution of ammonium chloride (50 ml). The water fraction was extracted with ethyl acetate (3×50 ml), dried (Na₂SO₄) and evaporated. The oil residue was chromatographed on column (50 cm³, protected from light) using hexane:ethyl acetate (10:1) as mobile phase. Fractions containing product were pooled and evaporated to give colorless oil (ca. 349 mg) which was treated with 10 ml of 1M tetrabutylammonium fluoride in tetrahydrofurane. The reaction mixture was stirred at room temperature for 63 h. The mixture was dissolved by the addition of 150 ml of ethyl acetate and extracted six times with 50 ml of water:brine (1:1) and 50 ml of brine, dried over Na₂SO₄ and evaporated. The oil residue was chromatographed on column (50 cm³, protected from light) using hexane:tetra hydrofurane (1:1) as mobile phase. Fractions containing product were pooled and evaporated to give product 207 mg (86%) as white solid.

[α]_{D}³⁰=+44.7 c=0.51, EtOH

UV λmax (EtOH): 242 nm (ε 30834)

¹H NMR (DMSO-D6): 8.96 (1H, s), 6.08 (1H, d, J=10.9 Hz), 5.78 (1H, d, J=11.3 Hz), 4.48 (1H, d, J=4.3 Hz), 4.38 (1H, d, J=4.1 Hz), 4.07 (1H, s), 3.91-3.85 (1H, m), 3.84-3.77 (1H, m), 2.74 (1H, d, J=13.6 Hz), 2.43 (1H, dd, J=13.4, 3.4 Hz), 2.28-2.20 (3H, m), 2.07-1.93 (4H, m), 1.84-1.79 (1H, m), 1.69-1.21 (16H, m), 1.06 (3H, s), 1.06 (3H, s), 0.97 (3H, s), 0.60 (3H, s)

¹³C NMR (D6-DMSO): 139.09, 134.88, 121.60 (q, J=286.0 Hz), 120.90, 116.56, 89.61, 70.64, 70.45 (sep, J=33.3 Hz), 68.77, 65.57, 65.30, 56.00, 55.92, 45.93, 44.66, 44.59, 42.22, 36.95, 29.27, 29.02, 28.78, 28.14, 22.87, 22.38, 21.93, 21.40, 18.24, 14.35

| | | | | |
|---|---|---|---|---|
| MS HRES | Calculated for: | C₃₂H₄₆F₆O₄ | [M + Na]⁺ | 631.3192 |
| | Observed: | | [M + Na]⁺ | 631.3195 |

### Example 15

### Synthesis of (20R)-1α-Fluoro-25-hydroxy-20-(5,5,5-trifluoro-4-hydroxy-4-trifluoromethyl-pent-2-ynyl)-cholecalciferol (37)

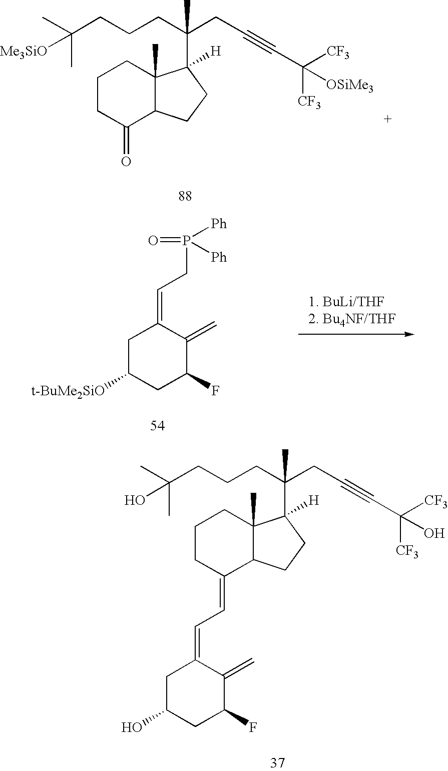

### (20R)-1α-Fluoro-25-hydroxy-20-(5,5,5-trifluoro-4-hydroxy-4-trifluoromethyl-pent-2-ynyl)-cholecalciferol (37)

A 25 ml round bottom flask equipped with stir bar and Claisen adapter with rubber septum was charged with 460 mg (0.977 mmol) of (1S,5R)-1-((tert-butyldimethyl)silanyloxy)-3-[2-(diphenylfosphinoyl)-eth-(Z)-ylidene]-5-fluoro-2-methylene-cyclohexane and 8 ml of tetrahydrofurane. The reaction mixture was cooled to −70° C. and 0.61 ml (0.976 mmol) of 1.6M n-butyllithium was added dropwise. The resulting deep red solution was stirred at −70° C. for 20 min and 240 mg (0.382 mmol) of (1R,3aR,4S,7aR)-7a-methyl-1-[(1R)-6,6,6-trifluoro-1-methyl-1-(4-methyl-4-trimethylsilanyloxy-pentyl)-5-trifluoromethyl-5-trimethylsilanyloxy-hex-3-ynyl]-octahydro-inden-4-one was added dropwise in 1.5 ml of tetrahydrofurane. The reaction mixture was stirred for 4.5 h and then the dry ice was removed from bath and the solution was allowed to warm up to −40° C. in 1.5 h. The mixture was poured into ethyl acetate (50 ml) and saturated solution of ammonium chloride (50 ml). The water fraction was extracted with ethyl acetate (3×50 ml), dried (Na₂SO₄) and evaporated. The oil residue was chromatographed on column (50 cm³, protected from light) using hexane:ethyl acetate (10:1) as mobile phase. Fractions containing product were pooled and evaporated to give colorless oil (ca. 239 mg) which was treated with 8 ml of 1M tetrabutylammonium fluoride in tetrahydrofurane. The reaction mixture was stirred at room temperature for 17 h. The mixture was dissolved by the addition of 150 ml of ethyl acetate and extracted six times with 50 ml of water:brine (1:1) and 50 ml of brine, dried over Na₂SO₄ and evaporated. The oil residue was chromatographed on column (50 cm³, protected from light) using ethyl acetate:hexane (1:2 and 1:1) as mobile phase. Fractions containing product were pooled and evaporated to give product as colorless oil. The product was dissolved in methyl acetate and evaporated (2 times) to give 196 mg (82%) of product as white foam.

[α]_{D}³⁰=+24.4 c=0.45, EtOH

UV λmax (EtOH): 241 nm (ε 17260), 273 nm (ε 16624)

¹H NMR (DMSO-D6): 8.95 (1H, s), 6.37 (1H, d, J=11.5 Hz), 5.93 (1H, d, J=11.1 Hz), 5.39 (1H, s), 5.14 (1H, br d, J=47.1 Hz), 4.99 (1H, d, J=1.9 Hz), 4.86 (1H, d, J=4.3 Hz), 4.07 (1H, s), 3.94-3.87 (1H, m), 2.83-2.80 (1H, m), 2.28-2.05 (4H, m), 2.00-1.93 (2H, m), 1.83-1.21 (17H, m), 1.06 (3H, s), 1.06 (3H, s), 0.96 (3H, s), 0.59 (3H, s)

¹³C NMR (D6-DMSO): 143.27 (d, J=16.7 Hz), 141.62, 133.20, 124.14, 121.59 (q, J=286.0 Hz), 117.49, 115.34 (d, J=9.8 Hz), 92.05 (d, J=166.9 Hz), 89.60, 70.64, 70.44 (sep, J=32.6 Hz), 68.77, 64.55 (d, J=4.5 Hz), 55.99, 55.92, 46.15, 44.83, 44.65, 40.68 (d, J=20.5 Hz), 40.05, 39.79, 39.41, 29.27, 29.02, 28.76, 28.30, 22.95, 22.33, 21.87, 21.39, 18.24, 14.28

| | | | | |
|---|---|---|---|---|
| MS HRES | Calculated for: | C₃₃H₄₅F₇O₃ | [M + Na]⁺ | 645.3149 |
| | Observed: | | [M + Na]⁺ | 645.3155 |

### Example 16

### Synthesis of (20)-1,25-Dihydroxy-20-[(2Z)-5,5,5-trifluoro-4-hydroxy-4-trifluoromethyl-pent-2-enyl]-cholecalciferol (8)

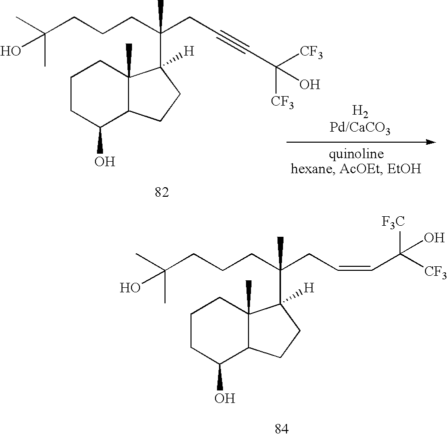

### (3Z,6R)-1,1,1-Trifluoro-6-[(1R,3aR,4S,7aR)-4-hydroxy-7a-methyl-octahydro-inden-1-yl]-6,10-dimethyl-2-trifluoromethyl-undec-3-ene-2,10-diol (84)

A 25 ml round bottom flask was charged with 340 mg (0.699 mmol) of (6R)-1,1,1-trifluoro-6-[(1R,3aR,4S,7aR)-4-hydroxy-7a-methyl-octahydro-inden-1-yl]-6,10-dimethyl-2-trifluoromethyl-undec-3-yne-2,10-diol, 100 mg of 5% Pd/CaCO₃, 8.0 ml of hexane, 3.3 ml of ethyl acetate and 0.32 ml of solution of quinoline in ethanol (prepared from 3.1 ml of ethanol and 168 μl of quinoline). The substrate was hydrogenated at ambient temperature and atmospheric pressure of hydrogen. The reaction was monitoring by TLC (hexane:ethyl acetate—2:1). After 7 h the catalyst was filtered off and solvent evaporated. The residue was purified over silica gel (50 cm³) using hexane:ethyl acetate (2:1). Fractions containing product were pooled and evaporated to give 320 mg (94%) of product as colorless oil.

¹H NMR (CDCl₃): 6.12-6.03 (1H, m), 5.46 (1H, d, J=13.2 Hz), 4.08 (1H, br s), 2.46-2.40 (2H, m), 2.06-1.95 (1H, m), 1.86-1.76 (2H, m), 1.66-1.20 (18H, m), 1.21 (6H, s), 1.09 (3H, s), 0.99 (3H, s)

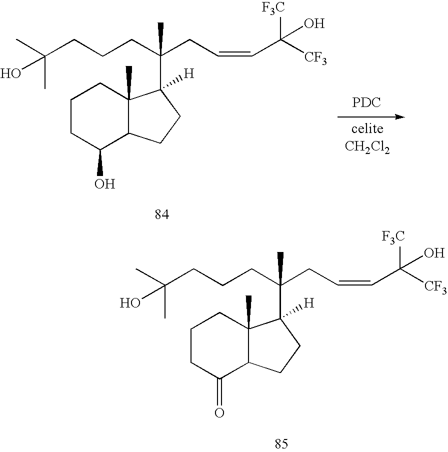

### (1R,3aR,4S,7aR)-7a-Methyl-1-[(1R,3Z)-6,6,6-trifluoro-5-hydroxy-1-(4-hydroxy-4-methyl-pentyl)-1-methyl-5-trifluoromethyl-hex-3-enyl]-octahydro-inden-4-one (85)

A 25 ml round bottom flask equipped with stir bar and Claisen adapter with rubber septum was charged with 315 mg (0.645 mmol) of (1R,3Z)-1,1,1-trifluoro-6-[(1R,3aR,4S,7aR)-4-hydroxy-7a-methyl-octahydro-inden-1-yl]-6,10-dimethyl-2-trifluoromethyl-undec-3-ene-2,10-diol and 12.0 ml of dichloromethane. A 780 mg (1.861 mmol) of pyridinium dichromate was added and mixture was stirred in room temperature for 3 h. The reaction mixture was filtrated through column with silica gel (100 cm³) using dichloromethane, dichloromethane:ethyl acetate (4:1, 3:1). The fractions containing product were pooled and evaporated to give 305 mg (97%) of product as yellow oil.

[α]_{D}³⁰=−25.9° c=0.37, CHCl₃

¹H NMR (CDCl₃): 6.07 (1H, dt, J=12.4, 7.3 Hz), 5.49 (1H, d, J=11.9 Hz), 4.33 (1H, br s), 2.52 (1H, dd, J=16.2, 7.7 Hz), 2.45-2.38 (2H, m), 2.31-2.10 (3H, m), 2.06-1.98 (1H, m), 1.96-1.81 (1H, m), 1.79-1.35 (12H, m), 1.23 (6H, s), 0.99 (3H, s), 0.75 (3H, s)

| | | | | |
|---|---|---|---|---|
| MS HRES | Calculated for: | C₂₄H₃₆F₆O₃ | [M + Na]⁺ | 509.2461 |
| | Observed: | | [M + Na]⁺ | 509.2463 |

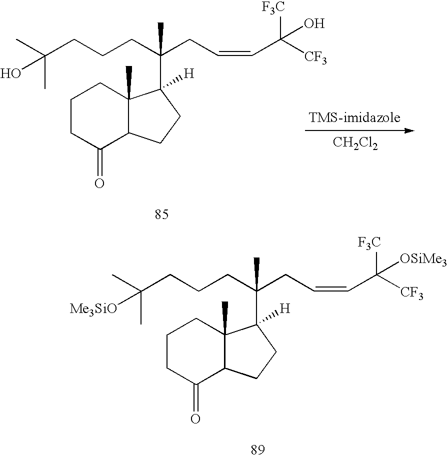

### (1R,3aR,4S,7aR)-7a-Methyl-1-[(1R,3Z)-6,6,6-trifluoro-1-methyl-1-(4-methyl-4-trimethylsilanyloxy-pentyl)-5-trifluoromethyl-5-trimethylsilanyloxy-hex-3-enyl]-octahydro-inden-4-one (89)

A 25 ml round bottom flask equipped with stir bar and Claisen adapter with rubber septum was charged with 295 mg (0.606 mmol) of (1R,3aR,4S,7aR)-7a-methyl-1-[(1R,3Z)-6,6,6-trifluoro-5-hydroxy-1-(4-hydroxy-4-methyl-pentyl)-1-methyl-5-trifluoromethyl-hex-3-enyl]-octahydro-inden-4-one and 8.0 ml of dichloromethane. A 0.7 ml (4.8 mmol) of 1-(trimethylsilyl)imidazole was added dropwise. The mixture was stirred at room temperature for 3 h. A 100 ml of water was added and the mixture was extracted three times with 50 ml of ethyl acetate, dried over Na₂SO₄ and evaporated. The oil residue was chromatographed on column (50 cm³) using hexane:ethyl acetate (10:1) as mobile phase. Fractions containing product were pooled and evaporated to give 362 mg (95%) of product as colorless oil.

¹H NMR (CDCl₃): 6.02-5.94 (1H, m), 5.42 (1H, d, J=11.0 Hz), 2.50-2.40 (2H, m), 2.35-2.14 (4H, m), 2.06-1.55 (7H, m), 1.43-1.14 (7H, m), 1.21 (6H, s), 0.96 (3H, s), 0.74 (3H, s), 0.24 (9H, s), 0.10 (9H, s)

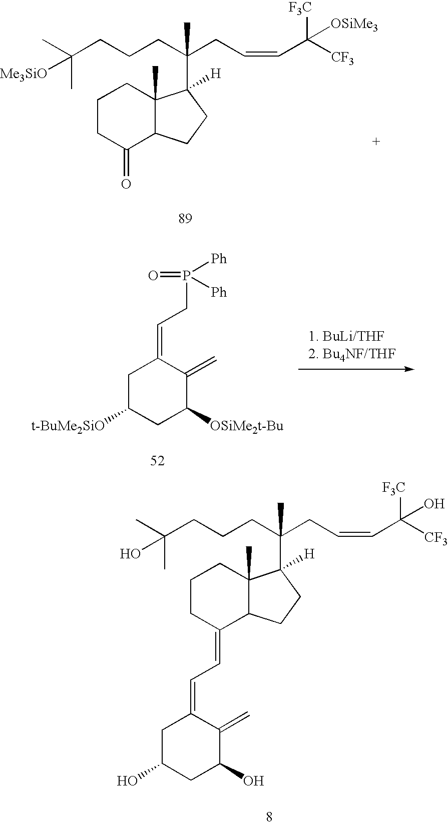

### (20)-1,25-Dihydroxy-20-[(2Z)-5,5,5-trifluoro-4-hydroxy-4-trifluoromethyl-pent-2-enyl]-cholecalciferol (8)

A 25 ml round bottom flask equipped with stir bar and Claisen adapter with rubber septum was charged with 757 mg (1.299 mmol) of (1S,5R)-1,5-bis-((tert-butyl dimethyl)silanyloxy)-3-[2-(diphenylfosphinoyl)eth-(Z)-ylidene]-2-methylene-cyclohexane and 10 ml of tetrahydrofurane. The reaction mixture was cooled to −78° C. and 0.8 ml (1.28 mmol) of 1.6M n-butyllithium in tetrahydrofurane was added dropwise. The resulting deep red solution was stirred at −78° C. for 20 min and 360 mg (0.571 mmol) of (1R,3aR,4S,7aR)-7a-methyl-1-[(1R,3Z)-6,6,6-trifluoro-1-methyl-1-(4-methyl-4-trimethylsilanyloxy-pentyl)-5-trifluoromethyl-5-trimethylsilanyloxy-hex-3-enyl]-octahydro-inden-4-one was added dropwise in 1.5 ml of tetrahydrofurane. The reaction mixture was stirred for 4 h 30 min (last 0.5 h at −30° C.) and then the bath was removed and the mixture was poured into 50 ml of ethyl acetate and 100 ml of brine. The water fraction was extracted three times with 50 ml of ethyl acetate, dried over Na₂SO₄ and evaporated. The oil residue was chromatographed on column (50 cm³, protected from light) using hexane:ethyl acetate (15:1) as mobile phase. Fractions containing product and some mono deprotected compound were pooled and evaporated to give colorless oil (430 mg) which was treated with 10 ml of 1M tetrabutylammonium fluoride in tetrahydrofurane. The reaction mixture was stirred at room temperature for 6 h 40 min. The mixture was dissolved by the addition of 150 ml of ethyl acetate and extracted six times with 50 ml of water:brine. (1:1) and 50 ml of brine, dried over Na₂SO₄ and evaporated. The oil residue was chromatographed on column (50 cm³, protected from light) using ethyl acetate as mobile phase. Fractions containing product were pooled and evaporated to give product as colorless oil. Oil was dissolved in methyl acetate and evaporated (4 times) to give 278 mg (78%, two steps) of product as white foam.

[α]_{D}³¹=+6.50° c=0.51, EtOH

UV λmax (EtOH): 212.67 nm (ε 15573), 265.17 nm (ε 17296)

¹H NMR (D-6-DMSO): 7.97 (1H, s), 6.18 (1H, d, J=11.3 Hz), 6.09 (1H, dt, J=12.1, 6.3 Hz), 5.96 (1H, d, J=11.3 Hz), 5.42 (1H, d, J=12.1 Hz), 5.22 (1H, s), 4.86 (1H, d, J=4.8 Hz), 4.75 (1H, s), 4.54 (1H, d, J=3.6 Hz), 4.20-4.36 (1H, m), 4.04 (1H, s), 4.00-3.96 (1H, m), 2.77 (1H, br d, J=11.1 Hz), 2.49-2.39 (2H, m), 2.3591H, d, J=11.9 Hz), 2.16 (1H, dd, J=13.4, 5.3 Hz), 2.00-1.86 (2H, m), 1.83-1.77 (1H, m), 1.70-1.15 (16H, m), 1.04 (3H, s), 1.04 (3H, s), 0.90 (3H, s), 0.60 (3H, s)

¹³C NMR (D6-DMSO): 149.40, 139.75, 139.21, 135.81, 122.94 (q, J=287.7 Hz), 122.36, 117.87, 117.15, 109.75, 68.72, 68.34, 65.08, 56.56, 55.98, 46.15, 44.85, 44.69, 43.11, 40.35, 38.85, 36.04, 29.43, 29.12, 28.34, 23.13, 22.79, 21.83, 21.50, 17.96, 14.55

| | | | | |
|---|---|---|---|---|
| MS HRES | Calculated for: | C₃₃H₄₈F₆O₄ | [M + Na]⁺ | 645.3349 |
| | Observed: | | [M + Na]⁺ | 645.3337 |

### Example 17

### Synthesis of (20)-1,25-Dihydroxy-20-[(2Z)-5,5,5-trifluoro-4-hydroxy-4-trifluoromethyl-pent-2-enyl]-19-nor-cholecalciferol (35)

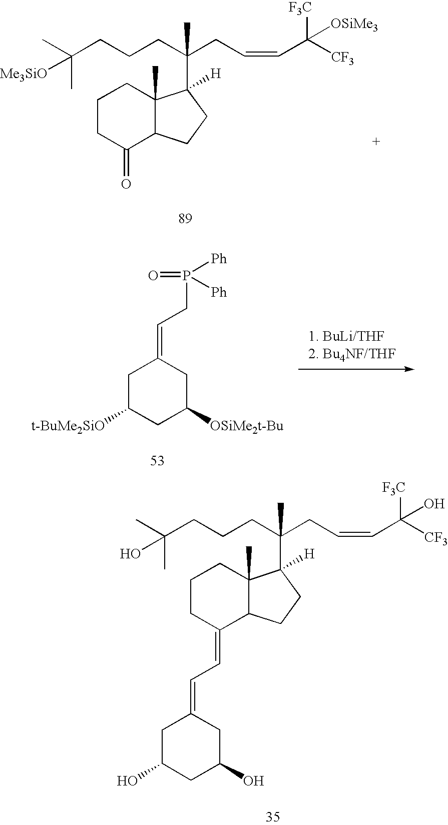

### (20)-1,25-Dihydroxy-20-[(2Z)-5,5,5-trifluoro-4-hydroxy-4-trifluoromethyl-pent-2-enyl]-19-nor-cholecalciferol (35)

A 25 ml round bottom flask equipped with stir bar and Claisen adapter with rubber septum was charged with 804 mg (1.408 mmol) of (1R,3R)-1,3-bis-((tert-butyldimethyl)silanyloxy)-5-[2-(diphenylfosphinoyl)ethylidene]-cyclohexane and 8 ml of tetrahydrofurane. The reaction mixture was cooled to −70° C. and 0.88 ml (1.41 mmol) of 1.6M n-butyllithium BuLi was added dropwise. The resulting deep red solution was stirred at −70° C. for 25 min and 441 mg (0.699 mmol) of (1R,3aR,4S,7aR)-7a-methyl-1-[(1R,3Z)-6,6,6-trifluoro-1-methyl-1-(4-methyl-4-trimethylsilanyloxy-pentyl)-5-trifluoromethyl-5-trimethylsilanyloxy-hex-3-enyl]-octahydro-inden-4-one in 1.5 ml of tetrahydrofurane. The reaction mixture was stirred for 6 h at −70° C. The mixture was poured into ethyl acetate (50 ml) and saturated solution of ammonium chloride (50 ml). The water fraction was extracted with ethyl acetate (3×50 ml), dried (Na₂SO₄) and evaporated. The oil residue was chromatographed on column (50 cm³, protected from light) using hexane:ethyl acetate (25:1) as mobile phase. Fractions containing product were pooled and evaporated to give oil (ca. 615 mg) which was treated with 15 ml of 1M tetrabutylammonium fluoride in tetrahydrofurane. The reaction mixture was stirred at room temperature for 18 h. The new portion 5 ml of 1M tetrabutylammonium fluoride in tetrahydrofurane was added and the mixture was stirred for next 48 h. The mixture was dissolved by the addition of 150 ml of ethyl acetate and extracted six times with 50 ml of water:brine (1:1) and 50 ml of brine, dried over Na₂SO₄ and evaporated. The oil residue was chromatographed on column (50 cm³, protected from light) using ethyl acetate:hexane (1:2, 1:1 and 3:1) and ethyl acetate as mobile phase. Fractions containing product were pooled and evaporated to give product as colorless oil. Oil was dissolved in methyl acetate and evaporated (2 times) to give 395 mg (92%) of product as white foam.

[α]_{D}²⁶=+42.6° c=0.50, EtOH

UV λmax (EtOH): 244 nm (ε 35888), 252 nm (ε 41722), 262 nm (ε 28261)

¹H NMR (DMSO-D6): 7.99 (1H, s), 6.14-6.08 (1H, m), 6.08 (1H, d, J=12.4 Hz), 5.78 (1H, d, J=11.3 Hz), 5.44 (1H, d, J=12.4 Hz), 4.48 (1H, d, J=4.1 Hz), 4.38 (1H, d, J=4.1 Hz), 4.05 (1H, s), 3.89-3.84 (1H, m), 3.83-3.77 (1H, m), 2.73 (1H, d, J=13.2 Hz), 2.49-2.41 (2H, m), 2.26 (1H, d, J=10.4 Hz), 2.07-1.96 (4H, m), 1.72-1.20 (18H, m), 1.05 (3H, s), 1.05 (3H, s), 0.91 (3H, s), 0.61 (3H, s)

¹³C NMR (D6-DMSO): 139.41, 139.34, 134.75, 123.07 (q, J=288.2 Hz), 120.95, 117.26, 116.46, 76.83 (sep, J=28.1 Hz), 68.77, 65.59, 65.31, 56.56, 55.98, 46.01, 44.71, 44.61, 42.22, 40.35, 39.01, 38.78, 36.96, 36.07, 29.44, 29.11, 22.97, 22.78, 21.88, 21.38, 17.94, 14.64

| | | | | |
|---|---|---|---|---|
| MS HRES | Calculated for: | C₃₂H₄₈F₆O₄ | [M + Na]⁺ | 633.3349 |
| | Observed: | | [M + Na]⁺ | 633.3357 |

### Example 18

### Synthesis of (20)-1α-Fluoro-25-hydroxy-20-[(2Z)-5,5,5-trifluoro-4-hydroxy-4-trifluoromethyl-pent-2-enyl]-cholecalciferol (38)

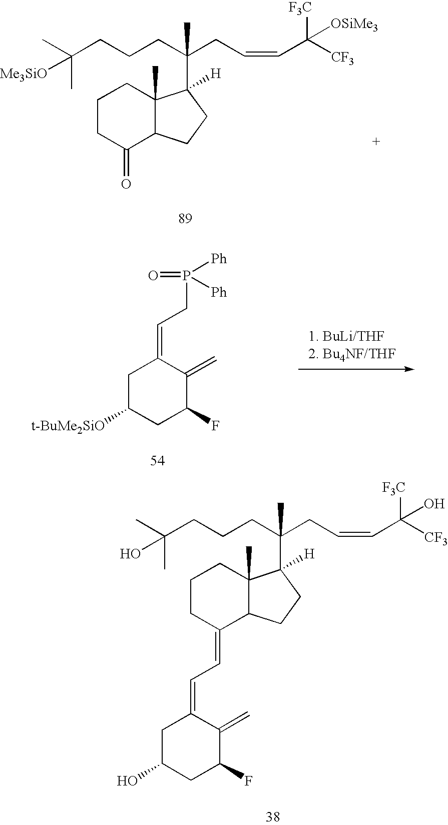

### (20)-1α-Fluoro-25-hydroxy-20-[(2Z)-5,5,5-trifluoro-4-hydroxy-4-trifluoromethyl-pent-2-enyl]-cholecalciferol (38)

A 25 ml round bottom flask equipped with stir bar and Claisen adapter with rubber septum was charged with 673 mg (1.430 mmol) of (1S,5R)-1-((tert-butyldimethyl)silanyloxy)-3-[2-(diphenylfosphinoyl)-eth-(Z)-ylidene]-5-fluoro-2-methylene-cyclohexane and 8 ml of tetrahydrofurane. The reaction mixture was cooled to −70° C. and 0.89 ml (1.42 mmol) of 1.6M n-butyllithium was added dropwise. The resulting deep red solution was stirred at −70° C. for 20 min and 320 mg (0.507 mmol) of (1R,3aR,4S,7aR)-7a-methyl-1-[(1R,3Z)-6,6,6-trifluoro-1-methyl-1-(4-methyl-4-trimethyl silanyloxy-pentyl)-5-trifluoromethyl-5-trimethylsilanyloxy-hex-3-enyl]-octahydro-inden-4-one was added dropwise in 1.5 ml of tetrahydrofurane. The reaction mixture was stirred for 4 h and then the dry ice was removed from bath and the solution was allowed to warm up to 40° C. in 2 h. The mixture was poured into ethyl acetate (50 ml) and saturated solution of ammonium chloride (50 ml). The water fraction was extracted with ethyl acetate (3×50 ml), dried (Na₂SO₄) and evaporated. The oil residue was chromatographed on column (50 cm³, protected from light) using hexane:ethyl acetate (25:1) as mobile phase. Fractions containing product were pooled and evaporated to give oil (568 mg) which was treated with 10 ml of 1M tetrabutylammonium fluoride in tetrahydrofurane. The reaction mixture was stirred at room temperature for 17 h. The mixture was dissolved by the addition of 150 ml of ethyl acetate and extracted six times with 50 ml of water:brine (1:1) and 50 ml of brine, dried over Na₂SO₄ and evaporated. The oil residue was chromatographed on two columns: 50 cm³ (protected from light) using ethyl acetate:hexane (1:1) as mobile phase and 50 cm³ (protected from light) using hexane:ethyl acetate (2:1 and 1:1) Fractions containing product were pooled and evaporated to give product as colorless oil. The product was dissolved in methyl acetate and evaporated (2 times) to give 365 mg 810%) of product as white foam.

[α]_{D}²⁶=+22.2° c=0.49, EtOH

UV λax (EtOH): 210 nm (ε 15393), 243 nm (ε 15181), 270 nm (ε 15115)

¹H NMR (DMSO-D6): 7.99 (1H, s), 6.36 (1H, d, J=11.3 Hz), 6.10 (1H, dt, J=12.2, 6.3 Hz), 5.93 (1H, d, J=11.3 Hz), 5.43 (1H, d, J=12.2 Hz), 5.39 (1H, s), 5.14 (1H, br d, J=47.5 Hz), 4.99 (1H, d, J=1.7 Hz), 4.85 (1H, d, J=4.3 Hz), 4.05 (1H, s), 3.94-3.88 (1H, m), 2.81 (1H, d, J=13.2 Hz), 2.47-2.41 (2H, m), 2.16-2.05 (2H, m), 2.01-1.96 (2H, m), 1.83-1.18 (17H, m), 1.05 (3H, s), 1.05 (3H, s), 0.90 (3H, s), 0.60 (3H, s)

¹³C NMR (DMSO-D6): 143.30 (d, J=16.7 Hz), 141.89, 139.35, 133.08, 124.18, 123.05 (q, J=288.2 Hz), 117.37, 117.24, 115.26 (d, J=9.1 Hz), 92.02 (d, J=167.6 Hz), 76.84 (sep, J=28.1 Hz), 68.76, 64.53, 56.55, 55.95, 46.25, 44.82, 44.70, 40.68 (d, J=20.5 Hz), 40.29, 38.95, 38.77, 36.06, 29.41, 29.12, 28.32, 23.03, 22.71, 21.81, 21.37, 17.93, 14.55

| | | | | |
|---|---|---|---|---|
| MS HRES | Calculated for: | C₃₃H₄₇F₇O₃ | [M + Na]⁺ | 647.3305 |
| | Observed: | | [M + Na]⁺ | 647.3297 |

### Example 19

### Synthesis of (20R)-1,25-Dihydroxy-20-[(2E)-5,5,5-trifluoro-4-hydroxy-4-trifluoromethyl-pent-2-enyl]-cholecalciferol (9)

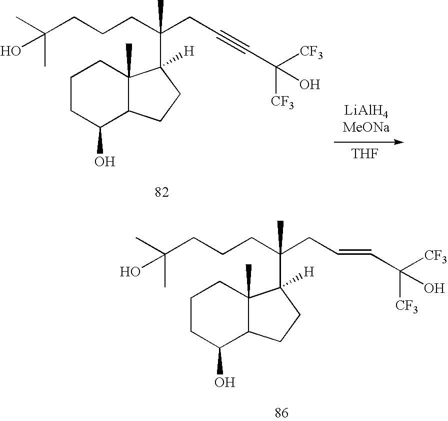

### (3E,6R)-1,1,1-Trifluoro-6-[(1R,3aR,4S,7aR)-4-hydroxy-7a-methyl-octahydro-inden-1-yl],10-dimethyl-2-trifluoromethyl-undec-3-ene-2,10-diol (86)

A 25 ml round bottom flask equipped with stir bar and condenser with nitrogen sweep was charged with 4.5 ml (4.5 mmol) of 1M lithium aluminum hydride in tetrahydrofurane and the mixture was cooled to 0° C. A 243 mg (4.50 mmol) of sodium methoxide was added slowly followed by substrate 337 mg (0.693 mmol) of (3E,6R)-1,1,1-trifluoro-6-[(1R,3aR,4S,7aR)-4-hydroxy-7a-methyl-octahydro-inden-1-yl]-6,10-dimethyl-2-trifluoromethyl-undec-3-yne-2,10-diol in 5 ml of tetrahydrofurane. The reaction mixture was stirred at 80° C. for 6 h 30 min and then was cooled to 0° C. A 1 ml of water, 1 ml of 2N NaOH and 20 ml of diethyl ether were added. The mixture was stirred at room temp for 30 min and 2.2 g of MgSO₄ was added and mixture was stirred for next 15 min. The suspension was filtrated and solvent evaporated. The oil residue was chromatographed on column (100 cm³) using dichloromethane:ethyl acetate (4:1) as mobile phase. Fractions containing product were pooled and evaporated to give 330 mg (97%) of product as colorless oil.

¹H NMR (CDCl₃): 6.28 (1H, dt, J=15.7, 7.3 Hz), 5.59 (1H, d, J=15.4 Hz), 6.12 (1H, br s), 2.12 (2H, d, J=7.7 Hz), 2.06-1.98 (1H, m), 1.85-1.74 (2H, m), 1.68-1.16 (18H, m), 1.22 (6H, s), 1.08 (3H, s), 0.98 (3H, s)

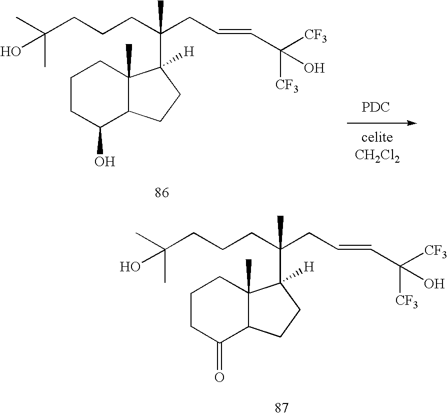

### (1R,3aR,4S,7aR)-7a-Methyl-1-[(1R,3E)-6,6,6-trifluoro-5-hydroxy-1-(4-hydroxy-4-methyl-pentyl)-1-methyl-5-trifluoromethyl-hex-3-enyl]-octahydro-inden-4-one (87)

A 25 ml round bottom flask equipped with stir bar and Claisen adapter with rubber septum was charged with 330 mg (0.675 mmol) of (3E,6Z)-1,1,1-trifluoro-6-[(1R,3aR,4S,7aR)-4-hydroxy-7a-methyloctahydro-inden-1-yl]-6,10-dimethyl-2-trifluoromethyl-undec-3-ene-2,10-diol and 10 ml of dichloromethane. A 920 mg (2.445 mmol) of pyridinium dichromate was added and mixture was stirred in room temperature for 7 h. The reaction mixture was filtrated through column with silica gel (60 cm³) using dichloromethane:ethyl acetate (4:1) as mobile phase. The fractions containing product were pooled and evaporated to give 302 mg (92%) of product as colorless oil.

[α]_{D}³⁰=−17.7° c=0.46, CHCl₃

¹H NMR (CDCl₃): 6.30 (1H, dt, J=15.6, 7.7 Hz), 5.60 (1H, d, J=15.6 Hz), 2.40 (1H, dd, J=11.1, 7.3 Hz), 2.30-2.14 (6H, m), 2.06-1.98 (1H, m), 1.96-1.81 (1H, m), 1.78-1.30 (13H, m), 1.24 (3H, s), 1.23 (3H, s), 0.98 (3H, s), 0.74 (3H, s)

¹³C NMR (CDCl₃): 212.12, 136.27, 120.28, 71.45, 62.27, 57.44, 50.69, 44.28, 42.02, 40.76, 40.17, 39.69, 39.65, 29.34, 29.23, 23.98, 22.66, 22.24, 18.67, 18.19, 15.47

| | | | | |
|---|---|---|---|---|
| MS HRES | Calculated for: | C₂₄H₃₆F₆O₃ | [M + Na]⁺ | 509.2461 |
| | Observed: | | [M + Na]⁺ | 509.2463 |

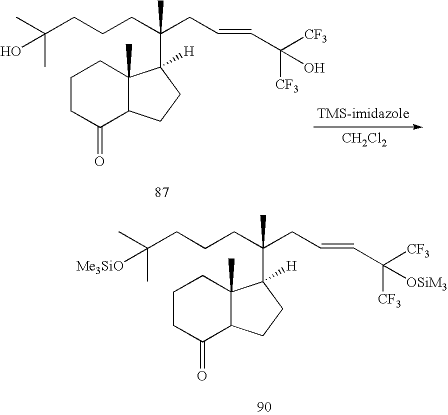

### (1R,3aR,4S,7aR)-7a-Methyl-1-[(1R,3E)-6,6,6-trifluoro-1-methyl-1-(4-methyl-4-trimethylsilanyloxy-pentyl)-5-trifluoromethyl-5-trimethylsilanyloxy-hex-3-enyl]-octahydro-inden-4-one (90)

A 25 ml round bottom flask equipped with stir bar and Claisen adapter with rubber septum was charged with 292 mg (0.600 mmol) of (1R,3aR,4S,7aR)-7a-methyl-1-[(1R,3E)-6,6,6-trifluoro-5-hydroxy-1-(4-hydroxy-4-methyl-pentyl)-1-methyl-5-trifluoromethyl-hex-3-enyl]-octahydro-inden-4-one and 8 ml of dichloromethane. A 0.7 ml (4.8 mmol) of 1-(trimethylsilyl)imidazole was added dropwise. The mixture was stirred at room temperature for 2 h. A 100 ml of water was added and the mixture was extracted three times with 50 ml of ethyl acetate, dried over Na₂SO₄ and evaporated. The oil residue was chromatographed on column (60 cm³) using hexane:ethyl acetate (10:1, 4:1) as mobile phase. Fractions containing product were pooled and evaporated to give 360 mg (95%) of product as colorless oil.

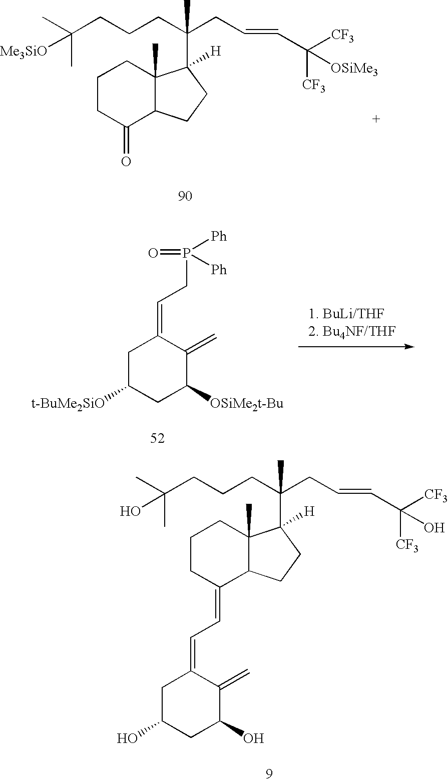

### (20R)-1,25-Dihydroxy-20-[(2E)-5,5,5-trifluoro-4-hydroxy-4-trifluoromethyl-pent-2-enyl]-cholecalciferol (9)

A 25 ml round bottom flask equipped with stir bar and Claisen adapter with rubber septum was charged with 760 mg (1.304 mmol) of (1S,5R)-1,5-bis-((tert-butyldimethyl)silanyloxy)-3-[2-(diphenylfosphinoyl)-eth-(Z)-ylidene]-2-methylene-cyclohexane and 10 ml of tetrahydrofurane. The reaction mixture was cooled to −78° C. and 0.8 ml (1.28 mmol) of 1.6M n-butyllithium in tetrahydrofurane was added dropwise. The resulting deep red solution was stirred at −78° C. for 20 min and 358 mg (0.567 mmol) of (1R,3aR,4S,7aR)-7a-methyl-1-[(1R,3E)-6,6,6-trifluoro-1-methyl-1-(4-methyl-4-trimethylsilanyloxy-pentyl)-5-trifluoromethyl-5-trimethylsilanyloxy-hex-3-enyl]-octahydro-inden-4-one was added dropwise in 1.5 ml of tetrahydrofurane. The reaction mixture was stirred for 4 h (last 0.5 h at −20° C.) and then the bath was removed and the mixture was poured into 50 ml of ethyl acetate and 100 ml of brine. The water fraction was extracted three times with 50 ml of ethyl acetate, dried over Na₂SO₄ and evaporated. The oil residue was chromatographed on column (50 cm³, protected from light) using hexane:ethyl acetate (10:1) as mobile phase. Fractions containing product and some mono deprotected compound were pooled and evaporated to give colorless oil (440 mg) which was treated with 10 ml of 1M tetrabutylammonium fluoride in tetrahydrofurane. The reaction mixture was stirred at room temperature for 21 h.

The mixture was dissolved by the addition of 150 ml of ethyl acetate and extracted six times with 50 ml of water:brine (1:1) and 50 ml of brine, dried over Na₂SO₄ and evaporated. The oil residue was chromatographed on column (50 cm³, protected from light) using ethyl acetate as mobile phase. Fractions containing product were pooled and evaporated to give 30 5 mg (86%, two steps) of product as colorless solid.

[α]_{D}³¹=+13.4° c=0.44, EtOH

UV λmax (EtOH): 212.76 nm (ε 15453), 265.03 (ε 17341)

¹H NMR (D6-DMSO): 8.04 (1H, s), 6.28 (1H, dt, J=15.5, 7.6 Hz), 6.18 (1H, d, J=11.1 Hz), 5.97 (1H, d, J=11.1 Hz), 5.61 (1H, d, J=15.5 Hz), 5.22 (1H, s), 4.75 (1H, s), 4.19-4.16 (1H, m), 3.98 (1H, br s), 2.77 (1H, d, 13.9 Hz), 2.35 (1H, d, J=11.7 Hz), 2.16 (1H, dd, J=13.6, 5.3 Hz), 2.07 (2H, d, J=7.3 Hz), 1.99-1.90 (2H, m), 1.81-1.78 (1H, m), 1.64-1.55 (6H, m), 1.48-1.17 (12H, m), 1.05 (6H, s), 0.90 (3H, s), 0.84 (1H, s), 0.61 (3H, s)

¹³C NMR (D6-DMSO): 149.34, 139.65, 136.40, 135.82, 122.60 (q, J=287.7 Hz), 122.32, 119.80, 117.90, 109.76, 68.68, 68.36, 65.04, 56.35, 56.00, 46.18, 44.85, 44.64, 43.09, 41.05, 40.42, 29.34, 29.12, 28.31, 23.08, 22.47, 21.79, 21.58, 17.91, 14.57

| | | | | |
|---|---|---|---|---|
| MS HRES | Calculated for: | C₃₃H₄₈F₆O₄ | [M + Na]⁺ | 645.3349 |
| | Observed: | | [M + Na]⁺ | 645.3355 |

### Example 20

### Synthesis of (20R)-1,25-Dihydroxy-20-[(2E)-5,5,5-trifluoro-4-hydroxy-4-trifluoromethyl-pent-2-enyl]-19-nor-cholecalciferol (36)

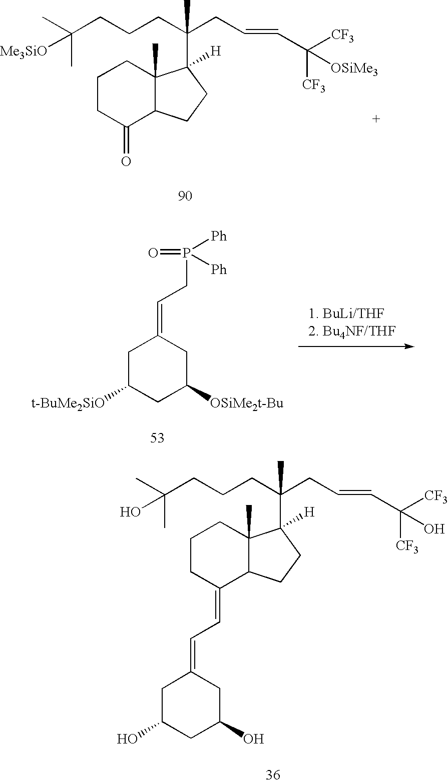

### (20R)-1,25-Dihydroxy-20-[(2E)-5,5,5-trifluoro-4-hydroxy-4-trifluoromethyl-pent-2-enyl]-19-nor-cholecalciferol (36)

A 25 ml round bottom flask equipped with stir bar and Claisen adapter with rubber septum was charged with 493 mg (0.864 mmol) of (1R,3R)-1,3-bis-((tert-butyl dimethyl)silanyloxy)-5-[2-(diphenylfosphinoyl)ethylidene]-cyclohexane and 8 ml of tetrahydrofurane. The reaction mixture was cooled to −70° C. and 0.54 ml (0.86 mmol) of 1.6M n-butyllithium BuLi was added dropwise. The resulting deep red solution was stiffed at −70° C. for 25 min and 240 mg (0.380 mmol) of (1R,3aR,4S,7aR)-7a-methyl-1-[(1R,3E)-6,6,6-trifluoro-1-methyl-1-(4-methyl-4-trimethylsilanyloxy-pentyl)-5-trifluoromethyl-5-trimethylsilanyloxy-hex-3-enyl]-octahydro-inden-4-one in 1.5 ml of tetrahydrofurane. The reaction mixture was stirred for 7 h and then the dry ice was removed from bath and the solution was allowed to warm up to −40° C. in 1 h. The mixture was poured into ethyl acetate (50 ml) and saturated solution of ammonium chloride (50 ml). The water fraction was extracted with ethyl acetate (3×50 ml), dried (Na₂SO₄) and evaporated. The oil residue was chromatographed on column (60 cm³, protected from light) using hexane:ethyl acetate (10:1) as mobile phase. Fractions containing product were pooled and evaporated to give colorless oil (ca. 380 mg) which was treated with 10 ml of 1M tetrabutylammonium fluoride in tetrahydrofurane. The reaction mixture was stirred at room temperature for 50 h. The mixture was dissolved by the addition of 150 ml of ethyl acetate and extracted six times with 50 ml of water, dried over Na₂SO₄ and evaporated. The oil residue was chromatographed on column (60 cm³, protected from light) using hexane:tetrahydrofurane (1:1, 1:2 and 1:2+10% methanol) as mobile phase. Fractions containing product were pooled and evaporated to give product 181 mg (78%) as colorless solid.

[α]_{D}³⁰=+52.8 c=0.50, EtOH

UV λmax (EtOH): 241 nm (ε 26823)

¹H NMR (DMSO-D6): 8.05 (1H, s), 6.29 (1H, dt, J=15.3, 7.7 Hz), 6.07 (1H, d, J=11.1 Hz), 5.78 (1H, d, J=11.1 Hz), 5.63 (1H, d, J=15.3 Hz), 4.48 (1H, s), 4.38 (1H, s), 4.06 (1H, s), 3.87 (1H, s), 3.80 (1H, s), 2.74 (1H, d, J=14.5 Hz), 2.43 (1H, dd, J=13.0, 3.4 Hz), 2.28-2.25 (1H, m), 2.10-1.91 (6H, m), 1.62-1.27 (17H, m), 1.06 (3H, s), 1.06 (3H, s), 0.91 (3H, s), 0.61 (3H, s)

¹³C NMR (D6-DMSO): 139.25, 136.60, 134.79, 122.73 (q, J=286.8 Hz), 120.93, 119.96, 116.50, 75.55 (sep, J=28.8 Hz), 68.74, 65.57, 65.29, 56.38, 56.00, 46.05, 44.67, 44.60, 42.22, 41.07, 40.43, 36.95, 29.35, 29.12, 28.14, 22.92, 22.47, 21.83, 21.47, 17.90, 14.66

| | | | | |
|---|---|---|---|---|
| MS HRES | Calculated for: | C₃₂H₄₈F₆O₄ | [M + Na]⁺ | 633.3349 |
| | Observed: | | [M + Na]⁺ | 633.3350 |

### Example 21

### Synthesis of (20R)-1α-Fluoro-25-hydroxy-20-[(2E)-5,5,5-trifluoro-4-hydroxy-4-trifluoromethyl-pent-2-enyl]-cholecalciferol (39)

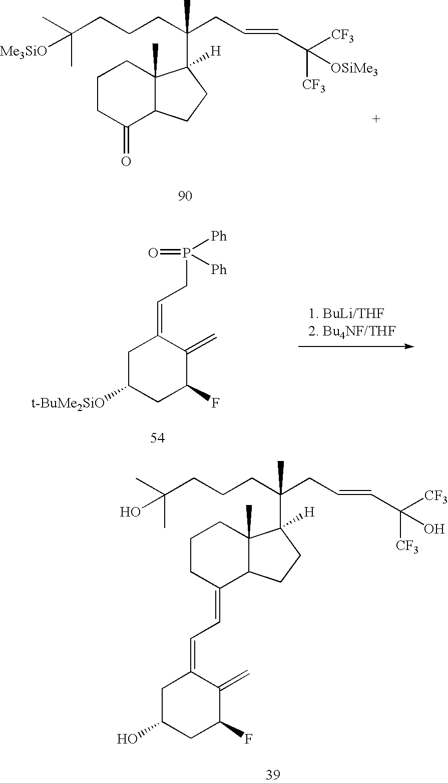

### (20R)-1α-Fluoro-25-hydroxy-20-[(2E)-5,5,5-trifluoro-4-hydroxy-4-trifluoromethyl-pent-2-enyl]-cholecalciferol (39)

A 25 ml round bottom flask equipped with stir bar and Claisen adapter with rubber septum was charged with 439 mg (0.933 mmol) of (1S,5R)-1-((tert-butyldimethyl)silanyloxy)-3-[2-(diphenylfosphinoyl)-eth-(Z)-ylidene]-5-fluoro-2-methylene-cyclohexane and 8 ml of tetrahydrofurane. The reaction mixture was cooled to −70° C. and 0.58 ml (0.93 mmol) of 1.6M n-butyllithium was added dropwise. The resulting deep red solution was stirred at −70° C. for 25 min and 238 mg (0.377 mmol) of (1R,3aR,4S,7aR)-7a-methyl-1-[(1R,3E)-6,6,6-trifluoro-1-methyl-1-(4-methyl-4-trimethyl silanyloxy-pentyl)-5-trifluoromethyl-5-trimethylsilanyloxy-hex-3-enyl]-octahydro-inden-4-one was added dropwise in 1.5 ml of tetrahydrofurane. The reaction mixture was stirred for 6 h and then the dry ice was removed from bath and the solution was allowed to warm up to 40° C. in 1 h. The mixture was poured into ethyl acetate (50 ml) and saturated solution of ammonium chloride (50 ml). The water fraction was extracted with ethyl acetate (3×50 ml), dried (Na₂SO₄) and evaporated. The oil residue was chromatographed on column (50 cm³, protected from light) using hexane:ethyl acetate (10:1) as mobile phase. Fractions containing product were pooled and evaporated to give colorless oil which was treated with 8 ml of 1M tetrabutylammonium fluoride in tetrahydrofurane. The reaction mixture was stirred at room temperature for 15 h. The mixture was dissolved by the addition of 150 ml of ethyl acetate and extracted six times with 50 ml of water, dried over Na₂SO₄ and evaporated. The oil residue was chromatographed on column (50 cm³, protected from light) using ethyl acetate:hexane (1:2 and 1:1) as mobile phase. Fractions containing product were pooled and evaporated to give product as colorless oil. The product was dissolved in methyl acetate and evaporated (2 times) to give 195 mg (83%) of product as white foam.

[α]_{D}²⁶=+29.3 c=0.43, EtOH

UV λmax (EtOH): 243 nm (ε 11639), 273 nm (ε 10871)

¹H NMR (DMSO-D6): 8.05 (1H, s), 6.37 (1H, d, J=11.3 Hz), 6.28 (1H, dt, J=15.3, 7.6 Hz), 5.93 (1H, d, J=11.3 Hz), 5.62 (1H, d, J=15.6 Hz), 5.39 (1H, s), 5.14 (1H, br d, J=47.7 Hz), 4.99 (1H, d, J=1.5 Hz), 4.87 (1H, br s), 4.06 (1H, br s), 3.93-3.88 (1H, m), 2.81 (1H, d, J=11.9 Hz), 2.16-2.06 (4H, m), 1.99-1.91 (2H, m), 1.82-1.26 (17H, m), 1.06 (3H, s), 1.06 (3H, s), 0.90 (3H, s), 0.60 (3H, s)

¹³C NMR (D6-DMSO): 143.26 (d, J=17.5 Hz), 141.80, 136.57, 133.12, 124.17, 122.73 (q, J=285.2 Hz), 119.96, 117.42, 115.37 (d, J=9.9 Hz), 92.06 (d, J=166.9 Hz), 75.54 (sep, J=28.8 Hz), 68.74, 64.55 (d, J=4.5 Hz), 56.38, 55.99, 46.28, 44.84, 44.67, 41.07, 40.69 (d, J=20.5 Hz), 40.39, 29.34, 29.14, 28.31, 22.99, 22.42, 21.76, 21.47, 17.90, 14.58

| | | | | |
|---|---|---|---|---|
| MS HRES | Calculated for: | C₃₃H₄₇F₇O₃ | [M + Na]⁺ | 647.3305 |
| | Observed: | | [M + Na]⁺ | 647.3313 |

### Example 22

### Synthesis of 1,25-Dihydroxy-20S-20-(4-hydroxy-5,5,5-trideutero-4-trideuteromethyl-pentyl)-23-yne-26,27-hexafluorocholecalciferol (22)

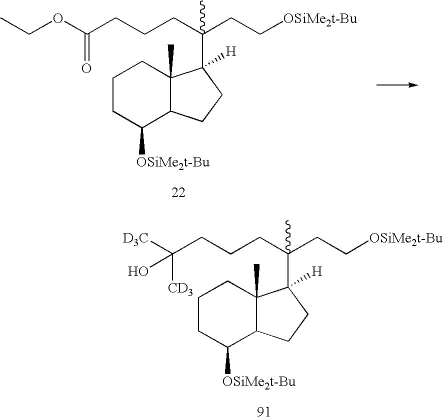

### 8-(tert-Butyl-dimethyl-silanyloxy)-6-[(1R,3aR,4S,7aR)-4-(tert-butyl-dimethyl-silanyloxy)-7a-methyl-octahydro-inden-1-yl]-1,1,1-trideutero-6-methyl-2-trideuteromethyl-octan-2-ol (91)

A 250 ml round bottom flask equipped with stir bar, Claisen adapter with rubber septum was charged with 7-(tert-butyl-dimethyl-silanyloxy)-5-[(1R,3aR,4S,7aR)-4-(tert-butyl-dimethyl-silanyloxy)-7a-methyl-octahydro-inden-1-yl]-5-methyl-heptanoic acid ethyl ester (8.770 g, 32.987 mmol) and ether (150 ml). The solution was cooled in ace-water bath and a 1.0M solution of methyl-d₃-magnesium iodide in diethyl ether (100.0 ml, 100.0 mmol) was added dropwise. After completion of the addition the mixture was stirred at room temperature for 3 h then cooled again in an ice bath. A saturated solution of ammonium chloride (10 ml) was added dropwise. The resulting precipitate was dissolved by the addition of saturated solution of ammonium chloride (100 ml). The aqueous layer was extracted with diethyl ether (3×100 ml). The combined organic layers were dried (Na₂SO₄) and evaporated. The oil residue was used to next reaction.

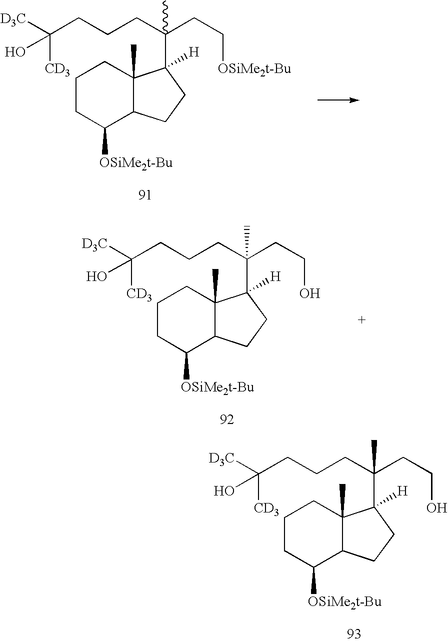

### (3S)-3-[(1R,3aR,4S,7aR)-4-(tert-Butyl-dimethyl-silanyloxy)-7a-methyl-octahydro-inden-1-yl]-8,8,8-trideutero-3-methyl-7-trideuteromethyl-octane-1,7-diol (92) and (3R)-3-[(1R,3aR,4S,7aR)-4-(tert-Butyl-dimethyl-silanyloxy)-7a-methyl-octahydro-inden-1-yl]-8,8,8-trideutero-3-methyl-7-trideuteromethyl-octane-1,7-diol (93)

A 250 ml round bottom flask equipped with stir bar and Claisen adapter with rubber septum was charged with 8-(tert-butyl-dimethyl-silanyloxy)-6-[(1R,3aR,4S,7aR)-4-(tert-butyl-dimethyl-silanyloxy)-7a-methyl-octahydro-inden-1-yl]-1,1,1-trideutero-6-methyl-2-trideuteromethyl-octan-2-ol (ca. 32.9 mmol), tetrahydrofuran (60 ml) and tetrabutylammonium fluoride (45.0 ml, 1M/tetrahydrofuran). The reaction mixture was stirred at room temperature for 2.5 h. The mixture was dissolved by the addition of ethyl acetate (150 ml) and washed six times with water:brine (1:1, 100 ml) and brine (50 ml), dried (Na₂SO₄) and evaporated. The oil residue was chromatographed 10 times on columns (VersaPak Cartridge, 80×10 mm and 40×10 mm, hexane/ethyl acetate—1:1) to give products (12.72 g, 87%):

### (3S)-3-[(1R,3aR,4S,7aR)-4-(tert-Butyl-dimethyl-silanyloxy)-7a-methyl-octahydro-inden-1-yl]-8,8,8-trideutero-3-methyl-7-trideuteromethyl-octane-1,7-diol (6.69 g, low polar epimer)

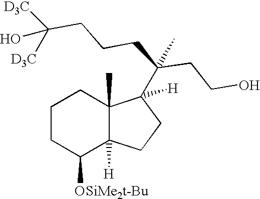

[α]_{D}³¹=+16.0 (c=0.60, EtOH)

¹H NMR (CDCl₃): 3.99 (1H, br s), 3.69-3.63 (2H, m), 2.02 (1H, br d, J=12.2 Hz), 1.82-1.48 (7H, m), 1.40-1.09 (14H, m), 1.06 (3H, s), 0.95 (3H, s), 0.88 (9H, s), 0.00 (3H, s), −0.01 (3H, s)

| | | | | |
|---|---|---|---|---|
| MS HRES | Calculated for: | C₂₆H₄₆D₆O₃Si | [M + Na]⁺ | 469.3954 |
| | Observed: | | [M + Na]⁺ | 469.3956 |

### (3R)-3-[(1R,3aR,4S,7aR-4-(tert-Butyl-dimethyl-silanyloxy)-7a-methyl-octahydro-inden-1-yl]-8,8,8-trideutero-3-methyl-7-trideuteromethyl-octane-1,7-diol (6.03 g, more polar epimer)

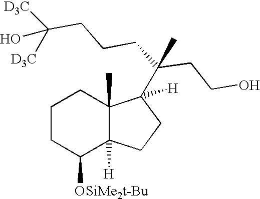

[α]_{D}³¹=+20.0 (c=0.54, EtOH)

¹H NMR (CDCl₃): 3.99-3.97 (1H, m), 3.66-3.62 (2H, m), 1.98 (11H, br d, J=12.8 Hz), 1.84-1.73 (1H, n), 1.67-1.51 (6H, m), 1.42-1.16 (14H, m), 1.05 (3H, s), 0.95 (3H, s), 0.88 (9H, s), 0.00 (3H, s), −0.01 (3H, s)

| | | | | |
|---|---|---|---|---|
| MS HRES | Calculated for: | C₂₆H₄₆D₆O₃Si | [M + Na]⁺ | 469.3954 |
| | Observed: | | [M + Na]⁺ | 469.3957 |

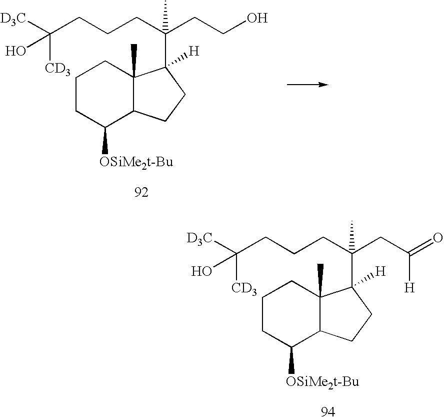

### (3S)-3-[(1R,3aR,4S,7aR)-4-(tert-Butyl-dimethyl-silanyloxy)-7a-methyl-octahydro-inden-1-yl]-8,8,8-trideutero-7-hydroxy-3-methyl-7-trideuteromethyl-octanal (94)

A 250 ml round bottom flask equipped with stir bar and Claisen adapter with rubber septum was charged with pyridinium chlorochromate (2.90 g, 13.45 mmol), celite (4.0 g) and dichloromethane (60 ml). The (3S)-3-[(1R,3aR,4S,7aR)-4-(tert-butyl-dimethyl-silanyloxy)-7a-methyl-octahydro-inden-1-yl]-8,8,8-trideutero-3-methyl-7-trideuteromethyl-octane-1,7-diol (4.00 g, 8.95 mmol) in dichloromethane (5 ml) was added dropwise and mixture was stirred in room temperature for 2 h 40 min. The reaction mixture was filtrated through column with silica gel (200 cm³) and celite (2 cm) using dichloromethane, dichloromethane:ethyl acetate 4:1. The fractions containing product were pooled and evaporated to give oil (3.61 g, 91%). Product was used to the next reaction without purification.

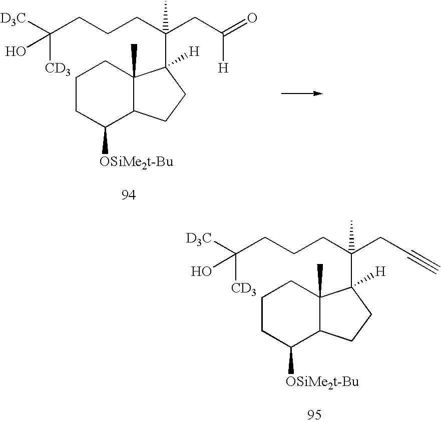

### (6S)-6-[(1R,3aR,4S,7aR)-4-(tert-Butyl-dimethyl-silanyloxy)-7a-methyl-octahydro-inden-1-yl]-1,1,1-trideutero-6-methyl-2-trideuteromethyl-non-8-yn-2-ol (95)

A 100 ml round bottom flask equipped with stir bar and Claisen adapter with rubber septum was charged with (3S)-3-[(1R,3aR,4S,7aR)-4-(tert-butyl-dimethyl-silanyloxy)-7a-methyl-octahydro-inden-1-yl]-8,8,8-trideutero-7-hydroxy-3-methyl-7-trideuteromethyl-octanal (3.61 g, 8.116 mmol) and methanol (65 ml). 1-diazo-2-oxo-propyl)-phosphonic acid dimethyl ester (3.00 g, 15.62 mmol) in methanol (3 ml) was added and the resulting mixture was cooled in an ice bath. Potassium carbonate (3.00 g, 21.74 mmol) was added and the reaction mixture was stirred in the ice bath for 30 min and then at room temperature for 4 h. Water (100 ml) was added and the mixture was extracted with ethyl acetate (4×80 ml), dried (Na₂SO₄) and evaporated.

The oil residue was chromatographed on column (300 cm³) using hexane:ethyl acetate—9:1 and 8:1 as mobile phase. Fractions containing product were pooled and evaporated to give product as colorless oil (3.131 g, 87.5%).

[α]_{D}²⁶=+17.6 (c=0.83, EtOH)

¹H NMR (CDCl₃): 3.98 (1H, br d, J=2.13 Hz), 2.28 (1H, AB, J=17.3 Hz), 2.26 (1H, AB, J=17.3 Hz), 1.96-1.91 (2H, m), 1.84-1.73 (1H, m), 1.67-1.48 (5H, m), 1.43-1.24 (12H, m), 1.04 (3H, s), 1.00 (3H, s), 0.88 (9H, s), 0.00 (3H, s), −0.01 (3H, s)

¹³C NMR (CDCl₃): 83.06, 76.41 (sep, J=29.6 Hz), 69.84, 69.55, 56.54, 52.87, 44.66, 43.68, 41.27, 40.16, 39.28, 34.32, 28.76, 25.87, 22.76, 22.69, 22.17, 18.10, 17.76, 16.78, −4.69, −5.05

| | | | | |
|---|---|---|---|---|
| MS HRES | Calculated for: | C₂₇H₄₄D₆O₂Si | [M + Na]⁺ | 463.3849 |
| | Observed: | | [M + Na]⁺ | 463.3848 |

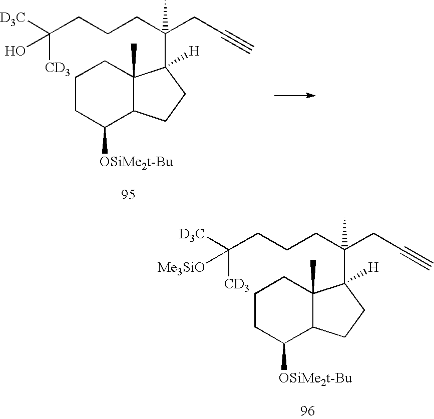

### (1R,3aR,4S,7aR)-4-(tert-Butyl-dimethyl-silanyloxy)-7a-methyl-1-[(1S)-6,6,6-trideutero-1-methyl-1-(prop-2-ynyl)-5-trideuteromethyl-5-trimethylsilanyloxy-hexyl]-octahydro-4-indene (96)

A 100 ml round bottom flask equipped with stir bar and Claisen adapter with rubber septum was charged with (6S)-6-[(1R,3aR,4S,7aR)-4-(tert-butyl-dimethyl-silanyloxy)-7a-methyl-octahydro-inden-1-yl]-1,1,1-trideutero-6-methyl-2-trideuteromethyl-non-8-yn-2-ol (3.100 g, 7.033 mmol) and dichloromethane (30 ml). 1-(trimethylsilyl)imidazole (3.0 ml, 20.45 mmol) was added dropwise. The mixture was stirred at room temperature for 1 h 45 min. Water (100 ml) was added and the mixture was extracted with ethyl acetate (3×100 ml), dried (Na₂SO₄) and evaporated. The oil residue was chromatographed on column (125 cm³) using hexane:ethyl acetate—10:1 as mobile phase. Fractions containing product were pooled and evaporated to give product as colorless oil (3.36 g, 93%).

[α]_{D}²⁶=+15.4 (c=0.52, CHCl₃)

¹H NMR (CDCl₃): 3.99 (1H, br s), 2.27 (2H, br s), 2.00-1.93 (2H, m), 1.84-1.73 (1H, m), 1.65 (1H, d, J=14.3 Hz), 1.59-1.49 (3H, m), 1.42-1.20 (12H, m), 1.05 (3H, s), 1.00 (3H, s), 0.88 (9H, s), 0.10 (9H, s), 0.00 (3H, s), −0.01 (3H, s)

¹³C NMR (CDCl₃): 83.18, 76.66 (sep, J=28.8 Hz), 69.74, 69.58, 56.62, 52.91, 45.38, 43.67, 41.27, 40.07, 39.28, 34.34, 28.77, 25.88, 22.76, 22.16, 18.13, 18.11, 17.77, 16.76, 2.74, −4.69, −5.05

| | | | | |
|---|---|---|---|---|
| MS HRES | Calculated for: | C₃₀H₅₂D₆O₂Si₂ | [M + Na]⁺ | 535.4244 |
| | Observed: | | [M + Na]⁺ | 535.4246 |

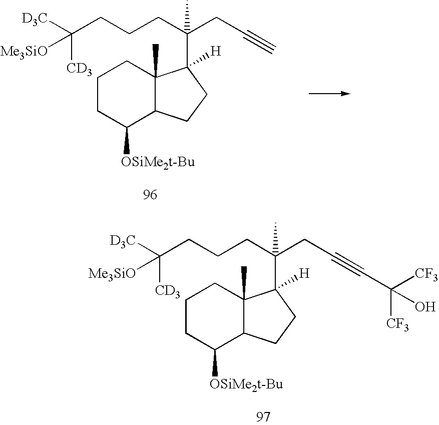

### (6S)-6-[(1R,3aR,4S,7aR)-4-(tert-Butyl-dimethyl-silanyloxy)-7a-methyl-octahydro-inden-1-yl]-6-methyl-11,11,11-trideutero-10-trideuteromethyl-1,1,1-trifluoro-2-trifluoromethyl-10-trimethylsilanyloxy-undec-3-yn-2-ol (97)

A two neck 100 ml round bottom flask equipped with stir bar, Claisen adapter with rubber septum and funnel (with cooling bath) was charged with (1R,3aR,4S,7aR)-4-(tert-butyl-dimethyl-silanyloxy)-7a-methyl-1-[(1S)-6,6,6-trideutero-1-methyl-1-(prop-2-ynyl)-5-trideuteromethyl-5-trimethylsilanyloxy-hexyl]-octahydro-indene (3.330 g, 6.491 mmol) and tetrahydrofuran (40 ml). The funnel was connected to container with hexafluoroacetone and cooled (acetone, dry ice). The reaction mixture was cooled to −70° C. and n-butyllithium (6.10 ml, 9.76 mmol) was added dropwise. After 30 min hexafluoroacetone was added (the container's valve was opened three times). The reaction was steered at −70° C. for 2 h then saturated solution of ammonium chloride (5 ml) was added. The mixture was dissolved by the addition of saturated solution of ammonium chloride (100 ml) and extracted with ethyl acetate (3×60 ml), dried (Na₂SO₄) and evaporated. The residue was chromatographed twice on columns (300 cm³, hexane:ethyl acetate—25:1 and 20:1) to give the mixture of product and polimer (from hexafluoroacetone) (4.33 g). Product was used to the next reaction without purification.

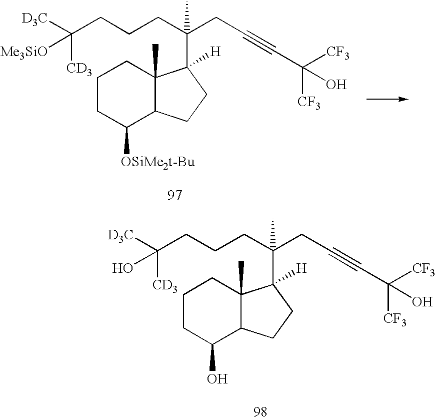

### (6S)-6-[(1R,3aR,4S,7aR)-4-hydroxy-7a-methyl-octahydro-inden-1-yl]-6-methyl-11,11,11-trideutero-10-trideuteromethyl-1,1,1-trifluoro-2-trifluoromethyl-undec-3-yne-2,10-diol (98)

A 25 ml round bottom flask equipped with stir bar and Claisen adapter with rubber septum was charged with (6S)-6-[(1R,3aR,4S,7aR)-4-(tert-butyl-dimethyl-silanyloxy)-7a-methyl-octahydro-inden-1-yl]-6-methyl-11,11,11-trideutero-10-trideuteromethyl-1,1,1-trifluoro-2-trifluoromethyl-10-trimethylsilanyloxy-undec-3-yn-2-ol (ca 3.3 mmol) and tetrabutylammonium fluoride (25 ml, 1M/tetrahydrofuran) and reaction was stirred at 70° C. for 113 h. The mixture was dissolved by the addition of ethyl acetate (150 ml) and extracted six times with water-brine (1:1, 50 ml) and dried (Na₂SO₄) and evaporated. Product was crystallized from hexane (1.996 g, 62%).

[α]_{D}³¹=−6.3 (c=0.46, EtOH)

¹H NMR (DMSO-D6): 8.92 (1H, s), 4.21 (1H, d, J=3.0 Hz), 4.04 (1H, s), 3.87 (1H, s), 2.37 (2H, s), 1.89 (1H, d, J=11.5 Hz), 1.76-1.48 (6H, m), 1.33-1.11 (11H, m), 1.02 (3H, s), 0.96 (3H, m)

¹³C NMR (DMSO-D6): 121.47 (q, J=286.8 Hz), 89.70, 70.71, 70.40 (sep, J=31.9 Hz), 68.41, 66.86, 56.24, 52.37, 44.45, 42.96, 40.44, 39.38, 33.70, 28.14, 22.43, 22.01, 21.68, 17.73, 17.46, 16.32

| | | | | |
|---|---|---|---|---|
| MS HRES | Calculated for: | C₂₄H₃₀D₆F₆O₃ | [M + Na]⁺ | 515.2837 |
| | Observed: | | [M + Na]⁺ | 515.2838 |

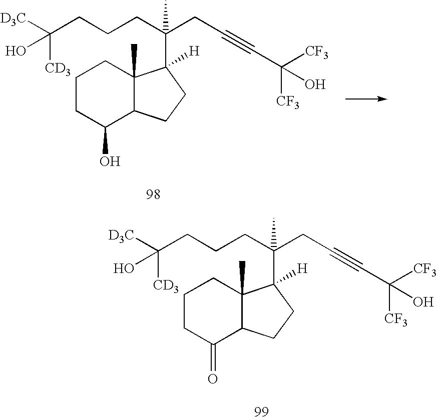

### (1R,3aR,7aR)-7a-Methyl-1-[(1S)-6,6,6-trifluoro-5-hydroxy-1-methyl-1-(5,5,5-trideutero-4-hydroxy-4-trideuteromethyl-pentyl)-5-trifluoromethyl-hex-3-ynyl]-octahydro-inden-4-one (99)

A 25 ml round bottom flask equipped with stir bar and Claisen adapter with rubber septum was charged with pyridinium dirochromate (1.51 g, 4.01 mmol) and dichloromethane (20 ml). The (6S)-6-[(1R,3aR,4S,7aR)-4-hydroxy-7a-methyl-octahydro-inden-1-yl]-6-methyl-11,11,11-trideutero-10-trideuteromethyl-1,1,1-trifluoro-2-trifluoromethyl-undec-3-yne-2,10-diol (712 mg, 1.445 mmol) in dichloromethane (5 ml) was added dropwise and mixture was stirred in room temperature for 2 h 45 min. The reaction mixture was filtrated through column with silica gel (50 cm³) using dichloromethane, dichloromethane:ethyl acetate 4:1. The fractions containing product were pooled and evaporated to give oil. The product was used to the next reaction without purification.

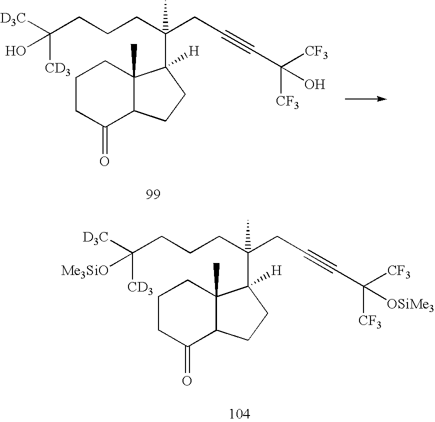

### (1R,3aR,7aR)-7a-Methyl-1-[(1S)-6,6,6-trifluoro-1-methyl-1-(5,5,5-trideutero-4-trideuteromethyl-4-trimethylsilanyloxy-pentyl)-5-trifluoromethyl-5-trimethylsilanyloxy-hex-3-ynyl]-octahydro-inden-4-one (104)

A 25 ml round bottom flask equipped with stir bar and Claisen adapter with rubber septum was charged with (1R,3aR,7aR)-7a-methyl-1-[(1S)-6,6,6-trifluoro-5-hydroxy-1-methyl-1-(5,5,5-trideutero-4-hydroxy-4-trideuteromethyl-pentyl)-5-trifluoromethyl-hex-3-ynyl]-octahydro-inden-4-one (ca. 1.445 mmol) and dichloromethane (10 ml). 1-(trimethylsilyl)imidazole (2.00 ml, 13.63 mmol) was added dropwise. The mixture was stirred at room temperature for 2 h. Ethyl acetate (150 ml) was added and the mixture was washed with water (3×50 ml), dried (Na₂SO₄) and evaporated. The oil residue was chromatographed on column (50 cm³) using hexane:ethyl acetate—5:1 as mobile phase. The product is unstable on the silica gel (the monoprotected compound was obtained (246 mg)). Fractions containing product were pooled and evaporated to give product as colorless oil (585 mg, 64%).

¹H NMR (CDCl₃): 2.44-2.37 (3H, m), 2.32-2.16 (2H, m), 2.11-1.99 (2H, m), 1.95-1.84 (2H, m), 1.81-1.52 (5H, m), 1.38-1.20 (6H, m), 1.03 (3H, s), 0.74 (3H, s), 0.28 (9H, s), 0.10 (9H, s)

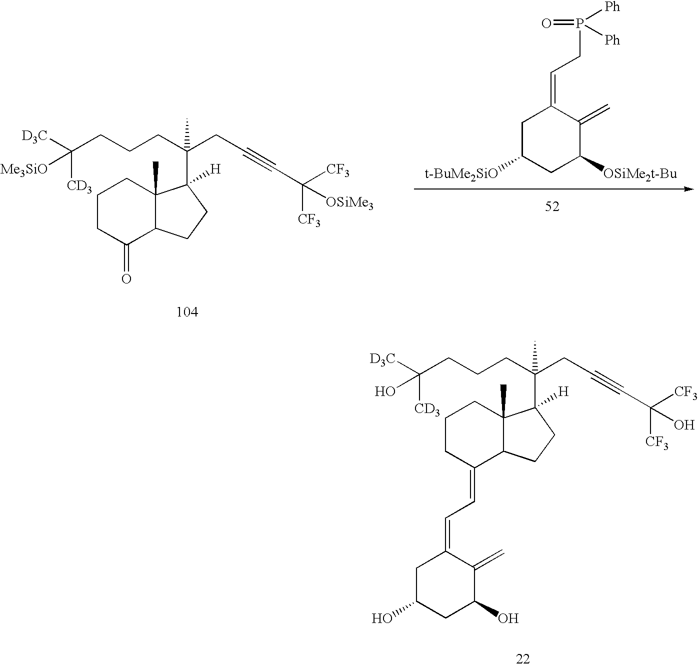

### 1,25-Dihydroxy-20S-20-(4-hydroxy-5,5,5-trideutero-4-trideuteromethyl-pentyl)-23-yne-26,27-hexafluorocholecalciferol (22)

A 25 ml round bottom flask equipped with stir bar and Claisen adapter with rubber septum was charged with (1S,5R)-1,5-bis-((tert-butyldimethyl)silanyloxy)-3-[2-(diphenylfosphinoyl)-eth-(Z)-ylidene]-2-methylene-cyclohexane (532 mg, 0.913 mmol) and tetrahydrofuran (8 ml). The reaction mixture was cooled to −78° C. and n-butyllithium (0.57 ml, 0.912 mmol)) was added dropwise. The resulting deep red solution was stirred at −70° C. for 20 min and (1R,3aR,7aR)-7a-methyl-1-[(1S)-6,6,6-trifluoro-1-methyl-1-(5,5,5-trideutero-4-trideuteromethyl-4-trimethylsilanyloxy-pentyl)-5-trifluoromethyl-5-trimethylsilanyloxy-hex-3-ynyl]-octahydro-inden-4-one (281 mg, 0.443 mmol) was added dropwise in tetrahydrofuran (1.5 ml). The reaction mixture was stirred for 5 h (in last hour the temperature was increased from −70 do −55° C.). The bath was removed and the mixture was poured into ethyl acetate (50 ml) and saturated solution of ammonium chloride (50 ml). The water fraction was extracted with ethyl acetate (3×50 ml), dried (Na₂SO₄) and evaporated. The oil residue was chromatographed on column (50 cm³, protected from light) using hexane:ethyl acetate—10:1 as mobile phase. Fractions containing product were pooled and evaporated to give colorless oil. The oil residue was used to next reaction. A 25 ml round bottom flask equipped with stir bar and Claisen adapter with rubber septum was charged with substrate and tetrabutylammonium fluoride (15 ml, 1M/tetrahydrofuran). The mixture was stirred for next 25 h. The mixture was dissolved by the addition of ethyl acetate (150 ml) and washed 6 times with water (50 ml) and brine (50 ml), dried (Na₂SO₄) and evaporated. The oil residue was chromatographed on column (50 cm³, protected from light) using ethyl acetate as mobile phase. Fractions containing product were pooled and evaporated to give product as colorless oil. There was an impurity (Bu₃N) in the product (¹H, ¹³C NMR). Material was chromatographed on column (70 cm³, protected from light) using hexane:ethyl acetate 1:1 and ethyl acetate as mobile phase. Oil was dissolved in methyl acetate and evaporated (4 times) to give product as white foam (191 mg, 69%).

[α]_{D}²⁵+3.6 (c=0.44, EtOH)

UV λmax (EtOH): 213 mm (ε 15402), 264 nm (ε 17663)

¹H NMR (DMSO-D6): 8.95 (1H, br s), 6.18 (1H, d, J=11.1 Hz), 5.97 (1H, d, J=11.1 Hz), 5.23 (1H, d, J=1.1 Hz), 4.88 (1H, d, J=3.4 Hz), 4.75 (1H, d, J=1.7 Hz), 4.56 (1H, s), 4.19 (1H, br s), 4.06 (1H, br s), 3.99 (1H, br s), 2.78 (1H, d, J=12.2 Hz), 2.45-2.29 (2H, m), 2.17 (1H, dd, J=13.2, 5.4 Hz), 1.96-1.91 (2H, m), 1.84-1.73 (2H, m), 1.65-1.18 (17H, m), 0.96 (3H, s), 0.61 (3H, s)

¹³C NMR (DMSO-D6): 149.40, 139.51, 135.95, 122.33, 121.49 (q, J=286.0 Hz), 118.02, 109.77, 89.59, 70.84, 70.43 (sep, J=31.9 Hz), 68.42, 68.37, 65.09, 56.36, 55.94, 45.97, 44.87, 44.43, 43.12, 39.98, 39.85, 39.43, 28.35, 28.27, 23.11, 22.51, 22.02, 21.42, 17.77, 14.44

| | | | | |
|---|---|---|---|---|
| MS HRES | Calculated for: | C₃₃H₄₀D₆F₆O₄ | [M + Na]⁺ | 649.3569 |
| | Observed: | | [M + Na]⁺ | 649.3572 |

### Example 23

### Synthesis of 1,25-Dihydroxy-20S-20-(4-hydroxy-5,5,5-trideutero-4-trideuteromethyl-pentyl)-23-yne-26,27-hexafluoro-19-nor-cholecalciferol (23)

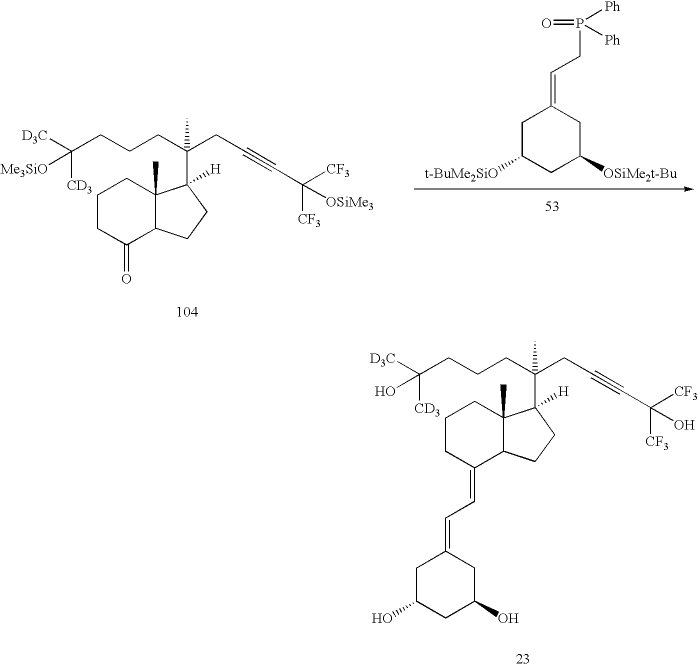

### 1,25-Dihydroxy-20S-20-(4-hydroxy-5,5,5-trideutero-4-trideuteromethyl-pentyl)-23-yne-26,27-hexafluoro-19-nor-cholecalciferol (23)

A 25 ml round bottom flask equipped with stir bar and Claisen adapter with rubber septum was charged with (1R,3R)-1,3-bis-((tert-butyldimethyl)silanyloxy)-5-[2-(diphenylfosphinoyl)ethylidene]-cyclohexane (562 mg, 0.984 mmol) and tetrahydrofuran (8 ml). The reaction mixture was cooled to −70° C. and n-butyllithium (0.61 ml, 0.98 mmol)) was added dropwise. The resulting deep red solution was stirred at −70° C. for 20 min and (1R,3aR,7aR)-7a-methyl-1-[(1S)-6,6,6-trifluoro-1-methyl-1-(5,5,5-trideutero-4-trideuteromethyl-4-trimethylsilanyloxy-pentyl)-5-trifluoromethyl-5-trimethylsilanyloxy-hex-3-ynyl]-octahydro-inden-4-one (296 mg, 0.466 mmol) was added dropwise in tetrahydrofuran (1.5 ml). The reaction mixture was stirred for 4 h 40 min (in last hour the temperature was increased from −70 do −55° C.). The bath was removed and the mixture was poured into ethyl acetate (50 ml) and saturated solution of ammonium chloride (50 ml). The water fraction was extracted with ethyl acetate (3×50 ml), dried (Na₂SO₄) and evaporated. The oil residue was chromatographed on column (50 cm³, protected from light) using hexane:ethyl acetate—10:1 as mobile phase. Fractions containing product and some mono deprotected compound were pooled and evaporated to give colorless oil (380 mg). A 25 ml round bottom flask equipped with stir bar and Claisen adapter with rubber septum was charged with substrate and tetrabutylammonium fluoride (15 ml), 1M/tetrahydrofuran). The mixture was stirred for next 49 h. The mixture was dissolved by the addition of ethyl acetate (150 ml) and extracted 6 times with water (50 ml) and brine (50 ml), dried Na₂SO₄) and evaporated. The oil residue was chromatographed on column (50 cm³, protected from light) using ethyl acetate as mobile phase. Fractions containing product were pooled and evaporated to give colorless oil. There was an impurity (Bu₃N) in the product (¹H, ¹³C NMR). Material was chromatographed twice on columns (60 cm³, protected from light) using hexane:ethyl acetate 2:1 and ethyl acetate as mobile phase. Oil was dissolved in methyl acetate and evaporated (4 times) to give product as white foam (251 mg, 87%).

[α]_{D}²²=+33.5 (c=0.48, EtOH)

UV λmax (EtOH): 243 nm (ε 29859), 252 nm (ε 34930), 262 nm (ε 23522)

¹H NMR (DMSO-D6): 8.94 (1H, s), 6.07 (1H, d, J=11.0 Hz), 5.78 (1H, d, J=11.0 Hz), 4.48 (1H, d, J=4.0 Hz), 4.38 (1H, d, J=4.0 Hz), 4.04 (1H, s), 3.92-3.76 (2H, m), 2.77 (1H, br d, J=11.0 Hz), 2.49-2.25 (2H, m),2.05-1.95 (4H, m), 1.76-1.20 (19H, m), 0.97 (3H, s), 0.60 (3H, s)

¹³C NMR (DMSO-D6): 138.95, 134.73, 121.50 (q, J=286.0 Hz), 120.80, 116.47, 89.59, 70.84, 70.44 (sep, J=31.9 Hz), 68.43, 65.57, 65.45, 65.28, 56.37, 55.91, 45.82, 44.59, 44.45, 42.23, 40.01, 39.43, 36.98, 28.29, 28.19, 22.98, 22.54, 22.08, 21.33, 17.78, 14.55

| | | | | |
|---|---|---|---|---|
| MS HRES | Calculated for: | C₃₂H₄₀D₆F₆O₄ | [M + Na]⁺ | 637.3569 |
| | Observed: | | [M + Na]⁺ | 637.3570 |

### Example 24

### Synthesis of 1α-Fluoro-25-hydroxy-20S-20-(4-hydroxy-5,5,5-trideutero-4-trideuteromethyl-pentyl)-23-yne-26,27-hexafluorocholecalciferol (24)

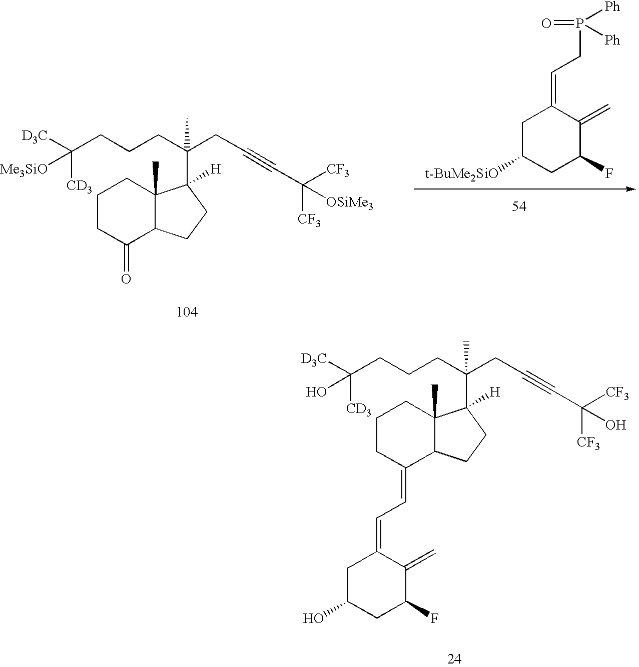

### 1α-Fluoro-25-hydroxy-20S-20-(4-hydroxy-5,5,5-trideutero-4-trideuteromethyl-pentyl)-23-yne-26,27-hexafluorocholecalciferol (24)

A 25 ml round bottom flask equipped with stir bar and Claisen adapter with rubber septum was charged with (1S,5R)-1-((tert-butyldimethyl)silanyloxy)-3-[2-(diphenylfosphinoyl)-eth-(Z)-ylidene]-5-fluoro-2-methylene-cyclohexane (500 mg, 1.062 mmol) and tetrahydrofuran (8 ml). The reaction mixture was cooled to −70° C. and n-butyllithium (0.66 ml, 1.06 mmol)) was added dropwise. The resulting deep red solution was stirred at −70° C. for 20 min and (1R,3aR,7aR)-7a-methyl-1-[(1S)-6,6,6-trifluoro-1-methyl-1-(5,5,5-trideutero-4-trideuteromethyl-4-trimethylsilanyloxy-pentyl)-5-trifluoromethyl-5-trimethylsilanyloxy-hex-3-ynyl]-octahydro-inden-4-one (269 mg, 0.424 mmol) was added dropwise in tetrahydrofuran (1.5 ml). The reaction mixture was stirred for 5 h (in last hour the temperature was increased from −70 do −55° C.). The bath was removed and the mixture was poured into ethyl acetate (50 ml) and saturated solution of ammonium chloride (10 ml). The water fraction was extracted with ethyl acetate (3×50 ml), dried (Na₂SO₄) and evaporated. The oil residue was chromatographed on column (50 cm³, protected from light) using hexane:ethyl acetate—10:1 as mobile phase. Fractions containing product were pooled and evaporated to give colorless oil. The oil residue was used to next reaction. A 25 ml round bottom flask equipped with stir bar and Claisen adapter with rubber septum was charged with substrate and tetrabutylammonium fluoride (15 ml, 1M/tetrahydrofuran). The mixture was stirred for 6 h. The mixture was dissolved by the addition of ethyl acetate (150 ml) and washed 6 times with water (50 ml) and brine (50 ml), dried Na₂SO₄) and evaporated. The oil residue was chromatographed on column (50 cm³, protected from light) using hexane:ethyl acetate—1:1 as mobile phase. Fractions containing product were pooled and evaporated to give product as colorless oil. There was an impurity (Bu₃N) in the product (¹H, ¹³C NMR). Material was chromatographed on column (60 cm³, protected from light) using hexane:ethyl acetate 2:1 and 1:1 as mobile phase. Oil was dissolved in methyl acetate and evaporated (4 times) to give product as white foam (229 mg, 86%).

[α]_{D}²⁵=+20.9 (c=0.45, EtOH)

UV λmax (EtOH): 211 nm (ε 15893), 243 nm (ε 16109), 270 nm (ε 16096)

¹H NMR (DMSO-D6): 8.93 (1H, s), 6.36 (1H, d, J=11.1 Hz), 5.93 (1H, d, J=11.3 Hz), 5.38 (1H, s), 5.14 (1H, ddd, J=49.6, 3.4, 2.0 Hz), 4.98 (1H, d, J=1.5 Hz), 4.86 (1H, d, J=4.3 Hz), 4.05 (1H, s), 3.94-3.88 (1H, m), 2.81 (1H, d, J=13.2 Hz), 2.44-2.35 (2H, m), 2.16-2.08 (2H, m), 1.98-1.93 (2H, m), 1.84-1.17 (17H, m), 0.95 (3H, s), 0.59 (3H, s)

¹³C NMR (DMSO-D6): 143.15 (d, J=16.7 Hz), 141.49, 133.06, 124.03, 121.49 (q, J=286.0 Hz), 117.40, 115.18 (d, J=9.9 Hz), 91.97 (d, J=166.9 Hz), 89.61, 70.85, 70.44 (sep, J=31.9 Hz), 68.43, 64.55 (d, J=4.6 Hz), 56.37, 55.91, 46.06, 44.84, 44.44, 40.70 (d, J=20.5 Hz), 39.97, 39.81, 39.43, 28.37, 28.26, 23.06, 22.52, 22.02, 21.32, 17.77, 14.48

| | | | | |
|---|---|---|---|---|
| MS HRES | Calculated for: | C₃₃H₃₉D₆F₇O₃ | [M + Na]⁺ | 651.3526 |
| | Observed: | | [M + Na]⁺ | 651.3528 |

### Example 25

### Synthesis of 1,25-Dihydroxy-20S-20-(4-hydroxy-5,5,5-trideutero-4-trideuteromethyl-pentyl)-23Z-ene-26,27-hexafluorocholecalciferol (16)

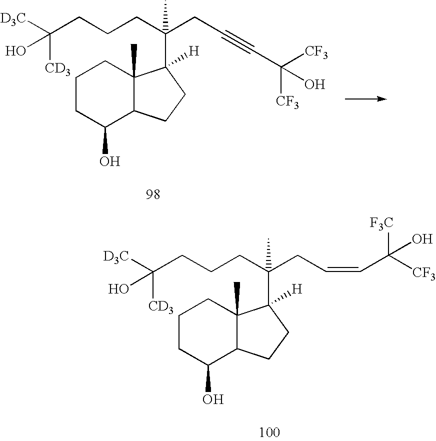

### (6S,3Z)-6-[(1R,3aR,4S,7aR)-4-hydroxy-7a-methyl-octahydro-inden-1-yl]-6-methyl-11,11,11-trideutero-10-trideuteromethyl-1,1,1-trifluoro-2-trifluoromethyl-undec-3-ene-2,10-diol (100)

A 50 ml round bottom flask was charged with (6S)-6-[(1R,3aR,4S,7aR)-4-hydroxy-7a-methyl-octahydro-inden-1-yl]-6-methyl-11,11,11-trideutero-10-trideuteromethyl-1,1,1-trifluoro-2-trifluoromethyl-undec-3-yne-2,10-diol (722 mg, 1.466 mmol), Pd/CaCO₃ (180 mg, 5%), hexane (16.8 ml), ethyl acetate (6.8 ml) and solution of quinoline in ethanol (0.65 ml, prepared from ethanol (3.1 ml) and quinoline (168 μl)).

The substrate was hydrogenated at ambient temperature and atmospheric pressure of hydrogen. The reaction was monitoring by TLC (dichloromethane:ethyl acetate 4:1, 3×). After 5 h 10 min the catalyst was filtered off (celite) and solvent evaporated. The residue was purified over silica gel (50 cm³) using dichloromethane:ethyl acetate 4:1. Fractions containing product were pooled and evaporated to give product as colorless oil (720 mg, 99%).

[α]_{D}³¹=+3.3 (c=0.49, EtOH)

¹H NMR (CDCl₃): 6.14-6.05 (1H, m), 5.48 (1H, d, J=12.8 Hz), 4.08 (1H, s), 2.83 (1H, dd, J=15.6, 9.0 Hz), 2.48-2.40 (1H, m), 2.00 (1H, d, J=11.4 Hz), 1.85-1.73 (2H, m), 1.64-1.24 (18H, m), 1.08 (3H, s), 0.99 (3H, s)

¹³C NMR (CDCl₃): 140.29, 117.60, 71.72, 69.91, 56.94, 52.76, 44.28, 43.62, 41.36, 40.39, 39.79, 36.97, 33.53, 22.78, 22.40, 21.88, 17.81, 13.73

| | | | | |
|---|---|---|---|---|
| MS HRES | Calculated for: | C₂₄H₃₂D₆F₆O₃ | [M + Na]⁺ | 517.2994 |
| | Observed: | | [M + Na]⁺ | 517.2997 |

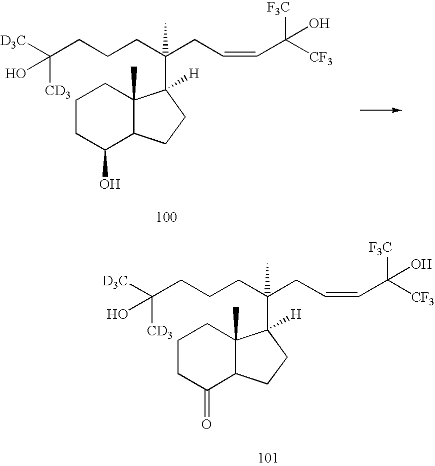

### (1R,3aR,7aR)-7a-Methyl-1-[(1S,3Z)-6,6,6-trifluoro-5-hydroxy-1-methyl-1-(5,5,5-trideutero-4-hydroxy-4-trideuteromethyl-pentyl)-5-trifluoromethyl-hex-3-enyl]-octahydro-inden-4-one (101)

A 25 ml round bottom flask equipped with stir bar and Claisen adapter with rubber septum was charged with pyridinium dichromate (1.50 g, 3.99 mmol) and dichloromethane (15 ml). The (6S,3Z)-6-[(1R,3aR,4S,7aR)-4-hydroxy-7a-methyl-octahydro-inden-1-yl]-6-methyl-11,11,11-trideutero-10-trideuteromethyl-1,1,1-trifluoro-2-trifluoromethyl-undec-3-ene-2,10-diol (710 mg, 1.436 mmol) in dichloromethane (5 ml) was added dropwise and mixture was stirred in room temperature for 6 h. The reaction mixture was filtrated through column with silica gel (50 cm³) using dichloromethane, dichloromethane:ethyl acetate 4:1, 3:1. The fractions containing product were pooled and evaporated to give oil (694 mg, 98%)

¹H NMR (CDCl₃): 6.10 (1H, m), 5.52 (1H, d, J=12.4 Hz), 5.07 (1H, br s), 2.92 (1H, dd, J=16.1, 9.9 Hz), 2.48-2.38 (2H, m), 2.91-1.25 (18H, m), 0.99 (3H, s), 0.74 (3H, s)

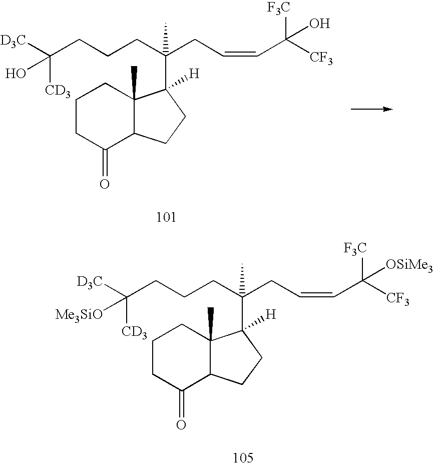

### (1R,3aR,7aR)-7a-Methyl-1-[(1S,3Z)6,6,6-trifluoro-1-methyl-1-(5,5,5-trideutero-4-trideuteromethyl-4-trimethylsilanyloxy-pentyl)-5-trifluoromethyl-5-trimethylsilanyloxy-hex-3-enyl]-octahydro-inden-4-one (105)

A 25 ml round bottom flask equipped with stir bar and Claisen adapter with rubber septum was charged with (1R,3aR,7aR)-7a-methyl-1-[(1S,3Z)-6,6,6-trifluoro-5-hydroxy-1-methyl-1-(5,5,5-trideutero-4-hydroxy-4-trideuteromethyl-pentyl)-5-trifluoromethyl-hex-3-enyl]-octahydro-inden-4-one (690 mg, 1.401 mmol) and dichloromethane (8 ml). 1-(Trimethylsilyl)imidazole (1.8 ml, 12.3 mmol) was added dropwise. The mixture was stirred at room temperature for 1.5 h. Ethyl acetate (150 ml) was added and the mixture was washed three times with water (50 ml), dried (Na₂SO₄) and evaporated. The oil residue was chromatographed on column (50 cm³) using hexane:ethyl acetate—10:1 as mobile phase. Fractions containing product were pooled and evaporated to give product as colorless oil (854 mg, 96%).

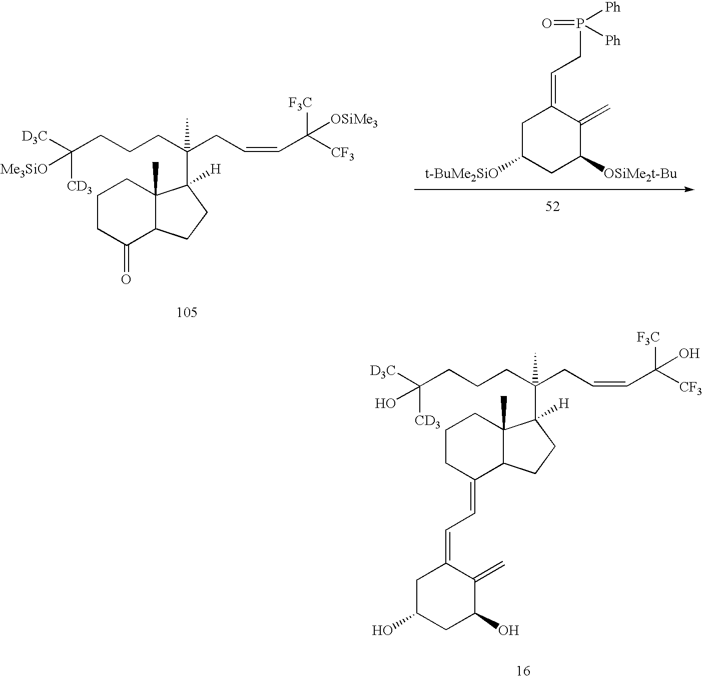

### 1,25-Dihydroxy-20S-20-(4-hydroxy-5,5,5-trideutero-4-trideuteromethyl-pentyl)-23Z-ene-26,27-hexafluorocholecalciferol (16)

A 25 ml round bottom flask equipped with stir bar and Claisen adapter with rubber septum was charged with (1S,5R)-1,5-bis-((tert-butyldimethyl)silanyloxy)-3-[2-(diphenylfosphinoyl)-eth-(Z)-ylidene]-2-methylene-cyclohexane (539 mg, 0.925 mmol) and tetrahydrofuran (8 ml). The reaction mixture was cooled to −78° C. and n-butyllithium (0.58 ml, 0.93 mmol) was added dropwise. The resulting deep red solution was stirred at −78° C. for 20 min and (1R,3aR,7aR)-7a-methyl-1-[(1S,3Z)6,6,6-trifluoro-1-methyl-1-(5,5,5-trideutero-4-trideuteromethyl-4-trimethylsilanyloxy-pentyl)-1-trifluoromethyl-5-trimethylsilanyloxy-hex-3-enyl]-octahydro-inden-4-one (270 mg, 0.424 mmol) was added dropwise in tetrahydrofuran (1.5 ml). The reaction mixture was stirred for 4 h 30 min and then the bath was removed and the mixture was poured into ethyl acetate (50 ml) and saturated solution of ammonium chloride (60 ml). The water fraction was extracted three times with ethyl acetate (50 ml), dried (Na₂SO₄) and evaporated. The oil residue was chromatographed on column (50 cm³, protected from light) using hexane:ethyl acetate—10:1 as mobile phase. Fractions containing product and some mono deprotected compound were pooled and evaporated to give colorless oil (350 mg).

A 25 ml round bottom flask equipped with stir bar and Claisen adapter with rubber septum was charged with oil and tetrabutylammonium fluoride (15 ml, 1M/tetrahydrofuran). The mixture was stirred for next 24 h. The mixture was dissolved by the addition of ethyl acetate (150 ml) and extracted 6 times with water and brine (30 ml+20 ml), dried (Na₂SO₄) and evaporated. The oil residue was chromatographed on column (50 cm³, protected from light) using ethyl acetate as mobile phase. Fractions containing product were pooled and evaporated to give product as colorless oil. Oil was dissolved in methyl acetate and evaporated (4 times) to give product as white foam (232 mg, 87%).

[α]_{D}²⁷=−5.4 (c=0.46, EtOH)

UV λmax (EtOH): 213 nm (ε 15177), 266 nm (ε 18553)

¹H NMR (DMSO-D6): 8.02 (1H, s), 6.19 (1H, d, J=31.3 Hz), 6.11 (1H, dt, J=12.1, 6.3 Hz), 5.98 (1H, d, J=1.1 Hz), 5.42 (1H, d, J=12.4 Hz), 5.23 (1H, s), 4.87 (1H, d, J=4.7 Hz), 4.76 (1H, s), 4.55 (1H, d, J=3.4 Hz), 4.20-4.17 (1H, m), 4.03 (1H, s), 3.98 (1H, br s), 2.82-2.75 (2H, m), 2.45 (1H, dd, J=16.6, 4.9 Hz), 2.36 (1H, d, J=11.9 Hz), 2.17 (1H, dd, J=13.04, 5.3 Hz), 2.04-1.95 (2H, m), 1.84-1.79 (1H, m), 1.73-1.54 (6H, m), 1.48-1.31 (4H, m), 1.22-1.17 (6H, m), 0.86 (3H, s), 0.61 (3H, s)

¹³C NMR (DMSO-D6): 149.41, 139.79, 139.46, 135.80, 122.95 (q, J=186.7 Hz), 122.37, 117.85, 117.01, 109.75, 76.76 (sep, J=28.9 Hz), 68.41, 68.37, 65.10, 56.45, 56.02, 51.21, 46.09, 44.87, 44.55, 43.12, 40.31, 39.37, 38.74, 35.68, 28.37, 23.21, 22.88, 21.81, 21.55, 17.60, 14.58

| | | | | |
|---|---|---|---|---|
| MS HRES | Calculated for | C₃₃H₄₂D₆F₆O₄ | [M + Na]⁺ | 651.3725 |
| | Observed: | | [M + Na]⁺ | 651.3728 |

### Example 26

### Synthesis of 1,25-Dihydroxy-20S-20-(4-hydroxy-5,5,5-trideutero-4-trideutero-4-trideuteromethyl-pentyl)-23Z-ene-26,27-hexafluoro-19-nor-cholecalciferol (17)

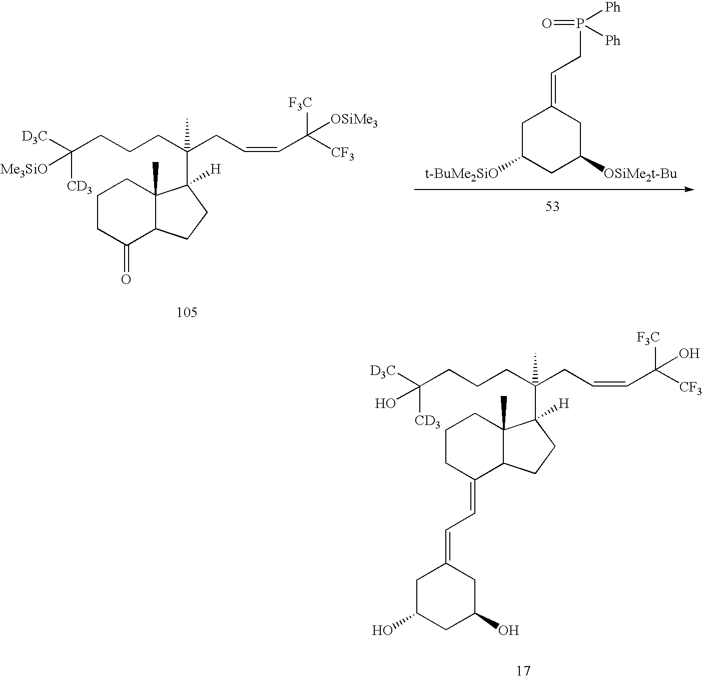

### 1,25-Dihydroxy-20S-20-(4-hydroxy-5,5,5-trideutero-4-trideuteromethyl-pentyl)-23Z-ene-26,27-hexafluoro-19-nor-cholecalciferol (17)

A 25 ml round bottom flask equipped with stir bar and Claisen adapter with rubber septum was charged with (1R,3R)-1,3-bis-((tert-butyldimethyl)silanyloxy)-5-[2-(diphenylfosphinoyl)ethylidene]-cyclohexane (541 mg, 0.948 mmol) and tetrahydrofuran (8 ml). The reaction mixture was cooled to −78° C. and n-butyllithium (0.59 ml, 0.94 mmol) was added dropwise. The resulting deep red solution was stirred at −78° C. for 20 min and (1R,3aR,7aR)-7a-methyl-1-[(1S,3Z)6,6,6-trifluoro-1-methyl-1-(5,5,5-trideutero-4-trideuteromethyl-4-trimethylsilanyloxy-pentyl)-5-trifluoromethyl-5-trimethylsilanyloxy-hex-3-enyl]-octahydro-inden-4-one (286 mg, 0.449 mmol) was added dropwise in tetrahydrofuran (1.5 ml). The reaction mixture was stirred for 4 h 10 min and then the bath was removed and the mixture was poured into ethyl acetate (50 ml) and saturated solution of ammonium chloride (60 ml). The water fraction was extracted three times with ethyl acetate (50 ml), dried (Na₂SO₄) and evaporated. The oil residue was chromatographed on column (50 cm³, protected from light) using hexane:ethyl acetate—10:1 as mobile phase. Fractions containing product and some mono deprotected compound were pooled and evaporated to give colorless oil (390 mg). A 25 ml round bottom flask equipped with stir bar and Claisen adapter with rubber septum was charged with oil and tetrabutylammonium fluoride (15 ml, 1M/tetrahydrofuran). The mixture was stirred for next 30 h. The mixture was dissolved by the addition of ethyl acetate (150 ml) and extracted 6 times with water and brine (30 ml+20 ml), dried (Na₂SO₄) and evaporated. The oil residue was chromatographed on column (60 cm³, protected from light) using ethyl acetate as mobile phase. Fractions containing product were pooled and evaporated to give product as colorless oil. Oil was dissolved in methyl acetate and evaporated (4 times) to give product as white foam (264 mg, 95%).

[α]_{D}²⁶=+32.0 (c=0.47, EtOH)

UV λmax (EtOH): 244 nm (ε 31469), 252 nm (ε 36060), 262 nm (ε 24658)

¹H NMR (DMSO-D6): 8.02 (1H, s), 6.14-6.08 (1H, m), 6.08 (1H, d, J=11.9 Hz), 5.78 (1H, d, J=11.1 Hz), 5.43 (1H, d, J=12.2 Hz), 4.49 (1H, d, J=4.1 Hz), 4.39 (1H, d, J=4.1 Hz), 4.04 (1H, s), 3.88-3.78 (2H, m), 2.82-2.72 (2H, m), 2.48-2.42 (2H, m), 2.31-2.25 (1H, m), 2.07-1.90 (4H, m), 1.73-1.18 (17H, m), 0.87 (3H, s), 0.61 (3H, s)

¹³C NMR (DMSO-D6): 139.45, 139.19, 134.57, 122.94 (q, J=286.8 Hz), 120.84, 117.02, 116.29, 76.75 (sep, J=28.8 Hz), 68.41, 65.55, 65.27, 56.43, 55.98, 45.94, 44.60, 44.55, 42.23, 40.32, 39.38, 38.74, 36.97, 35.69, 28.21, 23.07, 22.89, 21.85, 21.44, 17.59, 14.69

| | | | | |
|---|---|---|---|---|
| MS HRES | Calculated for: | C₃₂H₄₂D₆F₆O₄ | [M + Na]⁺ | 639.3725 |
| | Observed: | | [M + Na]⁺ | 639.3724 |

### Example 27

### Synthesis of 1α-Fluoro-25-hydroxy-20S-20-(4-hydroxy-5,5,5-trideutero-4-trideuteromethyl-pentyl)-23Z-ene-26,27-hexafluorocholecalciferol (18)

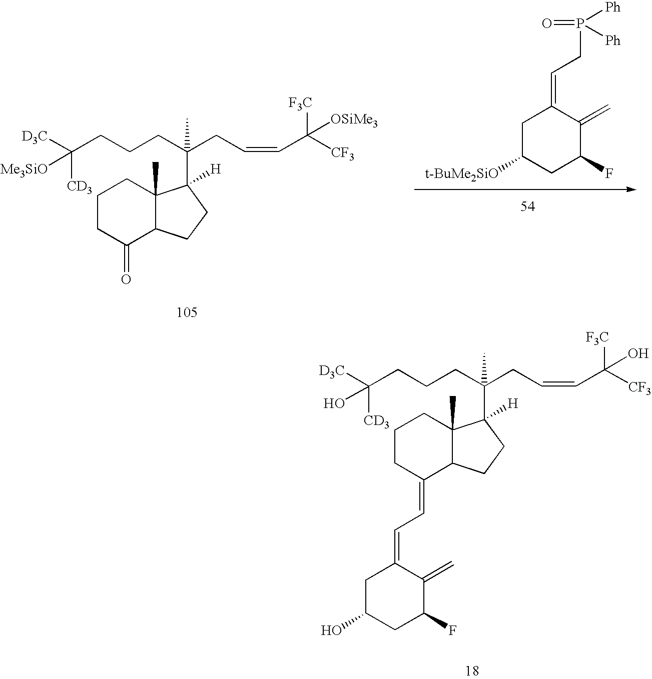

### 1α-Fluoro-25-hydroxy-20S-20-(4-hydroxy-5,5,5-trideutero-4-trideuteromethyl-pentyl)-23Z-ene-26,27-hexafluorocholecalciferol (18)

A 25 ml round bottom flask equipped with stir bar and Claisen adapter with rubber septum was charged with (1S,5R)-1-((tert-butyldimethyl)silanyloxy)-3-[2-(diphenylfosphinoyl)-eth-(Z)-ylidene]-5-fluoro-2-methylene-cyclohexane (462 mg, 0.982 mmol) and tetrahydrofuran (8 ml). The reaction mixture was cooled to −78° C. and n-butyllithium (0.61 ml, 0.98 mmol)) was added dropwise. The resulting deep red solution was stirred at −78° C. for 20 min and (1R,3aR,7aR)-7a-methyl-1-[(1S,3Z)6,6,6-trifluoro-1-methyl-1-(5,5,5-trideutero-4-trideuteromethyl-4-trimethylsilanyloxy-pentyl)-5-trifluoromethyl-5-trimethylsilanyloxy-hex-3-enyl]-octahydro-inden-4-one (267 mg, 0.419 mmol) was added dropwise in tetrahydrofuran (1.5 ml). The reaction mixture was stirred for 5 h and then the bath was removed and the mixture was poured into ethyl acetate (50 ml) and saturated solution of ammonium chloride (60 ml). The water fraction was extracted with ethyl acetate (3×50 ml), dried (Na₂SO₄) and evaporated. The oil residue was chromatographed on column (50 cm³, protected from light) using hexane:ethyl acetate—10:1 as mobile phase. Fractions containing product and some mono deprotected compound were pooled and evaporated to give colorless oil.

A 25 ml round bottom flask equipped with stir bar and Claisen adapter with rubber septum was charged with substrate and tetrabutylammonium fluoride (15 ml, 1M/tetrahydrofuran). The mixture was stirred for next 5 h. The mixture was dissolved by the addition of ethyl acetate (150 ml) and extracted 6 times with water and brine (30 ml+20 ml), dried (Na₂SO₄) and evaporated. The oil residue was chromatographed on column (50 cm³, protected from light) using hexane:ethyl acetate (1:1) as mobile phase. Product contained some impurities and was rechromatographed on column (VersaPak, 40×75 mm) using hexane:ethyl acetate (1:1) s mobile phase. Fractions containing product were pooled and evaporated to give product as colorless oil. Oil was dissolved in methyl acetate and evaporated (4 times) to give product as white foam (244 mg, 92%).

[α]_{D}²⁶=+11.8 (c=0.51, EtOH)

UV λmax (EtOH): 244 nm (ε 15004), 270 nm (ε 15084)

¹H NMR (DMSO-D6): 8.02 (1H, s), 6.36 (1H, d, J=11.3 Hz), 6.14-6.07 (1H, m), 5.39 (1H, d, J=1.3 Hz), 5.42 (1H, d, J=11.9 Hz), 5.39 (1H, s), 5.14 (1H, br d, J=49.7 Hz), 4.99 (1H, d, J=1.7 Hz), 4.86 (1H, d, J=4.3 Hz), 4.03 (1H, s), 3.93-3.88 (1H, m), 2.82-2.74 (2H, m), 2.48-2.43 (2H, m), 2.17-1.97 (4H, m), 1.84-1.55 (6H, m), 1.46-1.32 (4H, m), 1.29-1.16 (7H, m), 0.86 (3H, s), 0.60 (3H, s)

¹³C NMR (DMSO-D6): 143.18 (d, J=16.7 Hz), 141.74, 139.43, 132.93, 124.08, 122.95 (q, J=286.7 Hz), 117.22, 117.01, 115.08 (d, J=9.1 Hz), 91.93 (d, J=166.9 Hz), 76.76 (sep, J=28.0 Hz), 68.41, 64.56, 56.43, 55.96, 46.18, 44.82, 44.54, 40.69 (d, J=20.5 Hz), 40.27, 38.73, 35.68, 28.38, 23.15, 22.85, 21.80, 21.45, 17.59, 14.61

| | | | | |
|---|---|---|---|---|
| MS HRES | Calculated for: | C₃₃H₄₁D₆F₇O₃ | [M + Na]⁺ | 653.3682 |
| | Observed: | | [M + Na]⁺ | 653.3689 |

### Example 28

### Synthesis of 1,25-Dihydroxy-20S-20-(4-hydroxy-5,5,5-trideutero-4-trideuteromethyl-pentyl)-23E-ene-26,27-hexafluorocholecalciferol (19)

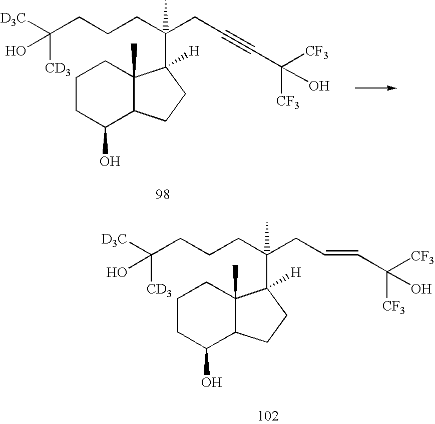

### (6S,3E)₆-[(1R,3aR,4S,7aR)-4-hydroxy-7a-methyl-octahydro-inden-1-yl]-6-methyl-11,11,11-trideutero-10-trideuteromethyl-1,1,1-trifluoro-2-trifluoromethyl-undec-3-ene-2,10-diol (102)

A 25 ml round bottom flask equipped with stir bar and condenser with nitrogen sweep was charged with lithium aluminum hydride (12.0 ml, 12.0 mmol, 1M/tetrahydrofuran) and the mixture was cooled to 0° C. Sodium methoxide (648 mg, 12.0 mmol) was added slowly followed by (6S)-6-[(1R,3aR,4S,7aR)-4-hydroxy-7a-methyl-octahydro-inden-1-yl]-6-methyl-11,11,11-trideutero-10-trideuteromethyl-1,1,1-trifluoro-2-trifluoromethyl-undec-3-yne-2,10-diol (740 mg, 1.502 mmol) in tetrahydrofuran (8 ml). The reaction mixture was stirred at 80° C. for 4 h and then was cooled to 0° C. Saturated solution of ammonium chloride (5 ml) was added slowly followed by saturated solution of ammonium chloride (60 ml) and 2N HCl (20 ml). The mixture was extracted with ethyl acetate (3×50 ml), dried (Na₂SO₄) and evaporated. The oil residue was chromatographed on columns (50 cm³) using hexane:ethyl acetate—4:1 as mobile phase. Fractions containing product were pooled and evaporated to give colorless oil (727 mg, 98%).

[α]_{D}³⁰=−0.64 (c=0.47, EtOH)

¹H NMR (CDCl₃): 6.32 (1H, dt, J=15.4, 7.9), 5.58 (1H, d, J=15.8 Hz), 4.09 (1H, br s), 2.29 (2H, d, J=8.1 Hz), 2.04-1.97 (1H, m), 1.84-1.76 (2H, m), 1.63-1.18 (18H, m), 1.09 (3H, s), 0.98 (3H, 6)

¹³C NMR (CDCl₃): 137.23, 120.09, 71.53, 69.83, 57.36, 52.71, 44.27, 43.69, 42.44, 41.61, 40.22, 33.54, 23.20, 22.36, 21.88, 18.02, 17.70, 17.31, 16.77

| | | | | |
|---|---|---|---|---|
| MS HRES | Calculated for: | C₂₄H₃₂D₆F₆O₃ | [M + Na]⁺ | 517.2994 |
| | Observed: | | [M + Na]⁺ | 517.2994 |

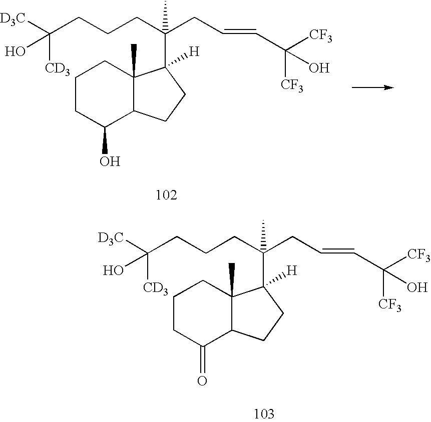

### (1R,3aR,7aR)-7a-Methyl-1-[(1S,3E)-6,6,6-trifluoro-5-hydroxy-1-methyl-1-(5,5,5-trideutero-4-hydroxy-4-trideuteromethyl-pentyl)-5-trifluoromethyl-hex-3-enyl]-octahydro-inden-4-one (103)

A 25 ml round bottom flask equipped with stir bar and Claisen adapter with rubber septum was charged with pyridinium dichromate (1.50 g, 3.99 mmol) and dichloromethane (15 ml). The (6S,3E)-6-[(1R,3aR,4S,7aR)-4-hydroxy-7a-methyl-octahydro-inden-1-yl]-6-methyl-11,11,11-trideutero-10-trideuteromethyl-1,1,1-trifluoro-2-trifluoromethyl-undec-3-ene-2,10-diol (730 mg, 1.476 mmol) in dichloromethane (5 ml) was added dropwise and mixture was stirred in room temperature for 4.5 h. The reaction mixture was filtrated through column with silica gel (50 cm³) using dichloromethane, dichloromethane:ethyl acetate 4:1. The fractions containing product were pooled and evaporated to give oil (706 mg, 97%).

[α]_{D}³⁰=−20.0 (c=0.46, EtOH)

¹H NMR (CDCl₃): 6.33 (1H, dt, J=15.3, 7.7 Hz), 5.61 (1H, d, J=15.6 Hz), 2.43 (1H, dd, J=1.1.2, 7.1 Hz), 2.33-2.19 (4H, m), 2.17-2.12 (1H, m), 2.06-2.00 (1H, m), 1.95-1.84 ((1H, m), 1.80-1.54 (7H, m), 1.40-1.20 (5H, m), 1.15-1.09 (1H, m), 0.98 (3H, s), 0.75 (3H, s)

¹³C NMR (CDCl₃): 211.74, 136.54, 119.96, 71.25, 62.22, 57.49, 50.59, 43.80, 42.54, 40.85, 39.97, 39.80, 24.04, 23.03, 22.10, 18.67, 17.72, 15.71

| | | | | |
|---|---|---|---|---|
| MS HRES | Calculated for: | C₂₄H₃₀D₆F₆O₃ | [M + Na]⁺ | 515.2837 |
| | Observed: | | [M + Na]⁺ | 515.2837 |

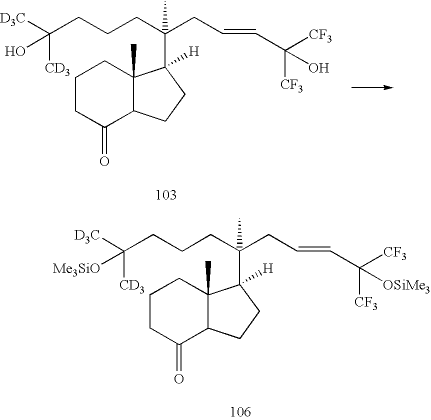

### (1R,3aR,7aR)-7a-Methyl-1-[(1S,3E)-6,6,6-trifluoro-1-methyl-1-(5,5,5-trideutero-4-trideuteromethyl-4-trimethylsilanyloxy-pentyl)-5-trifluoromethyl-5-trimethylsilanyloxy-hex-3-enyl]-octahydro-inden-4-one (106)

A 25 ml round bottom flask equipped with stir bar and Claisen adapter with rubber septum was charged with (1R,3aR,7aR)-7a-methyl-1-[(1S,3E)-6,6,6-trifluoro-5-hydroxy-1-methyl-1-(5,5,5-trideutero-4-hydroxy-4-trideuteromethyl-pentyl)-5-trifluoromethyl-hex-3-enyl]-octahydro-inden-4-one (698 mg, 1.417 mmol) and dichloromethane (8 ml). 1-(trimethylsilyl)imidazole (1.8 ml, 12.3 mmol) was added dropwise. The mixture was stirred at room temperature for 2 h. Ethyl acetate (150 ml) was added and the mixture was washed with water (4×50 ml), dried (Na₂SO₄) and evaporated. The oil residue was chromatographed on column (60 cm³) using hexane:ethyl acetate—10:1 as mobile phase. Fractions containing product were pooled and evaporated to give product as colorless oil (871 mg, 96%).

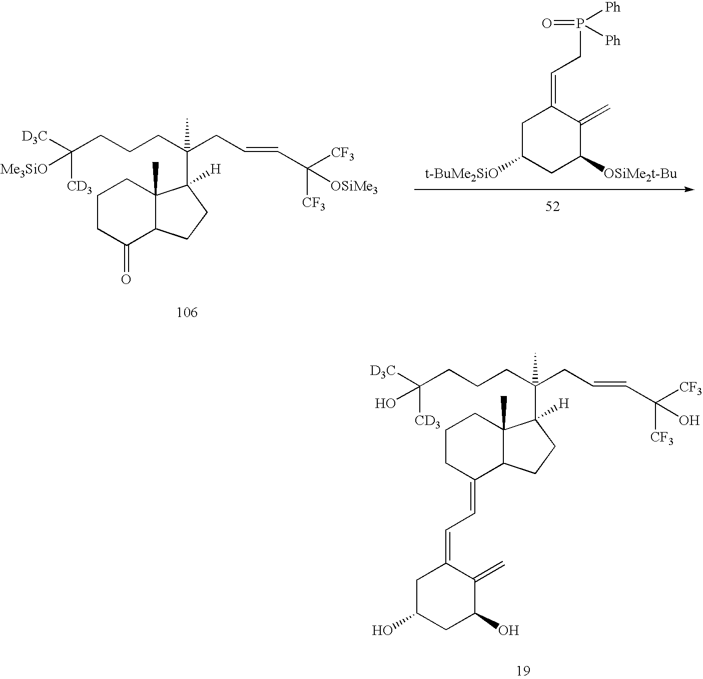

### 1,25-Dihydroxy-20S-20-(4-hydroxy-5,5,5-trideutero-4-trideuteromethyl-pentyl)-23E-ene-26,27-hexafluorocholecalciferol (19)

A 25 ml round bottom flask equipped with stir bar and Claisen adapter with rubber septum was charged with (1S,5R)-1,5-bis-((tert-butyldimethyl)silanyloxy)-3-[2-(diphenylfosphinoyl)-eth-(Z)-ylidene]-2-methylene-cyclohexane (531 mg, 0.911 mmol) and tetrahydrofuran (8 ml). The reaction mixture was cooled to −78° C. and n-butyllithium (0.57 ml, 0.91 mmol)) was added dropwise. The resulting deep red solution was stirred at −78° C. for 20 min and (1R,3aR,7aR)-7a-methyl-1-[(1S,3E)-6,6,6-trifluoro-1-methyl-1-(5,5,5-trideutero-4-trideuteromethyl-4-trimethylsilanyloxy-pentyl)-5-trifluoromethyl-5-trimethylsilanyloxy-hex-3-enyl]octahydro-inden-4-one (260 mg, 0.408 mmol) was added dropwise in tetrahydrofuran (1.5 ml). The reaction mixture was stirred for 5 h 30 min and then the bath was removed and the mixture was poured into ethyl acetate (50 ml) and saturated solution of ammonium chloride (60 ml). The water fraction was extracted with ethyl acetate (3×50 ml), dried (Na₂SO₄) and evaporated. The oil residue was chromatographed on column (50 cm³, protected from light) using hexane:ethyl acetate—10:1 as mobile phase. Fractions containing product and some mono deprotected compound were pooled and evaporated to give colorless oil. A 25 ml round bottom flask equipped with stir bar and Claisen adapter with rubber septum was charged with substrate and tetrahydrofuran (5 ml). Tetrabutylammonium fluoride (2.10 g, 6.66 mmol) was added. The mixture was stirred for next 6 h and tetrabutylammonium fluoride (5 ml, 1M/tetrahydrofuran) was added. The reaction was stirred for next 15 h. The mixture was dissolved by the addition of ethyl acetate (150 ml) and extracted 6 times with water and brine (30 ml+20 ml), dried (Na₂SO₄) and evaporated. The oil residue was chromatographed on column (50 cm³, protected from light) using ethyl acetate as mobile phase. Fractions containing product were pooled and evaporated to give product as colorless oil. Oil was dissolved in methyl acetate and evaporated (4 times) to give product as white foam (186 mg, 73%).

[α]_{D}³⁰=+4.5 (c=0.44, EtOH)

UV λmax (EtOH): 213 nm (ε 13978), 265 nm (ε 16276)

¹H NMR (CDCl₃): 6.37 (1H, d, J=11.1 Hz), 6.31 (1H, dd, J=15.6, 7.9 Hz), 6.00 (1H, d, J=11.1 Hz), 5.59 (1H, d, J=15.6 Hz), 5.33 (1H, s), 4.99 (1H, s), 4.43 (1H, br s), 4.23 (1H, br s), 2.81 (1H, dd, J=12.2, 3.4 Hz), 2.59 (1H, br d, J=10.5 Hz), 2.34-2.29 (3H, m), 2.06-1.98 (3H, m), 1.93-1.87 (1H, m), 1.76-1.18 (18H, m), 1.12-1.06 (1H, m), 0.95 (3H, s), 0.66 (3H, s)

¹³C NMR (DMSO-D6): 149.41, 139.75, 136.73, 135.85, 122.63 (q, J=285.2 Hz), 122.39, 119.72, 117.94, 109.79, 75.51 (sep, J=29.6 Hz), 68.41, 65.11, 56.54, 56.02, 46.13, 44.87, 44.43, 43.11, 41.20, 40.48, 28.37, 23.14, 22.90, 21.72, 21.52, 17.56, 14.70

| | | | | |
|---|---|---|---|---|
| MS HRES | Calculated for: | C₃₃H₄₂D₆F₆O₄ | [M + Na]⁺ | 651.3725 |
| | Observed: | | [M + Na]⁺ | 651.3727 |

### Example 29

### Synthesis of 1,25-Dihydroxy-20S-20-(4-hydroxy-5,5,5-trideutero-4-trideuteromethyl-pentyl)-23E-ene-26,27-hexafluoro-19-nor-cholecalciferol (20)

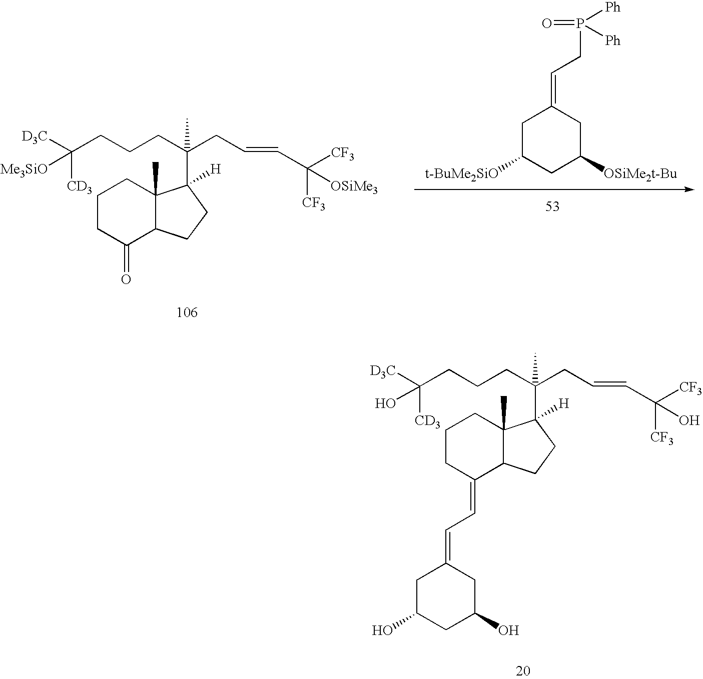

### 1,25-Dihydroxy-20S-20-(4-hydroxy-5,5,5-trideutero-4-trideuteromethyl-pentyl)-23E-ene-26,27-hexafluoro-19-nor-cholecalciferol (20)

A 25 ml round bottom flask equipped with stir bar and Claisen adapter with rubber septum was charged with (1R,3R)-1,3-bis-((tert-butyldimethyl)silanyloxy)-5-[2-(diphenylfosphinoyl)ethylidene]-cyclohexane (546 mg, 0.956 mmol) and tetrahydrofuran (8 ml). The reaction mixture was cooled to −78° C. and n-butyllithium (0.60 ml, 0.96 mmol)) was added dropwise. The resulting deep red solution was stirred at −78° C. for 20 min and (1R,3aR,7aR)-7a-methyl-1-[(1S,3E)-6,6,6-trifluoro-1-methyl-1-(5,5,5-trideutero-4-trideuteromethyl-4-trimethylsilanyloxy-pentyl)-5-trifluoromethyl-5-trimethylsilanyloxy-hex-3-enyl]-octahydro-inden-4-one (295 mg, 0.463 mmol) was added dropwise in tetrahydrofuran (1.5 ml). The reaction mixture was stirred for 5 h 30 min and then the bath was removed and the mixture was poured into ethyl acetate (50 ml) and saturated solution of ammonium chloride (60 ml). The water fraction was extracted with ethyl acetate (3×50 ml), dried (Na₂SO₄) and evaporated. The oil residue was chromatographed on column (50 cm³, protected from light) using hexane:ethyl acetate—10:1 as mobile phase. Fractions containing product and some mono deprotected compound were pooled and evaporated to give colorless oil. A 25 ml round bottom flask equipped with stir bar and Claisen adapter with rubber septum was charged with substrate and tetrabutylammonium fluoride (15 ml, 1M/tetrahydrofuran). The mixture was stirred for next 42 h. The mixture was dissolved by the addition of ethyl acetate (150 ml) and extracted 6 times with water and brine (30 ml+20 ml), dried (Na₂SO₄) and evaporated. The oil residue was chromatographed on column (50 cm³, protected from light) using ethyl acetate as mobile phase. Fractions containing product were pooled and evaporated to give product as colorless oil. Oil was dissolved in methyl acetate and evaporated (4 times) to give product as white foam (280 mg, 98%).

[α]_{D}³⁰=+41.1 (c=0.46, EtOH)

UV λmax (EtOH): 244 nm (ε 32355), 252 nm (ε 37697), 262 nm (ε 25353)

¹H NMR (DMSO-D6): 8.04 (1H, s), 6.32 (1H, dt, J=15.6, 7.7 Hz), 6.07 (1H, d, J=11.1 Hz), 5.78 (1H, d, J=11.1 Hz), 5.63 (1H, d, J=15.3 Hz), 4.50 (1H, d, J=3.4 Hz), 4.39 (1H, d, J=3.4 Hz), 4.04 (1H, s), 3.88 (1H, br s), 3.80 (1H, br s), 2.74 (1H, br d, J=13.9 Hz), 2.44 (1H, dd, J=13.0, 3.0 Hz), 2.33-2.21 (2H, m), 2.07-1.95 (2H, m), 1.69-1.04 (17H, m), 0.90 (3H, s), 0.62 (3H, s)

¹³C NMR (DMSO-D6): 139.13, 136.71, 134.63, 122.44 (q, J=285.2 Hz), 120.83, 119.71, 116.38, 75.51 (sep, J=28.9 Hz), 68.37, 65.57, 65.28, 56.52, 55.97, 45.96, 44.59, 44.44, 42.23, 41.18, 40.48, 39.62, 39.58, 37.00, 28.19, 22.99, 22.91, 21.76, 21.42, 17.55, 14.79

| | | | | |
|---|---|---|---|---|
| MS HRES | Calculated for: | C₃₂H₄₂D₆F₆O₄ | [M + Na]⁺ | 639.3725 |
| | Observed: | | [M + Na]⁺ | 639.3724 |

### Example 30

### Synthesis of 1α-Fluoro-25-hydroxy-20S-20-(4-hydroxy-5,5,5-trideutero-4-trideuteromethyl-pentyl)-23E-ene-26,27-hexafluorocholecalciferol (21)

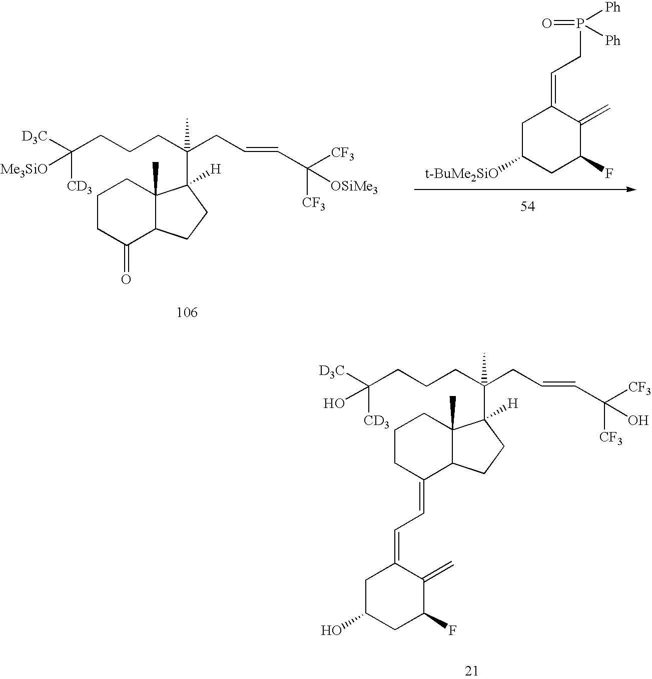

### 1α-Fluoro-25-hydroxy-20S-20-(4-hydroxy-5,5,5-trideutero-4-trideuteromethyl-pentyl)-23E-ene-26,27-hexafluorocholecalciferol (21)

A 25 ml round bottom flask equipped with stir bar and Claisen adapter with rubber septum was charged with (1S,5R)-1-((tert-butyldimethyl)silanyloxy)-3-[2-(diphenylfosphinoyl)-eth-(Z)-ylidene]-5-fluoro-2-methylene-cyclohexane (473 mg, 1.005 mmol) and tetrahydrofuran (8 ml). The reaction mixture was cooled to −78° C. and n-butyllithium (0.63 ml, 1.01 mmol)) was added dropwise. The resulting deep red solution was stirred at −78° C. for 20 min and (1R,3aR,7aR)-7a-methyl-1-[(1S,3E)-6,6,6-trifluoro-1-methyl-1-(5,5,5-trideutero-4-trideuteromethyl-4-trimethylsilanyloxy-pentyl)-5-trifluoromethyl-5-trimethylsilanyloxy-hex-3-enyl]-octahydro-inden-4-one (271 mg, 0.426 mmol) was added dropwise in tetrahydrofuran (1.5 ml). The reaction mixture was stirred for 4.5 h and then the bath was removed and the mixture was poured into ethyl acetate (50 ml) and saturated solution of ammonium chloride (60 ml). The water fraction was extracted with ethyl acetate (3×50 ml), dried (Na₂SO₄) and evaporated. The oil residue was chromatographed on column (50 cm³, protected from light) using hexane:ethyl acetate—10:1 as mobile phase. Fractions containing product and some mono deprotected compound were pooled and evaporated to give colorless oil. A 25 ml round bottom flask equipped with stir bar and Claisen adapter with rubber septum was charged with substrate and tetrabutylammonium fluoride (10 ml, 1M/tetrahydrofuran). The mixture was stirred for next 17 h. The mixture was dissolved by the addition of ethyl acetate (150 ml) and extracted 6 times with water and brine (30 ml+20 ml), dried (Na₂SO₄) and evaporated. The oil residue was chromatographed on column (50 cm³, protected from light) using hexane:ethyl acetate (1:1) as mobile phase. Fractions containing product were pooled and evaporated to give product as colorless oil. Oil was dissolved in methyl acetate and evaporated (4 times) to give product as white foam (226 mg, 84%).

[α]_{D}²⁸=+25.3 (c=0.45, EtOH)

UV λmax (EtOH): 243 nm (ε 14182), 269 nm (ε 14044)

¹H NMR (DMSO-D6): 8.03 (1H, s), 6.36 (1H, d, J=10.9 Hz), 6.33-6.27 (1H, m), 5.93 (1H, d, J=11.1 Hz), 5.63 (1H, d, J=15.4 Hz), 5.38 (1H, s), 5.14 (1H, br d, J=49.7 Hz), 4.99 (1H, s), 4.86 (1H, d, J=4.3 Hz), 4.03 (1H, s), 3.94-3.88 (1H, m), 2.81 (1H, br d, J=12.4 Hz), 2.34-2.20 (2H, m), 2.16-2.06 (2H, m), 2.00-1.95 (1H, m), 1.84-1.02 (18H, m), 0.89 (3H, s), 0.61 (3H, s)

¹³C NMR (DMSO-D6): 143.17 (d, J=16.7 Hz), 141.68, 136.70, 132.97, 124.05, 122.62 (q, J=286.7 Hz), 119.71, 117.29, 115.16, 91.95 (d, J=166.9 Hz), 75.50 (sep, J=28.8 Hz), 68.36, 64.56, 56.51, 55.95, 46.19, 44.83, 44.42, 41.15, 40.69 (d, J=20.5 Hz), 40.41, 39.61, 28.36, 23.06, 22.88, 21.70, 21.40, 17.54, 14.71

| | | | | |
|---|---|---|---|---|
| MS HRES | Calculated for: | C₃₃H₄₁D₆F₇O₃ | [M + Na]⁺ | 653.3682 |
| | Observed: | | [M + Na]⁺ | 653.3686 |

### Example 31

### Synthesis of 1,25-Dihydroxy-20R-20-(4-hydroxy-5,5,5-trideutero-4-trideuteromethyl-pentyl)-23-yne-26,27-hexafluorocholecalciferol (31)

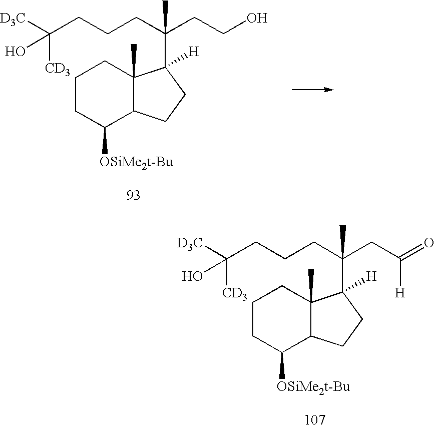

### (3R)-3-[(1R,3aR,4S,7aR)-4-(tert-Butyl-dimethyl-silanyloxy)-7a-methyl-octahydro-inden-1-yl]-8,8,8-trideutero-7-hydroxy-3-methyl-trideuteromethyl-octanal (107)

A 250 ml round bottom flask equipped with stir bar and Claisen adapter with rubber septum was charged with pyridinium chlorochromate (3.858 g, 17.898 mmol), celite (3.93 g) and dichloromethane (70 ml). The (3R)-3-[(1R,3aR,4S,7aR)-4-(tert-butyl-dimethyl-silanyloxy)-7a-methyl-octahydro-inden-1-yl]-8,8,8-trideutero-3-methyl-7-trideuteromethyl-octane-1,7-diol (5.00 g, 11.190 mmol) in dichloromethane (10 ml) was added dropwise and mixture was stirred in room temperature for 3 h 45 min. The reaction mixture was filtrated through column with silica gal (250 cm³) and celite (1 cm) and using dichloromethane, dichloromethane:ethyl acetate 4:1. The fractions containing product were pooled and evaporated to give oil (4.42 g, 89%).

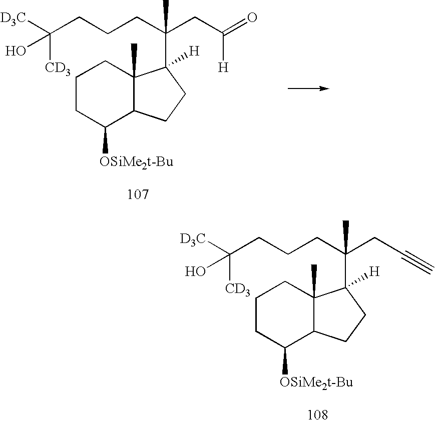

### (6R)-6-[(1R,3aR,4S,7aR)-4-(tert-Butyl-dimethyl-silanyloxy)-7a-methyl-octahydro-inden-1-yl]-1,1,1-trideutero-6-methyl-2-trideuteromethyl-non-8-yn-2-ol (108)

A 250 ml round bottom flask equipped with stir bar and Claisen adapter with rubber septum was charged with (3R)-3-[(1R,3aR,4S,7aR)-4-(tert-butyl-dimethyl-silanyloxy)-7a-methyl-octahydro-inden-1-yl]-8,8,8-trideutero-7-hydroxy-3-methyl-7-trideuteromethyl-octanal (4.42 g, 9.937 mmol) and methanol (65 ml). 1-diazo-2-oxo-propyl)-phosphonic acid dimethyl ester (3.75 g, 19.52 mmol) in methanol (3 ml) was added and the resulting mixture was cooled in an ice bath. Potassium carbonate (3.75 g, 27.13 mmol) was added and the reaction mixture was stirred in the ice bath for 30 min and then at room temperature for 4 h. Water (100 ml) was added and the mixture was extracted with ethyl acetate (4×80 ml), dried (Na₂SO₄) and evaporated. The residue was filtrated through silica gel (50 cm³) using hexane:ethyl acetate—5:1 and evaporated. The oil residue was chromatographed on column (VersaPak Cartridge 80×150 mm) using hexane:ethyl acetate—5:1 and 4:1 as mobile phase. Fractions containing product were pooled and evaporated to give product as colorless oil (3.83 g, 87%).

¹H NMR (CDCl₃): 3.99 (1H, br s), 2.12-1.92 (4H, m), 1.83-1.75 (1H, m), 1.68-1.22 (17H, m), 1.04 (3H, s), 0.99 (3H, s), 0.88 (9H, s), 0.00 (3H, s), −0.01 (3H, s)

¹³C NMR (CDCl₃): 82.90, 70.75, 69.67, 69.60, 60.33, 56.61, 52.99, 44.73, 43.71, 41.35, 39.55, 39.51, 34.34, 29.51, 25.83, 22.77, 22.39, 22.03, 18.49, 18.03, 17.73, 16.48, 14.19, −4.79, −5.14

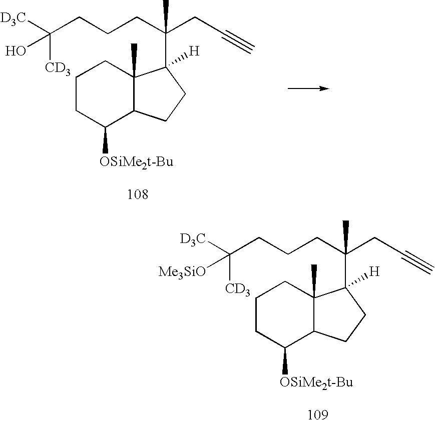

### (1R,3aR,4S,7aR)-4-(tert-Butyl-dimethyl-silanyloxy)-7a-methyl-1-[(1R)-6,6,6-trideutero-1-methyl-1-(prop-2-ynyl)-5-trideuteromethyl-5-trimethylsilanyloxy-hexyl]-octahydro-indene (109)

A 100 ml round bottom flask equipped with stir bar and Claisen adapter with rubber septum was charged with (6R)-6-[(1R,3aR,4S,7aR)-4-(tert-butyl-dimethyl-silanyloxy)-7a-methyl-octahydro-inden-1-yl]-1,1,1-trideutero-6-methyl-2-trideuteromethyl-non-8-yn-2-ol (3.80 g, 8.62 mmol) and dichloromethane (30 ml). 1-(trimethylsilyl)imidazole (3.7 ml, 25.22 mmol) was added dropwise. The mixture was stirred at room temperature for 1 h 35 min. Water (100 ml) was added and the mixture was extracted with hexane (3×70 ml), dried (Na₂SO₄) and evaporated. The oil residue was chromatographed on column (250 cm³) using hexane:ethyl acetate—20:1 as mobile phase. Fractions containing product were pooled and evaporated to give product as colorless oil (4.09 g, 93%).

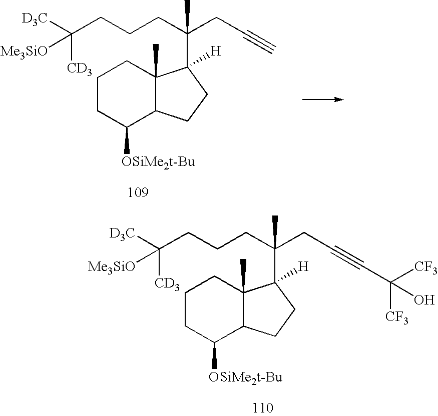

### (6R)-6-[(1R,3aR,4S,7aR)-4-(tert-Butyl-dimethyl-silanyloxy)-7a-methyl-octahydro-inden-1-yl]-6-methyl-11,11,11-trideutero-10-trideuteromethyl-1,1,1-trifluoro-2-trifluoromethyl-10-trimethylsilanyloxy-undec-3-yn-2-ol (110)

A two neck 10 ml round bottom flask equipped with stir bar, Claisen adapter with rubber septum and funnel (with cooling bath) was charged with (1R,3aR,4S,7aR)-4-(tert-butyl-dimethyl-silanyloxy)-7a-methyl-1-[(1R)-6,6,6-trideutero-1-methyl-1-(prop-2-ynyl)-5-trideuteromethyl-5-trimethylsilanyloxy-hexyl]-octahydro-indene (4.09 g, 7.97 mmol) and tetrahydrofuran (50 ml). The funnel was connected to container with hexafluoroacetone and cooled (acetone, dry ice). The reaction mixture was cooled to −70° C. and n-butyllithium (7.5 ml, 12.00 mmol) was added dropwise. After 30 min hexafluoroacetone was added (the container's valve was opened three times). The reaction was steered at −70° C. for 2 h then saturated solution of ammonium chloride (5 ml) was added. The mixture was dissolved by the addition of saturated solution of ammonium chloride (100 ml) and extracted with ethyl acetate (3×80 ml), dried (Na₂SO₄) and evaporated. The residue was chromatographed twice on columns (300 cm³, hexane:ethyl acetate—20:1) to give the mixture of product and polymer (from hexafluoroacetone) (5.56 g). Product was used to the next reaction without purification.

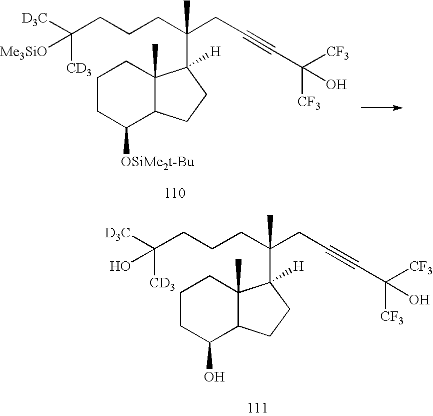

### (6R)-6-[(1R,3aR,4S,7aR-4-hydroxy-7a-methyl-octahydro-inden-1-yl]-6-methyl-11,11,11-trideutero-10-trideuteromethyl-1,1,1-trifluoro-2-trifluoromethyl-undec-3-yne-2,10-diol (111)

A 100 ml round bottom flask equipped with stir bar and rubber septum was charged with (6R)-6-[(1R,3aR,4S,7aR)-4-(tert-butyl-dimethyl-silanyloxy)-7a-methyl-octahydro-inden-1-yl]-6-methyl-11,11,11-trideutero-10-trideuteromethyl-1,1,1-trifluoro-2-trifluoromethyl-10-trimethylsilanyloxy-undec-3-yn-2-ol (5.56 g), acetonitrile (48 ml) and tetrahydrofuran (12 ml). A solution of H₂SiF₆ (35%) was added in small portion: 5 ml, 2 ml (after 1 h 20 min), 4 ml (after 50 min), 5 ml (after 1 h 40 min), 5 ml (after 1 h 30 min), 5 ml (after 16 h). After next 5 h the resulting mixture was diluted with water (50 ml) and poured into a mixture of ethyl acetate (50 ml) and water (50 ml). The organic phase was collected and the aqueous phase was re-extracted with ethyl acetate (2×50 ml). The combined organic layers were dried (Na₂SO₄) and evaporated. The oil residue was chromatographed on column (450 cm³) using dichloromethane:ethyl acetate (5:1) as mobile phase. The mixture fractions were purified on column (VersaPak Cartridge 40×150 mm) using hexane:ethyl acetate—2:1 and 1:1 as mobile phase. Fractions containing product were pooled and evaporated to give product (3.303 g, 84% two steps).

[α]_{D}³⁰=+1.4 (c=0.59, EtOH)

¹H NMR (CDCl₃): 4.09 (1H, br s), 2.16 (1H, AB, J=17.2 Hz), 2.23 (1H, AB, J=17.2 Hz), 2.05-2.01 (1H, m), 1.85-1.76 (2H, m), 1.65-1.21 (18H, m), 1.06 (3H, s), 1.01 (3H, s)

¹³C NMR (CDCl₃): 121.35 (q, J=286.0 Hz), 90.34, 72.39, 71.06 (sep, J=32.6 Hz), 69.48, 56.99, 52.48, 43.51, 43.13, 40.91, 40.39, 39.97, 33.35, 30.05, 22.54, 22.14, 21.92, 18.09, 17.47, 16.10

| | | | | |
|---|---|---|---|---|
| MS HRES | Calculated for: | C₂₄H₃₀D₆F₆O₃ | [M + Na]⁺ | 515.2837 |
| | Observed: | | [M + Na]⁺ | 515.2836 |

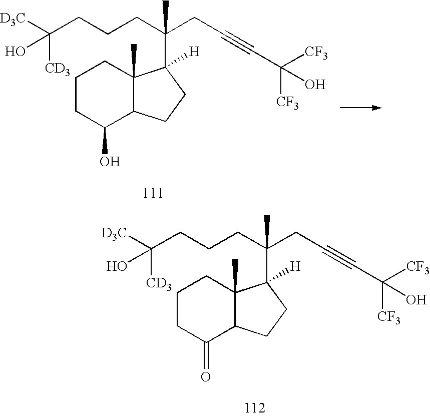

### (1R,3aR,7aR)-7a-Methyl-1-[(1R)-6,6,6-trifluoro-5-hydroxy-1-methyl-1-(5,5,5-trideutero-4-hydroxy-4-trideuteromethyl-pentyl)-5-trifluoromethyl-hex-3-ynyl]-octahydro-inden-4-one (112)

A 25 ml round bottom flask equipped with stir bar and Claisen adapter with rubber septum was charged with pyridinium dichromate (1.620 g, 4.306 mmol) and dichloromethane (15 ml). The (6R)-6-[(1R,3aR,4S,7aR)-4-hydroxy-7a-methyl-octahydro-inden-1-yl]-6-methyl-11,11,11-trideutero-10-trideuteromethyl-1,1,1-trifluoro-2-trifluoromethyl-undec-3-yne-2,10-diol (783 mg, 1.583 mmol) in dichloromethane (2 ml) and DMF (0.5 ml) was added dropwise and mixture was stirred in room temperature for 5 h. The reaction mixture was filtrated through column with silica gel (50 cm³) using dichloromethane, dichloromethane:ethyl acetate 4:1. The fractions containing product were pooled and evaporated to give product as yellow oil. The oil residue was used to next reaction.

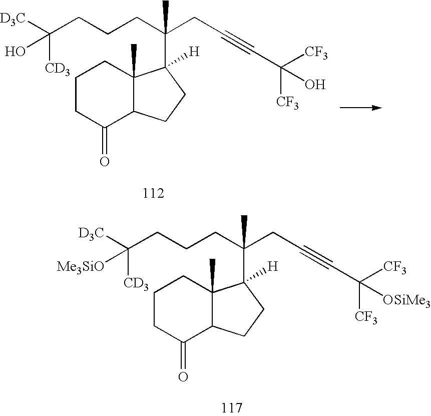

### (1R,3aR,7aR)-7a-Methyl-1-[(1R)-6,6,6-trifluoro-1-methyl-1-(5,5,5-trideutero-4-trideuteromethyl-4-trimethylsilanyloxy-pentyl)-5-trifluoromethyl-5-trimethylsilanyloxy-hex-3-ynyl]-octahydro-inden-4-one (117)

A 25 ml round bottom flask equipped with stir bar and Claisen adapter with rubber septum was charged with (1R,3aR,7aR)-7a-methyl-1-[(1R)-6,6,6-trifluoro-5-hydroxy-1-methyl-1-(5,5,5-trideutero-4-hydroxy-4-trideuteromethyl-pentyl)-5-trifluoromethyl-hex-3-ynyl]-octahydro-inden-4-one (ca. 1.58 mmol) and dichloromethane (8 ml). 1-trimethylsilyl)imidazole (1.90 ml, 12.95 mmol) was added dropwise. The mixture was stirred at room temperature for 1.5 h. Hexane (150 ml) was added and the mixture was washed with water (3×50 ml), dried (Na₂SO₄) and evaporated. The oil residue was chromatographed on column (50 cm³) using hexane:ethyl acetate—5:1 as mobile phase. Fractions containing product were pooled and evaporated to give product as colorless oil (918 mg, 95%).

[α]_{D}³⁰=−20.8 (c=0.61, DMSO)

¹H NMR (CDCl₃): 2.41 (1H, dd, J=11.3, 7.2 Hz), 2.31-2.12 (4H, m), 2.05-1.24 (15H, m), 1.00 (3H, s), 0.73 (3H, s), 0.27 (9H, s), 0.10 (9H, s)

| | | | | |
|---|---|---|---|---|
| MS HRES | Calculated for: | C₃₀H₄₄D₆F₆O₃Si₂ | [M + Na]⁺ | 657.3471 |
| | Observed: | | [M + Na]⁺ | 657.3467 |

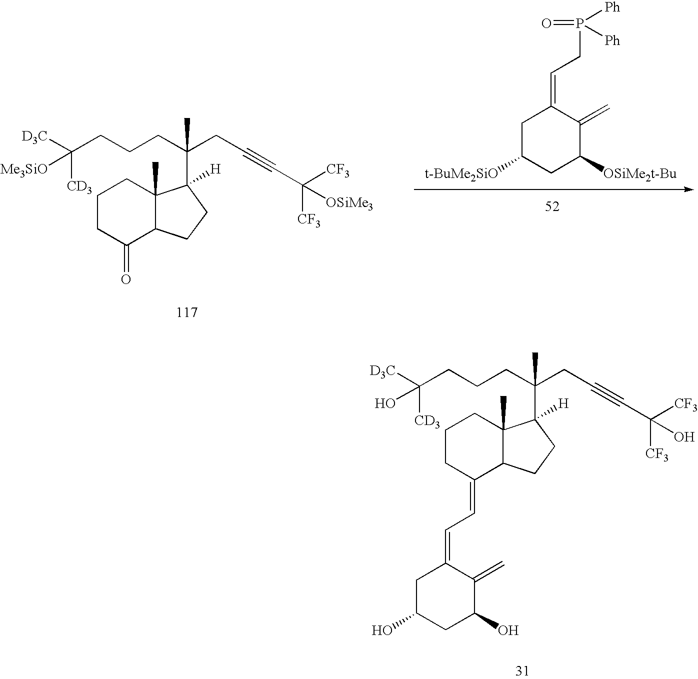

### 1,25-Dihydroxy-20R-20-(4-hydroxy-5,5,5-trideutero-4-trideuteromethyl-pentyl)-23-yne-26,27-hexafluorocholecalciferol (31)

A 25 ml round bottom flask equipped with stir bar and Claisen adapter with rubber septum was charged with (1S,5R)-1,5-bis-((tert-butyldimethyl)silanyloxy)-3-[2-(diphenylfosphinoyl)-eth-(Z)-ylidene]-2-methylene-cyclohexane (500 mg, 0.858 mmol) and tetrahydrofuran (8 ml). The reaction mixture was cooled to −70° C. and n-butyllithium (0.53 ml, 0.85 mmol)) was added dropwise. The resulting deep red solution was stirred at −70° C. for 20 min and (1R,3aR,7aR)-7a-Methyl-1-[(1R)-6,6,6-trifluoro-1-methyl-1-(5,5,5-trideutero-4-trideuteromethyl-4-trimethylsilanyloxy-pentyl)-5-trifluoromethyl-5-trimethylsilanyloxy-hex-3-ynyl]-octahydro-inden-4-one (314 mg, 0.495 mmol) was added dropwise in tetrahydrofuran (1.5 ml). The reaction mixture was stirred for 8 h (in last hour the temperature was increased from −70 do −50° C.). Saturated solution of ammonium chloride (1 ml) was added and the bath was removed. The mixture was poured into ethyl acetate (50 ml) and saturated solution of ammonium chloride (50 ml). The water fraction was extracted with ethyl acetate (3×60 ml), dried (Na₂SO₄) and evaporated. The oil residue was chromatographed on column (50 cm³, protected from light) using hexane:ethyl acetate—10:1 as mobile phase. Fractions containing product and some mono deprotected compound were pooled and evaporated to give oil.

A 25 ml round bottom flask equipped with stir bar and Claisen adapter with rubber septum was charged with substrate and tetrabutylammonium fluoride (10 ml, 1M/tetrahydrofuran). The mixture was stirred for next 41 h. The mixture was dissolved by the addition of ethyl acetate (150 ml) and extracted 6 times with water and brine (30 ml+20 ml), dried Na₂SO₄) and evaporated. The oil residue was chromatographed on column (70 cm³, protected from light) using ethyl acetate as mobile phase. Fraction containing impurity was chromatographed on next column (70 cm³, protected from light) using ethyl acetate as mobile phase. Fractions containing product were pooled and evaporated to give product as colorless oil. Oil was dissolved in methyl acetate and evaporated (4 times) to give product as white foam (198 mg, 64%).

[α]_{D}²⁸=+11.0 (c=0.50, EtOH)

UV λmax (EtOH): 213 nm (ε 17813), 264 nm (ε 20804)

¹H NMR (DMSO-D6): 8.95 (1H, s), 6.19 (1H, d, J=11.3 Hz), 5.97 (1H, d, J=11.3 Hz), 5.22 (1H, s), 4.86 (1H, d, J=4.9 Hz), 4.75 (1H, d, J=1.9 Hz), 4.55 (1H, d, J=3.8 Hz), 4.20-4.18 (1H, m), 4.04 (1H, s), 4.01-3.98 (1H, m), 2.78 (1H, d, J=13.6 Hz), 2.35 (1H, d, J=13.4 Hz), 2.28-2.14 (3H, m), 1.99-1.92 (2H, m), 1.83-1.78 (2H, m), 1.64-1.57 (5H, m), 1.47-1.21 (10H, m), 0.96 (3H, s), 0.60 (3H, s)

¹³C NMR (DMSO)-D6): 149.56, 139.66, 136.09, 122.45, 121.61 (q, J=286.7 Hz), 118.13, 109.87, 89.59, 70.67, 70.46 (sep, J=31.9 Hz), 68.48, 68.42, 65.13, 56.05, 55.96, 46.09, 44.88, 44.55, 43.13, 40.12, 38.88, 28.77, 28.31, 23.03, 22.37, 21.89, 21.51, 18.21, 14.25

| | | | | |
|---|---|---|---|---|
| MS HRES | Calculated for: | C₃₃H₄₀D₆F₆O₄ | [M + Na]⁺ | 649.3569 |
| | Observed: | | [M + Na]⁺ | 649.3569 |

### Example 32

### Synthesis of 1,25-Dihydroxy-20R-20-(4-hydroxy-5,5,5-trideutero-4-trideuteromethyl-pentyl)-23-yne-26,27-hexafluoro-19-nor-cholecalciferol (32)

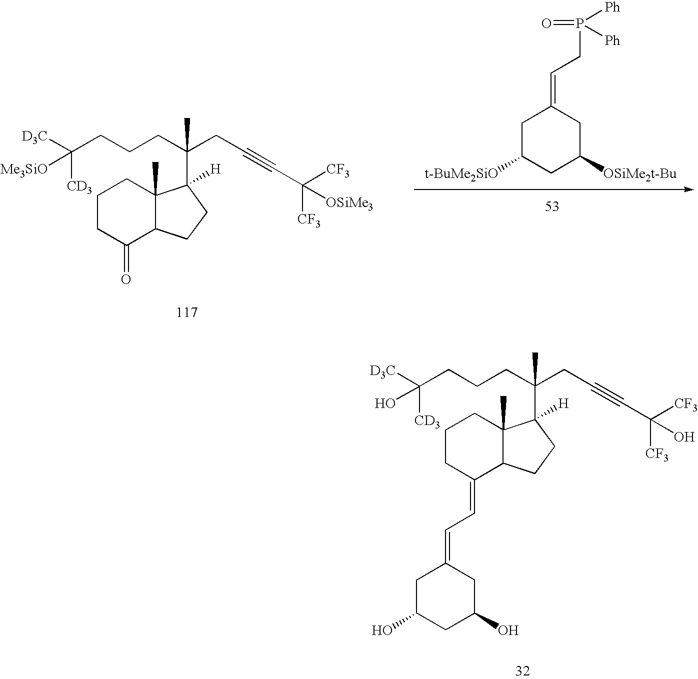

### 1,25-Dihydroxy-20R-20-(4-hydroxy-5,5,5-trideutero-4-trideuteromethyl-pentyl)-23-yne-26,27-hexafluoro-19-nor-cholecalciferol (32)

A 25 ml round bottom flask equipped with stir bar and Claisen adapter with rubber septum was charged with (1R,3R)-1,3-bis-((tert-butyldimethyl)silanyloxy)-5-[2-(diphenylfosphinoyl)ethylidene]-cyclohexane (568 mg, 0.995 mmol) and tetrahydrofuran (8 ml). The reaction mixture was cooled to −70° C. and n-butyllithium (0.62 ml, 0.99 mmol) was added dropwise. The resulting deep red solution was stirred at −70° C. for 20 min and (1R,3aR,7aR)-7a-Methyl-1-[(1R)-6,6,6-trifluoro-1-methyl-1-(5,5,5-trideutero-4-trideuteromethyl-4-trimethylsilanyloxy-pentyl)-5-trifluoromethyl-5-trimethylsilanyloxy-hex-3-ynyl]-octahydro-inden-4-one (306 mg, 0.482 mmol) was added dropwise in tetrahydrofuran (1.5 ml). The reaction mixture was stirred for 6 h and then saturated solution of ammonium chloride (1 ml) was added and the bath was removed. The mixture was poured into ethyl acetate (50 ml) and saturated solution of ammonium chloride (50 ml). The water fraction was extracted with ethyl acetate (3×50 ml), dried (Na₂SO₄) and evaporated. The oil residue was chromatographed on column (50 cm³, protected from light) using hexane:ethyl acetate—10:1 as mobile phase. Fractions containing product and some mono deprotected compound were pooled and evaporated to give oil. A 25 ml round bottom flask equipped with stir bar and Claisen adapter with rubber septum was charged with substrate and tetrabutylammonium fluoride (15 ml, 1M/tetrahydrofuran). The mixture was stirred for next 96 h.

The mixture was dissolved by the addition of ethyl acetate (150 ml) and extracted 6 times with water and brine (30 ml+20 ml), dried (Na₂SO₄) and evaporated. The oil residue was chromatographed on column (60 cm³, protected from light) using ethyl acetate as mobile phase. Fractions containing product were pooled and evaporated to give product as colorless oil. Oil was dissolved in methyl acetate and evaporated (4 times) to give product as white foam (223 mg, 75%).

[α]_{D}²⁷=+45.5 (c=0.42, EtOH)

UV λmax (EtOH): 244 nm (ε 36685), 252 nm (ε 42933), 262 nm (ε 28904)

¹H NMR (DMSO-D6): 8.95 (1H, s), 6.07 (1H, d, J=11.1 Hz), 5.78 (1H, d, J=11.1 Hz), 4.48 (1H, d, J=4.3 Hz), 4.38 (1H, d, J=3.8 Hz), 4.04 (1H, s), 3.90-3.76 (2H, m), 2.74 (1H, d, J=13.4 Hz), 2.43 (1H, d, J=14.1 Hz), 2.28-2.19 (3H, m), 2.07-1.93 (3H, m), 1.81 (1H, dd, J=9.6, 9.2 Hz), 1.68-1.22 (17H, m), 0.96 (3H, s), 0.59 (3H, s)

¹³C NMR (DMSO-D6): 139.10, 134.88, 121.61 (q, J=286.7 Hz), 120.92, 116.57, 89.60, 70.67, 68.49, 65.60, 65.32, 56.01, 55.94, 45.94, 44.60, 44.55, 42.23, 39.80, 36.96, 28.80, 28.15, 22.89, 22.39, 21.94, 21.42, 18.22, 14.37

| | | | | |
|---|---|---|---|---|
| MS HRES | Calculated for: | C₃₂H₄₀D₆F₆O₄ | [M + Na]⁺ | 637.3569 |
| | Observed: | | [M + Na]⁺ | 637.3565 |

### Example 33

### Synthesis of 1α-Fluoro-25-hydroxy-20R-20-(4-hydroxy-5,5,5-trideutero-4-trideuteromethyl-pentyl)-23-yne-26,27-hexafluorocholecalciferol (33)

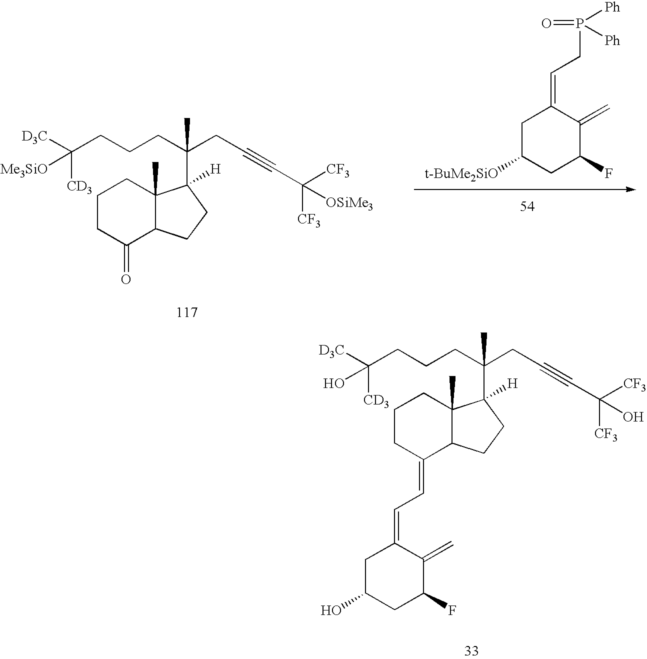

### 1α-Fluoro-25-hydroxy-20R-20-(4-hydroxy-5,5,5-trideutero-4-trideuteromethyl-pentyl)-23-yne-26,27-hexafluorocholecalciferol (33)

A 25 ml round bottom flask equipped with stir bar and Claisen adapter with rubber septum was charged with (1S,5R)-1-((tert-butyldimethyl)silanyloxy)-3-[2-(diphenylfosphinoyl)-eth-(Z)-ylidene]-5-fluoro-2-methylene-cyclohexane] (542 mg, 1.152 mmol) and tetrahydrofuran (8 ml). The reaction mixture was cooled to −70° C. and n-butyllithium (0.71 ml, 1.14 mmol) was added dropwise. The resulting deep red solution was stirred at −70° C. for 20 min and (1R,3aR,7aR)-7a-Methyl-1-[(1R)-6,6,6-trifluoro-1-methyl-1-(5,5,5-trideutero-4-trideuteromethyl-4-trimethylsilanyloxy-pentyl)-5-trifluoromethyl-5-trimethylsilanyloxy-hex-3-ynyl]-octahydro-inden-4-one (292 mg, 0.460 mmol) was added dropwise in tetrahydrofuran (1.5 ml). The reaction mixture was stirred for 7 h (in last hour the temperature was increased from −70 do −50° C.). The bath was removed and the mixture was poured into ethyl acetate (50 ml) and saturated solution of ammonium chloride (50 ml). The water fraction was extracted with ethyl acetate (3×50 ml), dried (Na₂SO₄) and evaporated. The oil residue was chromatographed on column (50 cm³, protected from light) using hexane:ethyl acetate—10:1 as mobile phase. Fractions containing product were pooled and evaporated to give oil. The oil residue was used to next reaction. A 25 ml round bottom flask equipped with stir bar and Claisen adapter with rubber septum was charged with substrate and tetrabutylammonium fluoride (8 ml, 1M/tetrahydrofuran). The mixture was stirred for next 48 h. The mixture was dissolved by the addition of ethyl acetate (150 ml) and extracted 6 times with water and brine (30 ml+20 ml), dried (Na₂SO₄) and evaporated. The oil residue was chromatographed on column (50 cm³, protected from light) using hexane:ethyl acetate—1:1 as mobile phase. Fractions containing product were pooled and evaporated to give product as colorless oil. Oil was dissolved in methyl acetate and evaporated (4 times) to give product as white foam (278 mg, 96%);

[α]_{D}²⁷=+26.4 (c=0.50, EtOH)

UV λmax (EtOH): 210 nm (ε 14823), 244 nm (ε 14731), 270 nm (ε 14798)

¹H NMR (DMSO-D6): 8.95 (1H, s), 6.36 (1H, d, J=11.1 Hz), 5.93 (1H, d, J=11.3 Hz), 5.38 (1H, s), 5.14 (1H, br d, J=49.6 Hz), 4.98 (1H, d, J=1.9 Hz), 4.86 (1H, d, J=4.5 Hz), 4.04 (1H, s), 3.94-3.87 (1H, m), 2.82 (1H, d, J=10.2 Hz), 2.27-2.05 (4H, m), 2.00-1.93 (2H, m), 1.83-1.55 (7H, m), 1.48-1.21 (10H, m), 0.95 (3H, s), 0.58 (3H, s)

¹³C NMR (DMSO-D6): 143.31 (d, J=16.7 Hz), 141.67, 133.23 (d, J=1.5 Hz), 124.18, 121.64 (q, J=286.0 Hz), 117.53, 115.37 (d, J=9.2 Hz), 92.09 (167.6 Hz), 89.59, 70.70, 70.48 (sep, J=31.9 Hz), 68.51, 64.61, 64.57, 56.02, 55.96, 46.19, 44.86, 44.56, 40.71 (d, J=19.7 Hz), 39.82, 28.80, 28.34, 22.98, 22.35, 21.90, 21.43, 18.24, 14.31

| | | | | |
|---|---|---|---|---|
| MS HRES | Calculated for: | C₃₃H₃₉D₆F₇O₃ | [M + Na]⁺ | 651.3526 |
| | Observed: | | [M + Na]⁺ | 651.3530 |

### Example 34

### Synthesis of 1,25-Dihydroxy-20R-20-(4-hydroxy-5,5,5-trideutero-4-trideuteromethyl-pentyl)-23Z-ene-26,27-hexafluorocholecalciferol (25)

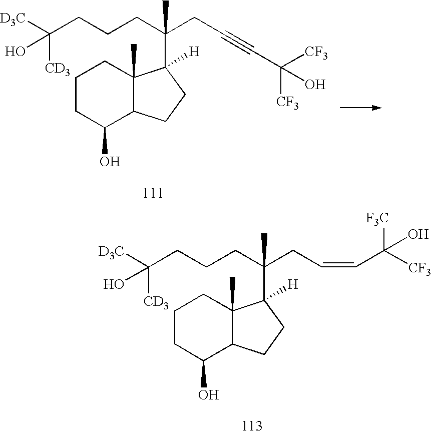

### (6R,3Z)-6-[(1R,3aR,4S,7aR)-4-hydroxy-7a-methyl-octahydro-inden-1-yl]-6-methyl-11,11,11-trideutero-10-trideuteromethyl-1,1,1-trifluoro-2-trifluoromethyl-undec-3-ene-2,10-diol (113)

A 50 ml round bottom flask was charged with (6R)-6-[(1R,3aR,4S,7aR)-4-hydroxy-7a-methyl-octahydro-inden-1-yl]-6-methyl-11,11,11-trideutero-10-trideuteromethyl-1,1,1-trifluoro-2-trifluoromethyl-undec-3-yne-2,10-diol (800 mg, 1.624 mmol), Pd/CaCO₃ (200 mg, 5%), hexane (18.6 ml), ethyl acetate (7.6 ml) and solution of quinoline in ethanol (0.72 ml, prepared from ethanol (3.1 ml) and quinoline (168 μl)). The substrate was hydrogenated at ambient temperature and atmospheric pressure of hydrogen. The reaction was monitoring by TLC (dichloromethane:ethyl acetate 4:1, 3×). After 5 h 10 min the catalyst was filtered off (silica gel 50 cm³, hexane:ethyl acetate 1:1) and solvent evaporated. Product was crystallized from hexane:ethyl acetate (750 mg, 93%).

[α]_{D}³⁰=−2.34 (c=0.47, EtOH)

¹H NMR (CDCl₃): 6.07 (1H, dt, J=12.4, 7.2 Hz), 5.45 (1H, d, J=12.4 Hz), 4.08 (1H, d, J=2.1 Hz), 2.50-2.39 (2H, m), 2.03 (1H, d, J=11.1 Hz), 1.88-1.79 (2H, m), 1.67-1.22 (18H, m), 1.09 (3H, s), 0.98 (3H, s)

¹³C NMR (CDCl₃): 139.98, 122.83 (q, J=286.7 Hz), 117.24, 71.45, 69.57, 56.67, 52.55, 44.08, 43.56, 41.21, 39.71, 39.13, 37.19, 33.39, 22.42, 22.15, 21.86, 17.92, 17.54, 16.47

| | | | | |
|---|---|---|---|---|
| MS HRES | Calculated for: | C₂₄H₃₂D₆F₆O₃ | [M + Na]⁺ | 517.2994 |
| | Observed: | | [M + Na]⁺ | 517.2992 |

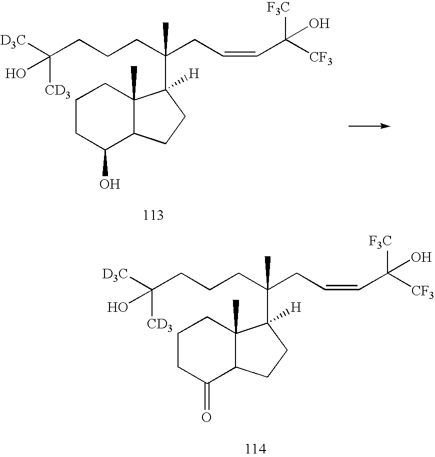

### (1R,3aR,7aR)-7a-Methyl-1-[(1R,3Z)-6,6,6-trifluoro-5-hydroxy-1-methyl-1-(5,5,5-trideutero-4-hydroxy-4-trideuteromethyl-pentyl)-5-trifluoromethyl-hex-3-enyl]-octahydro-inden-4-one (114)

A 50 ml round bottom flask equipped with stir bar and Claisen adapter with rubber septum was charged with pyridinium dichromate (1.520 g, 4.040 mmol) and dichloromethane (20 ml). The (6R,3Z)-6-[(1R,3aR,4S,7aR)-4-hydroxy-7a-methyl-octahydro-inden-1-yl]-6-methyl-11,11,11-trideutero-10-trideuteromethyl-11,11,11-trifluoro-2-trifluoromethyl-undec-3-ene-2,10-diol (730 mg, 1.476 mmol) in dichloromethane (5 ml) was added dropwise and mixture was stirred in room temperature for 4 h 20 min. The reaction mixture was filtrated through column with silica gel (50 cm³) using dichloromethane, dichloromethane:ethyl acetate 4:1. The fractions containing product were pooled and evaporated. The product was used to the next reaction without purification.

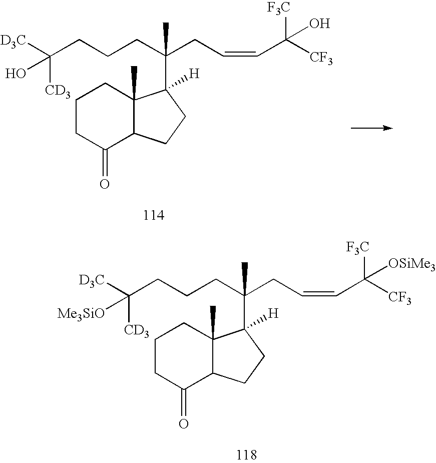

### (1R,3aR,7aR)-7a-Methyl-1-[(1R,3Z)6,6,6-trifluoro-1-methyl-1-(5,5,5-trideutero-4-trideuteromethyl-4-trimethylsilanyloxy-pentyl)-5-trifluoromethyl-5-trimethylsilanyloxy,-hex-3-enyl]-octahydro-inden-4-one (118)

A 25 ml round bottom flask equipped with stir bar and Claisen adapter with rubber septum was charged with (1R,3aR,7aR)-7a-methyl-1-[(1R,3Z)-6,6,6-trifluoro-5-hydroxy-1-methyl-1-(5,5,5-trideutero-4-hydroxy-4-trideuteromethyl-pentyl)-5-trifluoromethyl-hex-3-enyl]-octahydro-inden-4-one (ca. 1.47 mmol) and dichloromethane (8 ml). 1-(trimethylsilyl)imidazole (1.80 ml, 12.27 mmol) was added dropwise. The mixture was stirred at room temperature for 3 h. Water (50 ml) was added and the mixture was extracted with ethyl acetate (3×50 ml), dried (Na₂SO₄) and evaporated. The oil residue was chromatographed on column (75 cm³) using hexane:ethyl acetate—5:1 as mobile phase. Fractions containing product were pooled and evaporated to give product as colorless oil (766 mg, 81%)

¹H NMR (CDCl₃): 5.98 (1H, dt, J=12.5, 6.2 Hz), 5.42 (1H, d, J=11.4 Hz), 2.49-2.40 (2H, m), 2.34-2.15 (4H, m), 2.07-1.95 (1H, m), 1.93-1.60 (6H, m), 1.43-1.19 (7H, m), 0.95 (3H, s), 0.74 (3H, s), 0.24 (9H, s), 0.10 (9H, s)

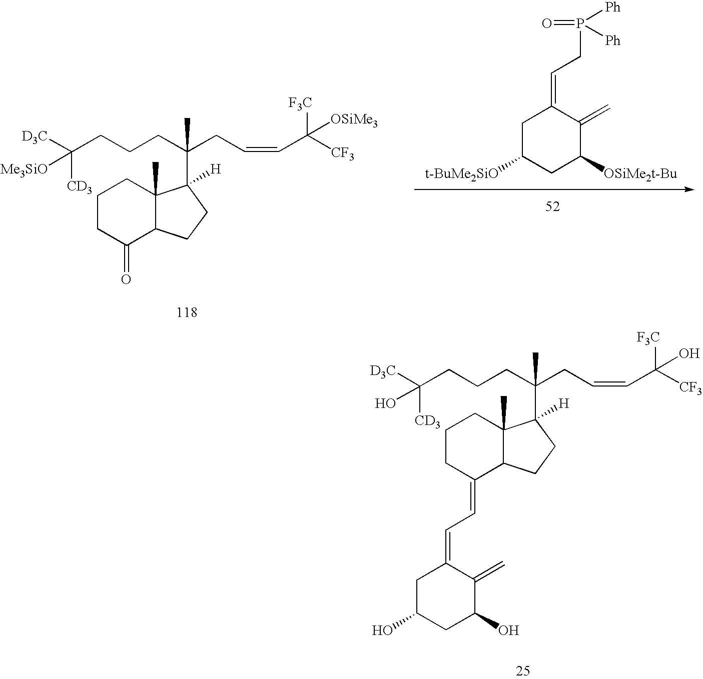

### 1,25-Dihydroxy-20R-20-(4-hydroxy-5,5,5-trideutero-4-trideuteromethyl-pentyl)-23Z-ene-26,27-hexafluorocholecalciferol (25)

A 25 ml round bottom flask equipped with stir bar and Claisen adapter with rubber septum was charged with (1S,5R)-1,5-bis-((tert-butyldimethyl)silanyloxy)-3-[2-(diphenylfosphinoyl)-eth-(Z)-ylidene]-2-methylene-cyclohexane (473 mg, 0.811 mmol) and tetrahydrofuran (8 ml). The reaction mixture was cooled to −70° C. and n-butyllithium (0.50 ml, 0.80 mmol)) was added dropwise. The resulting deep red solution was stirred at −70° C. for 20 min and (1R,3aR,7aR)-7a-methyl-1-[(1R,3Z)6,6,6-trifluoro-1-methyl-1-(5,5,5-trideutero-4-trideuteromethyl-4-trimethylsilanyloxy-pentyl)-5-trifluoromethyl-5-trimethylsilanyloxy-hex-3-enyl]-octahydro-inden-4-one (280 mg, 0.440 mmol) was added dropwise in tetrahydrofuran (1.5 ml). The reaction mixture was stirred for 6 h (in last hour the temperature was increased from −70 do −50° C.). Saturated solution of ammonium chloride (1 ml) was added and the bath was removed. The mixture was poured into ethyl acetate (50 ml) and saturated solution of ammonium chloride (100 ml). The water fraction was extracted with ethyl acetate (3×70 ml), dried (Na₂SO₄) and evaporated. The oil residue was chromatographed on column (50 cm³, protected from light) using hexane:ethyl acetate—10:1 as mobile phase. Fractions containing product and some mono deprotected compound were pooled and evaporated to give colorless oil. A 25 ml round bottom flask equipped with stir bar and Claisen adapter with rubber septum was charged with substrate and tetrabutylammonium fluoride (15 ml, 1M/tetrahydrofuran). The mixture was stirred for next 29 h. The mixture was dissolved by the addition of ethyl acetate (150 ml) and extracted 6 times with water and brine (30 ml+20 ml), dried (Na₂SO₄) and evaporated. The oil residue was chromatographed on column (50 cm³, protected from light) using hexane:ethyl acetate—1:2 as mobile phase. Fractions containing product were pooled and evaporated to give product as colorless oil. Oil was dissolved in methyl acetate and evaporated (4 times) to give product as white foam (224 mg, 81%).

[α]_{D}²⁹=+7.5 (c=0.48, EtOH)

UV λmax (EtOH): 213 nm (ε 15024), 265 nm (ε 17330)

¹H NMR (DMSO-D6): 7.98 (1H, s), 6.18 (1H, d, J=11.1 Hz), 6.10 (1H, dt, J=12.8, 6.4 Hz), 5.97 (1H, d, J=11.3 Hz), 5.43 (1H, d, J=11.9 Hz), 5.23 (1H, s), 4.86 (1H, d, J=4.7 Hz), 4.75 (1H, d, J=1.7 Hz), 4.54 (1H, d, J=3.6 Hz), 4.21-4.16 (1H, m), 4.02 (1H, s), 4.05-3.95 (1H, m), 2.77 (1H, d, J=11.7 Hz), 2.50-2.29 (2H, m), 2.16 (1H, dd, J=13.5, 5.2 Hz), 2.00-1.94 (2H, m), 1.82-1.78 (1H, m), 1.71-1.25 (17H, m), 0.90 (3H, s), 0.61 (3H, s)

¹³C NMR (DMSO-D6): 149.40, 139.76, 139.25, 135.81, 122.93 (q, J=287.5 Hz), 122.35, 117.88, 117.11, 109.75, 76.78 (sep, J=29.6 Hz), 68.41, 68.35, 65.07, 56.55, 55.98, 46.15, 44.86, 44.59, 43.11, 40.34, 38.76, 36.05, 28.98, 23.13, 22.80, 21.83, 29.50, 20.07, 17.93, 14.57

| | | | | |
|---|---|---|---|---|
| MS HRES | Calculated for: | C₃₃H₄₂D₆F₆O₄ | [M + Na]⁺ | 651.3725 |
| | Observed: | | [M + Na]⁺ | 651.3726 |

### Example 35

### Synthesis of 1,25-Dihydroxy-20R-20-(4-hydroxy-5,5,5-trideutero-4-trideuteromethyl-pentyl)-23Z-ene-26,27-hexafluoro-19-nor-cholecalciferol (26)

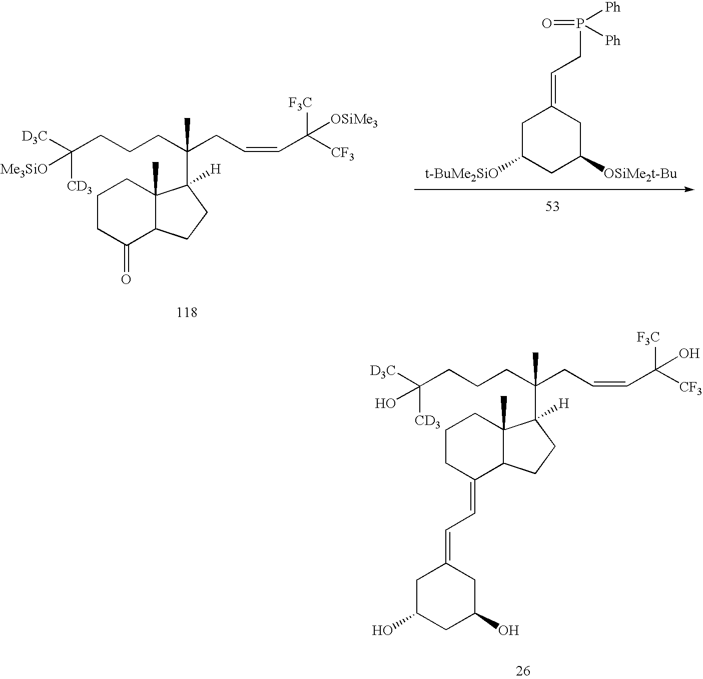

### 1,25-Dihydroxy-20R-20-(4-hydroxy-5,5,5-trideutero-4-trideuteromethyl-pentyl)-23Z-ene-26,27-hexafluoro-19-nor-cholecalciferol (26)

A 25 ml round bottom flask equipped with stir bar and Claisen adapter with rubber septum was charged with (1R,3R)-1,3-bis-((tert-butyldimethyl)silanyloxy)-5-[2-(diphenylfosphinoyl)ethylidene]-cyclohexane (575 mg, 1.007 mmol) and tetrahydrofuran (8 ml). The reaction mixture was cooled to −70° C. and n-butyllithium (0.61 ml, 0.98 mmol)) was added dropwise. The resulting deep red solution was stirred at −70° C. for 20 min and (1R,3aR,7aR)-7a-methyl-1-[(1R,3Z)6,6,6-trifluoro-1-methyl-1-(5,5,5-trideutero-4-trideuteromethyl-4-trimethylsilanyloxy-pentyl)-5-trifluoromethyl-5-trimethylsilanyloxy-hex-3-enyl]-octahydro-inden-4-one (303 mg, 0.476 mmol) was added dropwise in tetrahydrofuran (1.5 ml). The reaction mixture was stirred for 5 h and then saturated solution of ammonium chloride (1 ml) was added and the bath was removed. The mixture was poured into ethyl acetate (50 ml) and saturated solution of ammonium chloride (100 ml). The water fraction was extracted with ethyl acetate (3×70 ml), dried (Na₂SO₄) and evaporated. The oil residue was chromatographed on column (50 cm³, protected from light) using hexane:ethyl acetate—10:1 as mobile phase. Fractions containing product and some mono deprotected compound were pooled and evaporated to give colorless oil. A 25 ml round bottom flask equipped with stir bar and Claisen adapter with rubber septum was charged with substrate and tetrabutylammonium fluoride (15 ml, 1M/tetrahydrofuran). The mixture was stirred for next 64 h. The mixture was dissolved by the addition of ethyl acetate (150 ml) and extracted 6 times with water and brine (30 ml+20 ml), dried Na₂SO₄) and evaporated. The oil residue was chromatographed on column (60 cm³, protected from light) using ethyl acetate as mobile phase. Fractions containing product were pooled and evaporated to give product as colorless oil. Oil was dissolved in methyl acetate and evaporated (4 times) to give product as white foam (251 mg, 85%).

[α]_{D}²⁹=+44.3 (c=0.42, EtOH)

UV λmax (EtOH): 244 nm (ε 36100), 252 nm (ε 42319), 262 nm (s 28518)

¹H NMR (DMSO-D6): 7.99 (1H, s), 6.14-6.06 (1H, m), 6.07 (1H, d, J=12.4 Hz), 5.78 (1H, d, J=11.3 Hz), 5.43 (1H, d, J=12.2 Hz), 4.48 (1H, d, J=4.0 Hz), 4.38 (1H, d, J=4.1 Hz), 4.02 (1H, s), 3.90-3.84 (1H, m), 3.84-3.76 (1H, m), 2.73 (1H, d, J=13.6 Hz), 2.54-2.41 (2H, m), 2.26 (1H, br d, J=10.4 Hz), 2.07-1.97 (3H, m), 1.72-1.18 (19H, m), 0.90 (3H, s), 0.60 (3H, s)

¹³C NMR (DMSO-D6): 139.25, 139.18, 134.60, 122.94 (q, J=286.8 Hz), 120.82, 117.13, 116.33, 76.77 (sep, J=28.0 Hz), 68.41, 65.54, 65.26, 56.53, 55.95, 46.00, 44.59, 42.22, 40.34, 38.78, 36.96, 36.07, 28.17, 22.99, 22.80, 21.89, 21.40, 17.94, 14.67

| | | | | |
|---|---|---|---|---|
| MS HRES | Calculated for: | C₃₂H₄₂D₆F₆O₄ | [M + Na]⁺ | 639.3725 |
| | Observed: | | [M + Na]⁺ | 639.3717 |

### Example 36

### Synthesis of 1α-Fluoro-25-hydroxy-20R-20-(4-hydroxy-5,5,5-trideutero-4-trideuteromethyl-pentyl)-23Z-ene-26,27-hexafluorocholecalciferol (27)

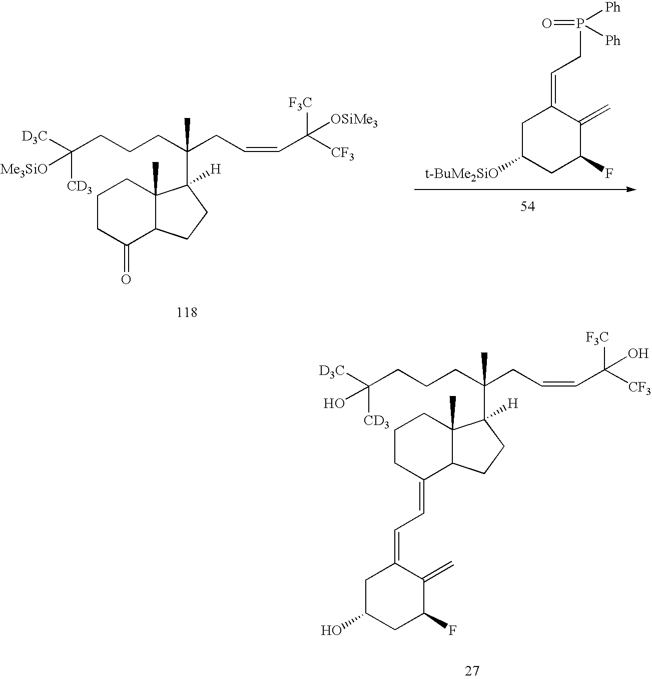

### 1α-Fluoro-25-hydroxy-20R-20-(4-hydroxy-5,5,5-trideutero-4-trideuteromethyl-pentyl)-23Z-ene-26,27-hexafluorocholecalciferol (27)

A 25 ml round bottom flask equipped with stir bar and Claisen adapter with rubber septum was charged with (1S,5R)-1-((tert-butyldimethyl)silanyloxy)-3-[2-(diphenylfosphinoyl)-eth-(Z)-ylidene]-5-fluoro-2-methylene-cyclohexane (520 mg, 1.105 mmol) and tetrahydrofuran (8 ml). The reaction mixture was cooled to −70° C. and n-butyllithium (0.69 ml, 1.10 mmol)) was added dropwise. The resulting deep red solution was stirred at −70° C. for 20 min and (1R,3aR,7aR)-7a-Methyl-1-[(1R,3Z)6,6,6-trifluoro-1-methyl-1-(5,5,5-trideutero-4-trideuteromethyl-4-trimethylsilanyloxy-pentyl)-5-trifluoromethyl-5-trimethylsilanyloxy-hex-3-enyl]-octahydro-inden-4-one (314 mg, 0.493-mmol) was added dropwise in tetrahydrofuran (1.5 mil). The reaction mixture was stirred for 5 h 30 min (in last hour the temperature was increased from −70 do −50° C.). The bath was removed and the mixture was poured into ethyl acetate (50 ml) and saturated solution of ammonium chloride (100 ml). The water fraction was extracted with ethyl acetate (3×50 ml), dried (Na₂SO₄) and evaporated. The oil residue was chromatographed on column (50 cm³, protected from light) using hexane:ethyl acetate—10:1 as mobile phase. Fractions containing product were pooled and evaporated to give colorless oil. The oil residue was used to next reaction. A 25 ml round bottom flask equipped with stir bar and Claisen adapter with rubber septum was charged with substrate and tetrabutylammonium fluoride (10 ml, 1M/tetrahydrofuran). The mixture was stirred for next 22 h. The mixture was dissolved by the addition of ethyl acetate (150 ml) and extracted 6 times with water and brine (30 ml+20 ml), dried (Na₂SO₄) and evaporated. The oil residue was chromatographed on column (50 cm³, protected from light) using hexane:ethyl acetate—1:1 as mobile phase. Fractions containing product and impurity were purified on column (50 cm³, protected from light) using hexane:ethyl acetate—2:1 and 1:1 as mobile phase. Fractions containing product were pooled and evaporated to give product as colorless oil. Oil was dissolved in methyl acetate and evaporated (4 times) to give product as white foam (258 mg, 83%).

[α]_{D}²³=+25.0 (c=0.44, EtOH)

UV λmax (EtOH): 210 nm (ε 15800), 245 nm (ε 15638), 269 nm (ε 15445)

¹H NMR (DMSO-D6): 7.99 (1H, s), 6.36 (1H, d, J=11.3 Hz), 6.10 (1H, dt, J=11.9, 6.3 Hz), 5.92 (1H, d, J=11.3 Hz), 5.43 (1H, d, J=12.4 Hz), 5.39 (1H, s), 5.14 (1H, ddd, J=49.4, 5.5, 3.7 Hz), 4.98 (1H, d, J=1.7 Hz), 4.85 (1H, d, J=4.5 Hz), 4.02 (1H, s), 3.93-3.87 (1H, m), 2.81 (1H, d, J=12.8 Hz), 2.54-2.40 (2H, m), 2.16-1.97 (4H, m), 1.82-1.17 (17H, m), 0.89 (3H, s), 0.59 (3H, s)

¹³C NMR (DMSO-D6): 143.13 (d, J=16.7 Hz), 141.74, 139.20, 132.94, 124.06, 122.93 (q, J=286.0 Hz), 117.26, 117.12, 115.18 (d, J=9.1 Hz), 91.95 (d, J=166.9 Hz), 76.78 (sep, J=28.8 Hz), 68.41, 64.54, 65.50, 56.51, 55.92, 46.24, 44.81, 44.58, 40.68 (d, J=20.5 Hz), 40.28, 38.97, 38.78, 36.07, 28.33, 23.06, 22.74, 21.83, 21.40, 17.93, 14.59

| | | | | |
|---|---|---|---|---|
| MS HRES | Calculated for: | C₃₃H₄₁D₆F₇O₃ | [M + Na]⁺ | 653.3682 |
| | Observed: | | [M + Na]⁺ | 653.3686 |

### Example 37

### Synthesis of 1,25-Dihydroxy-20R-20-(4-hydroxy-5,5,5-trideutero-4-trideuteromethyl-pentyl)-23E-ene-26,27-hexafluorocholecalciferol (28)

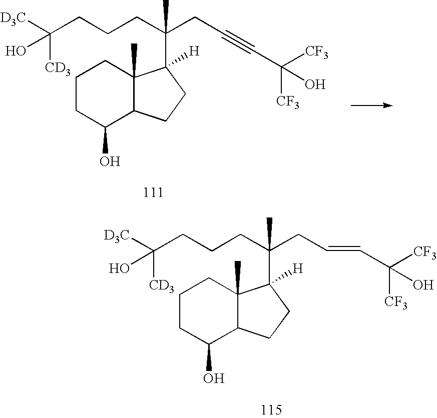

### (6R,3E)-6-[(1R,3aR,4S,7aR)-4-hydroxy-7a-methyl-octahydro-inden-1-yl]-6-methyl-11,11,11-trideutero-10-trideuteromethyl-1,1,1-trifluoro-2-trifluoromethyl-undec-3-ene-2,10-diol (115)

A 25 ml round bottom flask equipped with stir bar and condenser with nitrogen sweep was charged with lithium aluminum hydride (13.00 ml, 13.00 mmol, 1M/tetrahydrofuran) and the mixture was cooled to 0° C. Sodium methoxide (702 mg, 13.00 mmol) was added slowly followed by (6R)-6-[(1R,3aR,4S,7aR)-4-hydroxy-7a-methyl-octahydro-inden-1-yl]-6-methyl-11,11,11-trideutero-10-trideuteromethyl-1,1,1-trifluoro-2-trifluoromethyl-undec-3-yne-2,10-diol (810 mg, 1.665 mmol) in tetrahydrofuran (8 ml). The reaction mixture was stirred at 80° C. for 6.5 h and then was cooled to 0° C. Saturated solution of ammonium chloride (5 ml) was added slowly followed by saturated solution of ammonium chloride (60 ml) and 2N HCl (20 ml). The mixture was extracted with ethyl acetate (3×50 ml), dried (Na₂SO₄) and evaporated. The oil residue was chromatographed on columns (75 cm³) using hexane:ethyl acetate—2:1 and 1:1 as mobile phase. Fractions containing product were pooled and evaporated to give colorless oil (806 mg, 98%).

¹H NMR (CDCl₃): 6.28 (1H, dt, J=15.4, 7.7 Hz), 5.59 (1H, d, J=15.7 Hz), 4.08 (1H, br s), 2.13-2.00 (3H, m), 1.83-1.79 (2H, m), 1.63-1.24 (18H, m), 1.08 (3H, s), 0.97 (3H, s)

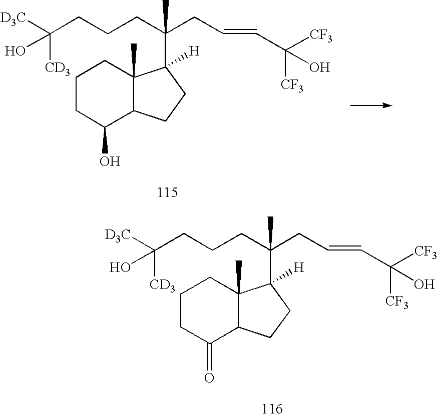

### (1R,3aR,7aR)-7a-Methyl-1-[(1R,3E)6,6,6-trifluoro-5-hydroxy-1-methyl-1-(5,5,5-trideutero-4-hydroxy-4-trideuteromethyl-pentyl)-5-trifluoromethyl-hex-3-enyl]-octahydro-inden-4-one (116)

A 25 ml round bottom flask equipped with stir bar and Claisen adapter with rubber septum was charged with pyridinium dichromate (1.600 g, 4.253 mmol) and dichloromethane (15 ml). The (6R,3E)-6-[(1R,3aR,4S,7aR)-4-hydroxy-7a-methyl-octahydro-inden-1-yl]-6-methyl-11,11,11-trideutero-10-trideuteromethyl-1,1,1-trifluoro-2-trifluoromethyl-undec-3-ene-2,10-diol (782 mg, 1.581 mmol) in dichloromethane (2 ml) was added dropwise and mixture was stirred in room temperature for 4 h 30 min. The reaction mixture was filtrated through column with silica gel (25 cm³) using dichloromethane, dichloromethane:ethyl acetate 4:1. The fractions containing product were pooled and evaporated to give product as colorless oil (746 mg, 96%).

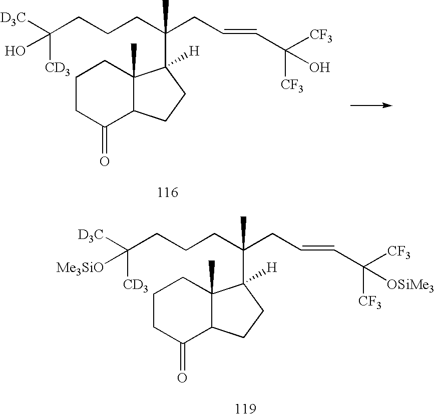

### (1R,3aR,7aR)-7a-Methyl-1-[(1R,3E)-6,6,6-trifluoro-1-methyl-1-(5,5,5-trideutero-4-trideuteromethyl-4-trimethylsilanyloxy-pentyl)-5-trifluoromethyl-5-trimethylsilanyloxy-hex-3-enyl]-octahydro-inden-4-one (119)

A 25 ml round bottom flask equipped with stir bar and Claisen adapter with rubber septum was charged with (1R,3aR,7aR)-7a-methyl-1-[(1R,3E)-6,6,6-trifluoro-5-hydroxy-1-methyl-1-(5,5,5-trideutero-4-hydroxy-4-trideuteromethyl-pentyl)-5-trifluoromethyl-hex-3-enyl]-octahydro-inden-4-one (746 mg, 1.515 mmol) and dichloromethane (10 ml). 1-(trimethylsilyl)imidazole (1.90 ml, 12.95 mmol) was added dropwise. The mixture was stirred at room temperature for 3 h. Hexane (150 ml) was added and the mixture was washed with water (3×50 ml), dried (Na₂SO₄) and evaporated. The oil residue was chromatographed on column (50 cm³) using hexane:ethyl acetate—5:1 as mobile phase. Fractions containing product were pooled and evaporated to give product as colorless oil (917 mg, 95%).

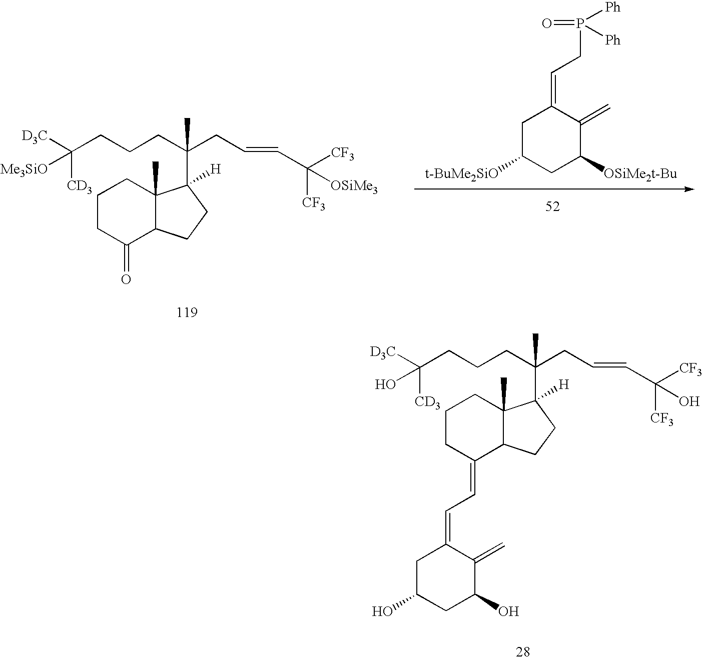

### 1,25-Dihydroxy-20R-20-(4-hydroxy-5,5,5-trideutero-4-trideuteromethyl-pentyl)-23E-ene-26,27-hexafluorocholecalciferol (28)

A 25 ml round bottom flask equipped with stir bar and Claisen adapter with rubber septum was charged with (1S,5R)-1,5-bis-((tert-butyldimethyl)silanyloxy)-3-[2-(diphenylfosphinoyl)-eth-Z)-ylidene]-2-methylene-cyclohexane (460 mg, 0.789 mmol) and tetrahydrofuran (8 ml). The reaction mixture was cooled to −70° C. and n-butyllithium (0.49 ml, 0.78 mmol)) was added dropwise. The resulting deep red solution was stirred at −70° C. for 20 min and (1R,3aR,7aR)-7a-Methyl-1-[(1R,3E)-6,6,6-trifluoro-1-methyl-1-(5,5,5-trideutero-4-trideuteromethyl-4-trimethylsilanyloxy-pentyl)-5-trifluoromethyl-5-trimethylsilanyloxy-hex-3-enyl]-octahydro-inden-4-one (302 mg, 0.474 mmol) was added dropwise in tetrahydrofuran (1.5 ml). The reaction mixture was stirred for 5.5 h (in last hour the temperature was increased from −70 do −50° C.). Saturated solution of ammonium chloride (1 ml) was added and the bath was removed. The mixture was poured into ethyl acetate (50 ml) and saturated solution of ammonium chloride (50 ml). The water fraction was extracted with ethyl acetate (3×50 ml), dried (Na₂SO₄) and evaporated. The oil residue was chromatographed on column (50 cm³, protected from light) using hexane:ethyl acetate—10:1 as mobile phase. Fractions containing product and some mono deprotected compound were pooled and evaporated to give colorless oil. A 25 ml round bottom flask equipped with stir bar and Claisen adapter with rubber septum was charged with substrate and tetrabutylammonium fluoride (15 ml, 1M/tetrahydrofuran). The mixture was stirred for next 18 h. The mixture was dissolved by the addition of ethyl acetate (150 ml) and washed 6 times with water (50 ml) and brine (50 ml), dried (Na₂SO₄) and evaporated. The oil residue was chromatographed on column (50 cm³, protected from light) using ethyl acetate as mobile phase (tetrahydrofuran was used to transfer material on column). Fractions with product contained some impurity. Fractions containing product were pooled and evaporated to give a white solid. The solid phase was transferred to Buchner funnel (10-15 μm) with hexane and washed with hexane (20 ml) to remove impurity. Then product was removed from funnel with ethanol (25 ml) and solution was evaporated to give product as white solid (215 mg, 71%).

[α]_{D}²⁷=+16.1 (c=0.44, EtOH)

UV λmax (EtOH): 214 nm (ε 1377), 265 nm (ε 1675)

¹H NMR (DMSO-D6): 8.05 (1H, s), 6.28 (1H, dt, J=15.3, 7.7 Hz), 6.18 (1H, d, J=11.1 Hz), 5.97 (1H, d, J=11.3 Hz), 5.62 (1H, d, J=15.3 Hz), 5.22 (1H, s), 4.87 (1H, d, J=4.7 Hz), 4.75 (1H, d, J=2.1 Hz), 4.55 (1H, d, J=3.6 Hz), 4.21-4.16 (1H, m), 4.04 (1H, s), 4.05-3.95 (1H, m), 2.79-2.76 (1H, m), 2.35 (1H, d, J=13.9 Hz), 2.16 (1H, dd, J=13.3, 5.2 Hz), 2.07 (2H, d, J=7.5 Hz), 2.00-1.90 (2H, m), 1.82-1.78 (1H, m), 1.65-1.55 (6H, m), 1.43-1.24 (10H, m), 0.90 (3H, s), 0.61 (3H, s)

¹³C NMR (DMSO-D6): 149.37, 139.67, 136.44, 135.84, 122.60 (q, J=286.8 Hz), 122.35, 119.82, 117.93, 109.79, 75.49 (sep, J=28.8 Hz), 68.39, 65.06, 56.36, 56.01, 46.20, 44.87, 44.56, 43.11, 41.06, 40.43, 28.33, 23.09, 22.49, 21.80, 21.60, 17.90, 14.59

| | | | | |
|---|---|---|---|---|
| MS HRES | Calculated for: | C₃₃H₄₂D₆F₆O₄ | [M + Na]⁺ | 651.3725 |
| | Observed: | | [M + Na]⁺ | 651.3729 |

### Example 38

### Synthesis of 1,25-Dihydroxy-20R-20-(4-hydroxy-5,5,5-trideutero-4-trideuteromethyl-pentyl)-23E-ene-26,27-hexafluoro-19-nor-cholecalciferol (29)

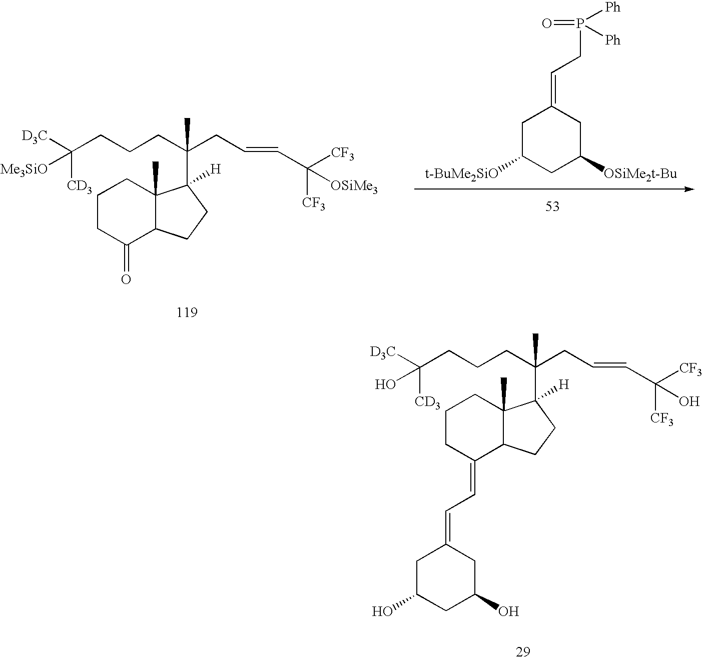

### 1,25-Dihydroxy-20R-20-(4-hydroxy-5,5,5-trideutero-4-trideuteromethyl-pentyl)-23E-ene-26,27-hexafluoro-19-nor-cholecalciferol (29)

A 25 ml round bottom flask equipped with stir bar and Claisen adapter with rubber septum was charged with (1R,3R)-1,3-bis-((tert-butyldimethyl)silanyloxy)-5-[2-(diphenylfosphinoyl)ethylidene]-cyclohexane (584 mg, 1.023 mmol) and tetrahydrofuran (8 ml). The reaction mixture was cooled to −70° C. and n-butyllithium (0.63 ml, 1.01 mmol)) was added dropwise. The resulting deep red solution was stirred at −70° C. for 20 min and (1R,3aR,7aR)-7a-Methyl-1-[(1R,3E)-6,6,6-trifluoro-1-methyl-1-(5,5,5-trideutero-4-trideuteromethyl-4-trimethylsilanyloxy-pentyl)-5-trifluoromethyl-5-trimethylsilanyloxy-hex-3-enyl]-octahydro-inden-4-one (308 mg, 0.484 mmol) was added dropwise in tetrahydrofuran (1.5 ml). The reaction mixture was stirred for 6 h and then saturated solution of ammonium chloride (1 ml) was added and the bath was removed. The mixture was poured into ethyl acetate (50 ml) and saturated solution of ammonium chloride (50 ml). The water fraction was extracted with ethyl acetate (3×50 ml), dried (Na₂SO₄) and evaporated. The oil residue was chromatographed on column (50 cm³, protected from light) using hexane:ethyl acetate—10:1 as mobile phase. Fractions containing product and some mono deprotected compound were pooled and evaporated to give colorless oil. A 25 ml round bottom flask equipped with stir bar and Claisen adapter with rubber septum was charged with substrate and tetrabutylammonium fluoride (15 ml, 1M/tetrahydrofuran). The mixture was stirred for next 96 h. The mixture was dissolved by the addition of ethyl acetate (150 ml) and washed 6 times with water (50 ml) and brine (50 ml), dried (Na₂SO₄) and evaporated. The oil residue was chromatographed on column (50 cm³, protected from light) using hexane:tetrahydrofuran—1:1, 1:2 as mobile phase. (tetrahydrofuran contained some impurity). Fractions containing product were pooled and evaporated to give a white solid. The solid phase was transferred to Buchner funnel (110-15 μm) with hexane and washed with hexane (20 ml) to remove impurity. Then product was removed from funnel with ethanol (25 ml) and solution was evaporated to give product as white solid (274 mg, 92%).

[α]_{D}²⁷=+48.2 (c=0.44, EtOH)

UV λmax (EtOH): 244 nm (ε 35585), 252 nm (ε 41634), 262 nm (ε 28023)

¹H NMR (DMSO-D6): 8.05 (1H, s), 6.29 (1H, dt, J=15.6, 7.7 Hz), 6.07 (1H, d, J=11.3 Hz), 5.78 (1H, d, J=11.3 Hz), 5.62 (1H, d, J=15.6 Hz), 4.48 (1H, d, J=4.1 Hz), 4.38 (1H, d, J=3.8 Hz), 4.04 (1H, s), 3.90-3.84 (1H, m), 3.83-3.76 (1H, m), 2.73 (1H, d, J=13.2 Hz), 2.43 (1H, dd, J=12.9, 3.3 Hz), 2.26 (1H, d, J=10.4 Hz), 2.09-1.91 (6H, m), 1.69-1.24 (17H, m), 0.91 (3H, s), 0.60 (3H, s)

¹³C NMR (DMSO-D6): 139.10, 136.46, 134.64, 122.59 (q, J=286.0 Hz), 120.80, 119.84, 116.38, 75.50 (sep, J=28.8 Hz), 68.40, 65.54, 65.25, 56.36, 55.98, 46.04, 44.56, 42.22, 41.07, 40.43, 36.96, 28.16, 22.95, 22.50, 21.85, 21.50, 17.90, 14.70

| | | | | |
|---|---|---|---|---|
| MS HRES | Calculated for: | C₃₂H₄₂D₆F₆O₄ | [M + Na]⁺ | 639.3725 |
| | Observed: | | [M + Na]⁺ | 639.3725 |

### Example 39

### Synthesis of 1α-Fluoro-25-hydroxy-20R-20-(4-hydroxy-5,5,5-trideutero-4-trideuteromethyl-pentyl)-23E-ene-26,27-hexafluorocholecalciferol (30)

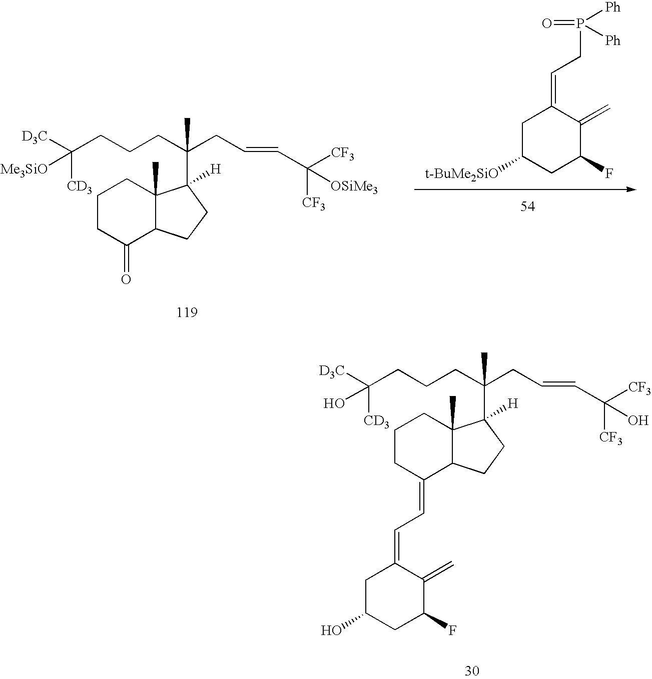

### 1α-Fluoro-25-hydroxy-20R-20-(4-hydroxy-5,5,5-trideutero-4-trideuteromethyl-pentyl)-23E-ene-26,27-hexafluorocholecalciferol (30)

A 25 ml round bottom flask equipped with stir bar and Claisen adapter with rubber septum was charged with (1S,5R)-1-((tert-butyldimethyl)silanyloxy)-3-[2-(diphenylfosphinoyl)-eth-(Z)-ylidene]-5-fluoro-2-methylene-cyclohexane (543 mg, 1.154 mmol) and tetrahydrofuran (8 ml). The reaction mixture was cooled to −70° C. and n-butyllithium (0.72 ml, 1.15 mmol)) was added dropwise. The resulting deep red solution was stirred at −70° C. for 20 min and (1R,3aR,7aR)-7a-Methyl-1-[(1R,3E)-6,6,6-trifluoro-1-methyl-1-(5,5,5-trideutero-4-trideuteromethyl-4-trimethylsilanyloxy-pentyl)-5-trifluoromethyl-5-trimethylsilanyloxy-hex-3-enyl]-octahydro-inden-4-one (279 mg, 0.438 mmol) was added dropwise in tetrahydrofuran (1.5 ml). The reaction mixture was stirred for 8 h (in last hour the temperature was increased from −70 do −50° C.). The bath was removed and the mixture was poured into ethyl acetate (50 ml) and saturated solution of ammonium chloride (50 ml). The water fraction was extracted with ethyl acetate (3×50 ml), dried (Na₂SO₄) and evaporated. The oil residue was chromatographed on column (50 cm³, protected from light) using hexane:ethyl acetate—10:1 as mobile phase. Fractions containing product were pooled and evaporated to give oil. The oil residue was used to next reaction. A 25 ml round bottom flask equipped with stir bar and Claisen adapter with rubber septum was charged with substrate and tetrabutylammonium fluoride (8 ml, 1M/tetrahydrofuran). The mixture was stirred for next 25 h. The mixture was dissolved by the addition of ethyl acetate (150 ml) and extracted 6 times with water and brine (30 ml+20 ml), dried (Na₂SO₄) and evaporated. The oil residue was chromatographed on column (50 cm³, protected from light) using hexane:ethyl acetate—2:1, 1:1 as mobile phase. Fractions containing product were pooled and evaporated to give product as colorless oil. Oil was dissolved in methyl acetate and evaporated (4 times) to give product as white foam (216 mg, 78%).

[α]_{D}²⁸=+32.5 (c=0.48, EtOH)

UV λmax (EtOH): 211 nm (ε 16931), 243 nm (ε 17696), 269 nm (ε 17736)

¹H NMR (DMSO-D6): 8.05 (1H, s), 6.36 (1H, d, J=11.3 Hz), 6.28 (1H, dt; J=15.6, 7.6 Hz), 5.92 (1H, d, J=11.3 Hz), 5.62 (1H, d, J=15.3 Hz), 5.39 (1H, s), 5.14 (1H, br d, J=49.7 Hz), 4.99 (1H, d, J=1.7 Hz), 4.86 (1H, d, J=4.3 Hz), 4.04 (1H, s), 3.94-3.86 (1H, m), 2.81 (1H, d, J=12.4 Hz), 2.15-2.06 (4H, m), 1.99-1.91 (3H, m), 1.82-1.55 (6H, m), 1.46-1.20 (10H, m), 0.90 (3H, s), 0.59 (3H, s)

¹³C NMR (DMSO-D6): 143.29 (d, J=17.4 Hz), 141.83, 136.58, 133.13 (d, J=1.5 Hz), 124.20, 122.76 (q, J=287.5 Hz), 119.99, 117.46, 115.39 (d, J=9.9 Hz), 92.09 (d, J=166.8 Hz), 75.57 (sep, J=28.8 Hz), 68.48, 64.60, 64.56, 56.40, 56.02, 46.31, 44.86, 44.58, 41.11, 40.71 (d, J=20.4 Hz), 40.43, 39.36, 28.34, 23.02, 22.44, 21.79, 21.50, 17.90, 14.60

| | | | | |
|---|---|---|---|---|
| MS HRES | Calculated for: | C₃₃H₄₁D₆F₇O₃ | [M + Na]⁺ | 653.3682 |
| | Observed: | | [M + Na]⁺ | 653.3684 |

### Example 40

### Determination of Maximum Tolerated Dose (MTD) of Vitamin D₃ Analogs

The maximum tolerated dose of the vitamin D₃ compounds of the invention were determined in eight week-old female C57BL/6 mice (3 mice/group) dosed orally (0.1 ml/mouse) with various concentrations of Vitamin D₃ analogs daily for four days. Analogs were formulated in miglyol for a final concentration of 10, 30, 100 and 300 μg/kg when given at 0.1 ml/mouse p.o. daily. Blood for serum calcium assay was drawn by tail bleed on day five, the final day of the study. Serum calcium levels were determined using a colorimetric assay (Sigma Diagnostics, procedure no. 597). The highest dose of analog tolerated without inducing hypercalcemia (serum calcium >10.7 mg/dl) was taken as the maximum tolerated does (MTD). Table 1 shows the relative MTD for vitamin D₃ compounds.

TABLE 1

| | MTD | |
|---|---|---|
| | (mice) | IFN-γ |
| COMPOUND | μg/kg | IC₅₀ pM |
| 1,25-Dihydroxy-20-(4-hydroxy-4-methyl-pentyl)- | 3 | 518.0 |
| cholecalciferol (1) | | |
| 1,25-Dihydroxy-20-(4-hydroxy-4-methyl-pentyl)- | <30 | 744.0 |
| 19-nor-cholecalciferol (2) | | |
| 1α-Fluoro-25-hydroxy-20-(4-hydroxy-4-methyl- | >300 | 6267.0 |
| pentyl)-cholecalciferol (3) | | |
| (20S)-1,25-Dihydroxy-20-(5,5,5-trifluoro-4- | 0.3 | 49.0 |
| hydroxy-4-trifluoromethyl-pent-2- | | |
| ynyl)cholecalciferol (4) | | |
| (20S)-1,25-Dihydroxy-20-(5,5,5-trifluoro-4- | 0.3 | 42.0 |
| hydroxy-4-trifluoromethyl-pent-(2Z)- | | |
| enyl)cholecalciferol (5) | | |
| (20S)-1,25-Dihydroxy-20-[(2E)-5,5,5-trifluoro-4- | 0.03 | 44.0 |
| hydroxy-4-trifluoromethyl-pent-2-enyl]- | | |
| cholecalciferol (6) | | |
| (20R)-1,25-Dihydroxy-20-(5,5,5-trifluoro-4- | 0.03 | 38.0 |
| hydroxy-4-trifluoromethyl-pent-2-ynyl)- | | |
| cholecalciferol (7) | | |
| (20R)-1,25-Dihydroxy-20-[(2Z)-5,5,5-trifluoro-4- | 0.3 | 57.0 |
| hydroxy-4-trifluoromethyl-pent-2-enyl]- | | |
| cholecalciferol (8) | | |
| (20R)-1,25-Dihydroxy-20-[(2E)-5,5,5-trifluoro-4- | 0.1 | 49.0 |
| hydroxy-4-trifluoromethyl-pent-2-enyl]- | | |
| cholecalciferol (9) | | |
| (20S)-1,25-Dihydroxy-20-(5-5,5-trifluoro-4- | 0.3 | 43.6 |
| hydroxy-4-trifluoromethyl-pent-2-ynyl)-19-nor- | | |
| cholecalciferol (10) | | |
| (20S)-1,25-Dihydroxy-20-[(2Z)-5,5,5-trifluoro-4- | 3 | 253.3 |
| hydroxy-4-trifluoromethyl-pent-2-enyl]-19-nor- | | |
| cholecalciferol (11) | | |
| (20S)-1,25-Dihydroxy-20-[(2E)-5,5,5-trifluoro-4- | 0.03 | <0.01 |
| hydroxy-4-trifluoromethyl-pent-2-enyl]-19-nor- | | |
| cholecalciferol (12) | | |
| (20S)-1α-Fluoro-25-hydroxy-20-(5,5,5-trifluoro- | 100 | 358.3 |
| 4-hydroxy-4-trifluoromethyl-pent-2-ynyl)- | | |
| cholecalciferol (13) | | |
| (20S)-1α-Fluoro-25-hydroxy-20-[(2Z)-5,5,5- | 100 | 1011.0 |
| trifluoro-4-hydroxy-4-trifluoromethyl- | | |
| pent-2-enyl]- | | |
| cholecalciferol (14) | | |
| (20S)-1α-Fluoro-25-hydroxy-20-[(2E)-5,5,5- | 10 | 118.6 |
| trifluoro-4-hydroxy-4-trifluoromethyl- | | |
| pent-2-enyl]- | | |
| cholecalciferol (15) | | |
| (20S)-1,25-Dihydroxy-20-((2Z)-5,5,5-trifluoro-4- | 0.3 | 11.1 |
| hydroxy-4-trifluoromethyl-pent-2-enyl)-26,27- | | |
| hexadeutero-cholecalciferol (16) | | |
| (20S)-1,25-Dihydroxy-20-((2Z)-5,5,5-trifluoro-4- | 0.3 | 2.5 |
| hydroxy-4-trifluoromethyl-pent-2-enyl)-26,27- | | |
| hexadeutero-19-nor-cholecalciferol (17) | | |
| (20S)-1α-Fluoro-25-hydroxy-20-((2Z)-5,5,5- | 100 | 219.0 |
| trifluoro-4-hydroxy-4- | | |
| trifluoromethyl-pent-2-enyl)- | | |
| 26,27-hexadeutero-cholecalciferol (18) | | |
| (20S)-1,25-Dihydroxy-20-((2E)-5,5,5-trifluoro-4- | 0.1 | 0.0025 |
| hydroxy-4-trifluoromethyl-pent-2-enyl)-26,27- | | |
| hexadeutero-cholecalciferol (19) | | |
| (20S)-1,25-Dihydroxy-20-((2E)-5,5-5-trifluoro-4- | 0.1 | 0.00007 |
| hydroxy-4-trifluoromethyl-pent-2-enyl)-26,27- | | |
| hexadeutero-19-nor-cholecalciferol (20) | | |
| (20S)-1α-Fluoro-25-hydroxy-20-((2E)-5,5,5- | 10 | 7.1 |
| trifluoro-4-hydroxy-4- | | |
| trifluoromethyl-pent-2-enyl)- | | |
| 26,27-hexadeutero-cholecalciferol (21) | | |
| (20S)-1,25-Dihydroxy-20-(5,5,5-trifluoro-4- | 0.3 | 0.028 |
| hydroxy-4-trifluoromethyl-pent-2-ynyl)-26,27- | | |
| hexadeutero-cholecalciferol (22) | | |
| (20S)-1,25-Dihydroxy-20-(5,5,5-trifluoro-4- | 1 | 0.026 |
| hydroxy-4-trifluoromethyl-pent-2-ynyl)-26,27- | | |
| hexadeutero-19-nor-cholecalciferol (23) | | |
| (20S)-1α-Fluoro-25-hydroxy-20-(5,5,5-trifluoro- | 10 | 21.3 |
| 4-hydroxy-4-trifluoromethyl-pent-2-ynyl)-26,27- | | |
| hexadeutero-cholecalciferol (24) | | |
| 1,25-Dihydroxy-20R-20-(4-hydroxy-5,5,5- | 3 | 1.0 |
| trideutero-4-trideuteromethyl-pentyl)-23Z-ene- | | |
| 26,27-hexafluorocholecalciferol (25) | | |
| 1,25-Dihydroxy-20R-20-(4-hydroxy-5,5,5- | 0.3 | 0.33 |
| trideutero-4-trideuteromethyl-pentyl)-23Z-ene- | | |
| 26,27-hexafluoro-19-nor-cholecalciferol (26) | | |
| 1α-Fluoro-25-hydroxy-20R-20-(4-hydroxy-5,5,5- | — | 307.0 |
| trideutero-4-trideuteromethyl-pentyl)-23Z-ene- | | |
| 26,27-hexafluorocholecalciferol (27) | | |
| 1,25-Dihydroxy-20R-20-(4-hydroxy-5,5,5,- | 0.3 | 0.0025 |
| trideutero-4-trideuteromethyl-pentyl)-23E-ene- | | |
| 26,27-hexafluorocholecalciferol (28) | | |
| 1,25-Dihydroxy-20R-20-(4-hydroxy-5,5,5- | 0.1 | <0.000001 |
| trideutero-4-trideuteromethyl-pentyl)-23E-ene- | | |
| 26,27-hexafluoro-19-nor-cholecalciferol (29) | | |
| 1α-Fluoro-25-hydroxy-20R-20-(4-hydroxy-5,5,5- | — | 12.0 |
| trideutero-4-trideuteromethyl-pentyl)-23E-ene- | | |
| 26,27-hexafluorocholecalciferol (30) | | |
| 1,25-Dihydroxy-20R-20-(4-hydroxy-5,5,5- | 0.3 | 0.09 |
| trideutero-4-trideuteromethyl- | | |
| pentyl)-23-yne-26,27- | | |
| hexafluorocholecalciferol (31) | | |
| 1,25-Dihydroxy-20R-20-(4-hydroxy-5,5,5- | 0.3 | 0.01 |
| trideutero-4-trideuteromethyl- | | |
| pentyl)-23-yne-26,27- | | |
| hexafluoro-19-nor-cholecalciferol (32) | | |
| 1α-Fluoro-25-hydroxy-20R-20-(4-hydroxy-5,5,5- | — | 13.2 |
| trideutero-4-trideuteromethyl- | | |
| pentyl)-23-yne-26,27- | | |
| hexafluorocholecalciferol (33) | | |

### Example 41

### Immunological Assay of Vitamin D₃ Compounds

Immature dendritic cells (DC) were prepared as described in Romani, N. et al., J. Immunol. Meth. 196:137. IFN-γ production by allogeneic T cell activation in the mixed leukocyte response (MLR) was determined as described in Penna, G., et al., J. Immunol., 164: 2405-2411 (2000).

Briefly, peripheral blood mononuclear cells (PBMC) were separated from buffy coats by Ficoll gradient and the same number (3×105) of allogeneic PBMC from 2 different donors were co-cultured in 96-well flat-bottom plates. After 5 days, IFN-γ production in the MLR assay was measured by ELISA and the results expressed as amount (nM) of test compound required to induce 50% inhibition of IFN-γ production (IC₅₀) (Table 1).

### Example 42

Soft Gelatin Capsule Formulation I

| Item | Ingredients | mg/Capsule |
|---|---|---|
| 1. | Compound 1 | 10.001-0.02 |
| 2. | Butylated Hydroxytoluene (BHT) | 0.016 |
| 3. | Butylated Hydroxyanisole (BHA) | 0.016 |
| 4. | Miglyol 812 qs. | 160.0 |

Manufacturing Procedure:
1. BHT and BHA is suspended in Miglyol 812 and warmed to about 50° C. with stirring, until dissolved.
2. A Gemini vitamin D₃ compound of the invention is dissolved in the solution from step 1 at 50° C..
3. The solution from Step 2 is cooled at room temperature.
4. The solution from Step 3 is filled into soft gelatin capsules.
Note:
All manufacturing steps are performed under a nitrogen atmosphere and protected from light.

### Example 43

Soft Gelatin Capsule Formulation II

| Item | Ingredients | mg/Capsule |
|---|---|---|
| 1. | Compound 1 | 10.001-0.02 |
| 2. | di-α-Tocopherol | 0.016 |
| 3. | Miglyol 812 qs. | 160.0 |

Manufacturing Procedure:
1. Di-α-Tocopherol is suspended in Miglyol 812 and warmed to about 50° C. with stirring, until dissolved.
2. A Gemini vitamin D₃ compound of the invention.
3. The solution from Step 2 is cooled at room temperature.
4. The solution from Step 3 is filled into soft gelatin capsules.

### INCORPORATION BY REFERENCE

The contents of all references (including literature references, issued patents, published patent applications, and co-pending patent applications) cited throughout this application are hereby expressly incorporated herein in their entireties by reference.

### EQUIVALENTS

Those skilled in the art will recognize, or be able to ascertain using no more than routine experimentation, many equivalents of the specific embodiments of the invention described herein. Such equivalents are intended to be encompassed by the following claims.

1. A vitamin D₃ compound having formula I: 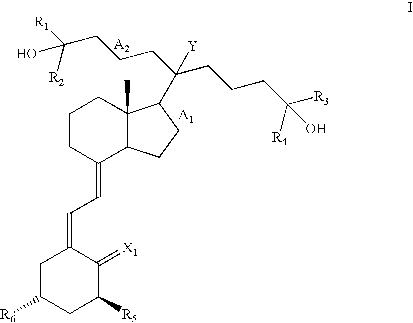
wherein:
A₁ is a single or double bond;
A₂ is a single, a double or a triple bond;
R₁, R₂, R₃ and R₄ are each independently alkyl, deuteroalkyl, hydroxyalkyl, or haloalkyl;
R₅ is halogen, hydroxyl, OC(O)alkyl, OC(O)hydroxyalkyl, or OC(O)haloalkyl;
R₆ is halogen, hydroxyl, OC(O)alkyl, OC(O)hydroxyalkyl, or OC(O)haloalkyl;
X₁ is H₂ or CH₂;
Y is alkyl;
and pharmaceutically acceptable esters, salts, and prodrugs thereof.

2-17. (canceled)

18. A vitamin D₃ compound having formula I-a: 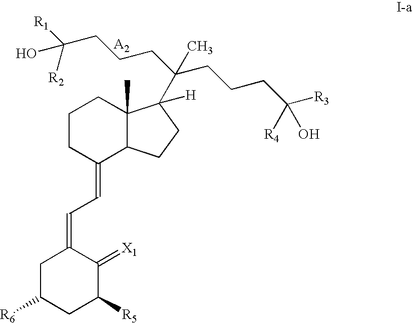
wherein:
A₂ is a single, a double or a triple bond;
R₁, R₂, R₃ and R₄ are each independently alkyl, hydroxyalkyl, or haloalkyl;
R₅ is halogen, hydroxyl, OC(O)alkyl, OC(O)hydroxyalkyl, or OC(O)haloalkyl;
R₆ is hydroxyl, OC(O)alkyl, OC(O)hydroxyalkyl, or OC(O)haloalkyl;
X₁ is H₂ or CH₂;
and pharmaceutically acceptable esters, salts, and prodrugs thereof.

19-47. (canceled)

48. A vitamin D₃ compound of claim 1 having formula I-b: 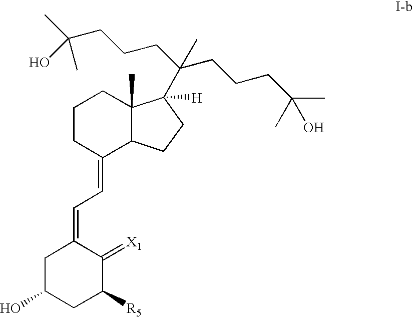
wherein:
R₅ is fluoro or hydroxyl;
X₁ is H₂ or CH₂;
and pharmaceutically acceptable esters, salts, and prodrugs thereof.

49-52. (canceled)

53. A vitamin D₃ compound of claim 1 having formula I-c: 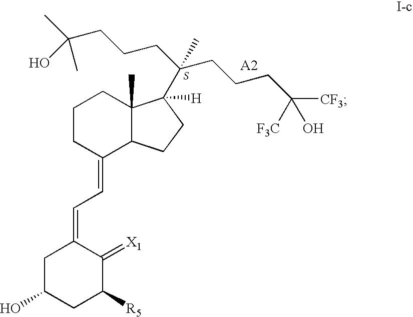
wherein:
A₂ is a single, a double or a triple bond;
R₅ is fluoro or hydroxyl;
X₁ is H₂ or CH₂;
and pharmaceutically acceptable esters, salts, and prodrugs thereof.

54. A vitamin D₃ compound of claim 1 having formula I-d: 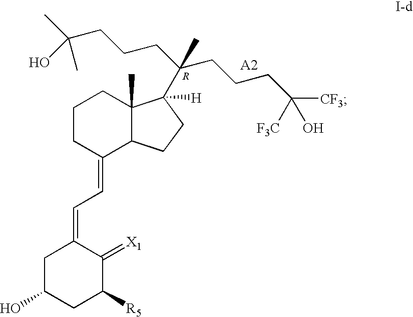
wherein:
A₂ is a single, a double or a triple bond;
R₅ is fluoro or hydroxyl;
X₁ is H₂ or CH₂;
and pharmaceutically acceptable esters, salts, and prodrugs thereof.

55. A vitamin D₃ compound of claim 1 having formula I-e: 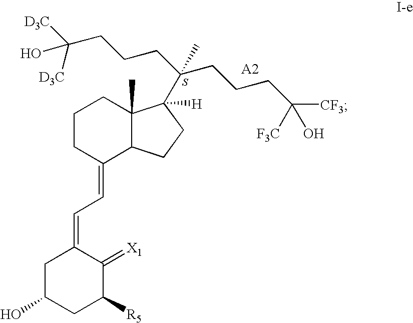
wherein:
A₂ is a single, a double or a triple bond;
R₅ is fluoro or hydroxyl;
X₁ is H₂ or CH₂;
and pharmaceutically acceptable esters, salts, and prodrugs thereof.

56. A vitamin D₃ compound of claim 1 having formula I-f: 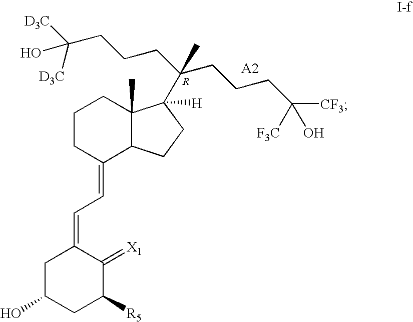
wherein:
A₂ is a single, a double or a triple bond;
R₅ is fluoro or hydroxyl;
X₁ is H₂ or CH₂;
and pharmaceutically acceptable esters, salts, and prodrugs thereof.

57. The compound of claim 53, wherein A₂ is a triple bond.

58. The compound of claim 54, wherein A₂ is a triple bond.

59. The compound of claim 55, wherein A₂ is a triple bond.

60. The compound of claim 56, wherein A₂ is a triple bond.

61. (canceled)

62. The compound of claim 53, wherein A₂ is a cis double bond.

63. The compound of claim 54, wherein A₂ is a triple bond.

64. The compound of claim 55, wherein A₂ is a triple bond.

65. The compound of claim 56, wherein A₂ is a triple bond.

66. (canceled)

67. The compound of claim 53, wherein A₂ is a trans double bond.

68. The compound of claim 54, wherein A₂ is a trans double bond.

69. The compound of claim 55, wherein A₂ is a trans double bond.

70. The compound of claim 55, wherein A₂ is a trans double bond.

71. (canceled)

72. The compound of claim 1, wherein said compound is
1,25-Dihydroxy-20-(4-hydroxy-4-methyl-pentyl)-cholecalciferol (1): 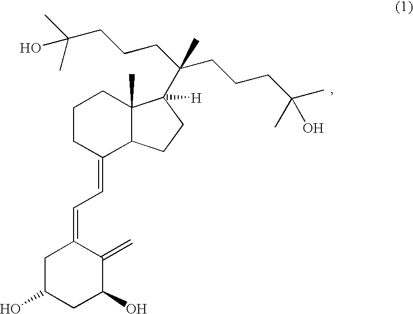
1,25-Dihydroxy-20-(4-hydroxy-4-methyl-pentyl)-19-nor-cholecalciferol (2): 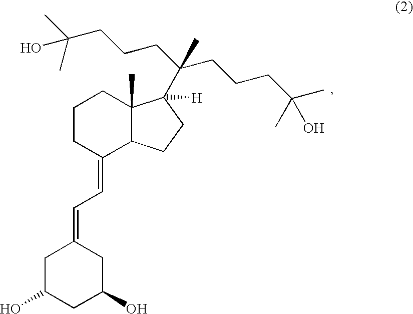
1α-Fluoro-25-hydroxy-20-(4-hydroxy-4-methyl-pentyl)-cholecalciferol (3): 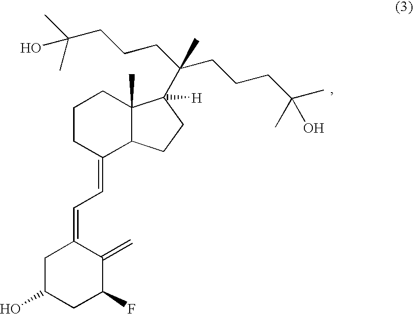
(20S)-1,25-Dihydroxy-20-(5,5,5-trifluoro-4-hydroxy-4-trifluoromethyl-pent-2-ynyl)cholecalciferol (4): 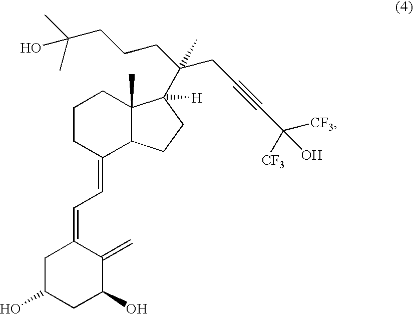
(20S)-1,25-Dihydroxy-20-((2Z)-5,5,5-trifluoro-4-hydroxy-4-trifluoromethyl-pent-2-enyl)cholecalciferol (5): 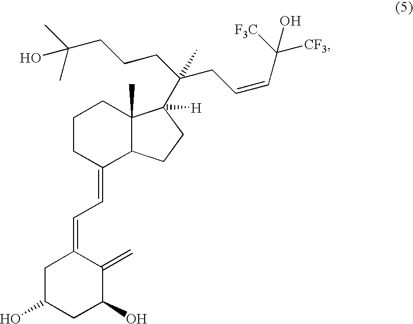
(20S)-1,25-Dihydroxy-20-[(2E)-5,5,5-trifluoro-4-hydroxy-4-trifluoromethyl-pent-2-enyl]-cholecalciferol (6): 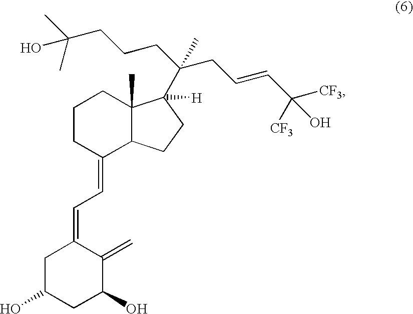
(20S)-1,25-Dihydroxy-20-(5,5,5-trifluoro-4-hydroxy-4-trifluoromethyl-pent-2-ynyl)-19-nor-cholecalciferol (10): 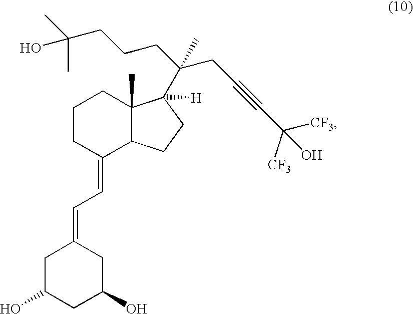
(20S)-1,25-Dihydroxy-20-[(2Z)-5,5,5-trifluoro-4-hydroxy-4-trifluoromethyl-pent-2-enyl]-19-nor-cholecalciferol (11): 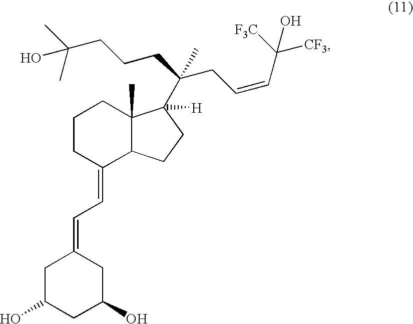
(20S)-1,25-Dihydroxy-20-[(2E)-5,5,5-trifluoro-4-hydroxy-4-trifluoromethyl-pent-2-enyl]-19-nor-cholecalciferol (12): 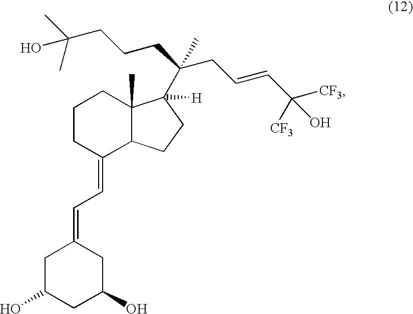
(20S)-1α-Fluoro-25-hydroxy-20-(5,5,5-trifluoro-4-hydroxy-4-trifluoromethyl-pent-2-ynyl)-cholecalciferol (13): 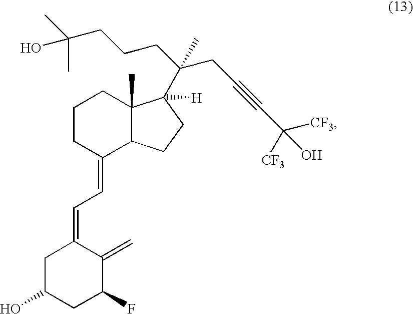
(20S)-1α-Fluoro-25-hydroxy-20-[(2Z)-5,5,5-trifluoro-4-hydroxy-4-trifluoromethyl-pent-2-enyl]-cholecalciferol (14): 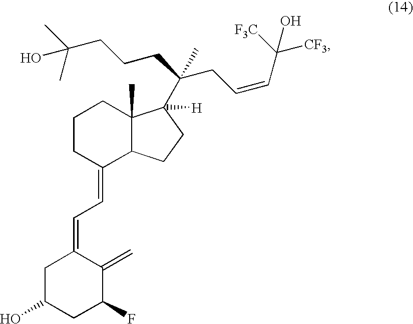
(20S)-1α-Fluoro-25-hydroxy-20-[(2E)-5,5,5-trifluoro-4-hydroxy-4-trifluoromethyl-pent-2-enyl]-cholecalciferol (15): 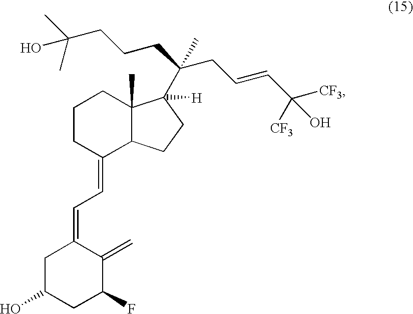
(20R)-1,25-Dihydroxy-20-(5,5,5-trifluoro-4-hydroxy-4-trifluoromethyl-pent-2-ynyl)-cholecalciferol (7): 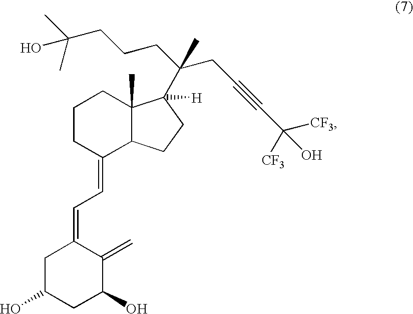
(20R)-1,25-Dihydroxy-20-[(2)-5,5,5-trifluoro-4-hydroxy-4-trifluoromethyl-pent-2-enyl]-cholecalciferol (8): 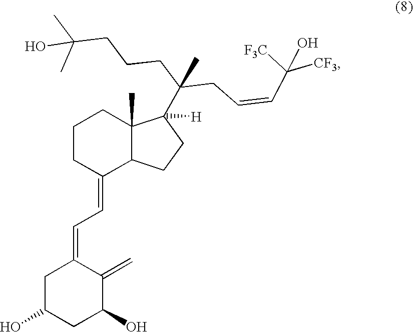
(20R)-1,25-Dihydroxy-20-[(2E)-5,5,5-trifluoro-4-hydroxy-4-trifluoromethyl-pent-2-enyl]-cholecalciferol (9): 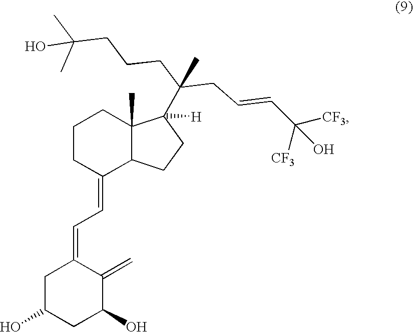
1,25-Dihydroxy-20R-20-(4-hydroxy-4-methyl-pentyl)-23-yne-26,27-hexafluoro-cholecalciferol (34): 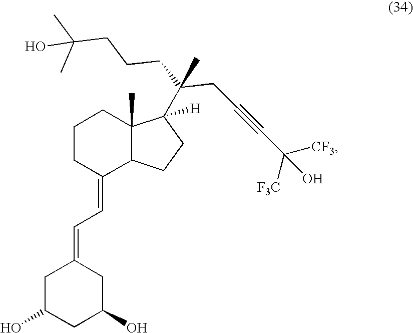
1,25-Dihydroxy-20R-20-(4-hydroxy-4-methyl-pentyl)-23Z-ene-26,27-hexafluoro-cholecalciferol (35): 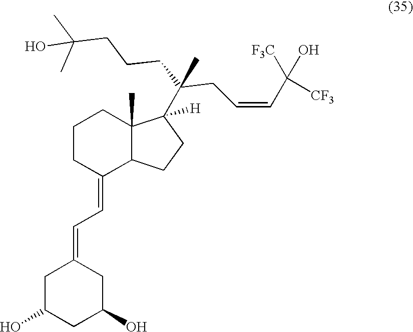
1,25-Dihydroxy-20R-20-(4-hydroxy-4-methyl-pentyl)-23E-ene-26,27-hexafluoro-cholecalciferol (36): 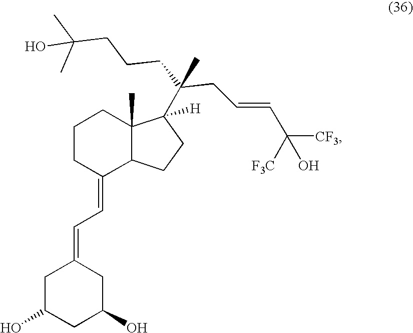
1α-Fluoro-25-hydroxy-20R-20-(4-hydroxy-4-methyl-pentyl)-23-yne-26,27-hexafluorocholecalciferol (37) 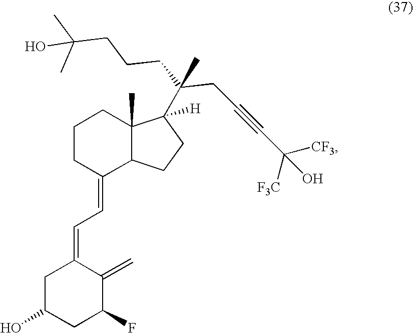
1α-Fluoro-25-hydroxy-20R-20-(4-hydroxy-4-methyl-pentyl)-23Z-ene-26,27-hexafluorocholecalciferol (38): 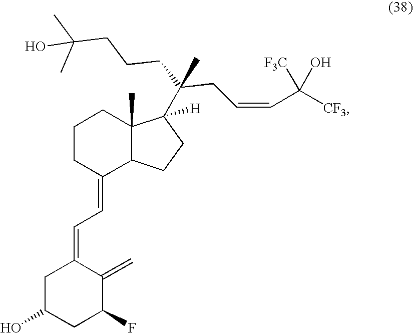
1α-Fluoro-25-hydroxy-20R-20-(4-hydroxy-4-methyl-pentyl)-23E-ene-26,27-hexafluorocholecalciferol (39): 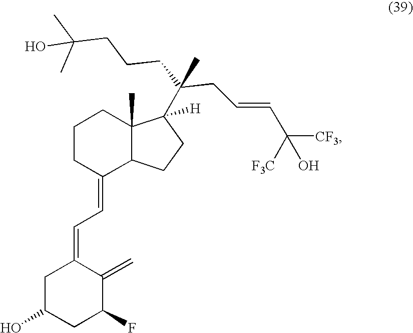
(20S)-1,25-Dihydroxy-20-((2Z)-5,5,5-trifluoro-4-hydroxy-4-trifluoromethyl-pent-2-enyl)-26,27-hexadeutero-cholecalciferol (16): 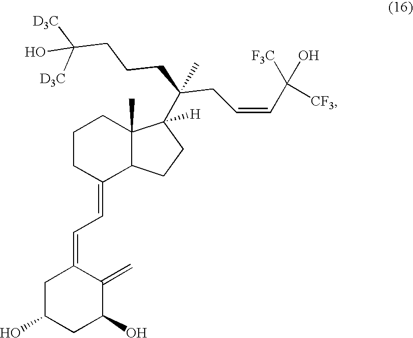
(20S)-1,25-Dihydroxy-20-((2Z)-5,5,5-trifluoro-4-hydroxy-4-trifluoromethyl-pent-2-enyl)-26,27-hexadeutero-19-nor-cholecalciferol (17): 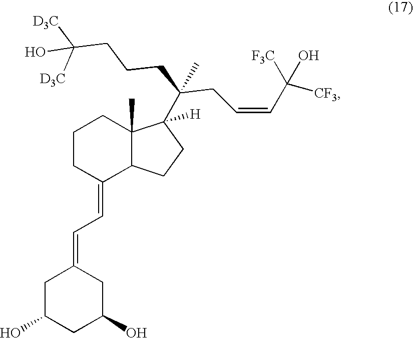
(20S)-1α-Fluoro-25-hydroxy-20-((2Z)-5,5,5-trifluoro-4-hydroxy-4-trifluoromethyl-pent-2-enyl)-26,27-hexadeutero-cholecalciferol (18): 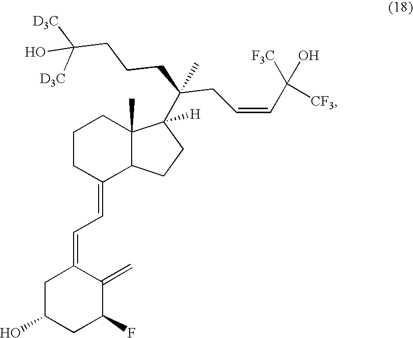
(20S)-1,25-Dihydroxy-20-((2E)-5,5,5-trifluoro-4-hydroxy-4-trifluoromethyl-pent-2-enyl)-26,27-hexadeutero-cholecalciferol (19): 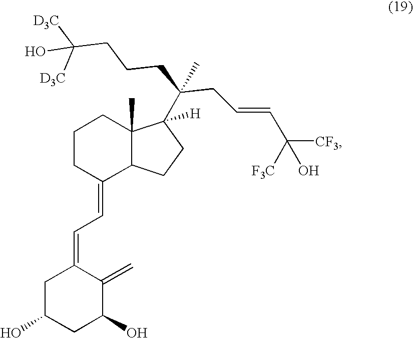
(20S)-1,25-Dihydroxy-20-((2E)-5,5,5-trifluoro-4-hydroxy-4-trifluoromethyl-pent-2-enyl)-26,27-hexadeutero-19-nor-cholecalciferol (20): 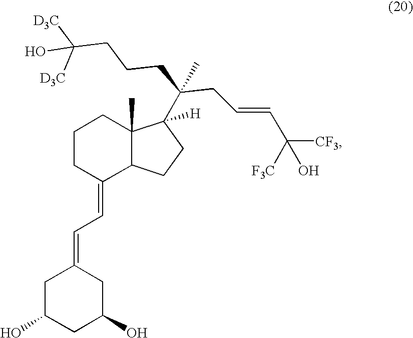
(20S)-1α-Fluoro-25-hydroxy-20-((2E)-5,5,5-trifluoro-4-hydroxy-4-trifluoromethyl-pent-2-enyl)-26,27-hexadeutero-cholecalciferol (21): 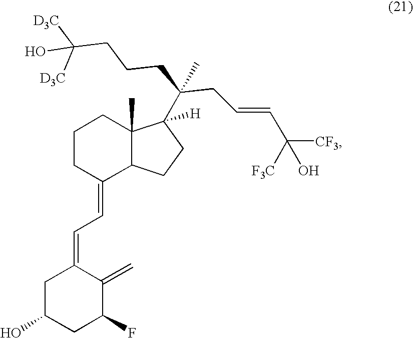
(20S)-1,25-Dihydroxy-20-(5,5,5-trifluoro-4-hydroxy-4-trifluoromethyl-pent-2-ynyl)-26,27-hexadeutero-cholecalciferol (22): 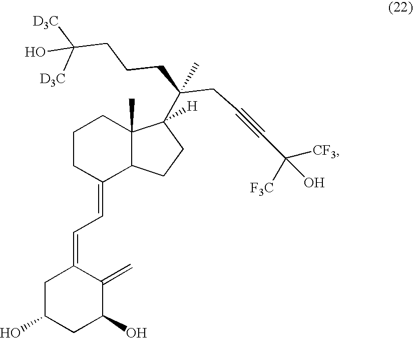
(20S)-1,25-Dihydroxy-20-(5,5,5-trifluoro-4-hydroxy-4-trifluoromethyl-pent-2-ynyl)-26,27-hexadeutero-19-nor-cholecalciferol (23): 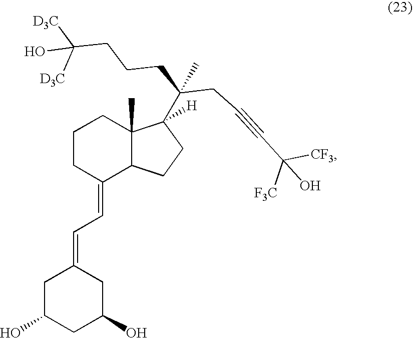
(20S)-1α-Fluoro-25-hydroxy-20-(5,5,5-trifluoro-4-hydroxy-4-trifluoromethyl-pent-2-ynyl)-26,27-hexadeutero-cholecalciferol (24): 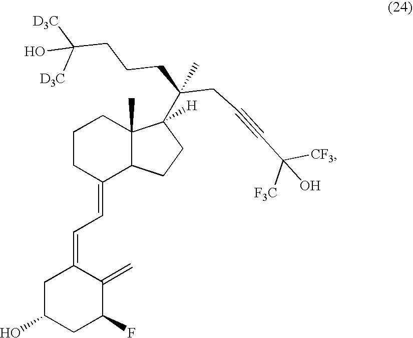
1,25-Dihydroxy-20R-20-(4-hydroxy-5,5,5-trideutero-4-trideuteromethyl-pentyl)-23Z-ene-26,27-hexafluorocholecalciferol (25): 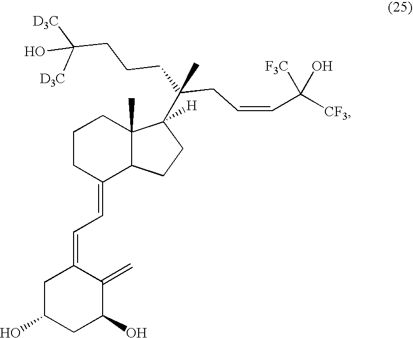
1,25-Dihydroxy-20R-20-(4-hydroxy-5,5,5-trideutero-4-trideuteromethyl-pentyl)-23Z-ene-26,27-hexafluoro-19-nor-cholecalciferol (26): 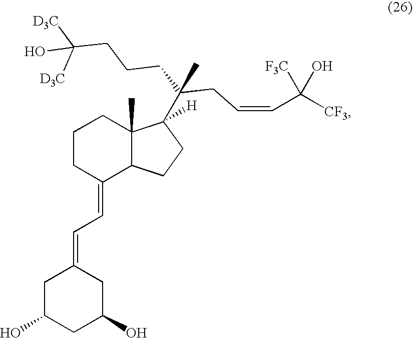
1α-Fluoro-25-hydroxy-20R-20-(4-hydroxy-5,5,5-trideutero-4-trideuteromethyl-pentyl)-23Z-ene-26,27-hexafluorocholecalciferol (27): 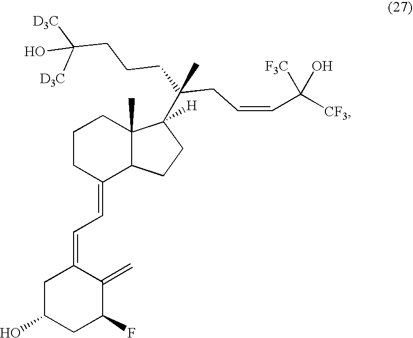
1,25-Dihydroxy-20R-20-(4-hydroxy-5,5,5-trideutero-4-trideuteromethyl-pentyl)-23E-ene-26,27-hexafluorocholecalciferol (28): 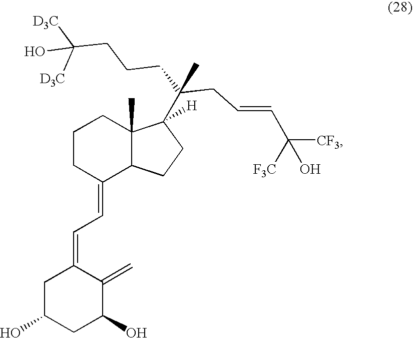
1,25-Dihydroxy-20R-20-(4-hydroxy-5,5,5-trideutero-4-trideuteromethyl-pentyl)-23E-ene-26,27-hexafluoro-19-nor-cholecalciferol (29): 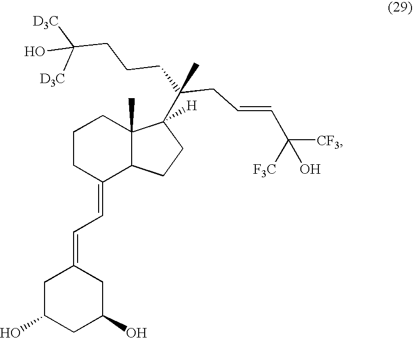
1α-Fluoro-25-hydroxy-20R-20-(4-hydroxy-5,5,5-trideutero-4-trideuteromethyl-pentyl)-23E-ene-26,27-hexafluorocholecalciferol (30): 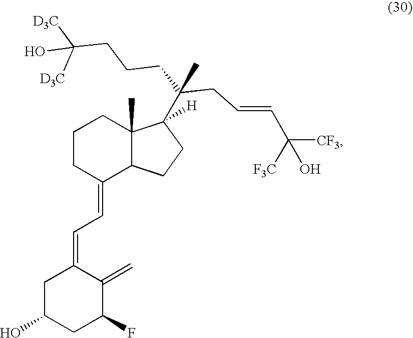
1,25-Dihydroxy-20R-20-(4-hydroxy-5,5,5-trideutero-4-trideuteromethyl-pentyl)-23-yne-26,27-hexafluorocholecalciferol (31): 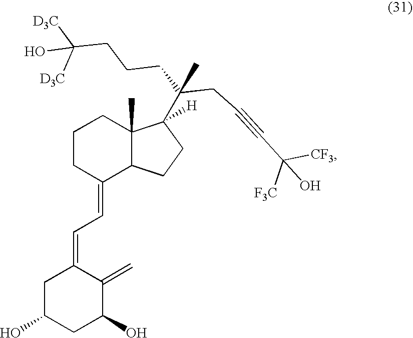
1,25-Dihydroxy-20R-20-(4-hydroxy-5,5,5-trideutero-4-trideuteromethyl-pentyl)-23-yne-26,27-hexafluoro-19-nor-cholecalciferol (32): 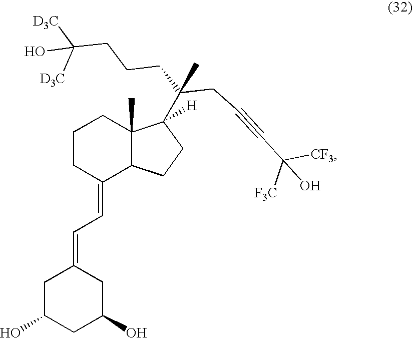
1α-Fluoro-25-hydroxy-20R-20-(4-hydroxy-5,5,5-trideutero-4-trideuteromethyl-pentyl)-23-yne-26,27-hexafluorocholecalciferol (33):

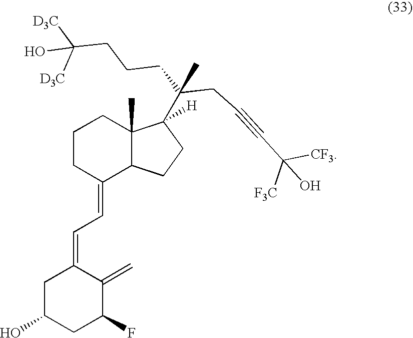

73-108. (canceled)

109. A method for treating a subject for a disorder characterized by an aberrant activity of vitamin D₃ responsive cell, comprising administering to a subject in need thereof a therapeutically effective amount of a vitamin D₃ compound having formula I: 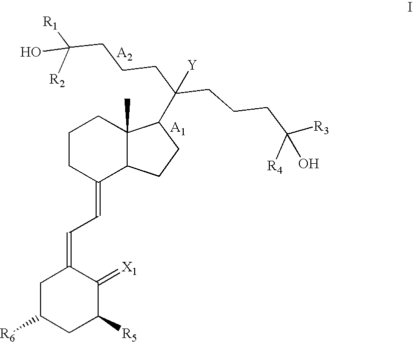
wherein:
A₁ is a single or double bond;
A₂ is a single, a double or a triple bond;
R₁, R₂, R₃ and R₄ are each independently alkyl, deuteroalkyl, hydroxyalkyl, or haloalkyl;
R₅ is halogen, hydroxyl, OC(O)alkyl, OC(O)hydroxyalkyl, or OC(O)haloalkyl;
R₆ is halogen, hydroxyl. OC(O)alkyl, OC(O)hydroxyalkyl, or OC(O)haloalkyl;
X₁ is H₂ or CH₂;
Y is alkyl; and pharmaceutically acceptable esters, salts, and prodrugs thereof; such that said subject is treated for said disorder.

110-136. (canceled)

137. The method for of claim 109, wherein the disorder is bladder dysfunction.

138-142. (canceled)

143. The method claim 109, wherein the disorder is a urogenital disorder.

144-174. (canceled)

175. A pharmaceutical composition comprising a therapeutically effective amount of a vitamin D₃ compound having formula I: 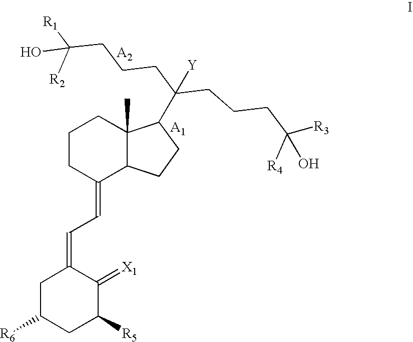
wherein:
A₁ is a single or double bond;
A₂ is a single, a double or a triple bond;
R₁, R₂, R₃, R₄ are each independently alkyl, deuteroalkyl, hydroxyalkyl, or haloalkyl;
R₅ is halogen, hydroxyl, OC(O)alkyl, OC(O)hydroxyalkyl, or OC(O)haloalkyl;
R₆ is halogen, hydroxyl, OC(O)alkyl, OC(O)hydroxyalkyl, or OC(O)haloalkyl;
X₂ is H₂ or CH₂;
Y is alkyl; and pharmaceutically acceptable esters, salts, and prodrugs thereof;
and a pharmaceutically acceptable diluent or carrier.

176-179. (canceled)

180. A packaged formulation for use in the treatment of a vitamin D₃ associated state, comprising a pharmaceutical composition comprising a vitamin D₃ compound having formula I: 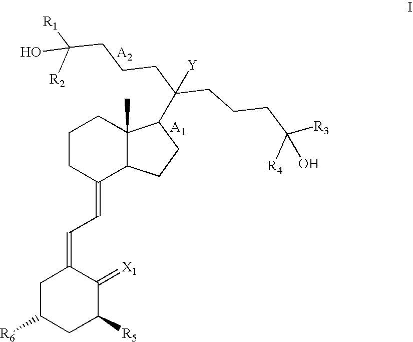
wherein:
A₁ is a single or double bond;
A₂ is a single, a double or a triple bond;
R₁, R₂, R₃, and R₄ are each independently alkyl, deuteroalkyl, hydroxyalkyl, or haloalkyl;
R₅ is halogen, hydroxyl, OC(O)alkyl, OC(O)hydroxyalkyl, or OC(O)haloalkyl;
R₆ is halogen, hydroxyl, OC(O)alkyl, OC(O)hydroxyalkyl, or OC(O)haloalkyl;
X₁ is H₂ or CH₂;
Y is alkyl; and pharmaceutically acceptable esters, salts, and prodrugs thereof;
and instructions for use in the treatment of a vitamin D₃ associated state.
